SEQUENCE LISTING (426 sequences)

SEQ ID NO: 1 - protein, 36 aa, Unknown
  Description of Unknown Scorpion or spider venom or toxin polypeptide
GSMCIPCFTTNPNMAAKCNACCGSRRGSCRGPQCIC

SEQ ID NO: 2 - protein, 37 aa, Unknown
  Description of Unknown Scorpion or spider venom or toxin polypeptide
GSGCLEFWWKCNPNDDKCCRPKLKCSKLFKLCNFSFG

SEQ ID NO: 3 - protein, 38 aa, Unknown
  Description of Unknown Scorpion or spider venom or toxin polypeptide
GSECRYWLGTCSKTGDCCSHLSCSPKHGWCVWDWTFRK

SEQ ID NO: 4 - protein, 38 aa, Unknown
  Description of Unknown Scorpion or spider venom or toxin polypeptide
GSMCMPCFTTDHQMARRCDDCCGGRGRGRCYGPQCLCR

SEQ ID NO: 5 - protein, 38 aa, Unknown
  Description of Unknown Scorpion or spider venom or toxin polypeptide
GSMCMPCFTTHHRMAENCDICCGGDGRGKCYGPQCLCR

SEQ ID NO: 6 - protein, 38 aa, Unknown
  Description of Unknown Scorpion or spider venom or toxin polypeptide
GSMCMPCFTTDHRMAENCDICCGGDGRGKCYGPQCLCR

SEQ ID NO: 7 - protein, 38 aa, Unknown
  Description of Unknown Scorpion or spider venom or toxin polypeptide
GSMCMPCFTTHHQMAENCDICCGGDGRGKCYGPQCLCR

SEQ ID NO: 8 - protein, 38 aa, Unknown
  Description of Unknown Scorpion or spider venom or toxin polypeptide
GSMCMPCFTTHHRMARNCDICCGGDGRGKCYGPQCLCR

SEQ ID NO: 9 - protein, 38 aa, Unknown
  Description of Unknown Scorpion or spider venom or toxin polypeptide
GSMCMPCFTTHHRMAERCDICCGGDGRGKCYGPQCLCR

SEQ ID NO: 10 - protein, 38 aa, Unknown
  Description of Unknown Scorpion or spider venom or toxin polypeptide
GSMCMPCFTTHHRMAENCDDCCGGDGRGKCYGPQCLCR

SEQ ID NO: 11 - protein, 38 aa, Unknown
  Description of Unknown Scorpion or spider venom or toxin polypeptide
GSRCMPCFTTDHQMARRCDDCCGGRGRGKCYGPQCLCR

SEQ ID NO: 12 - protein, 38 aa, Unknown
  Description of Unknown Scorpion or spider venom or toxin polypeptide
GSICIPCFTTDHQIARRCDDCCGGRGRGKCYGPQCLCR

SEQ ID NO: 13 - protein, 38 aa, Unknown
  Description of Unknown Scorpion or spider venom or toxin polypeptide
GSMCLPCFTTDHQLARRCDDCCGGRGRGKCYGPQCLCR

SEQ ID NO: 14 - protein, 38 aa, Unknown
  Description of Unknown Scorpion or spider venom or toxin polypeptide
GSMCMPCFTTEHQMARRCEECCGGRGRGKCYGPQCLCR

SEQ ID NO: 15 - protein, 38 aa, Unknown
  Description of Unknown Scorpion or spider venom or toxin polypeptide
GSMCIPCFTTDHQMARRCEECCGGRGRGKCYGPQCLCR

SEQ ID NO: 16 - protein, 38 aa, Unknown
  Description of Unknown Scorpion or spider venom or toxin polypeptide
GSICIPCFTTDHQMARRCDDCCGGRGDGKCYGPQCLCR

SEQ ID NO: 17 - protein, 38 aa, Unknown
  Description of Unknown Scorpion or spider venom or toxin polypeptide
GSICIPCFTTDHQIARRCDDCCGGRGRGKCYGPQCICR

SEQ ID NO: 18 - protein, 38 aa, Unknown
  Description of Unknown Scorpion or spider venom or toxin polypeptide
GSRCMPCFTTDHFMARFCDFCCGGRGRGKCYGPQCLCR

SEQ ID NO: 19 - protein, 38 aa, Unknown
  Description of Unknown Scorpion or spider venom or toxin polypeptide
GSRCMPCFTTDHYMARYCDYCCGGRGRGKCYGPQCLCR

SEQ ID NO: 20 - protein, 38 aa, Unknown
  Description of Unknown Scorpion or spider venom or toxin polypeptide
GSRCMPCFTTDHRMARRCDRCCGGRGRGKCYGPQCLCR

SEQ ID NO: 21 - protein, 38 aa, Unknown
  Description of Unknown Scorpion or spider venom or toxin polypeptide
GSRCMPCFTTDHEMARECDECCGGRGRGKCYGPQCLCR

SEQ ID NO: 22 - protein, 38 aa, Unknown
  Description of Unknown Scorpion or spider venom or toxin polypeptide
GSRCMPCFTTDHHMARHCDHCCGGRGRGKCYGPQCLCR

SEQ ID NO: 23 - protein, 38 aa, Unknown
  Description of Unknown Scorpion or spider venom or toxin polypeptide
GSLCLPCFTTHHRLADQCDICCGGDGRGKCYGPQCLCR

SEQ ID NO: 24 - protein, 38 aa, Unknown
  Description of Unknown Scorpion or spider venom or toxin polypeptide
GSICIPCFTTEHQIARRCEECCGGRGRGKCYGPQCLCR

SEQ ID NO: 25 - protein, 38 aa, Unknown
  Description of Unknown Scorpion or spider venom or toxin polypeptide
GSMCMPCFTTDTQMQERCDRCCGGGGRGKCWGPQCLCI

SEQ ID NO: 26 - protein, 38 aa, Unknown
  Description of Unknown Scorpion or spider venom or toxin polypeptide
GSMCMPCFTTIYRMAHECDECCGGRGRGKCYGPQCLCR

SEQ ID NO: 27 - protein, 38 aa, Unknown
  Description of Unknown Scorpion or spider venom or toxin polypeptide
GSMCMPCFTTGYRMAEYCDICCGGRGRGKCYGPQCLCR

SEQ ID NO: 28 - protein, 38 aa, Unknown
  Description of Unknown Scorpion or spider venom or toxin polypeptide
GSMCMPCFTTHRRMANTCDACCGGRSRGKCYGPQCLCR

SEQ ID NO: 29 - protein, 38 aa, Unknown
  Description of Unknown Scorpion or spider venom or toxin polypeptide
GSHCMPCFTTDHQMIRRCDDCCGGGSYGKCDGPQCLCF

SEQ ID NO: 30 - protein, 38 aa, Unknown
  Description of Unknown Scorpion or spider venom or toxin polypeptide
GSDCMPCFTTDHRMADHCDICCGGDDRGKCYGPQCLCR

SEQ ID NO: 31 - protein, 38 aa, Unknown
  Description of Unknown Scorpion or spider venom or toxin polypeptide
GSMCMPCFTTDHEMERRCDDCCGIGGGGKCHGPQCLCG

SEQ ID NO: 32 - protein, 38 aa, Unknown
  Description of Unknown Scorpion or spider venom or toxin polypeptide
GSMCMPCFTTEQRMAIICDDCCGGFGRGKCYGPQCLCR

SEQ ID NO: 33 - protein, 38 aa, Unknown
  Description of Unknown Scorpion or spider venom or toxin polypeptide
GSMCMPCFTTSEQMFRRCDDCCGGWGDGKCNGPHCLCR

SEQ ID NO: 34 - protein, 39 aa, Unknown
  Description of Unknown Scorpion or spider venom or toxin polypeptide
GSGVPINVKCRGSRDCLDPCKKAGMRFGKCINSKCHCTP

SEQ ID NO: 35 - protein, 38 aa, Unknown
  Description of Unknown Scorpion or spider venom or toxin polypeptide
GSMCMPCFTTEQRMAIICDDCCGGFGRGRCYGPQCLCR

SEQ ID NO: 36 - protein, 38 aa, Unknown
  Description of Unknown Scorpion or spider venom or toxin polypeptide
GSICIPCFTTDHQIARRCDDCCGGRGRGRCYGPQCICR

SEQ ID NO: 37 - protein, 38 aa, Unknown
  Description of Unknown Scorpion or spider venom or toxin polypeptide
GSMCMPCFTTDTQMQERCDRCCGGGGRGRCWGPQCLCI

SEQ ID NO: 38 - protein, 37 aa, Unknown
  Description of Unknown Scorpion or spider venom or toxin polypeptide
GSMCMPCFTTDTQMQERCDRCCGGGGRGRCWGPQCLC

SEQ ID NO: 39 - protein, 38 aa, Unknown
  Description of Unknown Scorpion or spider venom or toxin polypeptide
GSMCMPCFTTDHRMAENCDICCGGDGRGRCYGPQCLCR

SEQ ID NO: 40 - protein, 38 aa, Unknown
  Description of Unknown Scorpion or spider venom or toxin polypeptide
GSMCMPCFTTEQRMAIICDDCCGGFGRGKCYGPQCLCI

SEQ ID NO: 41 - protein, 38 aa, Unknown
  Description of Unknown Scorpion or spider venom or toxin polypeptide
GSMCMPCFTTEQRMAIICDDCCGGFGRGRCYGPQCLCI

SEQ ID NO: 42 - protein, 38 aa, Unknown
  Description of Unknown Scorpion or spider venom or toxin polypeptide
GSICIPCFTTDHQIARRCDDCCGGRGRGKCYGPQCICI

SEQ ID NO: 43 - protein, 38 aa, Unknown
  Description of Unknown Scorpion or spider venom or toxin polypeptide
GSICIPCFTTDHQIARRCDDCCGGRGRGRCYGPQCICI

SEQ ID NO: 44 - protein, 38 aa, Unknown
  Description of Unknown Scorpion or spider venom or toxin polypeptide
GSMCMPCFTTDTQMQEKCDRCCGGGGRGRCWGPQCLCI

SEQ ID NO: 45 - protein, 38 aa, Unknown
  Description of Unknown Scorpion or spider venom or toxin polypeptide
GSMCMPCFTTEQRMAIKCDDCCGGFGRGRCYGPQCLCR

SEQ ID NO: 46 - protein, 38 aa, Unknown
  Description of Unknown Scorpion or spider venom or toxin polypeptide
GSICIPCFTTDHQIARKCDDCCGGRGRGRCYGPQCICR

SEQ ID NO: 47 - protein, 38 aa, Unknown
  Description of Unknown Scorpion or spider venom or toxin polypeptide
GSMCMPCFTTDHRMAEKCDICCGGDGRGRCYGPQCLCR

SEQ ID NO: 48 - protein, 38 aa, Unknown
  Description of Unknown Scorpion or spider venom or toxin polypeptide
GSMCMPCFTTDTQMQERCDRCCGGKGRGRCWGPQCLCI

SEQ ID NO: 49 - protein, 38 aa, Unknown
  Description of Unknown Scorpion or spider venom or toxin polypeptide
GSMCMPCFTTEQRMAIICDDCCGGKGRGRCYGPQCLCR

SEQ ID NO: 50 - protein, 38 aa, Unknown
  Description of Unknown Scorpion or spider venom or toxin polypeptide
GSICIPCFTTDHQIARRCDDCCGGKGRGRCYGPQCICR

SEQ ID NO: 51 - protein, 38 aa, Unknown
  Description of Unknown Scorpion or spider venom or toxin polypeptide
GSMCMPCFTTDHRMAENCDICCGGKGRGRCYGPQCLCR

SEQ ID NO: 52 - protein, 38 aa, Unknown
  Description of Unknown Scorpion or spider venom or toxin polypeptide
GSMCMPCFTTDHRMAENCDICCGGDGRGKCYGPQCLCI

SEQ ID NO: 53 - protein, 38 aa, Unknown
  Description of Unknown Scorpion or spider venom or toxin polypeptide
GSMCMPCFTTDHRMAENCDICCGGDGRGRCYGPQCLCI

SEQ ID NO: 54 - protein, 38 aa, Unknown
  Description of Unknown Scorpion or spider venom or toxin polypeptide
GSMCMPCFTTHHRMAENCDICCGGDGRGRCYGPQCLCR

SEQ ID NO: 55 - protein, 30 aa, Unknown
  Description of Unknown Scorpion or spider venom or toxin polypeptide
GSVGCEECPMHCKGKNANPTCDDGVCNCNV

SEQ ID NO: 56 - protein, 30 aa, Unknown
  Description of Unknown Scorpion or spider venom or toxin polypeptide
GSVGCEECPMHCKGKNAKPTCDDGVCNCNV

SEQ ID NO: 57 - protein, 30 aa, Unknown
  Description of Unknown Scorpion or spider venom or toxin polypeptide
GSVGCEECPMHCKGKNAKPTCDNGVCNCNV

SEQ ID NO: 58 - protein, 30 aa, Unknown
  Description of Unknown Scorpion or spider venom or toxin polypeptide
GSVGCEECPMHCKGKHAVPTCDDGVCNCNV

SEQ ID NO: 59 - protein, 30 aa, Unknown
  Description of Unknown Scorpion or spider venom or toxin polypeptide
GSVGCEECPAHCKGKNAKPTCDDGVCNCNV

SEQ ID NO: 60 - protein, 30 aa, Unknown
  Description of Unknown Scorpion or spider venom or toxin polypeptide
GSVGCEECPAHCKGKNAIPTCDDGVCNCNV

SEQ ID NO: 61 - protein, 30 aa, Unknown
  Description of Unknown Scorpion or spider venom or toxin polypeptide
GSVGCEECPMHCKGKMAKPTCDDGVCNCNV

SEQ ID NO: 62 - protein, 30 aa, Unknown
  Description of Unknown Scorpion or spider venom or toxin polypeptide
GSVGCEECPMHCKGKNAVPTCDNGVCNCNA

SEQ ID NO: 63 - protein, 30 aa, Unknown
  Description of Unknown Scorpion or spider venom or toxin polypeptide
GSVGCEECPMHCKGKMAKPTCYDGVCNCNV

SEQ ID NO: 64 - protein, 30 aa, Unknown
  Description of Unknown Scorpion or spider venom or toxin polypeptide
GSVGCEECPMYCKGKNAVPTCDGGVCNCNA

SEQ ID NO: 65 - protein, 30 aa, Unknown
  Description of Unknown Scorpion or spider venom or toxin polypeptide
GSVGCEECPKYCKGKNAVPTCDGGVCNCNA

SEQ ID NO: 66 - protein, 30 aa, Unknown
  Description of Unknown Scorpion or spider venom or toxin polypeptide
GSVGCEECPVYCKGKKALPTCDGGVCNCNA

SEQ ID NO: 67 - protein, 30 aa, Unknown
  Description of Unknown Scorpion or spider venom or toxin polypeptide
GSVGCEEDPMHCKGKQAKPTCCNGVCNCNV

SEQ ID NO: 68 - protein, 34 aa, Unknown
  Description of Unknown Scorpion or spider venom or toxin polypeptide
GSVGCAECPMHCKGKMAKPTCENEVCKCNIGKKD

SEQ ID NO: 69 - protein, 29 aa, Unknown
  Description of Unknown Scorpion or spider venom or toxin peptide
GSVGCEECPMHCKGKKALPTCDYGCECND

SEQ ID NO: 70 - protein, 33 aa, Unknown
  Description of Unknown Scorpion or spider venom or toxin polypeptide
GSIVCKVCKIICGMQGKKVNICKAPIKCKCKKG

SEQ ID NO: 71 - protein, 31 aa, Unknown
  Description of Unknown Scorpion or spider venom or toxin polypeptide
GSVSCEDCPDHCSTQKARAKCDNDKCVCEPK

SEQ ID NO: 72 - protein, 31 aa, Unknown
  Description of Unknown Scorpion or spider venom or toxin polypeptide
GSVSCEDCPEHCSTQKARAKCDNDKCVCESV

SEQ ID NO: 73 - protein, 31 aa, Unknown
  Description of Unknown Scorpion or spider venom or toxin polypeptide
GSVSCEDCPEHCSTQKAQAKCDNDKCVCEPI

SEQ ID NO: 74 - protein, 31 aa, Unknown
  Description of Unknown Scorpion or spider venom or toxin polypeptide
GSATCEDCPEHCATQNARAKCDNDKCVCEPK

SEQ ID NO: 75 - protein, 31 aa, Unknown
  Description of Unknown Scorpion or spider venom or toxin polypeptide
GSVSCEDCPEHCATKDQRAKCDNDKCVCEPK

SEQ ID NO: 76 - protein, 31 aa, Unknown
  Description of Unknown Scorpion or spider venom or toxin polypeptide
GSVGCEDCPEHCSQQNARAKCENDKCVCEPK

SEQ ID NO: 77 - protein, 31 aa, Unknown
  Description of Unknown Scorpion or spider venom or toxin polypeptide
GSVSCEDCPEHCATKDQRAKCDNDRCVCEPK

SEQ ID NO: 78 - protein, 31 aa, Unknown
  Description of Unknown Scorpion or spider venom or toxin polypeptide
GSVSCEDCPPHCATKDQRAKCENDKCVCEPK

SEQ ID NO: 79 - protein, 31 aa, Unknown
  Description of Unknown Scorpion or spider venom or toxin polypeptide
GSVSCEDCPEHCSTQKARAKCDNDKCVCEAI

SEQ ID NO: 80 - protein, 37 aa, Unknown
  Description of Unknown Scorpion or spider venom or toxin polypeptide
GSMCMPCFTTEQRMAIICDDCCGGFGRGRCYGPQCLC

SEQ ID NO: 81 - protein, 37 aa, Unknown
  Description of Unknown Scorpion or spider venom or toxin polypeptide
GSICIPCFTTDHQIARRCDDCCGGRGRGRCYGPQCIC

SEQ ID NO: 82 - protein, 37 aa, Unknown
  Description of Unknown Scorpion or spider venom or toxin polypeptide
GSMCMPCFTTDHRMAENCDICCGGDGRGRCYGPQCLC

SEQ ID NO: 83 - protein, 30 aa, Unknown
  Description of Unknown Scorpion or spider venom or toxin polypeptide
GSVGCEECPMHCRGRNANPTCDDGVCNCNV

SEQ ID NO: 84 - protein, 28 aa, Unknown
  Description of Unknown Scorpion or spider venom or toxin peptide
GSVGCEECPMHCRGRNANPTCDDGVCNC

SEQ ID NO: 85 - protein, 36 aa, Unknown
  Description of Unknown Scorpion or spider venom or toxin polypeptide
GSCGPCFTTDHQMEQKCAECCGGIGKCYGPQCLCNR

SEQ ID NO: 86 - protein, 40 aa, Unknown
  Description of Unknown Scorpion or spider venom or toxin polypeptide
GSRCGPCFTTDPQTQAKCSECCGRKGGVCKGPQCICGIQY

SEQ ID NO: 87 - protein, 37 aa, Unknown
  Description of Unknown Scorpion or spider venom or toxin polypeptide
GSMCMPCFTTDPNMAKKCRDCCGGNGKCFGPQCLCNR

SEQ ID NO: 88 - protein, 36 aa, Unknown
  Description of Unknown Scorpion or spider venom or toxin polypeptide
GSMCMPCFTTDHNMAKKCNDCCGGYGKCFGPQCLCR

SEQ ID NO: 89 - protein, 38 aa, Unknown
  Description of Unknown Scorpion or spider venom or toxin polypeptide
GSRCPPCFTTNPNMEADCRKCCGGRGYCASYQCICPGG

SEQ ID NO: 90 - protein, 37 aa, Unknown
  Description of Unknown Scorpion or spider venom or toxin polypeptide
GSMCMPCFTTDPNMANKCRDCCGGGKKCFGPQCLCNR

SEQ ID NO: 91 - protein, 64 aa, Unknown
  Description of Unknown Scorpion or spider venom or toxin polypeptide
GSMKFLYGVILIALFLTVMTATLSEARCGPCFTTDPQTQAKCSECCGRKGGVCKGPQCICGIQY

SEQ ID NO: 92 - protein, 38 aa, Unknown
  Description of Unknown Scorpion or spider venom or toxin polypeptide
GSMCMPCFTTRPDMAQQCRACCKGRGKCFGPQCLCGYD

SEQ ID NO: 93 - protein, 62 aa, Unknown
  Description of Unknown Scorpion or spider venom or toxin polypeptide
GSMKFLYGIVFIALFLTVMTATLSDAMCMPCFTTDHNMAKKCRDCCGGNGKCFGPQCLCNRG

SEQ ID NO: 94 - protein, 37 aa, Unknown
  Description of Unknown Scorpion or spider venom or toxin polypeptide
GSMCMPCFTTDHNMAKKCRDCCGGNGKCFGPQCLCNR

SEQ ID NO: 95 - protein, 62 aa, Unknown
  Description of Unknown Scorpion or spider venom or toxin polypeptide
GSMKFLYGIVFITLFLTVMIATHTEAMCMPCFTTRPNMAQQCRDCCRGRGKCFGPQCLCGYD

SEQ ID NO: 96 - protein, 62 aa, Unknown
  Description of Unknown Scorpion or spider venom or toxin polypeptide
GSMKFLYGIVFIALFLTVMIATHTEAMCMPCFTTRPNMAQQCRDCCRGRGKCFGPQCLCGYD

SEQ ID NO: 97 - protein, 37 aa, Unknown
  Description of Unknown Scorpion or spider venom or toxin polypeptide
GSRCKPCFTTDPQMSKKCADCCGGKGKGKCYGPQCLC

SEQ ID NO: 98 - protein, 63 aa, Unknown
  Description of Unknown Scorpion or spider venom or toxin polypeptide
GSMKFLYGIVFITLFLTVMIATHTEAAMCMPCFTTNLNMEQECRDCCGGTGRCFGPQCLCGYD

SEQ ID NO: 99 - protein, 40 aa, Unknown
  Description of Unknown Scorpion or spider venom or toxin polypeptide
GSRCSPCFTTDQQMTKKCYDCCGGKGKGKCYGPQCICAPY

SEQ ID NO: 100 - protein, 37 aa, Unknown
  Description of Unknown Scorpion or spider venom or toxin polypeptide
GSCGPCFTTDPYTESKCATCCGGRGKCVGPQCLCNRI

SEQ ID NO: 101 - protein, 41 aa, Unknown
  Description of Unknown Scorpion or spider venom or toxin polypeptide
GSTEAMCMPCFTTDHNMAKKCRDCCGGNGKCFGYQCLCNRG

SEQ ID NO: 102 - protein, 61 aa, Unknown
  Description of Unknown Scorpion or spider venom or toxin polypeptide
GSMKFLYGIVFIALFLTVMFATQTDGCGPCFTTDANMARKCRECCGGIGKCFGPQCLCNRI

SEQ ID NO: 103 - protein, 61 aa, Unknown
  Description of Unknown Scorpion or spider venom or toxin polypeptide
GSMKFLYGIVFIALFLTVMFATQTDGCGPCFTTDANMARKCRECCGGNGKCFGPQCLCNRE

SEQ ID NO: 104 - protein, 62 aa, Unknown
  Description of Unknown Scorpion or spider venom or toxin polypeptide
GSMKFLYGTILIAFFLTVMIATHSEARCPPCFTTNPNMEADCRKCCGGRGYCASYQCICPGG

SEQ ID NO: 105 - protein, 41 aa, Unknown
  Description of Unknown Scorpion or spider venom or toxin polypeptide
GSTEAMCMPCFTTRPDMAQQCRDCCGGNGKCFGYQCLCNRG

SEQ ID NO: 106 - protein, 62 aa, Unknown
  Description of Unknown Scorpion or spider venom or toxin polypeptide
GSMKFLYGIVFIALFLTVMIATLTEAMCMPCFTTRPDMAQQCRDCCGGNGKCFGYQCLCNRG

SEQ ID NO: 107 - protein, 62 aa, Unknown
  Description of Unknown Scorpion or spider venom or toxin polypeptide
GSMKFLYGIVFIALFLTVMIATHTEAMCMPCFTTRPDMAQQCRDCCGGNGKCFGYQCLCNRG

SEQ ID NO: 108 - protein, 63 aa, Unknown
  Description of Unknown Scorpion or spider venom or toxin polypeptide
GSMKFLYGIILIALFLTVMIATHSEARCPNCFTTNPNAEADCKKCCGNRWGKCAGYQCVCPMK

SEQ ID NO: 109 - protein, 69 aa, Unknown
  Description of Unknown Scorpion or spider venom or toxin polypeptide
GSMKFLYGIVFIALFLTGMIATHTEAMCMPCFTTRPDMAQQCRDCCGGNGKCFGYQCLCNRGRIVIMYT

SEQ ID NO: 110 - protein, 37 aa, Unknown
  Description of Unknown Scorpion or spider venom or toxin polypeptide
GSMCMPCFTTRPGMAQQCRDCCGGNGKCFGYQCLCNR

SEQ ID NO: 111 - protein, 37 aa, Unknown
  Description of Unknown Scorpion or spider venom or toxin polypeptide
GSMCIPCFTTNPNMAAKCNACCGSRRGSCRGPQCICR

SEQ ID NO: 112 - protein, 37 aa, Unknown
  Description of Unknown Scorpion or spider venom or toxin polypeptide
GSMCIPCFTTNPNMAAKCNACCGSRRGSCRGPQCICN

SEQ ID NO: 113 - protein, 36 aa, Unknown
  Description of Unknown Scorpion or spider venom or toxin polypeptide
GSMCIPCFTTNPNMAAKCNACCGGNGSCRGPQCICN

SEQ ID NO: 114 - protein, 38 aa, Unknown
  Description of Unknown Scorpion or spider venom or toxin polypeptide
GSMCIPCFTTNPNMAAKCNACCGSRGRGSCRGPQCICN

SEQ ID NO: 115 - protein, 38 aa, Unknown
  Description of Unknown Scorpion or spider venom or toxin polypeptide
GSMCIPCFTTNPNMAAKCNACCGSRGRGKCRGPQCICN

SEQ ID NO: 116 - protein, 37 aa, Unknown
  Description of Unknown Scorpion or spider venom or toxin polypeptide
GSMCIPCFTTDHQMAAKCNACCGSRRGSCRGPQCICN

SEQ ID NO: 117 - protein, 37 aa, Unknown
  Description of Unknown Scorpion or spider venom or toxin polypeptide
GSMCIPCFTTNHQMAAKCNACCGSRRGSCRGPQCICN

SEQ ID NO: 118 - protein, 38 aa, Unknown
  Description of Unknown Scorpion or spider venom or toxin polypeptide
GSMCIPCFTTNPNMARKCNACCGSRGRGSCRGPQCICN

SEQ ID NO: 119 - protein, 38 aa, Unknown
  Description of Unknown Scorpion or spider venom or toxin polypeptide
GSMCIPCFTTNPNMAAKCNACCGGKGRGSCRGPQCICN

SEQ ID NO: 120 - protein, 37 aa, Unknown
  Description of Unknown Scorpion or spider venom or toxin polypeptide
GSMCIPCFTTNPNMAAKCNACCGSRRGSCFGPQCICN

SEQ ID NO: 121 - protein, 38 aa, Unknown
  Description of Unknown Scorpion or spider venom or toxin polypeptide
GSMCIPCFTTNPNMAAKCNACCGSRGRGKCFGPQCICN

SEQ ID NO: 122 - protein, 38 aa, Unknown
  Description of Unknown Scorpion or spider venom or toxin polypeptide
GSMCIPCFTTNPNMAAKCNACCGSRGRGSCFGPQCICN

SEQ ID NO: 123 - protein, 38 aa, Unknown
  Description of Unknown Scorpion or spider venom or toxin polypeptide
GSMCIPCFTTNPNMAAKCNACCGSRGRGSCYGPQCICN

SEQ ID NO: 124 - protein, 37 aa, Unknown
  Description of Unknown Scorpion or spider venom or toxin polypeptide
GSMCIPCFTTNPNMAAKCDACCGSRRGSCRGPQCICN

SEQ ID NO: 125 - protein, 37 aa, Unknown
  Description of Unknown Scorpion or spider venom or toxin polypeptide
GSMCIPCFTTNHQMAAKCDACCGSRRGSCRGPQCICN

SEQ ID NO: 126 - protein, 38 aa, Unknown
  Description of Unknown Scorpion or spider venom or toxin polypeptide
GSMCIPCFTTNHNMAAKCDACCGGRGRGSCRGPQCICN

SEQ ID NO: 127 - protein, 38 aa, Unknown
  Description of Unknown Scorpion or spider venom or toxin polypeptide
GSMCIPCFTTNPNMAAKCDACCGSRGRGSCRGPQCICN

SEQ ID NO: 128 - protein, 38 aa, Unknown
  Description of Unknown Scorpion or spider venom or toxin polypeptide
GSMCIPCFTTNPNMAAKCDACCGGKGRGSCRGPQCICN

SEQ ID NO: 129 - protein, 38 aa, Unknown
  Description of Unknown Scorpion or spider venom or toxin polypeptide
GSMCIPCFTTNHNMAAKCDACCGSRGRGSCRGPQCICN

SEQ ID NO: 130 - protein, 36 aa, Unknown
  Description of Unknown Scorpion or spider venom or toxin polypeptide
GSMCIPCFTTNPNMAAKCRDCCGGRGSCRGPQCICN

SEQ ID NO: 131 - protein, 38 aa, Unknown
  Description of Unknown Scorpion or spider venom or toxin polypeptide
GSMCMPCFTTNPNMAAKCDDCCGSRGRGSCRGPQCICN

SEQ ID NO: 132 - protein, 36 aa, Unknown
  Description of Unknown Scorpion or spider venom or toxin polypeptide
GSMCIPCFTTNPNMAARCNACCGSRRGSCRGPQCIC

SEQ ID NO: 133 - protein, 37 aa, Unknown
  Description of Unknown Scorpion or spider venom or toxin polypeptide
GSMCIPCFTTNPNMAAKCNACCGSRRGSCRGPQCICI

SEQ ID NO: 134 - protein, 38 aa, Unknown
  Description of Unknown Scorpion or spider venom or toxin polypeptide
GSGCLQFMWKCNPDNDKCCRPNLKCNTYHKWCEFVTGK

SEQ ID NO: 135 - protein, 36 aa, Unknown
  Description of Unknown Scorpion or spider venom or toxin polypeptide
GSDCLGFLWKCNPSNDKCCRPNLVCSRKDKWCKYQI

SEQ ID NO: 136 - protein, 38 aa, Unknown
  Description of Unknown Scorpion or spider venom or toxin polypeptide
GSDCLGFMRKCIPDNDKCCRPNLVCSRTHKWCKYVFGK

SEQ ID NO: 137 - protein, 37 aa, Unknown
  Description of Unknown Scorpion or spider venom or toxin polypeptide
GSECLEIFKACNPSNDQCCKSSKLVCSRKTRACKYQI

SEQ ID NO: 138 - protein, 36 aa, Unknown
  Description of Unknown Scorpion or spider venom or toxin polypeptide
GSECGGFWWKCGSGKPACCPKYVCSPKWGLCNFPMP

SEQ ID NO: 139 - protein, 37 aa, Unknown
  Description of Unknown Scorpion or spider venom or toxin polypeptide
GSGCLERWWKCNPNDDKCCRPKLKCSKLFKLCNRSRG

SEQ ID NO: 140 - protein, 37 aa, Unknown
  Description of Unknown Scorpion or spider venom or toxin polypeptide
GSGCLEEWWKCNPNDDKCCRPKLKCSKLFKLCNESEG

SEQ ID NO: 141 - protein, 37 aa, Unknown
  Description of Unknown Scorpion or spider venom or toxin polypeptide
GSGCLEIWWKCNPNDDKCCRPKLKCSKLFKLCNYSIG

SEQ ID NO: 142 - protein, 37 aa, Unknown
  Description of Unknown Scorpion or spider venom or toxin polypeptide
GSGCLEFWWKCNPNDDKCCRPKLKCSKLGKLCNFSFG

SEQ ID NO: 143 - protein, 37 aa, Unknown
  Description of Unknown Scorpion or spider venom or toxin polypeptide
GSGCLEFWWKCNPNDDKCCRPKLKCSPLGKLCNFSFG

SEQ ID NO: 144 - protein, 37 aa, Unknown
  Description of Unknown Scorpion or spider venom or toxin polypeptide
GSGCLEFWWKCNPNDDKCCRPKLKCSPNGKLCNFSFG

SEQ ID NO: 145 - protein, 37 aa, Unknown
  Description of Unknown Scorpion or spider venom or toxin polypeptide
GSGCLEFWWKCNPNDDKCCRPKLKCSRKTKLCNFSFG

SEQ ID NO: 146 - protein, 37 aa, Unknown
  Description of Unknown Scorpion or spider venom or toxin polypeptide
GSGCLEFWWKCNPNDDKCCRPKLKCGSNFKLCNFSFG

SEQ ID NO: 147 - protein, 37 aa, Unknown
  Description of Unknown Scorpion or spider venom or toxin polypeptide
GSGCLEFWWKCNPNDDKCCRPKLKCSTKHKLCNFSFG

SEQ ID NO: 148 - protein, 37 aa, Unknown
  Description of Unknown Scorpion or spider venom or toxin polypeptide
GSGCLEFWWKCNPNDDKCCRPKLKCSNDGKLCNFSFG

SEQ ID NO: 149 - protein, 37 aa, Unknown
  Description of Unknown Scorpion or spider venom or toxin polypeptide
GSGCLEFWWKCNPNDDKCCRPKLKCSKKTKLCNFSFG

SEQ ID NO: 150 - protein, 37 aa, Unknown
  Description of Unknown Scorpion or spider venom or toxin polypeptide
GSGCLEFWWKCNPNDDKCCRPKLKCHSNFKLCNFSFG

SEQ ID NO: 151 - protein, 37 aa, Unknown
  Description of Unknown Scorpion or spider venom or toxin polypeptide
GSGCLEFWWKCNPNDDKCCRPKLKCSKKFTACNFSFG

SEQ ID NO: 152 - protein, 37 aa, Unknown
  Description of Unknown Scorpion or spider venom or toxin polypeptide
GSGCLEIFKACNPNDDKCCRPKLKCSKLFKLCNFSFG

SEQ ID NO: 153 - protein, 37 aa, Unknown
  Description of Unknown Scorpion or spider venom or toxin polypeptide
GSGCLKFGWKCNPNDDKCCRPKLKCSKLFKLCNFSFG

SEQ ID NO: 154 - protein, 38 aa, Unknown
  Description of Unknown Scorpion or spider venom or toxin polypeptide
GSGCLEFWWKCNPNDDKCCKSSKLKCSKLFKLCNFSFG

SEQ ID NO: 155 - protein, 37 aa, Unknown
  Description of Unknown Scorpion or spider venom or toxin polypeptide
GSGCLEFWWKCNPNDDKCCRPKLKCNKLFKLCNISIG

SEQ ID NO: 156 - protein, 35 aa, Unknown
  Description of Unknown Scorpion or spider venom or toxin polypeptide
GSGCLEFWWKCNPNDDCCRKLKCSKLFKLCNFSFG

SEQ ID NO: 157 - protein, 37 aa, Unknown
  Description of Unknown Scorpion or spider venom or toxin polypeptide
GSGCLEFWWKCNPSNDQCCRPKLKCSKLFKLCNFSFG

SEQ ID NO: 158 - protein, 38 aa, Unknown
  Description of Unknown Scorpion or spider venom or toxin polypeptide
GSGCLEFWWKCNPNDDKCCRPSKLVCSKLFKLCNFSFG

SEQ ID NO: 159 - protein, 37 aa, Unknown
  Description of Unknown Scorpion or spider venom or toxin polypeptide
GSGCLEFLGECNPNDDKCCRPKLKCSKLFKLCNFSFG

SEQ ID NO: 160 - protein, 37 aa, Unknown
  Description of Unknown Scorpion or spider venom or toxin polypeptide
GSGCLWYLWKCNPNDDKCCRPKLKCSKLFKLCNFSFG

SEQ ID NO: 161 - protein, 37 aa, Unknown
  Description of Unknown Scorpion or spider venom or toxin polypeptide
GSGCLEFWWRCNPNDDRCCRPRLRCSRLFRLCNFSFG

SEQ ID NO: 162 - protein, 32 aa, Unknown
  Description of Unknown Scorpion or spider venom or toxin polypeptide
GSGCLEFWWRCNPNDDRCCRPRLRCSRLFRLC

SEQ ID NO: 163 - protein, 37 aa, Unknown
  Description of Unknown Scorpion or spider venom or toxin polypeptide
GSCRYFLGECKKTSECCEHLACHDKHKWCAWDWTIGK

SEQ ID NO: 164 - protein, 36 aa, Unknown
  Description of Unknown Scorpion or spider venom or toxin polypeptide
GSECRYWLGGCSAGQTCCKHLVCSRRHGWCVWDGTF

SEQ ID NO: 165 - protein, 38 aa, Unknown
  Description of Unknown Scorpion or spider venom or toxin polypeptide
GSECRWYLGECSQDGDCCKHLQCHSNYEWCIWDGTFSK

SEQ ID NO: 166 - protein, 38 aa, Unknown
  Description of Unknown Scorpion or spider venom or toxin polypeptide
GSECRWYLGGCSQDGDCCKHLQCHSNYEWCVWDGTFSK

SEQ ID NO: 167 - protein, 36 aa, Unknown
  Description of Unknown Scorpion or spider venom or toxin polypeptide
GSDCRKFLGACTQTSDCCKHLACHNKHKWCAWDWTI

SEQ ID NO: 168 - protein, 39 aa, Unknown
  Description of Unknown Scorpion or spider venom or toxin polypeptide
GSECRYLMGGCSKDGDCCEHLVCRTKWPYHCVWDWTFGK

SEQ ID NO: 169 - protein, 38 aa, Unknown
  Description of Unknown Scorpion or spider venom or toxin polypeptide
GSECRYRLGTCSKTGDCCSHLSCSPKHGWCVRDRTFRK

SEQ ID NO: 170 - protein, 38 aa, Unknown
  Description of Unknown Scorpion or spider venom or toxin polypeptide
GSECRYELGTCSKTGDCCSHLSCSPKHGWCVEDETFRK

SEQ ID NO: 171 - protein, 38 aa, Unknown
  Description of Unknown Scorpion or spider venom or toxin polypeptide
GSECRYILGTCSKTGDCCSHLSCSPKHGWCVYDITFRK

SEQ ID NO: 172 - protein, 38 aa, Unknown
  Description of Unknown Scorpion or spider venom or toxin polypeptide
GSECRYWLGTCSKTGDCCSHLSCSPKGGWCVWDWTFRK

SEQ ID NO: 173 - protein, 38 aa, Unknown
  Description of Unknown Scorpion or spider venom or toxin polypeptide
GSECRYWLGTCSKTGDCCSHLSCSPNHGWCVWDWTFRK

SEQ ID NO: 174 - protein, 38 aa, Unknown
  Description of Unknown Scorpion or spider venom or toxin polypeptide
GSECRYWLGTCSKTGDCCSHLSCSRKTGWCVWDWTFRK

SEQ ID NO: 175 - protein, 38 aa, Unknown
  Description of Unknown Scorpion or spider venom or toxin polypeptide
GSECRYWLGTCSKTGDCCSHLSCGSNHGWCVWDWTFRK

SEQ ID NO: 176 - protein, 38 aa, Unknown
  Description of Unknown Scorpion or spider venom or toxin polypeptide
GSECRYWLGTCSKTGDCCSHLSCSTKHGWCVWDWTFRK

SEQ ID NO: 177 - protein, 38 aa, Unknown
  Description of Unknown Scorpion or spider venom or toxin polypeptide
GSECRYWLGTCSKTGDCCSHLSCSSKHGWCVWDWTFRK

SEQ ID NO: 178 - protein, 38 aa, Unknown
  Description of Unknown Scorpion or spider venom or toxin polypeptide
GSECRYWLGTCSKTGDCCSHLSCSNDGGWCVWDWTFRK

SEQ ID NO: 179 - protein, 38 aa, Unknown
  Description of Unknown Scorpion or spider venom or toxin polypeptide
GSECRYWLGTCSKTGDCCSHLSCSPKTRACVWDWTFRK

SEQ ID NO: 180 - protein, 38 aa, Unknown
  Description of Unknown Scorpion or spider venom or toxin polypeptide
GSECRYWLGTCSKTGDCCSHLSCHSNHGWCVWDWTFRK

SEQ ID NO: 181 - protein, 38 aa, Unknown
  Description of Unknown Scorpion or spider venom or toxin polypeptide
GSECRYWLGTCSKTGDCCSHLSCSRKHRACVWDWTFRK

SEQ ID NO: 182 - protein, 41 aa, Unknown
  Description of Unknown Scorpion or spider venom or toxin polypeptide
GSECRYWLGTCSKTGDQCCKSSHLSCSPKHGWCVWDWTFRK

SEQ ID NO: 183 - protein, 38 aa, Unknown
  Description of Unknown Scorpion or spider venom or toxin polypeptide
GSECRYWLGTCSAGQDCCSHLSCSPKHGWCVWDWTFRK

SEQ ID NO: 184 - protein, 38 aa, Unknown
  Description of Unknown Scorpion or spider venom or toxin polypeptide
GSECRYWLGGCSATGDCCSHLSCSPKHGWCVWDWTFRK

SEQ ID NO: 185 - protein, 40 aa, Unknown
  Description of Unknown Scorpion or spider venom or toxin polypeptide
GSECRYWLGTCSKTGDCCKSSHLVCSPKHGWCVWDWTFRK

SEQ ID NO: 186 - protein, 38 aa, Unknown
  Description of Unknown Scorpion or spider venom or toxin polypeptide
GSECLEILGTCSKTGDCCSHLSCSPKHGWCVWDWTFRK

SEQ ID NO: 187 - protein, 38 aa, Unknown
  Description of Unknown Scorpion or spider venom or toxin polypeptide
GSECRYWFKACSKTGDCCSHLSCSPKHGWCVWDWTFRK

SEQ ID NO: 188 - protein, 38 aa, Unknown
  Description of Unknown Scorpion or spider venom or toxin polypeptide
GSECRYWLGTCSKTGDCCSHLSCSDGHGWCVWDWTFRK

SEQ ID NO: 189 - protein, 38 aa, Unknown
  Description of Unknown Scorpion or spider venom or toxin polypeptide
GSECRYWLGTCSKTGDCCSHLSCSKLHGWCVWDWTFRK

SEQ ID NO: 190 - protein, 38 aa, Unknown
  Description of Unknown Scorpion or spider venom or toxin polypeptide
GSECRYWLGTCSKTGDCCSHLQCHSKHGWCVWDWTFRK

SEQ ID NO: 191 - protein, 38 aa, Unknown
  Description of Unknown Scorpion or spider venom or toxin polypeptide
GSECRYWLGTCSRTGDCCSHLSCSPRHGWCVWDWTFRR

SEQ ID NO: 192 - protein, 30 aa, Unknown
  Description of Unknown Scorpion or spider venom or toxin polypeptide
GSECRYWLGTCSRTGDCCSHLSCSPRHGWC

SEQ ID NO: 193 - protein, 46 aa, Unknown
  Description of Unknown Scorpion or spider venom or toxin polypeptide
GSRCLPPGRPCYGATQRIPCCGVCSHNNCTGSSELYENKPRRPYIL

SEQ ID NO: 194 - protein, 46 aa, Unknown
  Description of Unknown Scorpion or spider venom or toxin polypeptide
GSVGCEECPMHCRGRNANPTCDDGVCNCNVGSSELYENKPRRPYIL

SEQ ID NO: 195 - protein, 54 aa, Unknown
  Description of Unknown Scorpion or spider venom or toxin polypeptide
GSMCMPCFTTDTQMQERCDRCCGGGGRGRCWGPQCLCIGSSELYENKPRRPYIL

SEQ ID NO: 196 - protein, 53 aa, Unknown
  Description of Unknown Scorpion or spider venom or toxin polypeptide
GSMCMPCFTTDPNMAKKCRDCCGGNGKCFGPQCLCNRGSSELYENKPRRPYIL

SEQ ID NO: 197 - protein, 53 aa, Unknown
  Description of Unknown Scorpion or spider venom or toxin polypeptide
GSSEKDCIKHLQRCRENKDCCSKKCSRRGTNPEKRCRGSSELYENKPRRPYIL

SEQ ID NO: 198 - protein, 38 aa, Unknown
  Description of Unknown Scorpion or spider venom or toxin polypeptide
  MOD_RES (3)..(3): Any amino acid or amino acid analogue
  MOD_RES (5)..(5): Any amino acid or amino acid analogue
  MOD_RES (11)..(17): Any amino acid or amino acid analogue
  MOD_RES (19)..(20): Any amino acid or amino acid analogue
  MOD_RES (24)..(27): Any amino acid or amino acid analogue
  MOD_RES (29)..(29): Any amino acid or amino acid analogue
  MOD_RES (31)..(31): Any amino acid or amino acid analogue
  MOD_RES (34)..(34): Any amino acid or amino acid analogue
  MOD_RES (36)..(36): Any amino acid or amino acid analogue
  MOD_RES (38)..(38): Any amino acid or amino acid analogue
GSXCXPCFTTXXXXXXXCXXCCGXXXXGXCXGPXCXCX

SEQ ID NO: 199 - protein, 38 aa, Unknown; identical to SEQ ID NO: 198
  Description of Unknown Scorpion or spider venom or toxin polypeptide
  MOD_RES (3)..(3): M, R, I, D, H, or L
  MOD_RES (5)..(5): M, I or L
  MOD_RES (11)..(11): D, H, E, S, G, or I
  MOD_RES (12)..(12): H, E, Q, R, Y, or T
  MOD_RES (13)..(13): Q, R, H, E, Y, or F
  MOD_RES (14)..(14): M, I, or L
  MOD_RES (15)..(15): A, F, E, I, or Q
  MOD_RES (16)..(16): R, E, I, D, N, or H
  MOD_RES (17)..(17): R, N, H, E, Y, F, I, T, or Q
  MOD_RES (19)..(19): D or E
  MOD_RES (20)..(20): D, I, H, E, R, Y, F, or A
  MOD_RES (24)..(24): G or I
  MOD_RES (25)..(25): R, D, W, F, or G
  MOD_RES (26)..(26): G, D, or S
  MOD_RES (27)..(27): R, D, G, or Y
  MOD_RES (29)..(29): K or R
  MOD_RES (31)..(31): Y, N, H, D, or W
  MOD_RES (34)..(34): Q or H
  MOD_RES (36)..(36): L or I
  MOD_RES (38)..(38): R, G, F, or I

SEQ ID NO: 200 - protein, 36 aa, Unknown
  Description of Unknown Scorpion or spider venom or toxin polypeptide
  MOD_RES (4)..(5): Any amino acid or amino acid analogue
  MOD_RES (7)..(16): Any amino acid or amino acid analogue
  MOD_RES (18)..(19): Any amino acid or amino acid analogue
  MOD_RES (22)..(28): Any amino acid or amino acid analogue
  MOD_RES (30)..(33): Any amino acid or amino acid analogue
  MOD_RES (35)..(35): Any amino acid or amino acid analogue
GSCXXCXXXXXXXXXXCXXCCXXXXXXXCXXXXCXC

SEQ ID NO: 201 - protein, 30 aa, Unknown
  Description of Unknown Scorpion or spider venom or toxin polypeptide
  MOD_RES (10)..(10): Any amino acid or amino acid analogue
  MOD_RES (13)..(13): Any amino acid or amino acid analogue
  MOD_RES (15)..(16): Any amino acid or amino acid analogue
  MOD_RES (18)..(18): Any amino acid or amino acid analogue
  MOD_RES (23)..(23): Any amino acid or amino acid analogue
GSVGCEECPXHCXGXXAXPTCDXGVCNCNV

SEQ ID NO: 202 - protein, 30 aa, Unknown; identical to SEQ ID NO: 201
  Description of Unknown Scorpion or spider venom or toxin polypeptide
  MOD_RES (10)..(10): M, A, V, I, or L
  MOD_RES (13)..(13): K or R
  MOD_RES (15)..(15): K or R
  MOD_RES (16)..(16): N, H, M, K, or Q
  MOD_RES (18)..(18): N, K, V, I, L, R or Q
  MOD_RES (23)..(23): D, N, G, Y, or E

SEQ ID NO: 203 - protein, 30 aa, Unknown
  Description of Unknown Scorpion or spider venom or toxin polypeptide
  MOD_RES (6)..(6): Any amino acid or amino acid analogue
  MOD_RES (8)..(8): Any amino acid or amino acid analogue
  MOD_RES (10)..(11): Any amino acid or amino acid analogue
  MOD_RES (16)..(16): Any amino acid or amino acid analogue
  MOD_RES (18)..(18): Any amino acid or amino acid analogue
  MOD_RES (22)..(25): Any amino acid or amino acid analogue
  MOD_RES (27)..(27): Any amino acid or amino acid analogue
  MOD_RES (30)..(30): Any amino acid or amino acid analogue
GSVGCXEXPXXCKGKXAXPTCXXXXCXCNX

SEQ ID NO: 204 - protein, 30 aa, Unknown
  Description of Unknown Scorpion or spider venom or toxin polypeptide
  MOD_RES (6)..(6): A or E
  MOD_RES (8)..(8): C or D
  MOD_RES (10)..(10): M, A, K, or V
  MOD_RES (11)..(11): H or Y
  MOD_RES (16)..(16): N, H, M, K, or Q
  MOD_RES (18)..(18): N, K, V, I, or L
  MOD_RES (22)..(22): D, Y, C, or E
  MOD_RES (23)..(23): D, N, G, or Y
  MOD_RES (24)..(24): G or E
  MISC_FEATURE (25)..(25): May or may not be present
  MOD_RES (27)..(27): N, K, or E
  MOD_RES (30)..(30): V, A, I, or D
GSVGCXEXPXXCKGKXAXPTCXXXVCXCNX

SEQ ID NO: 205 - protein, 31 aa, Unknown
  Description of Unknown Scorpion or spider venom or toxin polypeptide
  MOD_RES (3)..(4): Any amino acid or amino acid analogue
  MOD_RES (10)..(10): Any amino acid or amino acid analogue
  MOD_RES (13)..(18): Any amino acid or amino acid analogue
  MOD_RES (22)..(22): Any amino acid or amino acid analogue
  MOD_RES (25)..(25): Any amino acid or amino acid analogue
  MOD_RES (30)..(31): Any amino acid or amino acid analogue
GSXXCEDCPXHCXXXXXXAKCXNDXCVCEXX

SEQ ID NO: 206 - protein, 31 aa, Unknown; identical to SEQ ID NO: 205
  Description of Unknown Scorpion or spider venom or toxin polypeptide
  MOD_RES (3)..(3): V or A
  MOD_RES (4)..(4): S, T, or G
  MOD_RES (10)..(10): D or E
  MOD_RES (13)..(13): S or A
  MOD_RES (14)..(14): T or Q
  MOD_RES (15)..(15): Q or K
  MOD_RES (16)..(16): K, N, or D
  MOD_RES (17)..(17): A or Q
  MOD_RES (18)..(18): R or Q
  MOD_RES (22)..(22): D or E
  MOD_RES (25)..(25): K or R
  MOD_RES (30)..(30): P, S, or A
  MOD_RES (31)..(31): K, V, or I

SEQ ID NO: 207 - protein, 38 aa, Unknown
  Description of Unknown Scorpion or spider venom or toxin polypeptide
  MOD_RES (3)..(3): Any amino acid or amino acid analogue except P or C
  MOD_RES (5)..(5): A, L, V, I, or M
  MOD_RES (14)..(14): A, L, V, I, or M
  MOD_RES (29)..(29): K or R
  MOD_RES (36)..(36): A, L, V, I, or M
  MOD_RES (38)..(38): Any amino acid or amino acid analogue except C
GSXCXPCFTTDHQXARRCDDCCGGRGRGXCYGPQCXCX

SEQ ID NO: 208 - protein, 38 aa, Unknown
  Description of Unknown Scorpion or spider venom or toxin polypeptide
  MOD_RES (29)..(29): R or K
GSMCMPCFTTDHRMAENCDICCGGDGRGXCYGPQCLCR

SEQ ID NO: 209 - protein, 38 aa, Unknown
  Description of Unknown Scorpion or spider venom or toxin polypeptide
  MOD_RES (3)..(3): Any amino acid or amino acid analogue
  MOD_RES (11)..(13): Any amino acid or amino acid analogue
  MOD_RES (15)..(17): Any amino acid or amino acid analogue
  MOD_RES (20)..(20): Any amino acid or amino acid analogue
  MOD_RES (24)..(27): Any amino acid or amino acid analogue
  MOD_RES (29)..(29): Any amino acid or amino acid analogue
  MOD_RES (31)..(31): Any amino acid or amino acid analogue
  MOD_RES (34)..(34): Any amino acid or amino acid analogue
  MOD_RES (38)..(38): Any amino acid or amino acid analogue
GSXCMPCFTTXXXMXXXCDXCCGXXXXGXCXGPXCLCX

SEQ ID NO: 210 - protein, 34 aa, Unknown
  Description of Unknown Scorpion or spider venom or toxin polypeptide
MCIPCFTTNPNMAAKCNACCGSRRGSCRGPQCIC

SEQ ID NO: 211 - protein, 35 aa, Unknown
  Description of Unknown Scorpion or spider venom or toxin polypeptide
GCLEFWWKCNPNDDKCCRPKLKCSKLFKLCNFSFG

SEQ ID NO: 212 - protein, 36 aa, Unknown
  Description of Unknown Scorpion or spider venom or toxin polypeptide
ECRYWLGTCSKTGDCCSHLSCSPKHGWCVWDWTFRK

SEQ ID NO: 213 - protein, 36 aa, Unknown
  Description of Unknown Scorpion or spider venom or toxin polypeptide
MCMPCFTTDHQMARRCDDCCGGRGRGRCYGPQCLCR

SEQ ID NO: 214 - protein, 36 aa, Unknown
  Description of Unknown Scorpion or spider venom or toxin polypeptide
MCMPCFTTHHRMAENCDICCGGDGRGKCYGPQCLCR

SEQ ID NO: 215 - protein, 36 aa, Unknown
  Description of Unknown Scorpion or spider venom or toxin polypeptide
MCMPCFTTDHRMAENCDICCGGDGRGKCYGPQCLCR

SEQ ID NO: 216 - protein, 36 aa, Unknown
  Description of Unknown Scorpion or spider venom or toxin polypeptide
MCMPCFTTHHQMAENCDICCGGDGRGKCYGPQCLCR

SEQ ID NO: 217 - protein, 36 aa, Unknown
  Description of Unknown Scorpion or spider venom or toxin polypeptide
MCMPCFTTHHRMARNCDICCGGDGRGKCYGPQCLCR

SEQ ID NO: 218 - protein, 36 aa, Unknown
  Description of Unknown Scorpion or spider venom or toxin polypeptide
MCMPCFTTHHRMAERCDICCGGDGRGKCYGPQCLCR

SEQ ID NO: 219 - protein, 36 aa, Unknown
  Description of Unknown Scorpion or spider venom or toxin polypeptide
MCMPCFTTHHRMAENCDDCCGGDGRGKCYGPQCLCR

SEQ ID NO: 220 - protein, 36 aa, Unknown
  Description of Unknown Scorpion or spider venom or toxin polypeptide
RCMPCFTTDHQMARRCDDCCGGRGRGKCYGPQCLCR

SEQ ID NO: 221 - protein, 36 aa, Unknown
  Description of Unknown Scorpion or spider venom or toxin polypeptide
ICIPCFTTDHQIARRCDDCCGGRGRGKCYGPQCLCR

SEQ ID NO: 222 - protein, 36 aa, Unknown
  Description of Unknown Scorpion or spider venom or toxin polypeptide
MCLPCFTTDHQLARRCDDCCGGRGRGKCYGPQCLCR

SEQ ID NO: 223 - protein, 36 aa, Unknown
  Description of Unknown Scorpion or spider venom or toxin polypeptide
MCMPCFTTEHQMARRCEECCGGRGRGKCYGPQCLCR

SEQ ID NO: 224 - protein, 36 aa, Unknown
  Description of Unknown Scorpion or spider venom or toxin polypeptide
MCIPCFTTDHQMARRCEECCGGRGRGKCYGPQCLCR

SEQ ID NO: 225 - protein, 36 aa, Unknown
  Description of Unknown Scorpion or spider venom or toxin polypeptide
ICIPCFTTDHQMARRCDDCCGGRGDGKCYGPQCLCR

SEQ ID NO: 226 - protein, 36 aa, Unknown
  Description of Unknown Scorpion or spider venom or toxin polypeptide
ICIPCFTTDHQIARRCDDCCGGRGRGKCYGPQCICR

SEQ ID NO: 227 - protein, 36 aa, Unknown
  Description of Unknown Scorpion or spider venom or toxin polypeptide
RCMPCFTTDHFMARFCDFCCGGRGRGKCYGPQCLCR

SEQ ID NO: 228 - protein, 36 aa, Unknown
  Description of Unknown Scorpion or spider venom or toxin polypeptide
RCMPCFTTDHYMARYCDYCCGGRGRGKCYGPQCLCR

SEQ ID NO: 229 - protein, 36 aa, Unknown
  Description of Unknown Scorpion or spider venom or toxin polypeptide
RCMPCFTTDHRMARRCDRCCGGRGRGKCYGPQCLCR

SEQ ID NO: 230 - protein, 36 aa, Unknown
  Description of Unknown Scorpion or spider venom or toxin polypeptide
RCMPCFTTDHEMARECDECCGGRGRGKCYGPQCLCR

SEQ ID NO: 231 - protein, 36 aa, Unknown
  Description of Unknown Scorpion or spider venom or toxin polypeptide
RCMPCFTTDHHMARHCDHCCGGRGRGKCYGPQCLCR

SEQ ID NO: 232 - protein, 36 aa, Unknown
  Description of Unknown Scorpion or spider venom or toxin polypeptide
LCLPCFTTHHRLADQCDICCGGDGRGKCYGPQCLCR

SEQ ID NO: 233 - protein, 36 aa, Unknown
  Description of Unknown Scorpion or spider venom or toxin polypeptide
ICIPCFTTEHQIARRCEECCGGRGRGKCYGPQCLCR

SEQ ID NO: 234 - protein, 36 aa, Unknown
  Description of Unknown Scorpion or spider venom or toxin polypeptide
MCMPCFTTDTQMQERCDRCCGGGGRGKCWGPQCLCI

SEQ ID NO: 235 - protein, 36 aa, Unknown
  Description of Unknown Scorpion or spider venom or toxin polypeptide
MCMPCFTTIYRMAHECDECCGGRGRGKCYGPQCLCR

SEQ ID NO: 236 - protein, 36 aa, Unknown
  Description of Unknown Scorpion or spider venom or toxin polypeptide
MCMPCFTTGYRMAEYCDICCGGRGRGKCYGPQCLCR

SEQ ID NO: 237 - protein, 36 aa, Unknown
  Description of Unknown Scorpion or spider venom or toxin polypeptide
MCMPCFTTHRRMANTCDACCGGRSRGKCYGPQCLCR

SEQ ID NO: 238 - protein, 36 aa, Unknown
  Description of Unknown Scorpion or spider venom or toxin polypeptide
HCMPCFTTDHQMIRRCDDCCGGGSYGKCDGPQCLCF

SEQ ID NO: 239 - protein, 36 aa, Unknown
  Description of Unknown Scorpion or spider venom or toxin polypeptide
DCMPCFTTDHRMADHCDICCGGDDRGKCYGPQCLCR

SEQ ID NO: 240 - protein, 36 aa, Unknown
  Description of Unknown Scorpion or spider venom or toxin polypeptide
MCMPCFTTDHEMERRCDDCCGIGGGGKCHGPQCLCG

SEQ ID NO: 241 - protein, 36 aa, Unknown
  Description of Unknown Scorpion or spider venom or toxin polypeptide
MCMPCFTTEQRMAIICDDCCGGFGRGKCYGPQCLCR

SEQ ID NO: 242 - protein, 36 aa, Unknown
  Description of Unknown Scorpion or spider venom or toxin polypeptide
MCMPCFTTSEQMFRRCDDCCGGWGDGKCNGPHCLCR

SEQ ID NO: 243 - protein, 37 aa, Unknown
  Description of Unknown Scorpion or spider venom or toxin polypeptide
GVPINVKCRGSRDCLDPCKKAGMRFGKCINSKCHCTP

SEQ ID NO: 244 - protein, 36 aa, Unknown
  Description of Unknown Scorpion or spider venom or toxin polypeptide
MCMPCFTTEQRMAIICDDCCGGFGRGRCYGPQCLCR

SEQ ID NO: 245 - protein, 36 aa, Unknown
  Description of Unknown Scorpion or spider venom or toxin polypeptide
ICIPCFTTDHQIARRCDDCCGGRGRGRCYGPQCICR

SEQ ID NO: 246 - protein, 36 aa, Unknown
  Description of Unknown Scorpion or spider venom or toxin polypeptide
MCMPCFTTDTQMQERCDRCCGGGGRGRCWGPQCLCI

SEQ ID NO: 247 - protein, 35 aa, Unknown
  Description of Unknown Scorpion or spider venom or toxin polypeptide
MCMPCFTTDTQMQERCDRCCGGGGRGRCWGPQCLC

SEQ ID NO: 248 - protein, 36 aa, Unknown
  Description of Unknown Scorpion or spider venom or toxin polypeptide
MCMPCFTTDHRMAENCDICCGGDGRGRCYGPQCLCR

SEQ ID NO: 249 - protein, 36 aa, Unknown
  Description of Unknown Scorpion or spider venom or toxin polypeptide
MCMPCFTTEQRMAIICDDCCGGFGRGKCYGPQCLCI

SEQ ID NO: 250 - protein, 36 aa, Unknown
  Description of Unknown Scorpion or spider venom or toxin polypeptide
MCMPCFTTEQRMAIICDDCCGGFGRGRCYGPQCLCI

SEQ ID NO: 251 - protein, 36 aa, Unknown
  Description of Unknown Scorpion or spider venom or toxin polypeptide
ICIPCFTTDHQIARRCDDCCGGRGRGKCYGPQCICI

SEQ ID NO: 252 - protein, 36 aa, Unknown
  Description of Unknown Scorpion or spider venom or toxin polypeptide
ICIPCFTTDHQIARRCDDCCGGRGRGRCYGPQCICI

SEQ ID NO: 253 - protein, 36 aa, Unknown
  Description of Unknown Scorpion or spider venom or toxin polypeptide
MCMPCFTTDTQMQEKCDRCCGGGGRGRCWGPQCLCI

SEQ ID NO: 254 - protein, 36 aa, Unknown
  Description of Unknown Scorpion or spider venom or toxin polypeptide
MCMPCFTTEQRMAIKCDDCCGGFGRGRCYGPQCLCR

SEQ ID NO: 255 - protein, 36 aa, Unknown
  Description of Unknown Scorpion or spider venom or toxin polypeptide
ICIPCFTTDHQIARKCDDCCGGRGRGRCYGPQCICR

SEQ ID NO: 256 - protein, 36 aa, Unknown
  Description of Unknown Scorpion or spider venom or toxin polypeptide
MCMPCFTTDHRMAEKCDICCGGDGRGRCYGPQCLCR

SEQ ID NO: 257 - protein, 36 aa, Unknown
  Description of Unknown Scorpion or spider venom or toxin polypeptide
MCMPCFTTDTQMQERCDRCCGGKGRGRCWGPQCLCI

SEQ ID NO: 258 - protein, 36 aa, Unknown
  Description of Unknown Scorpion or spider venom or toxin polypeptide
MCMPCFTTEQRMAIICDDCCGGKGRGRCYGPQCLCR

SEQ ID NO: 259 - protein, 36 aa, Unknown
  Description of Unknown Scorpion or spider venom or toxin polypeptide
ICIPCFTTDHQIARRCDDCCGGKGRGRCYGPQCICR

SEQ ID NO: 260 - protein, 36 aa, Unknown
  Description of Unknown Scorpion or spider venom or toxin polypeptide
MCMPCFTTDHRMAENCDICCGGKGRGRCYGPQCLCR

SEQ ID NO: 261 - protein, 36 aa, Unknown
  Description of Unknown Scorpion or spider venom or toxin polypeptide
MCMPCFTTDHRMAENCDICCGGDGRGKCYGPQCLCI

SEQ ID NO: 262 - protein, 36 aa, Unknown
  Description of Unknown Scorpion or spider venom or toxin polypeptide
MCMPCFTTDHRMAENCDICCGGDGRGRCYGPQCLCI

SEQ ID NO: 263 - protein, 36 aa, Unknown
  Description of Unknown Scorpion or spider venom or toxin polypeptide
MCMPCFTTHHRMAENCDICCGGDGRGRCYGPQCLCR

SEQ ID NO: 264 - protein, 28 aa, Unknown
  Description of Unknown Scorpion or spider venom or toxin peptide
VGCEECPMHCKGKNANPTCDDGVCNCNV

SEQ ID NO: 265 - protein, 28 aa, Unknown
  Description of Unknown Scorpion or spider venom or toxin peptide
VGCEECPMHCKGKNAKPTCDDGVCNCNV

SEQ ID NO: 266 - protein, 28 aa, Unknown
  Description of Unknown Scorpion or spider venom or toxin peptide
VGCEECPMHCKGKNAKPTCDNGVCNCNV

SEQ ID NO: 267 - protein, 28 aa, Unknown
  Description of Unknown Scorpion or spider venom or toxin peptide
VGCEECPMHCKGKHAVPTCDDGVCNCNV

SEQ ID NO: 268 - protein, 28 aa, Unknown
  Description of Unknown Scorpion or spider venom or toxin peptide
VGCEECPAHCKGKNAKPTCDDGVCNCNV

SEQ ID NO: 269 - protein, 28 aa, Unknown
  Description of Unknown Scorpion or spider venom or toxin peptide
VGCEECPAHCKGKNAIPTCDDGVCNCNV

SEQ ID NO: 270 - protein, 28 aa, Unknown
  Description of Unknown Scorpion or spider venom or toxin peptide
VGCEECPMHCKGKMAKPTCDDGVCNCNV

SEQ ID NO: 271 - protein, 28 aa, Unknown
  Description of Unknown Scorpion or spider venom or toxin peptide
VGCEECPMHCKGKNAVPTCDNGVCNCNA

SEQ ID NO: 272 - protein, 28 aa, Unknown
  Description of Unknown Scorpion or spider venom or toxin peptide
VGCEECPMHCKGKMAKPTCYDGVCNCNV

SEQ ID NO: 273 - protein, 28 aa, Unknown
  Description of Unknown Scorpion or spider venom or toxin peptide
VGCEECPMYCKGKNAVPTCDGGVCNCNA

SEQ ID NO: 274 - protein, 28 aa, Unknown
  Description of Unknown Scorpion or spider venom or toxin peptide
VGCEECPKYCKGKNAVPTCDGGVCNCNA

SEQ ID NO: 275 - protein, 28 aa, Unknown
  Description of Unknown Scorpion or spider venom or toxin peptide
VGCEECPVYCKGKKALPTCDGGVCNCNA

SEQ ID NO: 276 - protein, 28 aa, Unknown
  Description of Unknown Scorpion or spider venom or toxin peptide
VGCEEDPMHCKGKQAKPTCCNGVCNCNV

SEQ ID NO: 277 - protein, 32 aa, Unknown
  Description of Unknown Scorpion or spider venom or toxin polypeptide
VGCAECPMHCKGKMAKPTCENEVCKCNIGKKD

SEQ ID NO: 278 - protein, 27 aa, Unknown
  Description of Unknown Scorpion or spider venom or toxin peptide
VGCEECPMHCKGKKALPTCDYGCECND

SEQ ID NO: 279 - protein, 31 aa, Unknown
  Description of Unknown Scorpion or spider venom or toxin polypeptide
IVCKVCKIICGMQGKKVNICKAPIKCKCKKG

SEQ ID NO: 280 - protein, 29 aa, Unknown
  Description of Unknown Scorpion or spider venom or toxin peptide
VSCEDCPDHCSTQKARAKCDNDKCVCEPK

SEQ ID NO: 281 - protein, 29 aa, Unknown
  Description of Unknown Scorpion or spider venom or toxin peptide
VSCEDCPEHCSTQKARAKCDNDKCVCESV

SEQ ID NO: 282 - protein, 29 aa, Unknown
  Description of Unknown Scorpion or spider venom or toxin peptide
VSCEDCPEHCSTQKAQAKCDNDKCVCEPI

SEQ ID NO: 283 - protein, 29 aa, Unknown
  Description of Unknown Scorpion or spider venom or toxin peptide
ATCEDCPEHCATQNARAKCDNDKCVCEPK

SEQ ID NO: 284 - protein, 29 aa, Unknown
  Description of Unknown Scorpion or spider venom or toxin peptide
VSCEDCPEHCATKDQRAKCDNDKCVCEPK

SEQ ID NO: 285 - protein, 29 aa, Unknown
  Description of Unknown Scorpion or spider venom or toxin peptide
VGCEDCPEHCSQQNARAKCENDKCVCEPK

SEQ ID NO: 286 - protein, 29 aa, Unknown
  Description of Unknown Scorpion or spider venom or toxin peptide
VSCEDCPEHCATKDQRAKCDNDRCVCEPK

SEQ ID NO: 287 - protein, 29 aa, Unknown
  Description of Unknown Scorpion or spider venom or toxin peptide
VSCEDCPPHCATKDQRAKCENDKCVCEPK

SEQ ID NO: 288 - protein, 29 aa, Unknown
  Description of Unknown Scorpion or spider venom or toxin peptide
VSCEDCPEHCSTQKARAKCDNDKCVCEAI

SEQ ID NO: 289 - protein, 35 aa, Unknown
  Description of Unknown Scorpion or spider venom or toxin polypeptide
MCMPCFTTEQRMAIICDDCCGGFGRGRCYGPQCLC

SEQ ID NO: 290 - protein, 35 aa, Unknown
  Description of Unknown Scorpion or spider venom or toxin polypeptide
ICIPCFTTDHQIARRCDDCCGGRGRGRCYGPQCIC

SEQ ID NO: 291 - protein, 35 aa, Unknown
  Description of Unknown Scorpion or spider venom or toxin polypeptide
MCMPCFTTDHRMAENCDICCGGDGRGRCYGPQCLC

SEQ ID NO: 292 - protein, 28 aa, Unknown
  Description of Unknown Scorpion or spider venom or toxin peptide
VGCEECPMHCRGRNANPTCDDGVCNCNV

SEQ ID NO: 293 - protein, 26 aa, Unknown
  Description of Unknown Scorpion or spider venom or toxin peptide
VGCEECPMHCRGRNANPTCDDGVCNC

SEQ ID NO: 294 - protein, 34 aa, Unknown
  Description of Unknown Scorpion or spider venom or toxin polypeptide
CGPCFTTDHQMEQKCAECCGGIGKCYGPQCLCNR

SEQ ID NO: 295 - protein, 38 aa, Unknown
  Description of Unknown Scorpion or spider venom or toxin polypeptide
RCGPCFTTDPQTQAKCSECCGRKGGVCKGPQCICGIQY

SEQ ID NO: 296 - protein, 35 aa, Unknown
  Description of Unknown Scorpion or spider venom or toxin polypeptide
MCMPCFTTDPNMAKKCRDCCGGNGKCFGPQCLCNR

SEQ ID NO: 297 - protein, 34 aa, Unknown
  Description of Unknown Scorpion or spider venom or toxin polypeptide
MCMPCFTTDHNMAKKCNDCCGGYGKCFGPQCLCR

SEQ ID NO: 298 - protein, 36 aa, Unknown
  Description of Unknown Scorpion or spider venom or toxin polypeptide
RCPPCFTTNPNMEADCRKCCGGRGYCASYQCICPGG

SEQ ID NO: 299 - protein, 35 aa, Unknown
  Description of Unknown Scorpion or spider venom or toxin polypeptide
MCMPCFTTDPNMANKCRDCCGGGKKCFGPQCLCNR

SEQ ID NO: 300 - protein, 62 aa, Unknown
  Description of Unknown Scorpion or spider venom or toxin polypeptide
MKFLYGVILIALFLTVMTATLSEARCGPCFTTDPQTQAKCSECCGRKGGVCKGPQCICGIQY

SEQ ID NO: 301 - protein, 36 aa, Unknown
  Description of Unknown Scorpion or spider venom or toxin polypeptide
MCMPCFTTRPDMAQQCRACCKGRGKCFGPQCLCGYD

SEQ ID NO: 302 - protein, 60 aa, Unknown
  Description of Unknown Scorpion or spider venom or toxin polypeptide
MKFLYGIVFIALFLTVMTATLSDAMCMPCFTTDHNMAKKCRDCCGGNGKCFGPQCLCNRG

SEQ ID NO: 303 - protein, 35 aa, Unknown
  Description of Unknown Scorpion or spider venom or toxin polypeptide
MCMPCFTTDHNMAKKCRDCCGGNGKCFGPQCLCNR

SEQ ID NO: 304 - protein, 60 aa, Unknown
  Description of Unknown Scorpion or spider venom or toxin polypeptide
MKFLYGIVFITLFLTVMIATHTEAMCMPCFTTRPNMAQQCRDCCRGRGKCFGPQCLCGYD

SEQ ID NO: 305 - protein, 60 aa, Unknown
  Description of Unknown Scorpion or spider venom or toxin polypeptide
MKFLYGIVFIALFLTVMIATHTEAMCMPCFTTRPNMAQQCRDCCRGRGKCFGPQCLCGYD

SEQ ID NO: 306 - protein, 35 aa, Unknown
  Description of Unknown Scorpion or spider venom or toxin polypeptide
RCKPCFTTDPQMSKKCADCCGGKGKGKCYGPQCLC

SEQ ID NO: 307 - protein, 61 aa, Unknown
  Description of Unknown Scorpion or spider venom or toxin polypeptide
MKFLYGIVFITLFLTVMIATHTEAAMCMPCFTTNLNMEQECRDCCGGTGRCFGPQCLCGYD

SEQ ID NO: 308 - protein, 38 aa, Unknown
  Description of Unknown Scorpion or spider venom or toxin polypeptide
RCSPCFTTDQQMTKKCYDCCGGKGKGKCYGPQCICAPY

SEQ ID NO: 309 - protein, 35 aa, Unknown
  Description of Unknown Scorpion or spider venom or toxin polypeptide
CGPCFTTDPYTESKCATCCGGRGKCVGPQCLCNRI

SEQ ID NO: 310 - protein, 39 aa, Unknown
  Description of Unknown Scorpion or spider venom or toxin polypeptide
TEAMCMPCFTTDHNMAKKCRDCCGGNGKCFGYQCLCNRG

SEQ ID NO: 311 - protein, 59 aa, Unknown
  Description of Unknown Scorpion or spider venom or toxin polypeptide
MKFLYGIVFIALFLTVMFATQTDGCGPCFTTDANMARKCRECCGGIGKCFGPQCLCNRI

SEQ ID NO: 312 - protein, 59 aa, Unknown
  Description of Unknown Scorpion or spider venom or toxin polypeptide
MKFLYGIVFIALFLTVMFATQTDGCGPCFTTDANMARKCRECCGGNGKCFGPQCLCNRE

SEQ ID NO: 313 - protein, 60 aa, Unknown
  Description of Unknown Scorpion or spider venom or toxin polypeptide
MKFLYGTILIAFFLTVMIATHSEARCPPCFTTNPNMEADCRKCCGGRGYCASYQCICPGG

SEQ ID NO: 314 - protein, 39 aa, Unknown
  Description of Unknown Scorpion or spider venom or toxin polypeptide
TEAMCMPCFTTRPDMAQQCRDCCGGNGKCFGYQCLCNRG

SEQ ID NO: 315 - protein, 60 aa, Unknown
  Description of Unknown Scorpion or spider venom or toxin polypeptide
MKFLYGIVFIALFLTVMIATLTEAMCMPCFTTRPDMAQQCRDCCGGNGKCFGYQCLCNRG

SEQ ID NO: 316 - protein, 60 aa, Unknown
  Description of Unknown Scorpion or spider venom or toxin polypeptide
MKFLYGIVFIALFLTVMIATHTEAMCMPCFTTRPDMAQQCRDCCGGNGKCFGYQCLCNRG

SEQ ID NO: 317 - protein, 61 aa, Unknown
  Description of Unknown Scorpion or spider venom or toxin polypeptide
MKFLYGIILIALFLTVMIATHSEARCPNCFTTNPNAEADCKKCCGNRWGKCAGYQCVCPMK

SEQ ID NO: 318 - protein, 67 aa, Unknown
  Description of Unknown Scorpion or spider venom or toxin polypeptide
MKFLYGIVFIALFLTGMIATHTEAMCMPCFTTRPDMAQQCRDCCGGNGKCFGYQCLCNRGRIVIMYT

SEQ ID NO: 319 - protein, 35 aa, Unknown
  Description of Unknown Scorpion or spider venom or toxin polypeptide
MCMPCFTTRPGMAQQCRDCCGGNGKCFGYQCLCNR

SEQ ID NO: 320 - protein, 35 aa, Unknown
  Description of Unknown Scorpion or spider venom or toxin polypeptide
MCIPCFTTNPNMAAKCNACCGSRRGSCRGPQCICR

SEQ ID NO: 321 - protein, 35 aa, Unknown
  Description of Unknown Scorpion or spider venom or toxin polypeptide
MCIPCFTTNPNMAAKCNACCGSRRGSCRGPQCICN

SEQ ID NO: 322 - protein, 34 aa, Unknown
  Description of Unknown Scorpion or spider venom or toxin polypeptide
MCIPCFTTNPNMAAKCNACCGGNGSCRGPQCICN

SEQ ID NO: 323 - protein, 36 aa, Unknown
  Description of Unknown Scorpion or spider venom or toxin polypeptide
MCIPCFTTNPNMAAKCNACCGSRGRGSCRGPQCICN

SEQ ID NO: 324 - protein, 36 aa, Unknown
  Description of Unknown Scorpion or spider venom or toxin polypeptide
MCIPCFTTNPNMAAKCNACCGSRGRGKCRGPQCICN

SEQ ID NO: 325 - protein, 35 aa, Unknown
  Description of Unknown Scorpion or spider venom or toxin polypeptide
MCIPCFTTDHQMAAKCNACCGSRRGSCRGPQCICN

SEQ ID NO: 326 - protein, 35 aa, Unknown
  Description of Unknown Scorpion or spider venom or toxin polypeptide
MCIPCFTTNHQMAAKCNACCGSRRGSCRGPQCICN

SEQ ID NO: 327 - protein, 36 aa, Unknown
  Description of Unknown Scorpion or spider venom or toxin polypeptide
MCIPCFTTNPNMARKCNACCGSRGRGSCRGPQCICN

SEQ ID NO: 328 - protein, 36 aa, Unknown
  Description of Unknown Scorpion or spider venom or toxin polypeptide
MCIPCFTTNPNMAAKCNACCGGKGRGSCRGPQCICN

SEQ ID NO: 329 - protein, 35 aa, Unknown
  Description of Unknown Scorpion or spider venom or toxin polypeptide
MCIPCFTTNPNMAAKCNACCGSRRGSCFGPQCICN

SEQ ID NO: 330 - protein, 36 aa, Unknown
  Description of Unknown Scorpion or spider venom or toxin polypeptide
MCIPCFTTNPNMAAKCNACCGSRGRGKCFGPQCICN

SEQ ID NO: 331 - protein, 36 aa, Unknown
  Description of Unknown Scorpion or spider venom or toxin polypeptide
MCIPCFTTNPNMAAKCNACCGSRGRGSCFGPQCICN

SEQ ID NO: 332 - protein, 36 aa, Unknown
  Description of Unknown Scorpion or spider venom or toxin polypeptide
MCIPCFTTNPNMAAKCNACCGSRGRGSCYGPQCICN

SEQ ID NO: 333 - protein, 35 aa, Unknown
  Description of Unknown Scorpion or spider venom or toxin polypeptide
MCIPCFTTNPNMAAKCDACCGSRRGSCRGPQCICN

SEQ ID NO: 334 - protein, 35 aa, Unknown
  Description of Unknown Scorpion or spider venom or toxin polypeptide
MCIPCFTTNHQMAAKCDACCGSRRGSCRGPQCICN

SEQ ID NO: 335 - protein, 36 aa, Unknown
  Description of Unknown Scorpion or spider venom or toxin polypeptide
MCIPCFTTNHNMAAKCDACCGGRGRGSCRGPQCICN

SEQ ID NO: 336 - protein, 36 aa, Unknown
  Description of Unknown Scorpion or spider venom or toxin polypeptide
MCIPCFTTNPNMAAKCDACCGSRGRGSCRGPQCICN

SEQ ID NO: 337 - protein, 36 aa, Unknown
  Description of Unknown Scorpion or spider venom or toxin polypeptide
MCIPCFTTNPNMAAKCDACCGGKGRGSCRGPQCICN

SEQ ID NO: 338 - protein, 36 aa, Unknown
  Description of Unknown Scorpion or spider venom or toxin polypeptide
MCIPCFTTNHNMAAKCDACCGSRGRGSCRGPQCICN

SEQ ID NO: 339 - protein, 34 aa, Unknown
  Description of Unknown Scorpion or spider venom or toxin polypeptide
MCIPCFTTNPNMAAKCRDCCGGRGSCRGPQCICN

SEQ ID NO: 340 - protein, 36 aa, Unknown
  Description of Unknown Scorpion or spider venom or toxin polypeptide
MCMPCFTTNPNMAAKCDDCCGSRGRGSCRGPQCICN

SEQ ID NO: 341 - protein, 34 aa, Unknown
  Description of Unknown Scorpion or spider venom or toxin polypeptide
MCIPCFTTNPNMAARCNACCGSRRGSCRGPQCIC

SEQ ID NO: 342 - protein, 35 aa, Unknown
  Description of Unknown Scorpion or spider venom or toxin polypeptide
MCIPCFTTNPNMAAKCNACCGSRRGSCRGPQCICI

SEQ ID NO: 343 - protein, 36 aa, Unknown
  Description of Unknown Scorpion or spider venom or toxin polypeptide
GCLQFMWKCNPDNDKCCRPNLKCNTYHKWCEFVTGK

SEQ ID NO: 344 - protein, 34 aa, Unknown
  Description of Unknown Scorpion or spider venom or toxin polypeptide
DCLGFLWKCNPSNDKCCRPNLVCSRKDKWCKYQI

SEQ ID NO: 345 - protein, 36 aa, Unknown
  Description of Unknown Scorpion or spider venom or toxin polypeptide
DCLGFMRKCIPDNDKCCRPNLVCSRTHKWCKYVFGK

SEQ ID NO: 346 - protein, 35 aa, Unknown
  Description of Unknown Scorpion or spider venom or toxin polypeptide
ECLEIFKACNPSNDQCCKSSKLVCSRKTRACKYQI

SEQ ID NO: 347 - protein, 34 aa, Unknown
  Description of Unknown Scorpion or spider venom or toxin polypeptide
ECGGFWWKCGSGKPACCPKYVCSPKWGLCNFPMP

SEQ ID NO: 348 - protein, 35 aa, Unknown
  Description of Unknown Scorpion or spider venom or toxin polypeptide
GCLERWWKCNPNDDKCCRPKLKCSKLFKLCNRSRG

SEQ ID NO: 349 - protein, 35 aa, Unknown
  Description of Unknown Scorpion or spider venom or toxin polypeptide
GCLEEWWKCNPNDDKCCRPKLKCSKLFKLCNESEG

SEQ ID NO: 350 - protein, 35 aa, Unknown
  Description of Unknown Scorpion or spider venom or toxin polypeptide
GCLEIWWKCNPNDDKCCRPKLKCSKLFKLCNYSIG

SEQ ID NO: 351 - protein, 35 aa, Unknown
  Description of Unknown Scorpion or spider venom or toxin polypeptide
GCLEFWWKCNPNDDKCCRPKLKCSKLGKLCNFSFG

SEQ ID NO: 352 - protein, 35 aa, Unknown
  Description of Unknown Scorpion or spider venom or toxin polypeptide
GCLEFWWKCNPNDDKCCRPKLKCSPLGKLCNFSFG

SEQ ID NO: 353 - protein, 35 aa, Unknown
  Description of Unknown Scorpion or spider venom or toxin polypeptide
GCLEFWWKCNPNDDKCCRPKLKCSPNGKLCNFSFG

SEQ ID NO: 354 - protein, 35 aa, Unknown
  Description of Unknown Scorpion or spider venom or toxin polypeptide
GCLEFWWKCNPNDDKCCRPKLKCSRKTKLCNFSFG

SEQ ID NO: 355 - protein, 35 aa, Unknown
  Description of Unknown Scorpion or spider venom or toxin polypeptide
GCLEFWWKCNPNDDKCCRPKLKCGSNFKLCNFSFG

SEQ ID NO: 356 - protein, 35 aa, Unknown
  Description of Unknown Scorpion or spider venom or toxin polypeptide
GCLEFWWKCNPNDDKCCRPKLKCSTKHKLCNFSFG

SEQ ID NO: 357 - protein, 35 aa, Unknown
  Description of Unknown Scorpion or spider venom or toxin polypeptide
GCLEFWWKCNPNDDKCCRPKLKCSNDGKLCNFSFG

SEQ ID NO: 358 - protein, 35 aa, Unknown
  Description of Unknown Scorpion or spider venom or toxin polypeptide
GCLEFWWKCNPNDDKCCRPKLKCSKKTKLCNFSFG

SEQ ID NO: 359 - protein, 35 aa, Unknown
  Description of Unknown Scorpion or spider venom or toxin polypeptide
GCLEFWWKCNPNDDKCCRPKLKCHSNFKLCNFSFG

SEQ ID NO: 360 - protein, 35 aa, Unknown
  Description of Unknown Scorpion or spider venom or toxin polypeptide
GCLEFWWKCNPNDDKCCRPKLKCSKKFTACNFSFG

SEQ ID NO: 361 - protein, 35 aa, Unknown
  Description of Unknown Scorpion or spider venom or toxin polypeptide
GCLEIFKACNPNDDKCCRPKLKCSKLFKLCNFSFG

SEQ ID NO: 362 - protein, 35 aa, Unknown
  Description of Unknown Scorpion or spider venom or toxin polypeptide
GCLKFGWKCNPNDDKCCRPKLKCSKLFKLCNFSFG

SEQ ID NO: 363 - protein, 36 aa, Unknown
  Description of Unknown Scorpion or spider venom or toxin polypeptide
GCLEFWWKCNPNDDKCCKSSKLKCSKLFKLCNFSFG

SEQ ID NO: 364 - protein, 35 aa, Unknown
  Description of Unknown Scorpion or spider venom or toxin polypeptide
GCLEFWWKCNPNDDKCCRPKLKCNKLFKLCNISIG

SEQ ID NO: 365 - protein, 33 aa, Unknown
  Description of Unknown Scorpion or spider venom or toxin polypeptide
GCLEFWWKCNPNDDCCRKLKCSKLFKLCNFSFG

SEQ ID NO: 366 - protein, 35 aa, Unknown
  Description of Unknown Scorpion or spider venom or toxin polypeptide
GCLEFWWKCNPSNDQCCRPKLKCSKLFKLCNFSFG

SEQ ID NO: 367 - protein, 36 aa, Unknown
  Description of Unknown Scorpion or spider venom or toxin polypeptide
GCLEFWWKCNPNDDKCCRPSKLVCSKLFKLCNFSFG

SEQ ID NO: 368 - protein, 35 aa, Unknown
  Description of Unknown Scorpion or spider venom or toxin polypeptide
GCLEFLGECNPNDDKCCRPKLKCSKLFKLCNFSFG

SEQ ID NO: 369 - protein, 35 aa, Unknown
  Description of Unknown Scorpion or spider venom or toxin polypeptide
GCLWYLWKCNPNDDKCCRPKLKCSKLFKLCNFSFG

SEQ ID NO: 370 - protein, 35 aa, Unknown
  Description of Unknown Scorpion or spider venom or toxin polypeptide
GCLEFWWRCNPNDDRCCRPRLRCSRLFRLCNFSFG

SEQ ID NO: 371 - protein, 30 aa, Unknown
  Description of Unknown Scorpion or spider venom or toxin polypeptide
GCLEFWWRCNPNDDRCCRPRLRCSRLFRLC

SEQ ID NO: 372 - protein, 35 aa, Unknown
  Description of Unknown Scorpion or spider venom or toxin polypeptide
CRYFLGECKKTSECCEHLACHDKHKWCAWDWTIGK

SEQ ID NO: 373 - protein, 34 aa, Unknown
  Description of Unknown Scorpion or spider venom or toxin polypeptide
ECRYWLGGCSAGQTCCKHLVCSRRHGWCVWDGTF

SEQ ID NO: 374 - protein, 36 aa, Unknown
  Description of Unknown Scorpion or spider venom or toxin polypeptide
ECRWYLGECSQDGDCCKHLQCHSNYEWCIWDGTFSK

SEQ ID NO: 375 - protein, 36 aa, Unknown
  Description of Unknown Scorpion or spider venom or toxin polypeptide
ECRWYLGGCSQDGDCCKHLQCHSNYEWCVWDGTFSK

SEQ ID NO: 376 - protein, 34 aa, Unknown
  Description of Unknown Scorpion or spider venom or toxin polypeptide
DCRKFLGACTQTSDCCKHLACHNKHKWCAWDWTI

SEQ ID NO: 377 - protein, 37 aa, Unknown
  Description of Unknown Scorpion or spider venom or toxin polypeptide
ECRYLMGGCSKDGDCCEHLVCRTKWPYHCVWDWTFGK

SEQ ID NO: 378 - protein, 36 aa, Unknown
  Description of Unknown Scorpion or spider venom or toxin polypeptide
ECRYRLGTCSKTGDCCSHLSCSPKHGWCVRDRTFRK

SEQ ID NO: 379 - protein, 36 aa, Unknown
  Description of Unknown Scorpion or spider venom or toxin polypeptide
ECRYELGTCSKTGDCCSHLSCSPKHGWCVEDETFRK

SEQ ID NO: 380 - protein, 36 aa, Unknown
  Description of Unknown Scorpion or spider venom or toxin polypeptide
ECRYILGTCSKTGDCCSHLSCSPKHGWCVYDITFRK

SEQ ID NO: 381 - protein, 36 aa, Unknown
  Description of Unknown Scorpion or spider venom or toxin polypeptide
ECRYWLGTCSKTGDCCSHLSCSPKGGWCVWDWTFRK

SEQ ID NO: 382 - protein, 36 aa, Unknown
  Description of Unknown Scorpion or spider venom or toxin polypeptide
ECRYWLGTCSKTGDCCSHLSCSPNHGWCVWDWTFRK

SEQ ID NO: 383 - protein, 36 aa, Unknown
  Description of Unknown Scorpion or spider venom or toxin polypeptide
ECRYWLGTCSKTGDCCSHLSCSRKTGWCVWDWTFRK

SEQ ID NO: 384 - protein, 36 aa, Unknown
  Description of Unknown Scorpion or spider venom or toxin polypeptide
ECRYWLGTCSKTGDCCSHLSCGSNHGWCVWDWTFRK

SEQ ID NO: 385 - protein, 36 aa, Unknown
  Description of Unknown Scorpion or spider venom or toxin polypeptide
ECRYWLGTCSKTGDCCSHLSCSTKHGWCVWDWTFRK

SEQ ID NO: 386 - protein, 36 aa, Unknown
  Description of Unknown Scorpion or spider venom or toxin polypeptide
ECRYWLGTCSKTGDCCSHLSCSSKHGWCVWDWTFRK

SEQ ID NO: 387 - protein, 36 aa, Unknown
  Description of Unknown Scorpion or spider venom or toxin polypeptide
ECRYWLGTCSKTGDCCSHLSCSNDGGWCVWDWTFRK

SEQ ID NO: 388 - protein, 36 aa, Unknown
  Description of Unknown Scorpion or spider venom or toxin polypeptide
ECRYWLGTCSKTGDCCSHLSCSPKTRACVWDWTFRK

SEQ ID NO: 389 - protein, 36 aa, Unknown
  Description of Unknown Scorpion or spider venom or toxin polypeptide
ECRYWLGTCSKTGDCCSHLSCHSNHGWCVWDWTFRK

SEQ ID NO: 390 - protein, 36 aa, Unknown
  Description of Unknown Scorpion or spider venom or toxin polypeptide
ECRYWLGTCSKTGDCCSHLSCSRKHRACVWDWTFRK

SEQ ID NO: 391 - protein, 39 aa, Unknown
  Description of Unknown Scorpion or spider venom or toxin polypeptide
ECRYWLGTCSKTGDQCCKSSHLSCSPKHGWCVWDWTFRK

SEQ ID NO: 392 - protein, 36 aa, Unknown
  Description of Unknown Scorpion or spider venom or toxin polypeptide
ECRYWLGTCSAGQDCCSHLSCSPKHGWCVWDWTFRK

SEQ ID NO: 393 - protein, 36 aa, Unknown
  Description of Unknown Scorpion or spider venom or toxin polypeptide
ECRYWLGGCSATGDCCSHLSCSPKHGWCVWDWTFRK

SEQ ID NO: 394 - protein, 38 aa, Unknown
  Description of Unknown Scorpion or spider venom or toxin polypeptide
ECRYWLGTCSKTGDCCKSSHLVCSPKHGWCVWDWTFRK

SEQ ID NO: 395 - protein, 36 aa, Unknown
  Description of Unknown Scorpion or spider venom or toxin polypeptide
ECLEILGTCSKTGDCCSHLSCSPKHGWCVWDWTFRK

SEQ ID NO: 396 - protein, 36 aa, Unknown
  Description of Unknown Scorpion or spider venom or toxin polypeptide
ECRYWFKACSKTGDCCSHLSCSPKHGWCVWDWTFRK

SEQ ID NO: 397 - protein, 36 aa, Unknown
  Description of Unknown Scorpion or spider venom or toxin polypeptide
ECRYWLGTCSKTGDCCSHLSCSDGHGWCVWDWTFRK

SEQ ID NO: 398 - protein, 36 aa, Unknown
  Description of Unknown Scorpion or spider venom or toxin polypeptide
ECRYWLGTCSKTGDCCSHLSCSKLHGWCVWDWTFRK

SEQ ID NO: 399 - protein, 36 aa, Unknown
  Description of Unknown Scorpion or spider venom or toxin polypeptide
ECRYWLGTCSKTGDCCSHLQCHSKHGWCVWDWTFRK

SEQ ID NO: 400 - protein, 36 aa, Unknown
  Description of Unknown Scorpion or spider venom or toxin polypeptide
ECRYWLGTCSRTGDCCSHLSCSPRHGWCVWDWTFRR

SEQ ID NO: 401 - protein, 28 aa, Unknown
  Description of Unknown Scorpion or spider venom or toxin peptide
ECRYWLGTCSRTGDCCSHLSCSPRHGWC

SEQ ID NO: 402 - protein, 41 aa, Unknown
  Description of Unknown Scorpion or spider venom or toxin polypeptide
RCLPPGRPCYGATQRIPCCGVCSHNNCTELYENKPRRPYIL

SEQ ID NO: 403 - protein, 41 aa, Unknown
  Description of Unknown Scorpion or spider venom or toxin polypeptide
VGCEECPMHCRGRNANPTCDDGVCNCNVELYENKPRRPYIL

SEQ ID NO: 404 - protein, 49 aa, Unknown
  Description of Unknown Scorpion or spider venom or toxin polypeptide
MCMPCFTTDTQMQERCDRCCGGGGRGRCWGPQCLCIELYENKPRRPYIL

SEQ ID NO: 405 - protein, 48 aa, Unknown
  Description of Unknown Scorpion or spider venom or toxin polypeptide
MCMPCFTTDPNMAKKCRDCCGGNGKCFGPQCLCNRELYENKPRRPYIL

SEQ ID NO: 406 - protein, 47 aa, Unknown
  Description of Unknown Scorpion or spider venom or toxin polypeptide
EKDCIKHLQRCRENKDCCSKKCSRRGTNPEKRCRELYENKPRRPYIL

SEQ ID NO: 407 - protein, 36 aa, Unknown
  Description of Unknown Scorpion or spider venom or toxin polypeptide
  MOD_RES (1)..(1): Any amino acid or amino acid analogue
  MOD_RES (3)..(3): Any amino acid or amino acid analogue
  MOD_RES (9)..(15): Any amino acid or amino acid analogue
  MOD_RES (17)..(18): Any amino acid or amino acid analogue
  MOD_RES (22)..(25): Any amino acid or amino acid analogue
  MOD_RES (27)..(27): Any amino acid or amino acid analogue
  MOD_RES (29)..(29): Any amino acid or amino acid analogue
  MOD_RES (32)..(32): Any amino acid or amino acid analogue
  MOD_RES (34)..(34): Any amino acid or amino acid analogue
  MOD_RES (36)..(36): Any amino acid or amino acid analogue
XCXPCFTTXXXXXXXCXXCCGXXXXGXCXGPXCXCX

SEQ ID NO: 408 - protein, 36 aa, Unknown; identical to SEQ ID NO: 407
  Description of Unknown Scorpion or spider venom or toxin polypeptide
  MOD_RES (1)..(1): M, R, I, D, H, or L
  MOD_RES (3)..(3): M, I, or L
  MOD_RES (9)..(9): D, H, E, S, G, or I
  MOD_RES (10)..(10): H, E, Q, R, Y, or T
  MOD_RES (11)..(11): Q, R, H, E, Y, or F
  MOD_RES (12)..(12): M, I, or L
  MOD_RES (13)..(13): A, F, E, I, or Q
  MOD_RES (14)..(14): R, E, I, D, N, or H
  MOD_RES (15)..(15): R, N, H, E, Y, F, I, T, or Q
  MOD_RES (17)..(17): D or E
  MOD_RES (18)..(18): D, I, H, E, R, Y, F, or A
  MOD_RES (22)..(22): G or I
  MOD_RES (23)..(23): R, D, W, F, or G
  MOD_RES (24)..(24): G, D, or S
  MOD_RES (25)..(25): R, D, G, or Y
  MOD_RES (27)..(27): K or R
  MOD_RES (29)..(29): Y, N, H, D, or W
  MOD_RES (32)..(32): Q or H
  MOD_RES (34)..(34): L or I
  MOD_RES (36)..(36): R, G, F, or I

SEQ ID NO: 409 - protein, 34 aa, Unknown
  Description of Unknown Scorpion or spider venom or toxin polypeptide
  MOD_RES (2)..(3): Any amino acid or amino acid analogue
  MOD_RES (5)..(14): Any amino acid or amino acid analogue
  MOD_RES (16)..(17): Any amino acid or amino acid analogue
  MOD_RES (20)..(26): Any amino acid or amino acid analogue
  MOD_RES (28)..(31): Any amino acid or amino acid analogue
  MOD_RES (33)..(33): Any amino acid or amino acid analogue
CXXCXXXXXXXXXXCXXCCXXXXXXXCXXXXCXC

SEQ ID NO: 410 - protein, 28 aa, Unknown
  Description of Unknown Scorpion or spider venom or toxin peptide
  MOD_RES (8)..(8): Any amino acid or amino acid analogue
  MOD_RES (11)..(11): Any amino acid or amino acid analogue
  MOD_RES (13)..(14): Any amino acid or amino acid analogue
  MOD_RES (16)..(16): Any amino acid or amino acid analogue
  MOD_RES (21)..(21): Any amino acid or amino acid analogue
VGCEECPXHCXGXXAXPTCDXGVCNCNV

SEQ ID NO: 411 - protein, 28 aa, Unknown
  Description of Unknown Scorpion or spider venom or toxin peptide
  MOD_RES (8)..(8): M, A, V, I, or L
  MOD_RES (11)..(11): K or R
  MOD_RES (13)..(13): K or R
  MOD_RES (14)..(14): N, H, M, K, or Q
  MOD_RES (16)..(16): N, K, V, I, L, R or Q
  MOD_RES (21)..(21): D, N, G, Y, or E
VGCEECPXHCXGXXAXPTCDXGVCNCNV

SEQ ID NO: 412 - protein, 28 aa, Unknown
  Description of Unknown Scorpion or spider venom or toxin peptide
  MOD_RES (4)..(4): Any amino acid or amino acid analogue
  MOD_RES (6)..(6): Any amino acid or amino acid analogue
  MOD_RES (8)..(9): Any amino acid or amino acid analogue
  MOD_RES (14)..(14): Any amino acid or amino acid analogue
  MOD_RES (16)..(16): Any amino acid or amino acid analogue
  MOD_RES (20)..(23): Any amino acid or amino acid analogue
  MOD_RES (25)..(25): Any amino acid or amino acid analogue
  MOD_RES (28)..(28): Any amino acid or amino acid analogue
VGCXEXPXXCKGKXAXPTCXXXXCXCNX

SEQ ID NO: 413 - protein, 28 aa, Unknown
  Description of Unknown Scorpion or spider venom or toxin peptide
  MOD_RES (4)..(4): A or E
  MOD_RES (6)..(6): C or D
  MOD_RES (8)..(8): M, A, K, or V
  MOD_RES (9)..(9): H or Y
  MOD_RES (14)..(14): N, H, M, K, or Q
  MOD_RES (16)..(16): N, K, V, I, or L
  MOD_RES (20)..(20): D, Y, C, or E
  MOD_RES (21)..(21): D, N, G, or Y
  MOD_RES (22)..(22): G or E
  MISC_FEATURE (23)..(23): May or may not be present
  MOD_RES (25)..(25): N, K, or E
  MOD_RES (28)..(28): V, A, I, or D
VGCXEXPXXCKGKXAXPTCXXXVCXCNX

SEQ ID NO: 414 - protein, 29 aa, Unknown
  Description of Unknown Scorpion or spider venom or toxin peptide
  MOD_RES (1)..(2): Any amino acid or amino acid analogue
  MOD_RES (8)..(8): Any amino acid or amino acid analogue
  MOD_RES (11)..(16): Any amino acid or amino acid analogue
  MOD_RES (20)..(20): Any amino acid or amino acid analogue
  MOD_RES (23)..(23): Any amino acid or amino acid analogue
  MOD_RES (28)..(29): Any amino acid or amino acid analogue
XXCEDCPXHCXXXXXXAKCXNDXCVCEXX

SEQ ID NO: 415 - protein, 29 aa, Unknown
  Description of Unknown Scorpion or spider venom or toxin peptide
  MOD_RES (1)..(1): V or A
  MOD_RES (2)..(2): S, T, or G
  MOD_RES (8)..(8): D or E
  MOD_RES (11)..(11): S or A
  MOD_RES (12)..(12): T or Q
  MOD_RES (13)..(13): Q or K
  MOD_RES (14)..(14): K, N, or D
  MOD_RES (15)..(15): A or Q
  MOD_RES (16)..(16): R or Q
  MOD_RES (20)..(20): D or E
  MOD_RES (23)..(23): K or R
  MOD_RES (28)..(28): P, S, or A
  MOD_RES (29)..(29): K, V, or I
XXCEDCPXHCXXXXXXAKCXNDXCVCEXX

SEQ ID NO: 416 - protein, 36 aa, Unknown
  Description of Unknown Scorpion or spider venom or toxin polypeptide
  MOD_RES (1)..(1): Any amino acid or amino acid analogue except P or C
  MOD_RES (3)..(3): A, L, V, I, or M
  MOD_RES (12)..(12): A, L, V, I, or M
  MOD_RES (27)..(27): K or R
  MOD_RES (34)..(34): A, L, V, I, or M
  MOD_RES (36)..(36): Any amino acid or amino acid analogue except C
XCXPCFTTDHQXARRCDDCCGGRGRGXCYGPQCXCX

SEQ ID NO: 417 - protein, 36 aa, Unknown
  Description of Unknown Scorpion or spider venom or toxin polypeptide
  MOD_RES (27)..(27): R or K
MCMPCFTTDHRMAENCDICCGGDGRGXCYGPQCLCR

SEQ ID NO: 418 - protein, 36 aa, Unknown
  Description of Unknown Scorpion or spider venom or toxin polypeptide
  MOD_RES (1)..(1): Any amino acid or amino acid analogue
  MOD_RES (9)..(11): Any amino acid or amino acid analogue
  MOD_RES (13)..(15): Any amino acid or amino acid analogue
  MOD_RES (18)..(18): Any amino acid or amino acid analogue
  MOD_RES (22)..(25): Any amino acid or amino acid analogue
  MOD_RES (27)..(27): Any amino acid or amino acid analogue
  MOD_RES (29)..(29): Any amino acid or amino acid analogue
  MOD_RES (32)..(32): Any amino acid or amino acid analogue
  MOD_RES (36)..(36): Any amino acid or amino acid analogue
XCMPCFTTXXXMXXXCDXCCGXXXXGXCXGPXCLCX

SEQ ID NO: 419 - protein, 30 aa, Unknown
  Description of Unknown GS-Hainantoxin polypeptide
GSKCLPPGKPCYGATQKIPCCGVCSHNNCT

SEQ ID NO: 420 - protein, 13 aa, Unknown
  Description of Unknown Neurotensin peptide
ELYENKPRRPYIL

SEQ ID NO: 421 - protein, 34 aa, Unknown
  Description of Unknown Imperatoxin polypeptide
GSDCLPHLRRCRADNDCCGRRCRRRGTNAERRCR

SEQ ID NO: 422 - protein, 28 aa, Unknown
  Description of Unknown Conotoxin peptide
GSCRGRGQSCSRLMYDCCTGSCSRRGRC

SEQ ID NO: 423 - protein, 34 aa, Unknown
  Description of Unknown GSS-Hadrucalcin polypeptide
EKDCIKHLQRCRENKDCCSKKCSRRGTNPEKRCR

SEQ ID NO: 424 - protein, 4 aa, Artificial Sequence
  Description of Artificial Sequence Synthetic peptide
GAGA

SEQ ID NO: 425 - protein, 35 aa, Unknown
  Description of Unknown Scorpion or spider venom or toxin polypeptide
  MOD_RES (35)..(35): Any amino acid
MCIPCFTTNPNMAAKCNACCGSRRGSCRGPQCICX

SEQ ID NO: 426 - protein, 36 aa, Unknown
  Description of Unknown Scorpion or spider venom or toxin polypeptide
MCMPCFTTDHQMARKCDDCCGGKGRGKCYGPQCLCR

(19) United States
(12) Patent Application Publication
(10) Pub. No.: US 2018/0371033 A1
(43) Pub. Date: Dec. 27, 2018

(54) THERAPEUTIC PEPTIDES AND METHODS OF USE THEREOF

(71) Applicant: FRED HUTCHINSON CANCER RESEARCH CENTER, Seattle, WA (US)

(72) Inventors: James Olson, Seattle, WA (US); Andrew David Strand, Seattle, WA (US); Emily June Girard, Seattle, WA (US); Roland Strong, Seattle, WA (US); Christopher Mehlin, Seattle, WA (US); Colin Correnti, Seattle, WA (US); Andrew James Mhyre, Kenmore, WA (US); Mi-Youn Brusniak, Lynnwood, WA (US); Theo Sottero, Seattle, WA (US)

(73) Assignee: Fred Hutchinson Cancer Research Center, Seattle, WA (US)

(21) Appl. No.: 15/739,669

(22) PCT Filed: Jun. 24, 2016
(86) PCT No.: PCT/US16/39431

§ 371 Date: Dec. 22, 2017

Related U.S. Application Data
(60) Provisional application No. 62/185,527, filed on Jun. 26, 2015.
(60) Provisional application No. 62/185,529, filed on Jun. 26, 2015.
(60) Provisional application No. 62/239,743, filed on Oct. 9, 2015.
(60) Provisional application No. 62/239,739, filed on Oct. 9, 2015.
(60) Provisional application No. 62/322,724, filed on Apr. 14, 2016.
(60) Provisional application No. 62/354,642, filed on Jun. 24, 2016.

(51) Int. Cl. C07K 14/435; A61P 35/02; A61P 35/04; C07K 4/12; G01N 33/58; G01N 33/60; A61K 47/64

(52) U.S. Cl. CPC C07K 14/43518; A61P 35/02; A61P 35/04; C07K 4/12; A61K 38/04; G01N 33/582; G01N 33/60; A61K 47/64; G01N 33/587

(57) ABSTRACT

Peptides that home, distribute to, target, are directed to, or accumulate in tumors, cancers, or diseased cells are disclosed. Peptides that cross the blood brain barrier and home, distribute to, target, are directed to, or accumulate in the brain and in a specific region of the brain are also disclosed. Pharmaceutical compositions and uses for peptides or peptide-active agent complexes comprising such peptides are additionally disclosed. Such compositions can be formulated for targeted or untargeted delivery of a drug to a target region, tissue, structure or cell. Targeted compositions of the disclosure can deliver peptide or peptide-active agent complexes to target regions, tissues, structures or cells targeted by the peptide.

### CROSS-REFERENCE TO RELATED APPLICATIONS

This application is a U.S. National Phase Application under 35 U.S.C. § 371 of International Application No. PCT/US2016/039431, filed Jun. 24, 2016; which is related to U.S. provisional patent application No. 62/185,529, filed Jun. 26, 2015; U.S. provisional patent application No. 62/185,527, filed Jun. 26, 2015; U.S. provisional patent application No. 62/239,743, filed Oct. 9, 2015; U.S. provisional patent application No. 62/239,739, filed Oct. 9, 2015; U.S. provisional patent application No. 62/322,724, filed Apr. 14, 2016; and U.S. provisional patent application No. 62/354,642, filed Jun. 24, 2016, each of which are herein incorporated by reference in their entirety.

### SEQUENCE LISTING

The instant application contains a Sequence Listing which has been submitted electronically in ASCII format and is hereby incorporated by reference in its entirety. Said ASCII copy, created on Jul. 29, 2016, is named 44189-712_601_SL.txt and is 216,304 bytes in size.

### BACKGROUND

For many types of cancers, patient prognosis is directly influenced by the efficacy of drug therapies and surgical access to the tumor. In particular, the precision of tumor resection is dependent on intra-operative imaging to detect tumor margins or small foci of cancer cells. However, current methods of intra-operative imaging cancerous tissues are imprecise.

Of these types of cancers, brain disorders are particularly difficult to treat. The blood-brain barrier (BBB) can exclude over 97% of small molecules from entering the brain, and larger molecules such as antibodies are excluded almost universally. Usually, most molecules that enter the brain are small, lipophilic, and lack target specificity. Few drugs aimed at treating brain disorders have proved therapeutically viable with lack of access to target tissue being a primary reason for failure. In addition, many drugs that could gain access to the brain are ill-suited for treating brain conditions. The lack of access to the target tissue and lack of specificity also lead to administration of doses that are higher than would be necessary if a drug could home, target, or be directed to, a target region, tissue, structure or cell in the brain.

Similarly, other types of cancers, particularly solid tumors of several types, are difficult to treat as it is difficult to achieve a high enough level of effective drug into such tumors while managing side effects of the drugs in normal tissues. Consequently, there is a need for targeting drugs to solid tumors specifically to achieve a higher effective dose of drug in tumor while minimizing the level of side effects in other tissues. Moreover, there is also a need for targeting drugs specifically to any cancerous cell, whether from solid tumors or otherwise. Typical cancer drug regimens are often limited by dose-limiting toxicities, and although some antibody-drug conjugates are used to target drugs to specific tumors in order to limit off-site toxicity, such specific therapies are not available for many tumor types. Herein, we provide new peptides that target to tumors.

### SUMMARY

The present disclosure relates to compositions and methods for treatment of tumors. Described herein are peptides that home, distribute to, target, are directed to, accumulate in, migrate to, and/or bind to cancerous cells following administration to a subject. In some embodiments, the compositions and methods herein utilize peptides that home, distribute to, target, are directed to, accumulate in, migrate to, and/or bind to cancerous or diseased cells in the brain following administration to a subject. In some embodiments, the homing peptides of the present disclosure are used to deliver an active agent to a tissue or cell thereof.

In various aspects, the present disclosure provides a peptide comprising a sequence of any one of SEQ ID NO: 198-SEQ ID NO: 209 or SEQ ID NO: 407-SEQ ID NO: 418 or a fragment thereof.

In various aspects, the present disclosure provides a peptide comprising a sequence that has at least 80% sequence identity with any one of SEQ ID NO: 1-SEQ ID NO: 192 or SEQ ID NO: 210-SEQ ID NO: 401, or a fragment thereof. In some aspects, the peptide comprises the sequence that has at least 85%, at least 90%, or at least 95% sequence identity with any one of SEQ ID NO: 1-SEQ ID NO: 192 or SEQ ID NO: 210-SEQ ID NO: 401, or a fragment thereof. In other aspects, the peptide comprises a sequence that is any one of SEQ ID NO: 1-SEQ ID NO: 192 or SEQ ID NO: 210-SEQ ID NO: 401, or fragment thereof.

In various aspects, the present disclosure provides a peptide comprising a sequence of any one of SEQ ID NO: 198-SEQ ID NO: 209, or a fragment thereof.

In various aspects, the present disclosure provides a peptide comprising a sequence that has at least 80% sequence identity with any one of SEQ ID NO: 1-SEQ ID NO: 192, or a fragment thereof. In some aspects, the peptide comprises the sequence that has at least 85%, at least 90%, or at least 95% sequence identity with any one of SEQ ID NO: 1-SEQ ID NO: 192, or a fragment thereof. In other aspects, the peptide comprises a sequence that is any one of SEQ ID NO: 1-SEQ ID NO: 192, or fragment thereof.

In various aspects, the present disclosure provides a peptide comprising a sequence of any one of SEQ ID NO: 407-SEQ ID NO: 418, or a fragment thereof.

In various aspects, the present disclosure provides a peptide comprising a sequence that has at least 80% sequence identity with any one of SEQ ID NO: 210-SEQ ID NO: 401, or a fragment thereof. In some aspects, the peptide comprises the sequence that has at least 85%, at least 90%, or at least 95% sequence identity with any one of SEQ ID NO: 210-SEQ ID NO: 401, or a fragment thereof. In other aspects, the peptide comprises the sequence that is any one of SEQ ID NO: 210-SEQ ID NO: 401, or a fragment thereof.

In some aspects, any peptide of the present disclosure is a knotted peptide. In other aspects, the peptide comprises at least 6, at least 8, at least 10, at least 12, at least 14, or at least 16 cysteine residues. In some aspects, the peptide comprises a plurality of disulfide bridges formed between cysteine residues. In further aspects, at least 5% or more of the residues are cysteines forming intramolecular disulfide bonds. In some aspects, the peptide comprises a disulfide through disulfide knot.

In some aspects, at least one amino acid residue of the peptide is in an L configuration, or wherein at least one amino acid residue of the peptide is in a D configuration. In some aspects, the sequence is at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 residues long.

In some aspects, the peptide is arranged in a multimeric structure with at least one other peptide.

In some aspects, the peptide has a positive net charge greater than +0.5 at physiological pH. In other aspects, the peptide has a negative net charge lower than −0.5 at physiological pH.

In some aspects, upon administration to a subject, the peptide homes, targets, accumulates in, migrates to, or is directed to a specific region, tissue, structure, or cell of the subject.

In some aspects, at least one residue of the peptide comprises a chemical modification. In some aspects, the chemical modification is blocking the N-terminus of the peptide. In some aspects, the modification is methylation, acetylation, or acylation. In other aspects, the chemical modification is: methylation of one or more lysine residues or analogue thereof; methylation of an N-terminus; or methylation of one or more lysine residue or analogue thereof and methylation of the N-terminus. In some aspects, the peptide is linked to an acyl adduct.

In some aspects, the peptide is linked to an active agent. In further aspects, the active agent is fused with the peptide at an N-terminus or a C-terminus of the peptide. In some aspects, the active agent is a neurotensin peptide. In further aspects, the neurotensin peptide has a sequence of SEQ ID NO: 420. In still further aspects, the peptide fused to neurotensin peptide comprises a contiguous sequence. In some aspects, 1, 2, 3, 4, 5, 6, 7, 8, 9, or 10 active agents are linked to the peptide.

In some aspects, the peptide is linked to the active agent via a cleavable linker. In other aspects, the peptide is linked to the active agent at an N-terminus, at the epsilon amine of an internal lysine residue, at the carboxylic acid of an asparagine or glutamine residue, or a C-terminus of the peptide by a linker. In further aspects, the internal lysine residue is located at a position corresponding to amino acid residue 17 of SEQ ID NO: 37, amino acid residue 25 of SEQ ID NO: 37, or amino acid residue 29 of SEQ ID NO: 37. In other aspects, the internal lysine residue is located at a position corresponding to amino acid residue 15 of SEQ ID NO: 246, amino acid residue 23 of SEQ ID NO: 246, or amino acid residue 27 of SEQ ID NO: 246.

In other aspects, the peptide further comprises a non-natural amino acid, wherein the non-natural amino acid is an insertion, appendage, or substitution for another amino acid.

In some aspects, the peptide is linked to the active agent at the non-natural amino acid by a linker. In other aspects, the linker comprises an amide bond, an ester bond, a carbamate bond, a carbonate bond, a hydrazone bond, an oxime bond, a disulfide bond, a thioester bond, or a carbon-nitrogen bond. In further aspects, the cleavable linker comprises a cleavage site for matrix metalloproteinases, thrombin, cathepsins, or beta-glucuronidase. In some aspects, the peptide is linked to the active agent via a noncleavable linker.

In some aspects, the active agent is selected from the group consisting of: a peptide, a polypeptide, a polynucleotide, an antibody, a single chain variable fragment (scFv), an antibody fragment, a cytokine, a hormone, a growth factor, a checkpoint inhibitor, an immune modulator, a neurotransmitter, a chemical agent, a cytotoxic molecule, a toxin, a radiosensitizer, a radioprotectant, a therapeutic small molecule, a nanoparticle, a liposome, a polymer, a dendrimer, a fatty acid, peptidomimetic, a complement fixing peptide or protein, polyethylene glycol, a lipid, or an Fc region. In other aspects, the active agent is a polydeoxyribonucleotide or a polyribonucleotide sequence. In additional aspects, the active agent is an anti-inflammatory agent, an antifungal agent, an antiviral agent, or an anti-infective agent. In some aspects, the active agent is a chemotherapeutic agent. In other aspects, the active agent is a knotted peptide.

In still other aspects, the active agent is a radiosensitizer or photosensitizer. In some aspects, the cytotoxic molecule is an auristatin, MMAE, a maytansinoid, DM1, DM4, doxorubicin, a calicheamicin, a platinum compound, cisplatin, a taxane, paclitaxel, SN-38, a BACE inhibitor, a Bcl-xL inhibitor, WEHI-539, venetoclax, ABT-199, navitoclax, AT-101, obatoclax, a pyrrolobenzodiazepine or pyrrolobenzodiazepine dimer, or dolastatin.

In other aspects, the peptide is linked to a detectable agent. In further aspects, the detectable agent is fused with the peptide at an N-terminus or a C-terminus of the peptide. In still further, aspects, 1, 2, 3, 4, 5, 6, 7, 8, 9, or 10 detectable agents are linked to the peptide.

In some aspects, the peptide is linked to the detectable agent via a cleavable linker. In other aspects, the peptide is linked to the detectable agent at an N-terminus, at the epsilon amine of an internal lysine residue, or a C-terminus of the peptide by a linker. In further aspects, the internal lysine is located at a position corresponding to amino acid residue 17 of SEQ ID NO: 37, amino acid residue 25 of SEQ ID NO: 37, or amino acid residue 29 of SEQ ID NO: 37. In other aspects, the internal lysine residue is located at a position corresponding to amino acid residue 15 of SEQ ID NO: 246, amino acid residue 23 of SEQ ID NO: 246, or amino acid residue 27 of SEQ ID NO: 246.

In some aspects, the peptide further comprises a non-natural amino acid, wherein the non-natural amino acid is an insertion, appendage, or substitution for another amino acid.

In some aspects, the peptide is linked to the active agent at the non-natural amino acid by a linker. In other aspects, the linker comprises an amide bond, an ester bond, a carbamate bond, a hydrazone bond, an oxime bond, or a carbon-nitrogen bond. In further aspects, the cleavable linker comprises a cleavage site for matrix metalloproteinases, thrombin, cathepsins, or beta-glucuronidase. In some aspects, the peptide is linked to the detectable agent via a noncleavable linker.

In other aspects, the detectable agent is a fluorophore, a near-infrared dye, a contrast agent, a nanoparticle, a metal-containing nanoparticle, a metal chelate, an X-ray contrast agent, a PET agent, a radioisotope, or a radionuclide chelator. In some aspects, the detectable agent is a fluorescent dye.

In some aspects, the peptide homes, targets, is directed to, accumulates in, or migrates to a tumor or cancerous cell. In some aspects, the tumor is a solid tumor. In other aspects, the tumor is a hematologic malignancy. In further aspects, the peptide penetrates the solid tumor. In still further aspects, the peptide is internalized into or penetrates a cancerous cell. In some aspects, the tumor or cancerous cell is from a brain cancer, a glioblastoma, a colon cancer, a triple-negative breast cancer, metastatic cancer, or a sarcoma.

In some aspects, the peptide crosses a blood brain barrier to access the tumor. In other aspects, the peptide crosses a blood cerebral spinal fluid barrier to access the tumor.

In some aspects, the peptide crosses a blood brain barrier or a blood cerebral spinal fluid barrier of a subject. In other aspects. In other aspects, the peptide crosses a blood cerebrospinal fluid barrier of a subject.

In some aspects, the peptide homes, targets, is directed to, accumulates in, or migrates to a tumor or diseased region, tissue, structure, or cell of the subject after crossing the blood brain barrier.

In other aspects, upon administration to a subject the peptide homes, targets, is directed to, accumulates in, or migrates to a specific brain region of the subject. In further aspects, the specific region of the brain comprises the ventricles, the cerebrospinal fluid, the hippocampus, the meninges, the rostral migratory system, the dentate gyrus, the subventricular zone, or any combination thereof.

In some aspects, the peptide affects neurological disorders, lysosomal storage diseases, epilepsy, meningitis, infections in the brain, stroke, and multiple sclerosis. In some aspects, the peptide affects aggregation of a protein associated with a neurodegenerative disease. In other aspects, the peptide inhibits a pathway associated with brain cancer. In still other aspects, the peptide inhibits or activates ion channels. In some aspects, the peptide exhibits protease inhibitor activity. In other aspects, the peptide has antibacterial, antifungal, or antiviral activity.

In various aspects, the present disclosure provides a pharmaceutical composition comprising a peptide of this disclosure or a salt thereof, and a pharmaceutically acceptable carrier. In some aspects, the pharmaceutical composition is formulated for administration to a subject. In further aspects, the pharmaceutical composition is formulated for inhalation, intranasal administration, oral administration, topical administration, intravenous administration, subcutaneous administration, intra-articular administration, intramuscular administration, intrathecal, intraperitoneal administration, or a combination thereof.

In various aspects, the present disclosure provides a method of treating a condition in a subject in need thereof, the method comprising administering to the subject a peptide or a pharmaceutical composition of this disclosure. In some aspects, the peptide or pharmaceutical composition is administered by inhalation, intranasally, orally, topically, intravenously, subcutaneously, intra-articularly, intramuscularly administration, intraperitoneally, or a combination thereof.

In some aspects, the peptide or pharmaceutical composition of the method. In some aspects, the condition is a tumor or cancer. In further aspects, the condition is a solid tumor. In some aspects, the tumor is a hematologic malignancy. In other aspects, the condition is a brain tumor, triple-negative breast cancer, colon cancer metastases, metastatic cancer or sarcoma. In further aspects, the brain tumor is inoperable.

In some aspects, the peptide of the method crosses a blood brain barrier to home, target, migrate to, accumulate in, or get directed to the tumor in the brain. In some aspects, the peptide crosses a blood cerebrospinal fluid barrier to home, target, migrate to, accumulate in, or get directed to the tumor in the brain.

In some aspects, the method is combined with other treatments. In further aspects, the other treatments comprise chemotherapy, radiation therapy, or immunomodulatory therapy.

In some aspects, the peptide of the method crosses the blood brain barrier of the subject following administration. In other aspects, the peptide crosses the blood cerebrospinal fluid barrier of the subject following administration.

In some aspects, the peptide of the method homes, targets, is directed to, accumulates in, or migrates to the ventricles, cerebrospinal fluid, meninges, rostral migratory system, or hippocampus of the subject following administration. In some aspects, the condition is a brain condition. In other aspects, the condition is associated with a function of the ventricles, cerebrospinal fluid, or hippocampus. In further aspects, the brain condition is associated with a function of the brain.

In some aspects, the peptide of the method diagnoses, prevents, or treats the brain condition. In further aspects, the brain condition is a brain tumor or brain cancer. In other aspects, the brain condition is memory loss or memory function, Alzheimer's disease, Parkinson's disease, multiple system atrophy (MSA), schizophrenia, epilepsy, progressive multifocal leukoencephalopathy, fungal infection, depression, bipolar disorder, post-traumatic stress disorder, stroke, traumatic brain injury, infection, or multiple sclerosis.

In various aspects, the present disclosure provides a method of imaging an organ or body region of a subject, the method comprising administering to the subject a peptide or pharmaceutical composition of this disclosure and imaging the organ or body region of the subject.

In some aspects, the method comprises detecting a cancer or diseased region, tissue, structure or cell of the subject. In further aspects, the method comprises performing surgery on the subject. In still further aspects, the method comprises treating the cancer.

In some aspects, the surgery of the method comprises removing the cancer or the diseased region, tissue, structure or cell of the subject. In further aspects, the method comprises imaging the cancer or diseased region, tissue, structure, or cell of the subject after surgical removal.

### INCORPORATION BY REFERENCE

All publications, patents, and patent applications mentioned, disclosed or referenced in this specification are herein incorporated by reference in their entirety and to the same extent as if each individual publication, patent, or patent application was specifically and individually indicated to be incorporated by reference.

### BRIEF DESCRIPTION OF THE FIGURES

The novel features of the invention are set forth with particularity in the appended claims.

A better understanding of the features and advantages of the present invention will be obtained by reference to the following detailed description that sets forth illustrative embodiments, in which the principles of the invention are utilized, and the accompanying drawings of which:

FIG. 1 illustrates a peptide that was radiolabeled by methylating the lysines. FIG. 1A illustrates a native lysine and FIG. 1B illustrates a dimethylated lysine.

FIG. 2 illustrates ¹⁴C signal in the brain and other tissues for the fluoxetine (top) and inulin (bottom) control groups.

FIG. 3 illustrates ¹⁴C signal in the brain and other tissues for radiolabeled peptides of SEQ ID NO: 1.

FIG. 4 illustrates ¹⁴C signal in the brain and other tissues for radiolabeled peptides of SEQ ID NO: 3.

FIG. 5 illustrates the HPLC profile of a peptide of SEQ ID NO: 1.

FIG. 6 illustrates an overlay of the HPLC profiles for both a nonreduced and a reduced sample of a peptide of SEQ ID NO: 2.

FIG. 7 illustrates an overlay of the HPLC profiles for both a nonreduced and a reduced sample of a peptide of SEQ ID NO: 3.

FIG. 8 illustrates the HPLC profile of a peptide of SEQ ID NO: 4.

FIG. 9 illustrates an exemplary architecture of constructs expressing SEQ ID NO: 1 through SEQ ID: NO. 4.

FIG. 10 illustrates a schematic of a method of manufacturing of a peptide of the disclosure.

FIG. 11 illustrates quality control data from small scale expression runs of peptides of SEQ ID NO: 4 (FIG. 11A), SEQ ID NO: 6 (FIG. 11B), SEQ ID NO: 17 (FIG. 11C), SEQ ID NO: 25 (FIG. 11D), and SEQ ID NO: 32 (FIG. 11E).

FIG. 12 illustrates HPLC data and non-reduced compared to reduced bands on SDS-PAGE gels of SEQ ID NO: 39 peptide, and MALDI mass spectrometry graphs of SEQ ID NO: 25 peptide.

FIG. 12A illustrates an HPLC profile of SEQ ID NO: 39.

FIG. 12B illustrates the nonreduced and reduced bands of SEQ ID NO: 39 on an SDS-PAGE gel.

FIG. 12C shows the full spectra of a MALDI mass spectrometry graph of SEQ ID NO: 25.

FIG. 12D shows a zoomed-in portion of the full spectra of a MALDI mass spectrometry graph of SEQ ID NO: 25.

FIG. 13 illustrates murine white light and corresponding autoradiographic images three hours after administration of 9 nmol of the radiolabeled peptide of SEQ ID NO: 5 conjugated to Alexa 647 fluorescent dye (SEQ ID NO: 5-RA peptide conjugate).

FIG. 13A illustrates a white light image of a frozen section of a mouse three hours after administration of 9 nmol of radiolabeled peptide of SEQ ID NO: 5 conjugated to Alexa 647 fluorescent dye (SEQ ID NO: 5-RA peptide conjugate).

FIG. 13B illustrates an autoradiographic image corresponding to FIG. 13A in which the ¹⁴C signal identifies the peptide distribution in the tissues, including the RH-28 tumor of a mouse, three hours after administration of 9 nmol of the radiolabeled peptide of SEQ ID NO: 5 conjugated to Alexa 647 fluorescent dye (SEQ ID NO: 5-RA peptide conjugate).

FIG. 13C illustrates a white light image of a different frozen section of the same mouse as in FIG. 13A and FIG. 13B three hours after administration of 9 nmol of the radiolabeled peptide of SEQ ID NO: 5 conjugated to Alexa 647 fluorescent dye (SEQ ID NO: 5-RA peptide conjugate).

FIG. 13D illustrates an autoradiographic image corresponding to FIG. 13C in which the ¹⁴C signal identifies the peptide distribution in the tissues, including RH-28 tumor, three hours after administration of 9 nmol of the radiolabeled peptide of SEQ ID NO: 5 conjugated to Alexa 647 fluorescent dye (SEQ ID NO: 5-RA peptide conjugate).

FIG. 13E illustrates a white light image of a frozen section of a different mouse than shown in FIG. 13A through FIG. 13D three hours after administration of 9 nmol of the radiolabeled peptide of SEQ ID NO: 5 conjugated to Alexa 647 fluorescent dye (SEQ ID NO: 5-RA peptide conjugate).

FIG. 13F illustrates an autoradiographic image corresponding to FIG. 13E in which the ¹⁴C signal identifies the peptide distribution in the tissues, including the RH-28 tumor, of a mouse three hours after administration of 9 nmol of the radiolabeled peptide of SEQ ID NO: 5 conjugated to Alexa 647 fluorescent dye (SEQ ID NO: 5-RA peptide conjugate).

FIG. 13G illustrates a white light image of a different frozen section of the same mouse as in FIG. 13E and FIG. 13F three hours after administration of 9 nmol of the radiolabeled peptide of SEQ ID NO: 5 conjugated to Alexa 647 fluorescent dye (SEQ ID NO: 5-RA peptide conjugate).

FIG. 13H illustrates an autoradiographic image corresponding to FIG. 13G in which the ¹⁴C signal identifies the peptide distribution in the tissues, including the RH-28 tumor, three hours after administration of 9 nmol of the radiolabeled peptide of SEQ ID NO: 5 conjugated to Alexa 647 fluorescent dye (SEQ ID NO: 5-RA peptide conjugate).

FIG. 14 illustrates murine white light and corresponding autoradiographic images twenty-four hours after administration of 9 nmol of the radiolabeled peptide of SEQ ID NO: 5 conjugated to Alexa 647 fluorescent dye (SEQ ID NO: 5-RA peptide conjugate).

FIG. 14A illustrates a white light image of a frozen section of a mouse twenty-four hours after administration of 9 nmol of the radiolabeled peptide of SEQ ID NO: 5 conjugated to Alexa 647 fluorescent dye (SEQ ID NO: 5-RA peptide conjugate).

FIG. 14B illustrates an autoradiographic image corresponding to FIG. 14A in which the ¹⁴C signal identifies the peptide distribution in the tissues, including the RH-28 tumor, of a mouse twenty-four hours after administration of 9 nmol of the radiolabeled peptide of SEQ ID NO: 5 conjugated to Alexa 647 fluorescent dye (SEQ ID NO: 5-RA peptide conjugate).

FIG. 14C illustrates a white light image of a different frozen section of the same mouse as in FIG. 14A and FIG. 14B twenty-four hours after administration of 9 nmol of the radiolabeled peptide of SEQ ID NO: 5 conjugated to Alexa 647 fluorescent dye (SEQ ID NO: 5-RA peptide conjugate).

FIG. 14D illustrates an autoradiographic image corresponding to FIG. 14C in which the ¹⁴C signal identifies the peptide distribution in the tissues, including the RH-28 tumor, twenty-four hours after administration of 9 nmol of the radiolabeled peptide of SEQ ID NO: 5 conjugated to Alexa 647 fluorescent dye (SEQ ID NO: 5-RA peptide conjugate).

FIG. 14E illustrates a white light image of a frozen section of a different mouse than shown in FIG. 14A through FIG. 14D twenty-four hours after administration of 9 nmol of the radiolabeled peptide of SEQ ID NO: 5 conjugated to Alexa 647 fluorescent dye (SEQ ID NO: 5-RA peptide conjugate).

FIG. 14F illustrates an autoradiographic image corresponding to FIG. 14E in which the ¹⁴C signal identifies the peptide distribution in the tissues, including the RH-28 tumor, of a mouse twenty-four hours after administration of 9 nmol of the radiolabeled peptide of SEQ ID NO: 5 conjugated to Alexa 647 fluorescent dye (SEQ ID NO: 5-RA peptide conjugate).

FIG. 14G illustrates a white light image of a different frozen section of the same mouse as in FIG. 14E and FIG. 14F twenty-four hours after administration of 9 nmol of the radiolabeled peptide of SEQ ID NO: 5 conjugated to Alexa 647 fluorescent dye (SEQ ID NO: 5-RA peptide conjugate).

FIG. 14H illustrates an autoradiographic image corresponding to FIG. 14G in which the ¹⁴C signal identifies the peptide distribution in the tissues, including RH-28 tumor, twenty-four hours after administration of 9 nmol of the radiolabeled peptide of SEQ ID NO: 5 conjugated to Alexa 647 fluorescent dye (SEQ ID NO: 5-RA peptide conjugate).

FIG. 15 illustrates murine white light and corresponding autoradiographic images three hours after administration of 11 nmol of the radiolabeled peptide of SEQ ID NO: 5 conjugated to MMAE via a valine-citrulline linker (SEQ ID NO: 5-RZ peptide conjugate).

FIG. 15A illustrates a white light image of a frozen section of a mouse three hours after administration of 11 nmol of the radiolabeled peptide of SEQ ID NO: 5 conjugated to MMAE with a valine-citrulline linker (SEQ ID NO: 5-RZ peptide conjugate)

FIG. 15B illustrates an autoradiographic image corresponding to FIG. 15A in which the ¹⁴C signal identifies the peptide distribution in the tissues, including the RH-28 tumor, of a mouse three hours after administration of 11 nmol of the radiolabeled peptide of SEQ ID NO: 5 conjugated to MMAE via a valine-citrulline linker (SEQ ID NO: 5-RZ peptide conjugate).

FIG. 15C illustrates a white light image of a different frozen section of the same mouse as in FIG. 15A and FIG. 15B three hours after administration of 11 nmol of the radiolabeled peptide of SEQ ID NO: 5 conjugated to MMAE via a valine-citrulline linker (SEQ ID NO: 5-RZ peptide conjugate).

FIG. 15D illustrates an autoradiographic image corresponding to FIG. 15C in which the ¹⁴C signal identifies the peptide distribution in the tissues, including the RH-28 tumor, three hours after administration of 11 nmol of the radiolabeled peptide of SEQ ID NO: 5 conjugated to MMAE via a valine-citrulline linker (SEQ ID NO: 5-RZ peptide conjugate).

FIG. 15E illustrates a white light image of a frozen section of a different mouse than shown in FIG. 15A through FIG. 15D three hours after administration of 11 nmol of the radiolabeled peptide of SEQ ID NO: 5 conjugated to MMAE via a valine-citrulline linker (SEQ ID NO: 5-RZ peptide conjugate).

FIG. 15F illustrates an autoradiographic image corresponding to FIG. 15E in which the ¹⁴C signal identifies the peptide distribution in the tissues, including the RH-28 tumor, of a mouse three hours after administration of 11 nmol of the radiolabeled peptide of SEQ ID NO: 5 conjugated to MMAE via a valine-citrulline linker (SEQ ID NO: 5-RZ peptide conjugate).

FIG. 15G illustrates a white light image of a different frozen section of the same mouse as in FIG. 15E and FIG. 15F three hours after administration of 11 nmol of the radiolabeled peptide of SEQ ID NO: 5 conjugated to MMAE via a valine-citrulline linker (SEQ ID NO: 5-RZ peptide conjugate).

FIG. 15H illustrates an autoradiographic image corresponding to FIG. 15G in which the ¹⁴C signal identifies the peptide distribution in the tissue, including the RH-28 tumor, three hours after administration of 11 nmol of the radiolabeled peptide of SEQ ID NO: 5 conjugated to MMAE via a valine-citrulline linker (SEQ ID NO: 5-RZ peptide conjugate).

FIG. 16 illustrates murine white light and corresponding autoradiographic images twenty-four hours after administration of 11 nmol of the radiolabeled peptide of SEQ ID NO: 5 conjugated to MMAE via a valine-citrulline linker (SEQ ID NO: 5-RZ peptide conjugate).

FIG. 16A illustrates a white light image of a frozen section of a mouse twenty-four hours after administration of 11 nmol of the radiolabeled peptide of SEQ ID NO: 5 conjugated to MMAE via a valine-citrulline linker (SEQ ID NO: 5-RZ peptide conjugate).

FIG. 16B illustrates an autoradiographic image corresponding to FIG. 16A in which the ¹⁴C signal identifies the peptide distribution in the tissue, including RH-28 tumor, of a mouse twenty-four hours after administration of 11 nmol of the radiolabeled peptide of SEQ ID NO: 5 conjugated to MMAE via a valine-citrulline linker (SEQ ID NO: 5-RZ peptide conjugate).

FIG. 16C illustrates a white light image of a different frozen section of the same mouse as in FIG. 16A and FIG. 16B twenty-four hours after administration of 11 nmol of the radiolabeled peptide of SEQ ID NO: 5 conjugated to MMAE via a valine-citrulline linker (SEQ ID NO: 5-Z peptide conjugate).

FIG. 16D illustrates an autoradiographic image corresponding to FIG. 16C in which the ¹⁴C signal identifies the peptide distribution in the tissues, including RH-28 tumor, twenty-four hours after administration of 11 nmol of the radiolabeled peptide of SEQ ID NO: 5 conjugated to MMAE via a valine-citrulline linker (SEQ ID NO: 5-RZ peptide conjugate).

FIG. 16E illustrates a white light image of a frozen section of a different mouse than shown in FIG. 16A through FIG. 16D twenty-four hours after administration of 11 nmol of the radiolabeled peptide of SEQ ID NO: 5 conjugated to MMAE via a valine-citrulline linker (SEQ ID NO: 5-RZ peptide conjugate).

FIG. 16F illustrates an autoradiographic image corresponding to FIG. 16E in which the ¹⁴C signal identifies the peptide distribution in the tissues, including the RH-28 tumor, of a mouse twenty-four hours after administration of 11 nmol of the radiolabeled peptide of SEQ ID NO: 5 conjugated to MMAE via a valine-citrulline linker (SEQ ID NO: 5-RZ peptide conjugate).

FIG. 16G illustrates a white light image of a different frozen section of the same mouse as in FIG. 16E and FIG. 16F twenty-four hours after administration of 11 nmol of the radiolabeled peptide of SEQ ID NO: 5 conjugated to MMAE via a valine-citrulline linker (SEQ ID NO: 5-RZ peptide conjugate).

FIG. 16H illustrates an autoradiographic image corresponding to FIG. 16G in which the ¹⁴C signal identifies the peptide distribution in the tissues, including the RH-28 tumor, twenty-four hours after administration of 11 nmol of the radiolabeled peptide of SEQ ID NO: 5 conjugated to MMAE via a valine-citrulline linker (SEQ ID NO: 5-RZ peptide conjugate).

FIG. 17 illustrates murine white light and corresponding autoradiographic images three hours after administration of 12.8 nmol of the radiolabeled peptide of SEQ ID NO: 5 peptide (SEQ ID NO: 5-R peptide).

FIG. 17A illustrates a white light image of a frozen section of a mouse three hours after administration of 12.8 nmol of the radiolabeled peptide of SEQ ID NO: 5 peptide (SEQ ID NO: 5-R peptide).

FIG. 17B illustrates an autoradiographic image corresponding to FIG. 17A in which the ¹⁴C signal identifies the peptide distribution in the tissues, including the RH-28 tumor, of a mouse three hours after administration of 12.8 nmol of the radiolabeled peptide of SEQ ID NO: 5 (SEQ ID NO: 5-R peptide).

FIG. 17C illustrates a white light image of a different frozen section of the same mouse as in FIG. 17A and FIG. 17B three hours after administration of 12.8 nmol of the radiolabeled peptide of SEQ ID NO: 5 (SEQ ID NO: 5-R peptide).

FIG. 17D illustrates an autoradiographic image corresponding to FIG. 17C in which the ¹⁴C signal identifies the peptide distribution in the tissues, including the RH-28 tumor, three hours after administration of 12.8 nmol of the radiolabeled peptide of SEQ ID NO: 5 (SEQ ID NO: 5-R peptide).

FIG. 17E illustrates a white light image of a frozen section of a different mouse than shown in FIG. 17A through FIG. 17D three hours after administration of 12.8 nmol of the radiolabeled peptide of SEQ ID NO: 5 (SEQ ID NO: 5-R peptide).

FIG. 17F illustrates an autoradiographic image corresponding to FIG. 17E in which the ¹⁴C signal identifies the peptide distribution in the tissues, including the RH-28 tumor, of a mouse three hours after administration of 12.8 nmol of the radiolabeled peptide of SEQ ID NO: 5 (SEQ ID NO: 5-R peptide).

FIG. 17G illustrates a white light image of a different frozen section of the same mouse as in FIG. 17E and FIG. 17F three hours after administration of 12.8 nmol of the radiolabeled peptide of SEQ ID NO: 5 (SEQ ID NO: 5-R peptide).

FIG. 17H illustrates an autoradiographic image corresponding to FIG. 17G in which the ¹⁴C signal identifies the peptide distribution in the tissues, including the RH-28 tumor, three hours after administration of 12.8 nmol of the radiolabeled peptide of SEQ ID NO: 5 (SEQ ID NO: 5-R peptide).

FIG. 18 illustrates murine white light and corresponding autoradiographic images three hours after administration of 14 nmol of the radiolabeled peptide of SEQ ID NO: 5 conjugated to DM-1 via a non-cleavable linker (SEQ ID NO: 5-RY peptide conjugate).

FIG. 18A illustrates a white light image of a frozen section of a mouse three hours after administration of 14 nmol of the radiolabeled peptide of SEQ ID NO: 5 conjugated to DM-1 via a non-cleavable linker (SEQ ID NO: 5-RY peptide conjugate).

FIG. 18B illustrates an autoradiographic image corresponding to FIG. 18A in which the ¹⁴C signal identifies the peptide distribution in the tissue, including the RH-28 tumor, of a mouse three hours after administration of 14 nmol of the radiolabeled peptide of SEQ ID NO: 5 conjugated to DM-1 via a non-cleavable linker (SEQ ID NO: 5-RY peptide conjugate).

FIG. 18C illustrates a white light image of a different frozen section of the same mouse as in FIG. 18A and FIG. 18B three hours after administration of 14 nmol of the radiolabeled peptide of SEQ ID NO: 5 conjugated to DM-1 via a non-cleavable linker (SEQ ID NO: 5-RY peptide conjugate).

FIG. 18D illustrates an autoradiographic image corresponding to FIG. 18C in which the ¹⁴C signal identifies the peptide distribution in the tissues, including the RH-28 tumor, three hours after administration of 14 nmol of the radiolabeled peptide of SEQ ID NO: 5 conjugated to DM-1 via a non-cleavable linker (SEQ ID NO: 5-RY peptide conjugate).

FIG. 18E illustrates a white light image of a frozen section of a different mouse than shown in FIG. 18A through FIG. 18D three hours after administration of 14 nmol of the radiolabeled peptide of SEQ ID NO: 5 conjugated to DM-1 via a non-cleavable linker (SEQ ID NO: 5-RY peptide conjugate).

FIG. 18F illustrates an autoradiographic image corresponding to FIG. 18E in which the ¹⁴C signal identifies the peptide distribution in the tissues, including the RH-28 tumor, of a mouse three hours after administration of 14 nmol of the radiolabeled peptide of SEQ ID NO: 5 conjugated to DM-1 via a non-cleavable linker (SEQ ID NO: 5-RY peptide conjugate).

FIG. 18G illustrates a white light image of a different frozen section of the same mouse as in FIG. 18E and FIG. 18F three hours after administration of 14 nmol of the radiolabeled peptide of SEQ ID NO: 5 conjugated to DM-1 via a non-cleavable linker (SEQ ID NO: 5-RY peptide conjugate).

FIG. 18H illustrates an autoradiographic image corresponding to FIG. 18G in which the ¹⁴C signal identifies the peptide distribution in the tissues, including the RH-28 tumor, three hours after administration of 14 nmol of the radiolabeled peptide of SEQ ID NO: 5 conjugated to DM-1 via a non-cleavable linker (SEQ ID NO: 5-RY peptide conjugate).

FIG. 19 illustrates murine white light and corresponding autoradiographic images twenty-four hours after administration of 14 nmol of the radiolabeled peptide of SEQ ID NO: 5 conjugated to DM-1 via a non-cleavable linker (SEQ ID NO: 5-RY peptide conjugate).

FIG. 19A illustrates a white light image of a frozen section of a mouse twenty-four hours after administration of 14 nmol of the radiolabeled peptide of SEQ ID NO: 5 conjugated to DM-1 via a non-cleavable linker (SEQ ID NO: 5-RY peptide conjugate).

FIG. 19B illustrates an autoradiographic image corresponding to FIG. 19A in which the ¹⁴C signal identifies the peptide distribution in the tissues, including the RH-28 tumor, of a mouse twenty-four hours after administration of 14 nmol of the radiolabeled peptide of SEQ ID NO: 5 conjugated to DM-1 via a non-cleavable linker (SEQ ID NO: 5-RY peptide conjugate).

FIG. 19C illustrates a white light image of a different frozen section of the same mouse as in FIG. 19A and FIG. 19B twenty-four hours after administration of 14 nmol of the radiolabeled peptide of SEQ ID NO: 5 conjugated to DM-1 via a non-cleavable linker (SEQ ID NO: 5-RY peptide conjugate).

FIG. 19D illustrates an autoradiographic image corresponding to FIG. 19C in which the ¹⁴C signal identifies the peptide distribution in the tissues, including the RH-28 tumor, twenty-four hours after administration of 14 nmol of the radiolabeled peptide of SEQ ID NO: 5 conjugated to DM-1 via a non-cleavable linker (SEQ ID NO: 5-RY peptide conjugate).

FIG. 19E illustrates a white light image of a frozen section of a different mouse than shown in FIG. 19A through FIG. 19D twenty-four hours after administration of 14 nmol of the radiolabeled peptide of SEQ ID NO: 5 conjugated to DM-1 via a non-cleavable linker (SEQ ID NO: 5-RY peptide conjugate).

FIG. 19F illustrates an autoradiographic image corresponding to FIG. 19E in which the ¹⁴C signal identifies the peptide distribution in the tissues, including the RH-28 tumor, of a mouse twenty-four hours after administration of 14 nmol of the radiolabeled peptide of SEQ ID NO: 5 conjugated to DM-1 via a non-cleavable linker (SEQ ID NO: 5-RY peptide conjugate).

FIG. 19G illustrates a white light image of a different frozen section of the same mouse as in FIG. 19E and FIG. 19F twenty-four hours after administration of 14 nmol of the radiolabeled peptide of SEQ ID NO: 5 conjugated to DM-1 via a non-cleavable linker (SEQ ID NO: 5-RY peptide conjugate).

FIG. 19H illustrates an autoradiographic image corresponding to FIG. 19G in which the ¹⁴C signal identifies the peptide distribution in the tissues, including the RH-28 tumor, twenty-four hours after administration of 14 nmol of the radiolabeled peptide of SEQ ID NO: 5 conjugated to DM-1 via a non-cleavable linker (SEQ ID NO: 5-RY peptide conjugate).

FIG. 20 illustrates white light and corresponding autoradiographic images from mice with intact kidneys 3 hours after administration of 100 nmol of the radiolabeled SEQ ID NO: 37 peptide.

FIG. 20A illustrates a white light image of a frozen section of a mouse with intact kidneys 3 hours after administration of 100 nmol of the radiolabeled SEQ ID NO: 37 peptide.

FIG. 20B illustrates an autoradiographic image corresponding to FIG. 20A in which the ¹⁴C signal identifies the peptide distribution in the tissues of a mouse with intact kidneys 3 hours after administration of 100 nmol of the radiolabeled SEQ ID NO: 37 peptide.

FIG. 20C illustrates a white light image of a different frozen section of the same mouse as in FIG. 20A and FIG. 20B 3 hours after administration of 100 nmol of the radiolabeled SEQ ID NO: 37 peptide.

FIG. 20D illustrates an autoradiographic image corresponding to FIG. 20C in which the ¹⁴C signal identifies the peptide distribution in the tissues of the mouse 3 hours after administration of 100 nmol of the radiolabeled SEQ ID NO: 37 peptide.

FIG. 20E illustrates a white light image of a different frozen section of the same mouse as in FIG. 20A through FIG. 20D 3 hours after administration of 100 nmol of the radiolabeled SEQ ID NO: 37 peptide.

FIG. 20F illustrates an autoradiographic image corresponding to FIG. 20E in which the ¹⁴C signal identifies the peptide distribution in tissues of the mouse 3 hours after administration of 100 nmol of the radiolabeled SEQ ID NO: 37 peptide.

FIG. 20G illustrates a white light image of a frozen section of a different mouse with intact kidneys than shown in FIG. 20A through FIG. 20F 3 hours after administration of 100 nmol of the radiolabeled SEQ ID NO: 37 peptide.

FIG. 20H illustrates an autoradiographic image corresponding to FIG. 20G in which the ¹⁴C signal identifies the peptide distribution in the tissues of a mouse 3 hours after administration of 100 nmol of the radiolabeled SEQ ID NO: 37 peptide.

FIG. 20I illustrates a white light image of a different frozen section of the same mouse as in FIG. 20G and FIG. 20H 3 hours after administration of 100 nmol of the radiolabeled SEQ ID NO: 37 peptide.

FIG. 20J illustrates an autoradiographic image corresponding to FIG. 20I in which the ¹⁴C signal identifies the peptide distribution in the tissues of the mouse 3 hours after administration of 100 nmol of the radiolabeled SEQ ID NO: 37 peptide.

FIG. 20K illustrates a white light image of a different frozen section of the same mouse as in FIG. 20G through FIG. 20J 3 hours after administration of 100 nmol of the radiolabeled SEQ ID NO: 37 peptide.

FIG. 20L illustrates an autoradiographic image corresponding to FIG. 20K in which the ¹⁴C signal identifies the peptide distribution in the mouse 3 hours after administration of 100 nmol of the radiolabeled SEQ ID NO: 37 peptide.

FIG. 21 illustrates white light and corresponding autoradiographic images from mice with ligated kidneys 3 hours after administration of 100 nmol of the radiolabeled SEQ ID NO: 37 peptide.

FIG. 21A illustrates a white light image of a frozen section of a mouse with ligated kidneys 3 hours after administration of 100 nmol of the radiolabeled SEQ ID NO: 37 peptide.

FIG. 21B illustrates an autoradiographic image corresponding to FIG. 21A in which the ¹⁴C signal identifies the peptide distribution in the tissues of the mouse with ligated kidneys 3 hours after administration of 100 nmol of the radiolabeled SEQ ID NO: 37 peptide.

FIG. 21C illustrates a white light image of a different frozen section of the same mouse as in FIG. 21A and FIG. 21B 3 hours after administration of 100 nmol of the radiolabeled SEQ ID NO: 37 peptide.

FIG. 21D illustrates an autoradiographic image corresponding to FIG. 21C in which the ¹⁴C signal identifies the peptide distribution in the tissues of the mouse 3 hours after administration of 100 nmol of the radiolabeled SEQ ID NO: 37 peptide.

FIG. 21E illustrates a white light image of a frozen section of a different mouse with ligated kidneys than shown in FIG. 21A through FIG. 21D 3 hours after administration of 100 nmol of the radiolabeled SEQ ID NO: 37 peptide.

FIG. 21F illustrates an autoradiographic image corresponding to FIG. 21E in which the ¹⁴C signal identifies the peptide distribution in the tissues of the mouse 3 hours after administration of 100 nmol of the radiolabeled SEQ ID NO: 37 peptide.

FIG. 21G illustrates a white light image of a different frozen section of the same mouse as in FIG. 21E and FIG. 21F 3 hours after administration of 100 nmol of the radiolabeled SEQ ID NO: 37 peptide.

FIG. 21H illustrates an autoradiographic image corresponding to FIG. 21G in which the ¹⁴C signal identifies the peptide distribution in the tissues of the mouse 3 hours after administration of 100 nmol of the radiolabeled SEQ ID NO: 37 peptide.

FIG. 22 illustrates white light and corresponding autoradiographic images from mice with intact kidneys 3 hours after administration of 100 nmol of the radiolabeled SEQ ID NO: 35 peptide.

FIG. 22A illustrates a white light image of a frozen section of a mouse with intact kidneys 3 hours after administration of 100 nmol of the radiolabeled SEQ ID NO: 35 peptide.

FIG. 22B illustrates an autoradiographic image corresponding to FIG. 22A in which the ¹⁴C signal identifies the peptide distribution in the tissues of the mouse with intact kidneys 3 hours after administration of 100 nmol of the radiolabeled SEQ ID NO: 35 peptide.

FIG. 22C illustrates a white light image of a different frozen section of the same mouse as in FIG. 22A and FIG. 22B 3 hours after administration of 100 nmol of the radiolabeled SEQ ID NO: 35 peptide.

FIG. 22D illustrates an autoradiographic image corresponding to FIG. 22C in which the ¹⁴C signal identifies the peptide distribution in the tissues of the mouse 3 hours after administration of 100 nmol of the radiolabeled SEQ ID NO: 35 peptide.

FIG. 22E illustrates a white light image of a frozen section of a different mouse with intact kidneys than shown in FIG. 22A through FIG. 22D 3 hours after administration of 100 nmol of the radiolabeled SEQ ID NO: 35 peptide.

FIG. 22F illustrates an autoradiographic image corresponding to FIG. 22E in which the ¹⁴C signal identifies the peptide distribution in the tissues of the mouse 3 hours after administration of 100 nmol of the radiolabeled SEQ ID NO: 35 peptide.

FIG. 22G illustrates a white light image of a different frozen section of the same mouse as in FIG. 22E and FIG. 22F 3 hours after administration of 100 nmol of the radiolabeled SEQ ID NO: 35 peptide.

FIG. 22H illustrates an autoradiographic image corresponding to FIG. 22G in which the ¹⁴C signal identifies the peptide distribution in the tissues of the mouse 3 hours after administration of 100 nmol of the radiolabeled SEQ ID NO: 35 peptide.

FIG. 23 illustrates white light and corresponding autoradiographic images from mice with ligated kidneys 3 hours after administration of 100 nmol of the radiolabeled SEQ ID NO: 35 peptide.

FIG. 23A illustrates a white light image of a frozen section of a mouse with ligated kidneys 3 hours after administration of 100 nmol of the radiolabeled SEQ ID NO: 35 peptide.

FIG. 23B illustrates an autoradiographic image corresponding to FIG. 23A in which the ¹⁴C signal identifies the peptide distribution in the tissues of the mouse with ligated kidneys 3 hours after administration of 100 nmol of the radiolabeled SEQ ID NO: 35 peptide.

FIG. 23C illustrates a white light image of a different frozen section of the same mouse as in FIG. 23A and FIG. 23B 3 hours after administration of 100 nmol of the radiolabeled SEQ ID NO: 35 peptide.

FIG. 23D illustrates an autoradiographic image corresponding to FIG. 23C in which the ¹⁴C signal identifies the peptide distribution in the tissues of the mouse 3 hours after administration of 100 nmol of the radiolabeled SEQ ID NO: 35 peptide.

FIG. 23E illustrates a white light image of a frozen section of a different mouse with ligated kidneys than shown in FIG. 23A through FIG. 23D 3 hours after administration of 100 nmol of the radiolabeled SEQ ID NO: 35 peptide.

FIG. 23F illustrates an autoradiographic image corresponding to FIG. 23E in which the ¹⁴C signal identifies the peptide distribution in the tissues of the mouse 3 hours after administration of 100 nmol of the SEQ ID NO: 35 peptide.

FIG. 23G illustrates a white light image of a different frozen section of the same mouse as in FIG. 23E and FIG. 23F 3 hours after administration of 100 nmol of the radiolabeled SEQ ID NO: 35 peptide.

FIG. 23H illustrates an autoradiographic image corresponding to FIG. 23G in which the ¹⁴C signal identifies the peptide distribution in the tissues of the mouse 3 hours after administration of 100 nmol of the radiolabeled SEQ ID NO: 35 peptide.

FIG. 24 shows a graph of the half-life of the SEQ ID NO: 5 peptide after administration.

FIG. 25 shows a comparison of near-infrared fluorescent images of Ewing's Sarcoma tumors excised either from mice 4 hours after administration of 10 nmol of SEQ ID NO: 4 peptide conjugated to AF647 fluorescent dye (SEQ ID NO: 4-A peptide conjugate), 10 nmol of Imperatoxin conjugated to AF647 fluorescent dye (Imperatoxin-A conjugate), 10 nmol of Conotoxin CVIC conjugated to AF647 fluorescent dye (Conotoxin-A conjugate), or 10 nmol of SEQ ID NO: 54 peptide conjugated to AF647 fluorescent dye (SEQ ID NO: 54-A peptide conjugate), or from mice that did not receive any peptide.

FIG. 25A shows a near-infrared fluorescence image of Ewing's Sarcoma tumor excised from a mouse 4 hours after administration of 10 nmol of SEQ ID NO: 4 peptide conjugated to AF647 fluorescent dye (SEQ ID NO: 4-A peptide conjugate).

FIG. 25B shows a near-infrared fluorescence image of Ewing's Sarcoma tumor excised from a different mouse than in FIG. 25A 4 hours after administration of 10 nmol of SEQ ID NO: 4 peptide conjugated to AF647 fluorescent dye (SEQ ID NO: 4-A peptide conjugate).

FIG. 25C shows a near-infrared fluorescence image of Ewing's Sarcoma tumor excised from a mouse 4 hours after administration of 10 nmol of Imperatoxin conjugated to AF647 fluorescent dye (Imperatoxin-A conjugate).

FIG. 25D shows a near-infrared fluorescence image of Ewing's Sarcoma tumor excised from a different mouse than in FIG. 25C 4 hours after administration of 10 nmol of Imperatoxin conjugated to AF647 fluorescent dye (Imperatoxin-A conjugate).

FIG. 25E shows a near-infrared fluorescence image of Ewing's Sarcoma tumor excised from a mouse 4 hours after administration of 10 nmol of Conotoxin CVIC conjugated to AF647 fluorescent dye (Conotoxin-A conjugate).

FIG. 25F shows a near-infrared fluorescence image of Ewing's Sarcoma tumor excised from a mouse 4 hours after administration of 10 nmol of SEQ ID NO: 54 peptide conjugated to AF647 fluorescent dye (SEQ ID NO: 54-A peptide conjugate).

FIG. 25G shows a near-infrared fluorescence image of Ewing's Sarcoma tumor excised from a mouse that did not receive any peptide as a negative control.

FIG. 26 shows a comparison of near-infrared fluorescent images of Ewing's Sarcoma tumors excised either from mice 4 hours after administration of 10 nmol of SEQ ID NO: 4 peptide conjugated to AF647 fluorescent dye (SEQ ID NO: 4-A peptide conjugate), 10 nmol of Imperatoxin conjugated to AF647 fluorescent dye (Imperatoxin-A conjugate), 10 nmol of Conotoxin CVIC conjugated to AF647 fluorescent dye (Conotoxin-A conjugate), or 10 nmol of SEQ ID NO: 54 peptide conjugated to AF647 fluorescent dye (SEQ ID NO: 54-A peptide conjugate), or from a mouse that did not receive any peptide.

FIG. 26A shows a near-infrared fluorescence image of the kidneys excised from a mouse 4 hours after administration of 10 nmol SEQ ID NO: 4 peptide conjugated to AF647 fluorescent dye (SEQ ID NO: 4-A peptide conjugate).

FIG. 26B shows a near-infrared fluorescence image of the kidneys excised from a different mouse than in FIG. 26A 4 hours after administration of 10 nmol of SEQ ID NO: 4 peptide conjugated to AF647 fluorescent dye (SEQ ID NO: 4-A peptide conjugate).

FIG. 26C shows a near-infrared fluorescence image of the kidneys excised from a mouse 4 hours after administration of 10 nmol of Imperatoxin conjugated to AF647 fluorescent dye (Imperatoxin-A conjugate).

FIG. 26D shows a near-infrared fluorescence image of the kidneys excised from a different mouse than in FIG. 26C 4 hours after administration of 10 nmol of Imperatoxin conjugated to AF647 fluorescent dye (Imperatoxin-A conjugate).

FIG. 26E shows a near-infrared fluorescence image of the kidneys excised from a mouse 4 hours after administration of 10 nmol Conotoxin CVIC conjugated to AF647 fluorescent dye (Conotoxin-A conjugate).

FIG. 26F shows a near-infrared fluorescence image of the kidneys excised from a mouse 4 hours after administration of 10 nmol of SEQ ID NO: 54 peptide conjugated to AF647 fluorescent dye (SEQ ID NO: 54-A peptide conjugate).

FIG. 26G shows a near-infrared fluorescence image of the kidneys excised from a mouse that did not receive any peptide as a negative control.

FIG. 27 shows a near-infrared fluorescence image of livers excised excised either from mice 4 hours after administration of 10 nmol of SEQ ID NO: 4 peptide conjugated to AF647 fluorescent dye (SEQ ID NO: 4-A peptide conjugate), 10 nmol of Imperatoxin conjugated to AF647 fluorescent dye (Imperatoxin-A conjugate), 10 nmol of Conotoxin CVIC conjugated to AF647 fluorescent dye (Conotoxin-A conjugate), or 10 nmol of SEQ ID NO: 54 peptide conjugated to AF647 fluorescent dye (SEQ ID NO: 54-A peptide conjugate), or from a mouse that did not receive any peptide.

FIG. 27A shows a near-infrared fluorescence image of the liver excised from a mouse 4 hours after administration of 10 nmol of SEQ ID NO: 4 peptide conjugated to AF647 fluorescent dye (SEQ ID NO: 4-A peptide conjugate).

FIG. 27B shows a near-infrared fluorescence image of the liver excised from a different mouse than in FIG. 27A 4 hours after administration of 10 nmol of SEQ ID NO: 4 peptide conjugated to AF647 fluorescent dye (SEQ ID NO: 4-A peptide conjugate).

FIG. 27C shows a near-infrared fluorescence image of the liver excised from a mouse 4 hours after administration of 10 nmol of Imperatoxin conjugated to AF647 fluorescent dye (Imperatoxin-A conjugate).

FIG. 27D shows a near-infrared fluorescence image of the liver excised from a different mouse than in FIG. 27C 4 hours after administration of 10 nmol of Imperatoxin conjugated to AF647 fluorescent dye (Imperatoxin-A peptide conjugate).

FIG. 27E shows a near-infrared fluorescence image of the liver excised from a mouse 4 hours after administration of 10 nmol of Conotoxin CVIC conjugated to AF647 fluorescent dye (Conotoxin-A conjugate).

FIG. 27F shows a near-infrared fluorescence image of the liver excised from a mouse 4 hours after administration of 10 nmol of SEQ ID NO: 54 peptide conjugated to AF647 fluorescent dye (SEQ ID NO: 54-A peptide conjugate).

FIG. 27G shows a near-infrared fluorescence image of the liver excised from a mouse that did not receive any peptide as a negative control.

FIG. 28 shows a near-infrared fluorescence image of different tissues that were excised from a mouse that did not receive any peptide or from a mouse 4 hours after the administration of 10 nmol of SEQ ID NO: 54 peptide conjugated to AF647 fluorescent dye (SEQ ID NO: 54-A peptide conjugate).

FIG. 28A shows a near-infrared fluorescence image of different tissues that were excised 4 hours after the administration of 10 nmol of SEQ ID NO: 54 peptide conjugated to AF647 fluorescent dye (SEQ ID NO: 54-A peptide conjugate). The tissues on the top row from left to right are tumor, kidneys, liver, heart, and the draining lymph node. The tissues on the bottom row from left to right are brain, spleen, skeletal muscle, lung, and the lateral lymph node. Tissue fluorescence indicates the presence of the peptide-conjugate.

FIG. 28B shows the near-infrared fluorescence image of FIG. 28A of different tissues that were excised 4 hours after the administration of 10 nmol of SEQ ID NO: 54 peptide conjugated to AF647 fluorescent dye (SEQ ID NO: 54-A peptide conjugate), but the image was taken without the kidneys. The tissues on the top row from left to right are tumor, liver, heart, and the draining lymph node. The tissues on the bottom row from left to right are brain, spleen, skeletal muscle, lung, and the lateral lymph node. Tissue fluorescence indicates the presence of the peptide-conjugate.

FIG. 28C shows a near-infrared fluorescence image of different tissues that were excised from a mouse that did not receive any peptide as a negative control. The tissues on the top row from left to right are tumor, kidneys, liver, and heart. The tissues on the bottom row from left to right are brain, spleen, skeletal muscle, and lung. Tissue fluorescence indicates autofluorescence.

FIG. 29 shows an ex vivo near-infrared fluorescence image of the internal body cavity of a mouse either with or without the kidneys removed, wherein that the mouse was euthanized 4 hours after administration of 10 nmol of SEQ ID NO: 54 peptide conjugated to AF647 fluorescent dye (SEQ ID NO: 54-A peptide conjugate).

FIG. 29A shows an ex vivo near-infrared fluorescence image of the internal body cavity of a mouse that was euthanized 4 hours after administration of 10 nmol of SEQ ID NO: 54 peptide conjugated to AF647 fluorescent dye (SEQ ID NO: 54-A peptide conjugate). Lv indicates the location of the liver. Tm indicates the location of the tumor. Kd indicates the location of the kidneys. B1 indicates the location of the bladder.

FIG. 29B shows an ex vivo near-infrared fluorescence image of the internal body cavity of a mouse that was euthanized 4 hours after administration of 10 nmol of SEQ ID NO: 54 peptide conjugated to AF647 fluorescent dye (SEQ ID NO: 54-A peptide conjugate) as shown in FIG. 29A, but with the kidneys removed. Lv indicates the location of the liver. Tm indicates the location of the tumor. B1 indicates the location of the bladder. Ht indicates the location of the heart. Lg indicates the location of the lung.

FIG. 30 illustrates ¹⁴C signal in the brain for peptides of SEQ ID NO: 55.

FIG. 31 illustrates the HPLC profile of a peptide of SEQ ID NO: 55 with reduced and non-reduced chromatograms overlaid.

FIG. 32 illustrates HPLC radiograms of a ¹⁴C-labeled peptide of SEQ ID NO: 55 in whole brain homogenates.

FIG. 32A shows the peptides spiked into a crude brain homogenate and run on a scintillation detector-equipped HPLC on a hydrophobic column using an acetonitrile gradient and 0.1% TFA.

FIG. 32B shows a scintillation HPLC trace of three mouse brains following systemic administration of the radiolabeled peptide. The arrow indicates the peak corresponding to the intact ¹⁴C-labeled peptide of SEQ ID NO: 55 at the same retention time as the spike control shown in FIG. 32A.

FIG. 33 illustrates sagittal (FIG. 33A) and coronal (FIG. 33B) brain sections indicating localization of a peptide of SEQ ID NO: 55 to specific structures in the brain, such as ventricles and CSF. In both FIG. 33A and FIG. 33B, the radioactivity scan is shown on the left, with dark areas having higher activity. Images of the tissue in normal light are shown on the right.

FIG. 34 illustrates a white light image and a corresponding autoradiographic image of a mouse with ligated kidneys 3 hours after administration of 100 nmol of the radiolabeled SEQ ID NO: 39 peptide.

FIG. 34A illustrates a white light image of a frozen section of a mouse with ligated kidneys 3 hours after administration of 100 nmol of the radiolabeled SEQ ID NO: 39 peptide.

FIG. 34B illustrates an autoradiographic image corresponding to FIG. 34A in which the ¹⁴C signal identifies the peptide distribution in the tissues of the mouse with ligated kidneys 3 hours after administration of 100 nmol of the radiolabeled SEQ ID NO: 39 peptide.

FIG. 35 illustrates a white light image and the corresponding autoradiographic image of a mouse with ligated kidneys 3 hours after administration of 100 nmol of the radiolabeled SEQ ID NO: 36 peptide.

FIG. 35A illustrates a white light image of a frozen section of a mouse with ligated kidneys 3 hours after administration of 100 nmol of the radiolabeled SEQ ID NO: 36 peptide.

FIG. 35B illustrates an autoradiographic image corresponding to FIG. 35A in which the ¹⁴C signal identifies the peptide distribution in the tissues of the mouse with ligated kidneys 3 hours after administration of 100 nmol of the radiolabeled SEQ ID NO: 36 peptide.

FIG. 36 illustrates white light and autoradiographic images of murine coronal brain sections, identifying peptide distribution 3 hours after administration of 100 nmol of the radiolabeled first purified fraction (first HPLC peptide peak) of a peptide of SEQ ID NO: 55 or the radiolabeled second purified fraction (second HPLC peptide peak) of a peptide of SEQ ID NO: 55 from the same HPLC.

FIG. 36A illustrates white light images of coronal brain sections of a mouse on the right and autoradiographic images that correspond to the white light images on the left. The ¹⁴C signal in the autographic images identifies the peptide distribution, indicating localization of the radiolabeled first purified fraction (first HPLC peptide peak) of a peptide of SEQ ID NO: 55, to specific structures in the brain, such as ventricles and CSF 3 hours after administration of 100 nmol of the peptide.

FIG. 36B illustrates white light images of coronal brain sections of a mouse on the right and autoradiographic images corresponding to the white light images on the left. The ¹⁴C signal in the autographic images identifies the peptide distribution, indicating localization of the second purified fraction (second HPLC peptide peak from the HPLC in FIG. 36A) of a peptide of SEQ ID NO: 55, to specific structures in the brain, such as ventricles and CSF 3 hours after administration of 100 nmol of the peptide.

FIG. 37 illustrates a white light image and the corresponding autoradiographic image of a mouse with ligated kidneys identifying peptide distribution 3 hours after administration of 100 nmol of the radiolabeled first purified fraction (first HPLC peptide peak) of a peptide of SEQ ID NO: 55.

FIG. 37A illustrates a white light image of a frozen section of a mouse with ligated kidneys 3 hours after administration of 100 nmol of the radiolabeled first purified fraction (first HPLC peptide peak) of a peptide of SEQ ID NO: 55.

FIG. 37B illustrates an autoradiographic image corresponding to FIG. 37A in which the ¹⁴C signal identifies the peptide distribution in the tissues of a mouse with ligated kidneys 3 hours after administration of 100 nmol of the radiolabeled first fraction (first HPLC peptide peak) of a peptide of SEQ ID NO: 55.

FIG. 38 illustrates a white light image and the corresponding autoradiographic image of a mouse with ligated kidneys identifying peptide distribution 3 hours after administration of 100 nmol of the radiolabeled second purified fraction (second HPLC peptide peak of the HPLC from FIG. 37) of a peptide of SEQ ID NO: 55.

FIG. 38A illustrates a white light image of a frozen section of a mouse with ligated kidneys 3 hours after administration of 100 nmol of the radiolabeled second purified fraction (second HPLC peptide peak of the HPLC from FIG. 37) of a peptide of SEQ ID NO: 55.

FIG. 38B illustrates an autoradiographic image corresponding to FIG. 38A in which the ¹⁴C signal identifies the peptide distribution in the tissues of the mouse with ligated kidneys 3 hours after administration of 100 nmol of the radiolabeld second purified fraction (second HPLC peptide peak of the HPLC from FIG. 37) of a peptide of SEQ ID NO: 55.

FIG. 39 illustrates a white light and the corresponding autoradiographic image of a mouse with ligated kidneys identifying peptide distribution 3 hours after administration of 100 nmol of the radiolabeled SEQ ID NO: 83 peptide.

FIG. 39A illustrates a white light image of a frozen section of a mouse with ligated kidneys 3 hours after administration of 100 nmol of the radiolabeled SEQ ID NO: 83 peptide

FIG. 39B illustrates an autoradiographic image corresponding to FIG. 39A in which the ¹⁴C signal identifies the peptide distribution in the tissues of the mouse with ligated kidneys 3 hours after administration of 100 nmol of the radiolabeled SEQ ID NO: 83 peptide.

FIG. 40 illustrates white light images of coronal brain sections on the right and autoradiographic images that correspond to the white light images on the left. The ¹⁴C signal in the autographic images identifies the peptide distribution 3 hours after administration of the radiolabeled SEQ ID NO: 34 and indicates the localization of the peptide to specific structures in the brain, such as ventricles and CSF.

FIG. 41 illustrates white light images of coronal brain sections on the right and autoradiographic images that correspond to the white light images on the left. The ¹⁴C signal in the autographic images identifies the peptide distribution 3 hours after administration of the radiolabeled SEQ ID NO: 83 and indicates the localization of the peptide to specific structures in the brain, such as ventricles and CSF.

FIG. 42 shows a near-infrared fluorescence image of Colo205 tumor (top left), colon (top middle), liver (top right), brain (middle left), spleen (middle right), muscle (bottom left), skin (bottom middle), and kidney (bottom right) that were excised 24 hours after administration of 10 nmol of a peptide of SEQ ID NO: 37 conjugated to Cy5.5 to Colo205 tumor-bearing Female Harlan athymic mice.

FIG. 43 shows a near-infrared fluorescence image of MDA-MB-231 tumor (top left), colon (top middle), liver (top right), brain (middle left), spleen (middle right), muscle (bottom left), skin (bottom middle), and kidney (bottom right) that were excised 24 hours after administration of 10 nmol of a peptide of SEQ ID NO: 37 conjugated to Cy5.5 to MDA-MB-231 tumor-bearing Female Harlan athymic mice.

FIG. 44 shows a near-infrared fluorescence image of U87 tumor (top left), colon (top middle), liver (top right), brain (middle left), spleen (middle right), muscle (bottom left), skin (bottom middle), and kidney (bottom right) that were excised 24 hours after administration of 10 nmol of a peptide of SEQ D NO: 37 conjugated to Cy5.5 to U87 tumor-bearing Female Harlan athymic mice.

FIG. 45 shows sequences of SEQ ID NO: 211 aligned with SEQ ID NO: 212 with annotation of the location of loops, and their corresponding 3D structures, with the SEQ ID NO: 211 structure on the left and the SEQ ID NO: 212 structure on the right.

FIG. 46 shows the sequence alignment of SEQ ID NO: 425 and SEQ ID NO: 426 with the location of the loops annotated.

### DETAILED DESCRIPTION

The present disclosure relates to compositions and methods for treatment of tumors. Furthermore, it relates to compositions that can cross the blood brain barrier, enabling treatment of brain tumors and other brain disorders and diseases. In some embodiments the compositions and methods herein utilize peptides that home, distribute to, target, are directed to, accumulate in, migrate to, and/or bind to cancerous cells following administration to a subject. In further embodiments the compositions and methods herein utilize peptides that home, distribute to, target, are directed to, accumulate in, migrate to, and/or bind to cancerous or diseased cells in the brain following administration to a subject. In other embodiments, peptides described herein cross the blood brain barrier into the neuronal parenchyma to deliver therapeutically active molecules to targets of neurological diseases including brain cancers. In some embodiments, the homing peptides of the present disclosure are used to deliver an active agent to a tissue or cell thereof. The active agent can exert a therapeutic effect on the targeted tissue or cell thereof. For example, in certain embodiments, the peptide allows for localized delivery of a chemotherapeutic agent to a cancerous tissue or cell thereof. As another example, in certain embodiments, the peptide allows for localized delivery of a therapeutic drug to a diseased tissue or cell of the brain. In certain embodiments, the homing peptides of the present disclosure are used to image the targeted tissue or cell. For example, the peptide allows for imaging using a fluorophore. In certain embodiments, the peptide itself possesses or induces therapeutic responses.

Many types of tumors are difficult to treat. Often, the prognosis of the patient is directly influenced by the ability drug therapies to effectively kill the cancerous cells and on the precision with which the cancer cells can be surgically resected. For example, one challenge in treating tumors is that many drugs treatments are systemic, and therefore, the efficacy of their use is constrained by the toxicity of systemic use. Another challenge is that the current methods of intra-operative imaging of cancerous tissues fail to precisely depict tumor margins or small foci of cancerous cells. Instead, resection is dependent upon the surgeon's ability to visually recognize tumor or physically locate it by touch in a surgical setting, which can be an imprecise method to identify the tumor margins or foci.

Treatment of brain tumors, as well as other brain disorders and disease, are also challenging and complicated to treat. One challenge is that many drugs administered into the circulatory system of patients fail to cross the blood-brain barrier (BBB) or the blood CSF barrier, which are selective barriers that separates the circulating blood from the brain extracellular fluid and the central nervous system tissue. Another challenge is that many drugs lack sufficient specificity to one or more target regions, tissues, structures or cells in the brain. Thus, treatment of brain conditions often requires the use of high concentrations of non-specific drugs, leading to suboptimal efficacy and systemic side effects. One other way to deliver drugs to the brain is to apply them directly in conjunction with surgical procedures. Another way is to identify specific drugs that cross the blood brain barrier. Specific and potent drugs that are capable of crossing the BBB can counteract the non-specificity of many treatments by selectively targeting and delivering compounds to specific tissues, cells, structures and regions. Such drugs can also be useful to modulate ion channels, protein-protein interactions, extracellular matrix remodeling (i.e., protease inhibition), intracellular signaling pathways, neurotransmitter signaling, infections, and the like. Such targeted therapy can allow for lower dosing, reduced side effects, and improvement in therapeutic outcomes, which would be advantageous not only in acute disease of the brain, but in chronic conditions as well.

The present disclosure describes a class of peptides derived from knottins that can home, distribute to, target, be directed to, accumulate in, migrate to, and/or bind to cancerous or diseased cells, and be used either directly or as carriers of active drugs, peptides or molecules to treat the cancerous or diseased cells. A peptide that homes, distributes to, targets, migrates to, or accumulates in one or more specific cancerous or diseased regions, tissues, structures or cells can have fewer off-target and potentially negative effects.

The present disclosure also provides a new kind of carrier that can deliver an active agent or detectable agent to a specific region, tissue, structure or cell that can be used for either or both therapeutic and imaging purposes. As described herein, an active agent or detectable agent can be linked to a peptide of the disclosure.

Furthermore, the present disclosure describes a class of peptides derived from knottins that can effectively cross the BBB or blood CSF barrier and be used either directly or as carriers of active drugs, peptides or molecules to treat a brain condition. For instance, Alzheimer's disease is a brain condition that is associated with the aggregation of amyloid beta peptide fragment. The accumulation of the amyloid beta peptide fragment is a result of proteolytic cleavage of the amyloid precursor protein (APP) by an enzyme known as beta-secretase. A therapeutic peptide that could cross the BBB to interact with and inhibit the beta-secretase protease could be used in the treatment and prevention of Alzheimer's disease by reducing aggregation of the amyloid beta fragment through, for example, binding or inhibiting the protease, antagonizing APP cleavage, regulating the amyloid beta fragment pathway, or other mechanism. Furthermore, acetylcholinesterase inhibitors such as rivastigmine have been used to treat Alzheimer's disease. However these are systemically dosed and often cause symptoms such as bradycardia and bronchoconstriction in the periphery. The opportunity to deliver more of the acetylcholineseterase across the BBB as a conjugate may allow for lower doses and side effects in the periphery. The peptides of the disclosure can be used to treat the symptoms of various conditions.

Also described herein are peptides that selectively home, distribute to, target, are directed to, migrate to, or accumulate in specific regions, tissues, structures or cells of the brain. In some cases, the peptides accumulate in one or more of: the hippocampus, the center of memory and learning and spatial navigation; the cerebrospinal fluid (CSF), which is found in the brain and spine; the ventricular system, the site of CSF production and circulation; the rostral migratory stream; the dentate gyrus; neural stem cells; or neuronal precursors. The dentate gyrus of the hippocampus and the subventricular zone are two locations of neurogenesis in the adult brain, and the rostral migratory stream is one mechanism for migration of new neurons. Thus, targeting those regions could allow for modulation of various aspects of neurogenesis, including repair or regeneration. A peptide that homes, distributes to, targets, migrates to, or accumulates in one or more specific regions, tissues, structures or cells of the brain can have fewer off-target and potentially negative effects, for example, side effects that often limit use and efficacy of drugs for neurological conditions. In addition, such peptides can increase the efficacy of existing drugs by directly targeting them to a specific region, tissue, structure or cell of the brain and helping the drug cross the blood brain barrier.

The present disclosure also provides a new kind of drug carrier that can deliver an active agent or detectable agent to the brain that can be used for either or both therapeutic and imaging purposes. The blood-brain barrier is formed by special tight junctions between the endothelial cells that surround the brain tissue, as well as a basement membrane and astrocyte protrusions.

Similarly the blood CSF barrier is formed by tight junctions between choroidal epithelial cells, a basement membrane, and endothelial cells. One of the functions of the BBB and the blood CSF barrier is to protect the brain and keep it isolated from harmful toxins that may be in the blood stream. As described herein, an active agent or a detectable agent can be linked to a peptide of the disclosure and the linked peptide-active agent or linked peptide-detectable agent compound can cross the blood brain barrier or blood CSF barrier.

The disclosure also provides a method for treating a condition of a subject, wherein the method comprises administrating to the subject a peptide that homes, targets, migrates to, is directed to a region, tissue, structure or cell in the brain of the subject, for example within the hippocampus, the CSF, the ventricular system, the meninges, the rostral migratory stream, or other specific region of the brain, for example, substantia nigra (which can be associated with Parkinsons disease). In some cases, the administered peptide can cross the blood brain barrier or blood CSF barrier of the subject. Additional aspects and advantages of the present disclosure will become apparent to those skilled in this art from the following detailed description, wherein illustrative embodiments of the present disclosure are shown and described. As will be realized, the present disclosure is capable of other and different embodiments, and its several details are capable of modifications in various respects, all without departing from the disclosure. Accordingly, the drawings and description are to be regarded as illustrative in nature, and not as restrictive.

Additional aspects and advantages of the present disclosure will become apparent to those skilled in this art from the following detailed description, wherein illustrative embodiments of the present disclosure are shown and described. As will be realized, the present disclosure is capable of other and different embodiments, and its several details are capable of modifications in various respects, all without departing from the disclosure. Accordingly, the drawings and description are to be regarded as illustrative in nature, and not as restrictive.

As used herein, the abbreviations for the natural L-enantiomeric amino acids are conventional and are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Typically, Xaa can indicate any amino acid. In some embodiments, X can be asparagine (N), glutamine (Q), histidine (H), lysine (K), or arginine (R).

Some embodiments of the disclosure contemplate D-amino acid residues of any standard or non-standard amino acid or analogue thereof. When an amino acid sequence is represented as a series of three-letter or one-letter amino acid abbreviations, the left-hand direction is the amino terminal direction and the right-hand direction is the carboxy terminal direction, in accordance with standard usage and convention.

### Peptides

Knottins are a class of peptides, usually ranging from about 11 to about 81 amino acids in length that are often folded into a compact structure. Knottins are typically assembled into a complex tertiary structure that is characterized by a number of intramolecular disulfide crosslinks and may contain beta strands and other secondary structures. The presence of the disulfide bonds gives knottins remarkable environmental stability, allowing them to withstand extremes of temperature and pH and to resist the proteolytic enzymes of the blood stream. The rigidity of knottins also allows them to bind to targets without paying the “entropic penalty” that a floppy peptide accrues upon binding a target. For example, binding is adversely affected by the loss of entropy that occurs when a peptide binds a target to form a complex. Therefore, “entropic penalty” is the adverse effect on binding, and the greater the entropic loss that occurs upon this binding, the greater the “entropic penalty.” Furthermore, unbound molecules that are flexible lose more entropy when forming a complex than molecules that are rigidly structured, because of the loss of flexibility when bound up in a complex. However, rigidity in the unbound molecule also generally increases specificity by limiting the number of complexes that molecule can form. The knotted peptides can bind targets with antibody-like affinity. A wider examination of the sequence structure and sequence identity or homology of knottins reveals that they have arisen by convergent evolution in all kinds of animals and plants. In animals, they are typically found in venoms, for example, the venoms of spiders and scorpions and have been implicated in the modulation of ion channels. The knottin proteins of plants can inhibit the proteolytic enzymes of animals or have antimicrobial activity, suggesting that knottins can function in the native defense of plants.

The present disclosure provides peptides that comprise or are derived from these knotted peptides (or knottins). As used herein, the term “knotted peptide” is considered to be interchangeable with the terms “knottin” and “optide.”

The peptides of the present disclosure can comprise cysteine amino acid residues. In some cases, the peptide has at least 6 cysteine amino acid residues. In some cases, the peptide has at least 8 cysteine amino acid residues. In other cases, the peptide has at least 10 cysteine amino acid residues, at least 12 cysteine amino acid residues, at least 14 cysteine amino acid residues or at least 16 cysteine amino acid residues.

A knotted peptide can comprise disulfide bridges. A knotted peptide can be a peptide wherein 5% or more of the residues are cysteines forming intramolecular disulfide bonds. A disulfide-linked peptide can be a drug scaffold. In some embodiments, the disulfide bridges form a knot. A disulfide bridge can be formed between cysteine residues, for example, between cysteines 1 and 4, 2 and 5, or, 3 and 6. In some cases, one disulfide bridge passes through a loop formed by the other two disulfide bridges, for example, to form the knot. In other cases, the disulfide bridges can be formed between any two cysteine residues.

The present disclosure further includes peptide scaffolds that, e.g., can be used as a starting point for generating additional peptides. In some embodiments, these scaffolds can be derived from a variety of knotted peptides (or knottins). In certain embodiments, knotted peptides are assembled into a complex tertiary structure that is characterized by a number of intramolecular disulfide crosslinks, and optionally contain beta strands and other secondary structures such as an alpha helix. For example, knotted peptides include, in some embodiments, small disulfide-rich proteins characterized by a disulfide through disulfide knot. This knot can be, e.g., obtained when one disulfide bridge crosses the macrocycle formed by two other disulfides and the interconnecting backbone. In some embodiments, the knotted peptides can include growth factor cysteine knots or inhibitor cysteine knots. Other possible peptide structures include peptide having two parallel helices linked by two disulfide bridges without β-sheets (e.g., hefutoxin).

A knotted peptide can comprise at least one amino acid residue in an L configuration. A knotted peptide can comprise at least one amino acid residue in a D configuration. In some embodiments, a knotted peptide is 15-40 amino acid residues long. In other embodiments, a knotted peptide is 11-57 amino acid residues long. In still other embodiments, a knotted peptide is 11-81 amino acid residues long. In further embodiments, a knotted peptide is at least 20 amino acid residues long.

These kinds of peptides can be derived from a class of proteins known to be present or associated with toxins or venoms. In some cases, the peptide can be derived from toxins or venoms associated with scorpions or spiders. The peptide can be derived from venoms and toxins of spiders and scorpions of various genus and species. For example, the peptide can be derived from a venom or toxin of the Leiurus quinquestriatus hebraeus, Buthus occitanus tunetanus, Hottentotta judaicus, Mesobuthus eupeus, Buthus occitanus israelis, Hadrurus gertschi, Androctonus australis, Centruroides noxius, Heterometrus laoticus, Opistophthalmus carinatus, Haplopelma schmidti, Isometrus maculatus, Haplopelma huwenum, Haplopelma hainanum, Haplopelma schmidti, Agelenopsis aperta, Haydronyche versuta, Selenocosmia huwena, Heteropoda venatoria, Grammostola rosea, Ornithoctonus huwena, Hadronyche versuta, Atrax robustus, Angelenopsis aperta, Psalmopoeus cambridgei, Hadronyche infensa, Paracoelotes luctosus, and Chilobrachys jingzhaoor another suitable genus or species of scorpion or spider. In some cases, a peptide can be derived from a Buthus martensii Karsh (scorpion) toxin. In some embodiments, a peptide can be derived from a member of the pfam005453: Toxin_6 class.

TABLE 1 lists exemplary peptides derived from venoms or toxins of scorpions or spiders and for use with the present disclosure.

TABLE 1
Exemplary peptides according to the present disclosure.

| SEQ ID NO: | Peptide Sequence |
|---|---|
| SEQ ID NO: 1 | GSMCIPCFTTNPNMAAKCNACCGSRRGSCRGPQCIC |
| SEQ ID NO: 2 | GSGCLEFWWKCNPNDDKCCRPKLKCSKLFKLCNFSFG |
| SEQ ID NO: 3 | GSECRYWLGTCSKTGDCCSHLSCSPKHGWCVWDWTFRK |
| SEQ ID NO: 4 | GSMCMPCFTTDHQMARRCDDCCGGRGRGRCYGPQCLCR |
| SEQ ID NO: 5 | GSMCMPCFTTHHRMAENCDICCGGDGRGKCYGPQCLCR |
| SEQ ID NO: 6 | GSMCMPCFTTDHRMAENCDICCGGDGRGKCYGPQCLCR |
| SEQ ID NO: 7 | GSMCMPCFTTHHQMAENCDICCGGDGRGKCYGPQCLCR |
| SEQ ID NO: 8 | GSMCMPCFTTHHRMARNCDICCGGDGRGKCYGPQCLCR |
| SEQ ID NO: 9 | GSMCMPCFTTHHRMAERCDICCGGDGRGKCYGPQCLCR |
| SEQ ID NO: 10 | GSMCMPCFTTHHRMAENCDDCCGGDGRGKCYGPQCLCR |
| SEQ ID NO: 11 | GSRCMPCFTTDHQMARRCDDCCGGRGRGKCYGPQCLCR |
| SEQ ID NO: 12 | GSICIPCFTTDHQIARRCDDCCGGRGRGKCYGPQCLCR |
| SEQ ID NO: 13 | GSMCLPCFTTDHQLARRCDDCCGGRGRGKCYGPQCLCR |
| SEQ ID NO: 14 | GSMCMPCFTTEHQMARRCEECCGGRGRGKCYGPQCLCR |
| SEQ ID NO: 15 | GSMCIPCFTTDHQMARRCEECCGGRGRGKCYGPQCLCR |
| SEQ ID NO: 16 | GSICIPCFTTDHQMARRCDDCCGGRGDGKCYGPQCLCR |
| SEQ ID NO: 17 | GSICIPCFTTDHQIARRCDDCCGGRGRGKCYGPQCICR |
| SEQ ID NO: 18 | GSRCMPCFTTDHFMARFCDFCCGGRGRGKCYGPQCLCR |
| SEQ ID NO: 19 | GSRCMPCFTTDHYMARYCDYCCGGRGRGKCYGPQCLCR |
| SEQ ID NO: 20 | GSRCMPCFTTDHRMARRCDRCCGGRGRGKCYGPQCLCR |
| SEQ ID NO: 21 | GSRCMPCFTTDHEMARECDECCGGRGRGKCYGPQCLCR |
| SEQ ID NO: 22 | GSRCMPCFTTDHHMARHCDHCCGGRGRGKCYGPQCLCR |
| SEQ ID NO: 23 | GSLCLPCFTTHHRLADQCDICCGGDGRGKCYGPQCLCR |
| SEQ ID NO: 24 | GSICIPCFTTEHQIARRCEECCGGRGRGKCYGPQCLCR |
| SEQ ID NO: 25 | GSMCMPCFTTDTQMQERCDRCCGGGGRGKCWGPQCLCI |
| SEQ ID NO: 26 | GSMCMPCFTTIYRMAHECDECCGGRGRGKCYGPQCLCR |
| SEQ ID NO: 27 | GSMCMPCFTTGYRMAEYCDICCGGRGRGKCYGPQCLCR |
| SEQ ID NO: 28 | GSMCMPCFTTHRRMANTCDACCGGRSRGKCYGPQCLCR |
| SEQ ID NO: 29 | GSHCMPCFTTDHQMIRRCDDCCGGGSYGKCDGPQCLCF |
| SEQ ID NO: 30 | GSDCMPCFTTDHRMADHCDICCGGDDRGKCYGPQCLCR |
| SEQ ID NO: 31 | GSMCMPCFTTDHEMERRCDDCCGIGGGGKCHGPQCLCG |
| SEQ ID NO: 32 | GSMCMPCFTTEQRMAIICDDCCGGFGRGKCYGPQCLCR |
| SEQ ID NO: 33 | GSMCMPCFTTSEQMFRRCDDCCGGWGDGKCNGPHCLCR |
| SEQ ID NO: 34 | GSGVPINVKCRGSRDCLDPCKKAGMRFGKCINSKCHCTP |
| SEQ ID NO: 35 | GSMCMPCFTTEQRMAIICDDCCGGFGRGRCYGPQCLCR |
| SEQ ID NO: 36 | GSICIPCFTTDHQIARRCDDCCGGRGRGRCYGPQCICR |
| SEQ ID NO: 37 | GSMCMPCFTTDTQMQERCDRCCGGGGRGRCWGPQCLCI |
| SEQ ID NO: 38 | GSMCMPCFTTDTQMQERCDRCCGGGGRGRCWGPQCLC |
| SEQ ID NO: 39 | GSMCMPCFTTDHRMAENCDICCGGDGRGRCYGPQCLCR |
| SEQ ID NO: 40 | GSMCMPCFTTEQRMAIICDDCCGGFGRGKCYGPQCLCI |
| SEQ ID NO: 41 | GSMCMPCFTTEQRMAIICDDCCGGFGRGRCYGPQCLCI |
| SEQ ID NO: 42 | GSICIPCFTTDHQIARRCDDCCGGRGRGKCYGPQCICI |
| SEQ ID NO: 43 | GSICIPCFTTDHQIARRCDDCCGGRGRGRCYGPQCICI |
| SEQ ID NO: 44 | GSMCMPCFTTDTQMQEKCDRCCGGGGRGRCWGPQCLCI |
| SEQ ID NO: 45 | GSMCMPCFTTEQRMAIKCDDCCGGFGRGRCYGPQCLCR |
| SEQ ID NO: 46 | GSICIPCFTTDHQIARKCDDCCGGRGRGRCYGPQCICR |
| SEQ ID NO: 47 | GSMCMPCFTTDHRMAEKCDICCGGDGRGRCYGPQCLCR |
| SEQ ID NO: 48 | GSMCMPCFTTDTQMQERCDRCCGGKGRGRCWGPQCLCI |
| SEQ ID NO: 49 | GSMCMPCFTTEQRMAIICDDCCGGKGRGRCYGPQCLCR |
| SEQ ID NO: 50 | GSICIPCFTTDHQIARRCDDCCGGKGRGRCYGPQCICR |
| SEQ ID NO: 51 | GSMCMPCFTTDHRMAENCDICCGGKGRGRCYGPQCLCR |
| SEQ ID NO: 52 | GSMCMPCFTTDHRMAENCDICCGGDGRGKCYGPQCLCI |
| SEQ ID NO: 53 | GSMCMPCFTTDHRMAENCDICCGGDGRGRCYGPQCLCI |
| SEQ ID NO: 54 | GSMCMPCFTTHHRMAENCDICCGGDGRGRCYGPQCLCR |
| SEQ ID NO: 55 | GSVGCEECPMHCKGKNANPTCDDGVCNCNV |
| SEQ ID NO: 56 | GSVGCEECPMHCKGKNAKPTCDDGVCNCNV |
| SEQ ID NO: 57 | GSVGCEECPMHCKGKNAKPTCDNGVCNCNV |
| SEQ ID NO: 58 | GSVGCEECPMHCKGKHAVPTCDDGVCNCNV |
| SEQ ID NO: 59 | GSVGCEECPAHCKGKNAKPTCDDGVCNCNV |
| SEQ ID NO: 60 | GSVGCEECPAHCKGKNAIPTCDDGVCNCNV |
| SEQ ID NO: 61 | GSVGCEECPMHCKGKMAKPTCDDGVCNCNV |
| SEQ ID NO: 62 | GSVGCEECPMHCKGKNAVPTCDNGVCNCNA |
| SEQ ID NO: 63 | GSVGCEECPMHCKGKMAKPTCYDGVCNCNV |
| SEQ ID NO: 64 | GSVGCEECPMYCKGKNAVPTCDGGVCNCNA |
| SEQ ID NO: 65 | GSVGCEECPKYCKGKNAVPTCDGGVCNCNA |
| SEQ ID NO: 66 | GSVGCEECPVYCKGKKALPTCDGGVCNCNA |
| SEQ ID NO: 67 | GSVGCEEDPMHCKGKQAKPTCCNGVCNCNV |
| SEQ ID NO: 68 | GSVGCAECPMHCKGKMAKPTCENEVCKCNIGKKD |
| SEQ ID NO: 69 | GSVGCEECPMHCKGKKALPTCDYGCECND |
| SEQ ID NO: 70 | GSIVCKVCKIICGMQGKKVNICKAPIKCKCKKG |
| SEQ ID NO: 71 | GSVSCEDCPDHCSTQKARAKCDNDKCVCEPK |
| SEQ ID NO: 72 | GSVSCEDCPEHCSTQKARAKCDNDKCVCESV |
| SEQ ID NO: 73 | GSVSCEDCPEHCSTQKAQAKCDNDKCVCEPI |
| SEQ ID NO: 74 | GSATCEDCPEHCATQNARAKCDNDKCVCEPK |
| SEQ ID NO: 75 | GSVSCEDCPEHCATKDQRAKCDNDKCVCEPK |
| SEQ ID NO: 76 | GSVGCEDCPEHCSQQNARAKCENDKCVCEPK |
| SEQ ID NO: 77 | GSVSCEDCPEHCATKDQRAKCDNDRCVCEPK |
| SEQ ID NO: 78 | GSVSCEDCPPHCATKDQRAKCENDKCVCEPK |
| SEQ ID NO: 79 | GSVSCEDCPEHCSTQKARAKCDNDKCVCEAI |
| SEQ ID NO: 80 | GSMCMPCFTTEQRMAIICDDCCGGFGRGRCYGPQCLC |
| SEQ ID NO: 81 | GSICIPCFTTDHQIARRCDDCCGGRGRGRCYGPQCIC |
| SEQ ID NO: 82 | GSMCMPCFTTDHRMAENCDICCGGDGRGRCYGPQCLC |
| SEQ ID NO: 83 | GSVGCEECPMHCRGRNANPTCDDGVCNCNV |
| SEQ ID NO: 84 | GSVGCEECPMHCRGRNANPTCDDGVCNC |
| SEQ ID NO: 85 | GSCGPCFTTDHQMEQKCAECCGGIGKCYGPQCLCNR |
| SEQ ID NO: 86 | GSRCGPCFTTDPQTQAKCSECCGRKGGVCKGPQCICGIQY |
| SEQ ID NO: 87 | GSMCMPCFTTDPNMAKKCRDCCGGNGKCFGPQCLCNR |
| SEQ ID NO: 88 | GSMCMPCFTTDHNMAKKCNDCCGGYGKCFGPQCLCR |
| SEQ ID NO: 89 | GSRCPPCFTTNPNMEADCRKCCGGRGYCASYQCICPGG |
| SEQ ID NO: 90 | GSMCMPCFTTDPNMANKCRDCCGGGKKCFGPQCLCNR |
| SEQ ID NO: 91 | GSMKFLYGVILIALFLTVMTATLSEARCGPCFTTDPQTQAKCSECCGRKGG |
| | VCKGPQCICGIQY |
| SEQ ID NO: 92 | GSMCMPCFTTRPDMAQQCRACCKGRGKCFGPQCLCGYD |
| SEQ ID NO: 93 | GSMKFLYGIVFIALFLTVMTATLSDAMCMPCFTTDHNMAKKCRDCCGGN |
| | GKCFGPQCLCNRG |
| SEQ ID NO: 94 | GSMCMPCFTTDHNMAKKCRDCCGGNGKCFGPQCLCNR |
| SEQ ID NO: 95 | GSMKFLYGIVFITLFLTVMIATHTEAMCMPCFTTRPNMAQQCRDCCRGRG |
| | KCFGPQCLCGYD |
| SEQ ID NO: 96 | GSMKFLYGIVFIALFLTVMIATHTEAMCMPCFTTRPNMAQQCRDCCRGRG |
| | KCFGPQCLCGYD |
| SEQ ID NO: 97 | GSRCKPCFTTDPQMSKKCADCCGGKGKGKCYGPQCLC |
| SEQ ID NO: 98 | GSMKFLYGIVFITLFLTVMIATHTEAAMCMPCFTTNLNMEQECRDCCGGT |
| | GRCFGPQCLCGYD |
| SEQ ID NO: 99 | GSRCSPCFTTDQQMTKKCYDCCGGKGKGKCYGPQCICAPY |
| SEQ ID NO: 100 | GSCGPCFTTDPYTESKCATCCGGRGKCVGPQCLCNRI |
| SEQ ID NO: 101 | GSTEAMCMPCFTTDHNMAKKCRDCCGGNGKCFGYQCLCNRG |
| SEQ ID NO: 102 | GSMKFLYGIVFIALFLTVMFATQTDGCGPCFTTDANMARKCRECCGGIGK |
| | CFGPQCLCNRI |
| SEQ ID NO: 103 | GSMKFLYGIVFIALFLTVMFATQTDGCGPCFTTDANMARKCRECCGGNGK |
| | CFGPQCLCNRE |
| SEQ ID NO: 104 | GSMKFLYGTILIAFFLTVMIATHSEARCPPCFTTNPNMEADCRKCCGGRGY |
| | CASYQCICPGG |
| SEQ ID NO: 105 | GSTEAMCMPCFTTRPDMAQQCRDCCGGNGKCFGYQCLCNRG |
| SEQ ID NO: 106 | GSMKFLYGIVFIALFLTVMIATLTEAMCMPCFTTRPDMAQQCRDCCGGNG |
| | KCFGYQCLCNRG |
| SEQ ID NO: 107 | GSMKFLYGIVFIALFLTVMIATHTEAMCMPCFTTRPDMAQQCRDCCGGNG |
| | KCFGYQCLCNRG |
| SEQ ID NO: 108 | GSMKFLYGIILIALFLTVMIATHSEARCPNCFTTNPNAEADCKKCCGNRWG |
| | KCAGYQCVCPMK |
| SEQ ID NO: 109 | GSMKFLYGIVFIALFLTGMIATHTEAMCMPCFTTRPDMAQQCRDCCGGNG |
| | KCFGYQCLCNRGRIVIMYT |
| SEQ ID NO: 110 | GSMCMPCFTTRPGMAQQCRDCCGGNGKCFGYQCLCNR |
| SEQ ID NO: 111 | GSMCIPCFTTNPNMAAKCNACCGSRRGSCRGPQCICR |
| SEQ ID NO: 112 | GSMCIPCFTTNPNMAAKCNACCGSRRGSCRGPQCICN |
| SEQ ID NO: 113 | GSMCIPCFTTNPNMAAKCNACCGGNGSCRGPQCICN |
| SEQ ID NO: 114 | GSMCIPCFTTNPNMAAKCNACCGSRGRGSCRGPQCICN |
| SEQ ID NO: 115 | GSMCIPCFTTNPNMAAKCNACCGSRGRGKCRGPQCICN |
| SEQ ID NO: 116 | GSMCIPCFTTDHQMAAKCNACCGSRRGSCRGPQCICN |
| SEQ ID NO: 117 | GSMCIPCFTTNHQMAAKCNACCGSRRGSCRGPQCICN |
| SEQ ID NO: 118 | GSMCIPCFTTNPNMARKCNACCGSRGRGSCRGPQCICN |
| SEQ ID NO: 119 | GSMCIPCFTTNPNMAAKCNACCGGKGRGSCRGPQCICN |
| SEQ ID NO: 120 | GSMCIPCFTTNPNMAAKCNACCGSRRGSCFGPQCICN |
| SEQ ID NO: 121 | GSMCIPCFTTNPNMAAKCNACCGSRGRGKCFGPQCICN |
| SEQ ID NO: 122 | GSMCIPCFTTNPNMAAKCNACCGSRGRGSCFGPQCICN |
| SEQ ID NO: 123 | GSMCIPCFTTNPNMAAKCNACCGSRGRGSCYGPQCICN |
| SEQ ID NO: 124 | GSMCIPCFTTNPNMAAKCDACCGSRRGSCRGPQCICN |
| SEQ ID NO: 125 | GSMCIPCFTTNHQMAAKCDACCGSRRGSCRGPQCICN |
| SEQ ID NO: 126 | GSMCIPCFTTNHNMAAKCDACCGGRGRGSCRGPQCICN |
| SEQ ID NO: 127 | GSMCIPCFTTNPNMAAKCDACCGSRGRGSCRGPQCICN |
| SEQ ID NO: 128 | GSMCIPCFTTNPNMAAKCDACCGGKGRGSCRGPQCICN |
| SEQ ID NO: 129 | GSMCIPCFTTNHNMAAKCDACCGSRGRGSCRGPQCICN |
| SEQ ID NO: 130 | GSMCIPCFTTNPNMAAKCRDCCGGRGSCRGPQCICN |
| SEQ ID NO: 131 | GSMCMPCFTTNPNMAAKCDDCCGSRGRGSCRGPQCICN |
| SEQ ID NO: 132 | GSMCIPCFTTNPNMAARCNACCGSRRGSCRGPQCIC |
| SEQ ID NO: 133 | GSMCIPCFTTNPNMAAKCNACCGSRRGSCRGPQCICI |
| SEQ ID NO: 134 | GSGCLQFMWKCNPDNDKCCRPNLKCNTYHKWCEFVTGK |
| SEQ ID NO: 135 | GSDCLGFLWKCNPSNDKCCRPNLVCSRKDKWCKYQI |
| SEQ ID NO: 136 | GSDCLGFMRKCIPDNDKCCRPNLVCSRTHKWCKYVFGK |
| SEQ ID NO: 137 | GSECLEIFKACNPSNDQCCKSSKLVCSRKTRACKYQI |
| SEQ ID NO: 138 | GSECGGFWWKCGSGKPACCPKYVCSPKWGLCNFPMP |
| SEQ ID NO: 139 | GSGCLERWWKCNPNDDKCCRPKLKCSKLFKLCNRSRG |
| SEQ ID NO: 140 | GSGCLEEWWKCNPNDDKCCRPKLKCSKLFKLCNESEG |
| SEQ ID NO: 141 | GSGCLEIWWKCNPNDDKCCRPKLKCSKLFKLCNYSIG |
| SEQ ID NO: 142 | GSGCLEFWWKCNPNDDKCCRPKLKCSKLGKLCNFSFG |
| SEQ ID NO: 143 | GSGCLEFWWKCNPNDDKCCRPKLKCSPLGKLCNFSFG |
| SEQ ID NO: 144 | GSGCLEFWWKCNPNDDKCCRPKLKCSPNGKLCNFSFG |
| SEQ ID NO: 145 | GSGCLEFWWKCNPNDDKCCRPKLKCSRKTKLCNFSFG |
| SEQ ID NO: 146 | GSGCLEFWWKCNPNDDKCCRPKLKCGSNFKLCNFSFG |
| SEQ ID NO: 147 | GSGCLEFWWKCNPNDDKCCRPKLKCSTKHKLCNFSFG |
| SEQ ID NO: 148 | GSGCLEFWWKCNPNDDKCCRPKLKCSNDGKLCNFSFG |
| SEQ ID NO: 149 | GSGCLEFWWKCNPNDDKCCRPKLKCSKKTKLCNFSFG |
| SEQ ID NO: 150 | GSGCLEFWWKCNPNDDKCCRPKLKCHSNFKLCNFSFG |
| SEQ ID NO: 151 | GSGCLEFWWKCNPNDDKCCRPKLKCSKKFTACNFSFG |
| SEQ ID NO: 152 | GSGCLEIFKACNPNDDKCCRPKLKCSKLFKLCNFSFG |
| SEQ ID NO: 153 | GSGCLKFGWKCNPNDDKCCRPKLKCSKLFKLCNFSFG |
| SEQ ID NO: 154 | GSGCLEFWWKCNPNDDKCCKSSKLKCSKLFKLCNFSFG |
| SEQ ID NO: 155 | GSGCLEFWWKCNPNDDKCCRPKLKCNKLFKLCNISIG |
| SEQ ID NO: 156 | GSGCLEFWWKCNPNDDCCRKLKCSKLFKLCNFSFG |
| SEQ ID NO: 157 | GSGCLEFWWKCNPSNDQCCRPKLKCSKLFKLCNFSFG |
| SEQ ID NO: 158 | GSGCLEFWWKCNPNDDKCCRPSKLVCSKLFKLCNFSFG |
| SEQ ID NO: 159 | GSGCLEFLGECNPNDDKCCRPKLKCSKLFKLCNFSFG |
| SEQ ID NO: 160 | GSGCLWYLWKCNPNDDKCCRPKLKCSKLFKLCNFSFG |
| SEQ ID NO: 161 | GSGCLEFWWRCNPNDDRCCRPRLRCSRLFRLCNFSFG |
| SEQ ID NO: 162 | GSGCLEFWWRCNPNDDRCCRPRLRCSRLFRLC |
| SEQ ID NO: 163 | GSCRYFLGECKKTSECCEHLACHDKHKWCAWDWTIGK |
| SEQ ID NO: 164 | GSECRYWLGGCSAGQTCCKHLVCSRRHGWCVWDGTF |
| SEQ ID NO: 165 | GSECRWYLGECSQDGDCCKHLQCHSNYEWCIWDGTFSK |
| SEQ ID NO: 166 | GSECRWYLGGCSQDGDCCKHLQCHSNYEWCVWDGTFSK |
| SEQ ID NO: 167 | GSDCRKFLGACTQTSDCCKHLACHNKHKWCAWDWTI |
| SEQ ID NO: 168 | GSECRYLMGGCSKDGDCCEHLVCRTKWPYHCVWDWTFGK |
| SEQ ID NO: 169 | GSECRYRLGTCSKTGDCCSHLSCSPKHGWCVRDRTFRK |
| SEQ ID NO: 170 | GSECRYELGTCSKTGDCCSHLSCSPKHGWCVEDETFRK |
| SEQ ID NO: 171 | GSECRYILGTCSKTGDCCSHLSCSPKHGWCVYDITFRK |
| SEQ ID NO: 172 | GSECRYWLGTCSKTGDCCSHLSCSPKGGWCVWDWTFRK |
| SEQ ID NO: 173 | GSECRYWLGTCSKTGDCCSHLSCSPNHGWCVWDWTFRK |
| SEQ ID NO: 174 | GSECRYWLGTCSKTGDCCSHLSCSRKTGWCVWDWTFRK |
| SEQ ID NO: 175 | GSECRYWLGTCSKTGDCCSHLSCGSNHGWCVWDWTFRK |
| SEQ ID NO: 176 | GSECRYWLGTCSKTGDCCSHLSCSTKHGWCVWDWTFRK |
| SEQ ID NO: 177 | GSECRYWLGTCSKTGDCCSHLSCSSKHGWCVWDWTFRK |
| SEQ ID NO: 178 | GSECRYWLGTCSKTGDCCSHLSCSNDGGWCVWDWTFRK |
| SEQ ID NO: 179 | GSECRYWLGTCSKTGDCCSHLSCSPKTRACVWDWTFRK |
| SEQ ID NO: 180 | GSECRYWLGTCSKTGDCCSHLSCHSNHGWCVWDWTFRK |
| SEQ ID NO: 181 | GSECRYWLGTCSKTGDCCSHLSCSRKHRACVWDWTFRK |
| SEQ ID NO: 182 | GSECRYWLGTCSKTGDQCCKSSHLSCSPKHGWCVWDWTFRK |
| SEQ ID NO: 183 | GSECRYWLGTCSAGQDCCSHLSCSPKHGWCVWDWTFRK |
| SEQ ID NO: 184 | GSECRYWLGGCSATGDCCSHLSCSPKHGWCVWDWTFRK |
| SEQ ID NO: 185 | GSECRYWLGTCSKTGDCCKSSHLVCSPKHGWCVWDWTFRK |
| SEQ ID NO: 186 | GSECLEILGTCSKTGDCCSHLSCSPKHGWCVWDWTFRK |
| SEQ ID NO: 187 | GSECRYWFKACSKTGDCCSHLSCSPKHGWCVWDWTFRK |
| SEQ ID NO: 188 | GSECRYWLGTCSKTGDCCSHLSCSDGHGWCVWDWTFRK |
| SEQ ID NO: 189 | GSECRYWLGTCSKTGDCCSHLSCSKLHGWCVWDWTFRK |
| SEQ ID NO: 190 | GSECRYWLGTCSKTGDCCSHLQCHSKHGWCVWDWTFRK |
| SEQ ID NO: 191 | GSECRYWLGTCSRTGDCCSHLSCSPRHGWCVWDWTFRR |
| SEQ ID NO: 192 | GSECRYWLGTCSRTGDCCSHLSCSPRHGWC |
| SEQ ID NO: 193 | GSRCLPPGRPCYGATQRIPCCGVCSHNNCTGSSELYENKPRRPYIL |
| SEQ ID NO: 194 | GSVGCEECPMHCRGRNANPTCDDGVCNCNVGSSELYENKPRRPYIL |
| SEQ ID NO: 195 | GSMCMPCFTTDTQMQERCDRCCGGGGRGRCWGPQCLCIGSSELYENKPR |
| | RPYIL |
| SEQ ID NO: 196 | GSMCMPCFTTDPNMAKKCRDCCGGNGKCFGPQCLCNRGSSELYENKPRR |
| | PYIL |
| SEQ ID NO: 197 | GSSEKDCIKHLQRCRENKDCCSKKCSRRGTNPEKRCRGSSELYENKPRRPY |
| | IL |
| SEQ ID NO: 210 | MCIPCFTTNPNMAAKCNACCGSRRGSCRGPQCIC |
| SEQ ID NO: 211 | GCLEFWWKCNPNDDKCCRPKLKCSKLFKLCNFSFG |
| SEQ ID NO: 212 | ECRYWLGTCSKTGDCCSHLSCSPKHGWCVWDWTFRK |
| SEQ ID NO: 213 | MCMPCFTTDHQMARRCDDCCGGRGRGRCYGPQCLCR |
| SEQ ID NO: 214 | MCMPCFTTHHRMAENCDICCGGDGRGKCYGPQCLCR |
| SEQ ID NO: 215 | MCMPCFTTDHRMAENCDICCGGDGRGKCYGPQCLCR |
| SEQ ID NO: 216 | MCMPCFTTHHQMAENCDICCGGDGRGKCYGPQCLCR |
| SEQ ID NO: 217 | MCMPCFTTHHRMARNCDICCGGDGRGKCYGPQCLCR |
| SEQ ID NO: 218 | MCMPCFTTHHRMAERCDICCGGDGRGKCYGPQCLCR |
| SEQ ID NO: 219 | MCMPCFTTHHRMAENCDDCCGGDGRGKCYGPQCLCR |
| SEQ ID NO: 220 | RCMPCFTTDHQMARRCDDCCGGRGRGKCYGPQCLCR |
| SEQ ID NO: 221 | ICIPCFTTDHQIARRCDDCCGGRGRGKCYGPQCLCR |
| SEQ ID NO: 222 | MCLPCFTTDHQLARRCDDCCGGRGRGKCYGPQCLCR |
| SEQ ID NO: 223 | MCMPCFTTEHQMARRCEECCGGRGRGKCYGPQCLCR |
| SEQ ID NO: 224 | MCIPCFTTDHQMARRCEECCGGRGRGKCYGPQCLCR |
| SEQ ID NO: 225 | ICIPCFTTDHQMARRCDDCCGGRGDGKCYGPQCLCR |
| SEQ ID NO: 226 | ICIPCFTTDHQIARRCDDCCGGRGRGKCYGPQCICR |
| SEQ ID NO: 227 | RCMPCFTTDHFMARFCDFCCGGRGRGKCYGPQCLCR |
| SEQ ID NO: 228 | RCMPCFTTDHYMARYCDYCCGGRGRGKCYGPQCLCR |
| SEQ ID NO: 229 | RCMPCFTTDHRMARRCDRCCGGRGRGKCYGPQCLCR |
| SEQ ID NO: 230 | RCMPCFTTDHEMARECDECCGGRGRGKCYGPQCLCR |
| SEQ ID NO: 231 | RCMPCFTTDHHMARHCDHCCGGRGRGKCYGPQCLCR |
| SEQ ID NO: 232 | LCLPCFTTHHRLADQCDICCGGDGRGKCYGPQCLCR |
| SEQ ID NO: 233 | ICIPCFTTEHQIARRCEECCGGRGRGKCYGPQCLCR |
| SEQ ID NO: 234 | MCMPCFTTDTQMQERCDRCCGGGGRGKCWGPQCLCI |
| SEQ ID NO: 235 | MCMPCFTTIYRMAHECDECCGGRGRGKCYGPQCLCR |
| SEQ ID NO: 236 | MCMPCFTTGYRMAEYCDICCGGRGRGKCYGPQCLCR |
| SEQ ID NO: 237 | MCMPCFTTHRRMANTCDACCGGRSRGKCYGPQCLCR |
| SEQ ID NO: 238 | HCMPCFTTDHQMIRRCDDCCGGGSYGKCDGPQCLCF |
| SEQ ID NO: 239 | DCMPCFTTDHRMADHCDICCGGDDRGKCYGPQCLCR |
| SEQ ID NO: 240 | MCMPCFTTDHEMERRCDDCCGIGGGGKCHGPQCLCG |
| SEQ ID NO: 241 | MCMPCFTTEQRMAIICDDCCGGFGRGKCYGPQCLCR |
| SEQ ID NO: 242 | MCMPCFTTSEQMFRRCDDCCGGWGDGKCNGPHCLCR |
| SEQ ID NO: 243 | GVPINVKCRGSRDCLDPCKKAGMRFGKCINSKCHCTP |
| SEQ ID NO: 244 | MCMPCFTTEQRMAIICDDCCGGFGRGRCYGPQCLCR |
| SEQ ID NO: 245 | ICIPCFTTDHQIARRCDDCCGGRGRGRCYGPQCICR |
| SEQ ID NO: 246 | MCMPCFTTDTQMQERCDRCCGGGGRGRCWGPQCLCI |
| SEQ ID NO: 247 | MCMPCFTTDTQMQERCDRCCGGGGRGRCWGPQCLC |
| SEQ ID NO: 248 | MCMPCFTTDHRMAENCDICCGGDGRGRCYGPQCLCR |
| SEQ ID NO: 249 | MCMPCFTTEQRMAIICDDCCGGFGRGKCYGPQCLCI |
| SEQ ID NO: 250 | MCMPCFTTEQRMAIICDDCCGGFGRGRCYGPQCLCI |
| SEQ ID NO: 251 | ICIPCFTTDHQIARRCDDCCGGRGRGKCYGPQCICI |
| SEQ ID NO: 252 | ICIPCFTTDHQIARRCDDCCGGRGRGRCYGPQCICI |
| SEQ ID NO: 253 | MCMPCFTTDTQMQEKCDRCCGGGGRGRCWGPQCLCI |
| SEQ ID NO: 254 | MCMPCFTTEQRMAIKCDDCCGGFGRGRCYGPQCLCR |
| SEQ ID NO: 255 | ICIPCFTTDHQIARKCDDCCGGRGRGRCYGPQCICR |
| SEQ ID NO: 256 | MCMPCFTTDHRMAEKCDICCGGDGRGRCYGPQCLCR |
| SEQ ID NO: 257 | MCMPCFTTDTQMQERCDRCCGGKGRGRCWGPQCLCI |
| SEQ ID NO: 258 | MCMPCFTTEQRMAIICDDCCGGKGRGRCYGPQCLCR |
| SEQ ID NO: 259 | ICIPCFTTDHQIARRCDDCCGGKGRGRCYGPQCICR |
| SEQ ID NO: 260 | MCMPCFTTDHRMAENCDICCGGKGRGRCYGPQCLCR |
| SEQ ID NO: 261 | MCMPCFTTDHRMAENCDICCGGDGRGKCYGPQCLCI |
| SEQ ID NO: 262 | MCMPCFTTDHRMAENCDICCGGDGRGRCYGPQCLCI |
| SEQ ID NO: 263 | MCMPCFTTHHRMAENCDICCGGDGRGRCYGPQCLCR |
| SEQ ID NO: 264 | VGCEECPMHCKGKNANPTCDDGVCNCNV |
| SEQ ID NO: 265 | VGCEECPMHCKGKNAKPTCDDGVCNCNV |
| SEQ ID NO: 266 | VGCEECPMHCKGKNAKPTCDNGVCNCNV |
| SEQ ID NO: 267 | VGCEECPMHCKGKHAVPTCDDGVCNCNV |
| SEQ ID NO: 268 | VGCEECPAHCKGKNAKPTCDDGVCNCNV |
| SEQ ID NO: 269 | VGCEECPAHCKGKNAIPTCDDGVCNCNV |
| SEQ ID NO: 270 | VGCEECPMHCKGKMAKPTCDDGVCNCNV |
| SEQ ID NO: 271 | VGCEECPMHCKGKNAVPTCDNGVCNCNA |
| SEQ ID NO: 272 | VGCEECPMHCKGKMAKPTCYDGVCNCNV |
| SEQ ID NO: 273 | VGCEECPMYCKGKNAVPTCDGGVCNCNA |
| SEQ ID NO: 274 | VGCEECPKYCKGKNAVPTCDGGVCNCNA |
| SEQ ID NO: 275 | VGCEECPVYCKGKKALPTCDGGVCNCNA |
| SEQ ID NO: 276 | VGCEEDPMHCKGKQAKPTCCNGVCNCNV |
| SEQ ID NO: 277 | VGCAECPMHCKGKMAKPTCENEVCKCNIGKKD |
| SEQ ID NO: 278 | VGCEECPMHCKGKKALPTCDYGCECND |
| SEQ ID NO: 279 | IVCKVCKIICGMQGKKVNICKAPIKCKCKKG |
| SEQ ID NO: 280 | VSCEDCPDHCSTQKARAKCDNDKCVCEPK |
| SEQ ID NO: 281 | VSCEDCPEHCSTQKARAKCDNDKCVCESV |
| SEQ ID NO: 282 | VSCEDCPEHCSTQKAQAKCDNDKCVCEPI |
| SEQ ID NO: 283 | ATCEDCPEHCATQNARAKCDNDKCVCEPK |
| SEQ ID NO: 284 | VSCEDCPEHCATKDQRAKCDNDKCVCEPK |
| SEQ ID NO: 285 | VGCEDCPEHCSQQNARAKCENDKCVCEPK |
| SEQ ID NO: 286 | VSCEDCPEHCATKDQRAKCDNDRCVCEPK |
| SEQ ID NO: 287 | VSCEDCPPHCATKDQRAKCENDKCVCEPK |
| SEQ ID NO: 288 | VSCEDCPEHCSTQKARAKCDNDKCVCEAI |
| SEQ ID NO: 289 | MCMPCFTTEQRMAIICDDCCGGFGRGRCYGPQCLC |
| SEQ ID NO: 290 | ICIPCFTTDHQIARRCDDCCGGRGRGRCYGPQCIC |
| SEQ ID NO: 291 | MCMPCFTTDHRMAENCDICCGGDGRGRCYGPQCLC |
| SEQ ID NO: 292 | VGCEECPMHCRGRNANPTCDDGVCNCNV |
| SEQ ID NO: 293 | VGCEECPMHCRGRNANPTCDDGVCNC |
| SEQ ID NO: 294 | CGPCFTTDHQMEQKCAECCGGIGKCYGPQCLCNR |
| SEQ ID NO: 295 | RCGPCFTTDPQTQAKCSECCGRKGGVCKGPQCICGIQY |
| SEQ ID NO: 296 | MCMPCFTTDPNMAKKCRDCCGGNGKCFGPQCLCNR |
| SEQ ID NO: 297 | MCMPCFTTDHNMAKKCNDCCGGYGKCFGPQCLCR |
| SEQ ID NO: 298 | RCPPCFTTNPNMEADCRKCCGGRGYCASYQCICPGG |
| SEQ ID NO: 299 | MCMPCFTTDPNMANKCRDCCGGGKKCFGPQCLCNR |
| SEQ ID NO: 300 | MKFLYGVILIALFLTVMTATLSEARCGPCFTTDPQTQAKCSECCGRKGGVC |
| | KGPQCICGIQY |
| SEQ ID NO: 301 | MCMPCFTTRPDMAQQCRACCKGRGKCFGPQCLCGYD |
| SEQ ID NO: 302 | MKFLYGIVFIALFLTVMTATLSDAMCMPCFTTDHNMAKKCRDCCGGNGK |
| | CFGPQCLCNRG |
| SEQ ID NO: 303 | MCMPCFTTDHNMAKKCRDCCGGNGKCFGPQCLCNR |
| SEQ ID NO: 304 | MKFLYGIVFITLFLTVMIATHTEAMCMPCFTTRPNMAQQCRDCCRGRGKC |
| | FGPQCLCGYD |
| SEQ ID NO: 305 | MKFLYGIVFIALFLTVMIATHTEAMCMPCFTTRPNMAQQCRDCCRGRGKC |
| | FGPQCLCGYD |
| SEQ ID NO: 306 | RCKPCFTTDPQMSKKCADCCGGKGKGKCYGPQCLC |
| SEQ ID NO: 307 | MKFLYGIVFITLFLTVMIATHTEAAMCMPCFTTNLNMEQECRDCCGGTGR |
| | CFGPQCLCGYD |
| SEQ ID NO: 308 | RCSPCFTTDQQMTKKCYDCCGGKGKGKCYGPQCICAPY |
| SEQ ID NO: 309 | CGPCFTTDPYTESKCATCCGGRGKCVGPQCLCNRI |
| SEQ ID NO: 310 | TEAMCMPCFTTDHNMAKKCRDCCGGNGKCFGYQCLCNRG |
| SEQ ID NO: 311 | MKFLYGIVFIALFLTVMFATQTDGCGPCFTTDANMARKCRECCGGIGKCF |
| | GPQCLCNRI |
| SEQ ID NO: 312 | MKFLYGIVFIALFLTVMFATQTDGCGPCFTTDANMARKCRECCGGNGKCF |
| | GPQCLCNRE |
| SEQ ID NO: 313 | MKFLYGTILIAFFLTVMIATHSEARCPPCFTTNPNMEADCRKCCGGRGYCA |
| | SYQCICPGG |
| SEQ ID NO: 314 | TEAMCMPCFTTRPDMAQQCRDCCGGNGKCFGYQCLCNRG |
| SEQ ID NO: 315 | MKFLYGIVFIALFLTVMIATLTEAMCMPCFTTRPDMAQQCRDCCGGNGKC |
| | FGYQCLCNRG |
| SEQ ID NO: 316 | MKFLYGIVFIALFLTVMIATHTEAMCMPCFTTRPDMAQQCRDCCGGNGKC |
| | FGYQCLCNRG |
| SEQ ID NO: 317 | MKFLYGIILIALFLTVMIATHSEARCPNCFTTNPNAEADCKKCCGNRWGKC |
| | AGYQCVCPMK |
| SEQ ID NO: 318 | MKFLYGIVFIALFLTGMIATHTEAMCMPCFTTRPDMAQQCRDCCGGNGKC |
| | FGYQCLCNRGRIVIMYT |
| SEQ ID NO: 319 | MCMPCFTTRPGMAQQCRDCCGGNGKCFGYQCLCNR |
| SEQ ID NO: 320 | MCIPCFTTNPNMAAKCNACCGSRRGSCRGPQCICR |
| SEQ ID NO: 321 | MCIPCFTTNPNMAAKCNACCGSRRGSCRGPQCICN |
| SEQ ID NO: 322 | MCIPCFTTNPNMAAKCNACCGGNGSCRGPQCICN |
| SEQ ID NO: 323 | MCIPCFTTNPNMAAKCNACCGSRGRGSCRGPQCICN |
| SEQ ID NO: 324 | MCIPCFTTNPNMAAKCNACCGSRGRGKCRGPQCICN |
| SEQ ID NO: 325 | MCIPCFTTDHQMAAKCNACCGSRRGSCRGPQCICN |
| SEQ ID NO: 326 | MCIPCFTTNHQMAAKCNACCGSRRGSCRGPQCICN |
| SEQ ID NO: 327 | MCIPCFTTNPNMARKCNACCGSRGRGSCRGPQCICN |
| SEQ ID NO: 328 | MCIPCFTTNPNMAAKCNACCGGKGRGSCRGPQCICN |
| SEQ ID NO: 329 | MCIPCFTTNPNMAAKCNACCGSRRGSCFGPQCICN |
| SEQ ID NO: 330 | MCIPCFTTNPNMAAKCNACCGSRGRGKCFGPQCICN |
| SEQ ID NO: 331 | MCIPCFTTNPNMAAKCNACCGSRGRGSCFGPQCICN |
| SEQ ID NO: 332 | MCIPCFTTNPNMAAKCNACCGSRGRGSCYGPQCICN |
| SEQ ID NO: 333 | MCIPCFTTNPNMAAKCDACCGSRRGSCRGPQCICN |
| SEQ ID NO: 334 | MCIPCFTTNHQMAAKCDACCGSRRGSCRGPQCICN |
| SEQ ID NO: 335 | MCIPCFTTNHNMAAKCDACCGGRGRGSCRGPQCICN |
| SEQ ID NO: 336 | MCIPCFTTNPNMAAKCDACCGSRGRGSCRGPQCICN |
| SEQ ID NO: 337 | MCIPCFTTNPNMAAKCDACCGGKGRGSCRGPQCICN |
| SEQ ID NO: 338 | MCIPCFTTNHNMAAKCDACCGSRGRGSCRGPQCICN |
| SEQ ID NO: 339 | MCIPCFTTNPNMAAKCRDCCGGRGSCRGPQCICN |
| SEQ ID NO: 340 | MCMPCFTTNPNMAAKCDDCCGSRGRGSCRGPQCICN |
| SEQ ID NO: 341 | MCIPCFTTNPNMAARCNACCGSRRGSCRGPQCIC |
| SEQ ID NO: 342 | MCIPCFTTNPNMAAKCNACCGSRRGSCRGPQCICI |
| SEQ ID NO: 343 | GCLQFMWKCNPDNDKCCRPNLKCNTYHKWCEFVTGK |
| SEQ ID NO: 344 | DCLGFLWKCNPSNDKCCRPNLVCSRKDKWCKYQI |
| SEQ ID NO: 345 | DCLGFMRKCIPDNDKCCRPNLVCSRTHKWCKYVFGK |
| SEQ ID NO: 346 | ECLEIFKACNPSNDQCCKSSKLVCSRKTRACKYQI |
| SEQ ID NO: 347 | ECGGFWWKCGSGKPACCPKYVCSPKWGLCNFPMP |
| SEQ ID NO: 348 | GCLERWWKCNPNDDKCCRPKLKCSKLFKLCNRSRG |
| SEQ ID NO: 349 | GCLEEWWKCNPNDDKCCRPKLKCSKLFKLCNESEG |
| SEQ ID NO: 350 | GCLEIWWKCNPNDDKCCRPKLKCSKLFKLCNYSIG |
| SEQ ID NO: 351 | GCLEFWWKCNPNDDKCCRPKLKCSKLGKLCNFSFG |
| SEQ ID NO: 352 | GCLEFWWKCNPNDDKCCRPKLKCSPLGKLCNFSFG |
| SEQ ID NO: 353 | GCLEFWWKCNPNDDKCCRPKLKCSPNGKLCNFSFG |
| SEQ ID NO: 354 | GCLEFWWKCNPNDDKCCRPKLKCSRKTKLCNFSFG |
| SEQ ID NO: 355 | GCLEFWWKCNPNDDKCCRPKLKCGSNFKLCNFSFG |
| SEQ ID NO: 356 | GCLEFWWKCNPNDDKCCRPKLKCSTKHKLCNFSFG |
| SEQ ID NO: 357 | GCLEFWWKCNPNDDKCCRPKLKCSNDGKLCNFSFG |
| SEQ ID NO: 358 | GCLEFWWKCNPNDDKCCRPKLKCSKKTKLCNFSFG |
| SEQ ID NO: 359 | GCLEFWWKCNPNDDKCCRPKLKCHSNFKLCNFSFG |
| SEQ ID NO: 360 | GCLEFWWKCNPNDDKCCRPKLKCSKKFTACNFSFG |
| SEQ ID NO: 361 | GCLEIFKACNPNDDKCCRPKLKCSKLFKLCNFSFG |
| SEQ ID NO: 362 | GCLKFGWKCNPNDDKCCRPKLKCSKLFKLCNFSFG |
| SEQ ID NO: 363 | GCLEFWWKCNPNDDKCCKSSKLKCSKLFKLCNFSFG |
| SEQ ID NO: 364 | GCLEFWWKCNPNDDKCCRPKLKCNKLFKLCNISIG |
| SEQ ID NO: 365 | GCLEFWWKCNPNDDCCRKLKCSKLFKLCNFSFG |
| SEQ ID NO: 366 | GCLEFWWKCNPSNDQCCRPKLKCSKLFKLCNFSFG |
| SEQ ID NO: 367 | GCLEFWWKCNPNDDKCCRPSKLVCSKLFKLCNFSFG |
| SEQ ID NO: 368 | GCLEFLGECNPNDDKCCRPKLKCSKLFKLCNFSFG |
| SEQ ID NO: 369 | GCLWYLWKCNPNDDKCCRPKLKCSKLFKLCNFSFG |
| SEQ ID NO: 370 | GCLEFWWRCNPNDDRCCRPRLRCSRLFRLCNFSFG |
| SEQ ID NO: 371 | GCLEFWWRCNPNDDRCCRPRLRCSRLFRLC |
| SEQ ID NO: 372 | CRYFLGECKKTSECCEHLACHDKHKWCAWDWTIGK |
| SEQ ID NO: 373 | ECRYWLGGCSAGQTCCKHLVCSRRHGWCVWDGTF |
| SEQ ID NO: 374 | ECRWYLGECSQDGDCCKHLQCHSNYEWCIWDGTFSK |
| SEQ ID NO: 375 | ECRWYLGGCSQDGDCCKHLQCHSNYEWCVWDGTFSK |
| SEQ ID NO: 376 | DCRKFLGACTQTSDCCKHLACHNKHKWCAWDWTI |
| SEQ ID NO: 377 | ECRYLMGGCSKDGDCCEHLVCRTKWPYHCVWDWTFGK |
| SEQ ID NO: 378 | ECRYRLGTCSKTGDCCSHLSCSPKHGWCVRDRTFRK |
| SEQ ID NO: 379 | ECRYELGTCSKTGDCCSHLSCSPKHGWCVEDETFRK |
| SEQ ID NO: 380 | ECRYILGTCSKTGDCCSHLSCSPKHGWCVYDITFRK |
| SEQ ID NO: 381 | ECRYWLGTCSKTGDCCSHLSCSPKGGWCVWDWTFRK |
| SEQ ID NO: 382 | ECRYWLGTCSKTGDCCSHLSCSPNHGWCVWDWTFRK |
| SEQ ID NO: 383 | ECRYWLGTCSKTGDCCSHLSCSRKTGWCVWDWTFRK |
| SEQ ID NO: 384 | ECRYWLGTCSKTGDCCSHLSCGSNHGWCVWDWTFRK |
| SEQ ID NO: 385 | ECRYWLGTCSKTGDCCSHLSCSTKHGWCVWDWTFRK |
| SEQ ID NO: 386 | ECRYWLGTCSKTGDCCSHLSCSSKHGWCVWDWTFRK |
| SEQ ID NO: 387 | ECRYWLGTCSKTGDCCSHLSCSNDGGWCVWDWTFRK |
| SEQ ID NO: 388 | ECRYWLGTCSKTGDCCSHLSCSPKTRACVWDWTFRK |
| SEQ ID NO: 389 | ECRYWLGTCSKTGDCCSHLSCHSNHGWCVWDWTFRK |
| SEQ ID NO: 390 | ECRYWLGTCSKTGDCCSHLSCSRKHRACVWDWTFRK |
| SEQ ID NO: 391 | ECRYWLGTCSKTGDQCCKSSHLSCSPKHGWCVWDWTFRK |
| SEQ ID NO: 392 | ECRYWLGTCSAGQDCCSHLSCSPKHGWCVWDWTFRK |
| SEQ ID NO: 393 | ECRYWLGGCSATGDCCSHLSCSPKHGWCVWDWTFRK |
| SEQ ID NO: 394 | ECRYWLGTCSKTGDCCKSSHLVCSPKHGWCVWDWTFRK |
| SEQ ID NO: 395 | ECLEILGTCSKTGDCCSHLSCSPKHGWCVWDWTFRK |
| SEQ ID NO: 396 | ECRYWFKACSKTGDCCSHLSCSPKHGWCVWDWTFRK |
| SEQ ID NO: 397 | ECRYWLGTCSKTGDCCSHLSCSDGHGWCVWDWTFRK |
| SEQ ID NO: 398 | ECRYWLGTCSKTGDCCSHLSCSKLHGWCVWDWTFRK |
| SEQ ID NO: 399 | ECRYWLGTCSKTGDCCSHLQCHSKHGWCVWDWTFRK |
| SEQ ID NO: 400 | ECRYWLGTCSRTGDCCSHLSCSPRHGWCVWDWTFRR |
| SEQ ID NO: 401 | ECRYWLGTCSRTGDCCSHLSCSPRHGWC |
| SEQ ID NO: 402 | RCLPPGRPCYGATQRIPCCGVCSHNNCTELYENKPRRPYIL |
| SEQ ID NO: 403 | VGCEECPMHCRGRNANPTCDDGVCNCNVELYENKPRRPYIL |
| SEQ ID NO: 404 | MCMPCFTTDTQMQERCDRCCGGGGRGRCWGPQCLCIELYENKPRRPYIL |
| SEQ ID NO: 405 | MCMPCFTTDPNMAKKCRDCCGGNGKCFGPQCLCNRELYENKPRRPYIL |
| SEQ ID NO: 406 | EKDCIKHLQRCRENKDCCSKKCSRRGTNPEKRCRELYENKPRRPYIL |

In some instances, a peptide of the disclosure can comprise the sequence GSXICX²PCFTTX³X⁴X⁵X⁶X⁷X⁸X⁹C X¹⁰X¹¹CCGX¹²X¹³X¹⁴X¹⁵GX¹⁶CX¹⁷GPX¹⁸CX¹⁹CX² (SEQ ID NO: 198) or a fragment thereof, where X¹, X², X³, X⁴, X⁵, X⁶, X⁷, X⁸, X⁹, X¹, X¹¹, X¹², X¹³, X¹⁴, X¹⁵, X¹⁶, X¹⁷, X¹⁸, X¹⁹, and X²⁰ are each individually any amino acid or amino acid analogue.

In some instances, a peptide of the disclosure can comprise the sequence X¹CX²PCFTTX³X⁴X⁵X⁶X⁷X⁸X⁹CX¹⁰X¹¹CCGX¹²X¹³X¹⁴X¹⁵GX¹⁶CX¹⁷GPX¹⁸CX¹⁹CX²⁰ (SEQ ID NO: 407) or a fragment thereof, where X¹, X², X³, X⁴, X⁵, X⁶, X⁷, X⁸, X⁹, X¹, X¹¹, X¹², X¹³, X¹⁴, X¹⁵, X¹⁶, X¹⁷, X¹⁸, X¹⁹, and X²⁰ are each individually any amino acid or amino acid analogue.

In some instances, the peptides of the disclosure can comprise the sequence GSX¹CX²PCFTTX³X⁴X⁵X⁶X⁷X⁸X⁹C X¹⁰X¹¹CCGX¹²X¹³X¹⁴X¹⁵GX¹⁶CX¹⁷GPX¹⁸CX¹⁹CX²⁰ (SEQ ID NO: 199) or a fragment thereof, where: X¹ is selected from M, R, I, D, H, or L; X² is selected from M, I or L; X³ is selected from D, H, E, S, G, or I; X⁴ is selected from H, E, Q, R, Y, or T; X⁵ is selected from Q, R, H, E, Y, or F; X⁶ is selected from M, I, or L; X⁷ is selected from A, F, E, I, or Q; X⁸ is selected from R, E, I, D, N, or H; X⁹ is selected from R, N, H, E, Y, F, I, T, or Q; X¹⁰ is selected from D or E; X¹¹ is selected from D, I H, E, R, Y, F, or A; X¹² is selected from G or I; X¹³ is selected from R, D, W, F, or G; X¹⁴ is selected from G, D, or S; X¹⁵ is selected from R, D, G, or Y; X¹⁶ is selected from K or R; X¹⁷ is selected from Y, N, H, D, or W; X¹⁸ is selected from Q or H; X¹⁹ is selected from L or I; and X²⁰ is selected from R, G, F, or I.

In some instances, the peptides of the disclosure can comprise the sequence X¹CX²PCFTTX³X⁴X⁵X⁶X⁷X⁸X⁹CX¹⁰X¹¹CCGX¹²X¹³X¹⁴X¹⁵GX¹⁶CX¹⁷GPX¹⁸CX¹⁹CX²⁰ (SEQ ID NO: 408) or a fragment thereof, where: X¹ is selected from M, R, I, D, H, or L; X² is selected from M, I or L; X³ is selected from D, H, E, S, G, or I; X⁴ is selected from H, E, Q, R, Y, or T; X⁵ is selected from Q, R, H, E, Y, or F; X⁶ is selected from M, I, or L; X⁷ is selected from A, F, E, I, or Q; X⁸ is selected from R, E, I, D, N, or H; X⁹ is selected from R, N, H, E, Y, F, I, T, or Q; X¹⁰ is selected from D or E; X¹¹ is selected from D, I H, E, R, Y, F, or A; X¹² is selected from G or I; X¹³ is selected from R, D, W, F, or G; X¹⁴ is selected from G, D, or S; X¹⁵ is selected from R, D, G, or Y; X¹⁶ is selected from K or R; X¹⁷ is selected from Y, N, H, D, or W; X¹⁸ is selected from Q or H; X¹⁹ is selected from L or I; and X²⁰ is selected from R, G, F, or I.

In some instances, a peptide of the disclosure can comprise the sequence GSVGCEECPX¹HCX²GX³X⁴AX⁵PTCDX⁶GVCNCNV (SEQ ID NO: 201) or a fragment thereof, wherein X¹, X², X³, X⁴, X⁵, and X⁶ are each individually any amino acid or amino acid analogue.

In some instances, a peptide of the disclosure can comprise the sequence VGCEECPX¹HCX²GX³X⁴AX⁵PTCDX⁶GVCNCNV (SEQ ID NO: 410) or a fragment thereof, wherein X¹, X², X³, X⁴, X⁵, and X⁶ are each individually any amino acid or amino acid analogue.

In other cases, a peptides can comprise the sequence GSVGCEECPX¹HCX²GX³X⁴AX⁵PTCDX⁶GVCNCNV (SEQ ID NO: 202) or a fragment thereof, where X¹ is selected from M, A, V, I, or L, wherein X² is selected from K or R, wherein X³ is selected from K or R, wherein X⁴ is selected from N, H, M, K, or Q, wherein X⁵ is selected from N, K, V, I, L, R or Q, and wherein X⁶ is selected from D, N, G, Y, or E.

In other cases, a peptides can comprise the sequence VGCEECPX¹HCX²GX³X⁴AX⁵PTCDX⁶GVCNCNV (SEQ ID NO: 411) or a fragment thereof, where X¹ is selected from M, A, V, I, or L, wherein X² is selected from K or R, wherein X³ is selected from K or R, wherein X⁴ is selected from N, H, M, K, or Q, wherein X⁵ is selected from N, K, V, I, L, R or Q, and wherein X⁶ is selected from D, N, G, Y, or E.

In some instances, a peptide of the disclosure can comprise the sequence GSVGCX¹EX²PX³X⁴CKGKX⁵AX⁶pTCX⁷X⁸X⁹X¹⁰CX¹¹CNX¹² (SEQ ID NO: 203) or a fragment thereof, where X, X², X³, X⁴, X⁵, X⁶, X⁷, X⁸, X⁹, X¹⁰, X¹¹, and X¹² are each individually any amino acid or amino acid analogue.

In some instances, a peptide of the disclosure can comprise the sequence VGCX¹EX²PX³X⁴CKGKXAX⁶pTCX⁷X⁸X⁹X¹⁰CX¹¹CNX¹² (SEQ ID NO: 412) or a fragment thereof, where X¹, X², X³, X⁴, X⁵, X⁶, X⁷, X⁸, X⁹, X¹, X¹¹, and X¹² are each individually any amino acid or amino acid analogue.

In some instances, a peptide of the disclosure can comprise the sequence GSVGCX¹EX²PX³X⁴CKGKX⁵AX⁶pTCX⁷X⁸X⁹X¹⁰CX¹¹CNX¹² (SEQ ID NO: 204) or a fragment thereof, where: X¹ is selected from A or E; X² is selected from C or D; X is selected from M, A, K, or V; X⁴ is selected from H or Y; X⁵ is selected from N, H, M, K, or Q; X⁶ is selected from N, K V, I, or L; X is selected from D, Y, C, or E; X is selected from D, N, G, or Y; X⁹ is selected from G or E; X¹⁰ is selected from V or is absent; X¹¹ is selected from N, K, or E; and X¹² is selected from V, A, I, or D.

In some instances, a peptide of the disclosure can comprise the sequence VGCX¹EX²PX³X⁴CKGKXAX⁶pTCX⁷X⁸X⁹X¹⁰CX¹¹CNX¹² (SEQ ID NO: 413) or a fragment thereof, where: X¹ is selected from A or E; X² is selected from C or D; X is selected from M, A, K, or V; X⁴ elected from H or Y; X¹ is selected from N, H, M, K, or Q; X⁶ is selected from N, K V, I, or L; X⁷ is selected from D, Y, C, or E; X⁸ is selected from D, N, G, or Y; X⁹ is selected from G or E; X¹⁰ is selected from V or is absent; X¹¹ is selected from N, K, or E; and X¹² is selected from V, A, I, or D.

In some instances, a peptide of the disclosure can comprise the sequence GSX¹X²CEDCPX³HCX⁴X⁵X⁶X⁷X⁸X⁹AKCX¹⁰NDX¹¹CVCEX²X¹³ (SEQ ID NO: 205) or a fragment thereof, where X, X², X³, X⁴, X⁵, X⁶, X⁷, X⁸, X⁹, X¹, X¹¹, X¹², and X¹³ are each individually any amino acid or amino acid analogue.

In some instances, a peptide of the disclosure can comprise the sequence X¹X²CEDCPX³HCX⁴X⁵X⁶X⁷X⁸X⁹AKCX¹⁰DX¹¹CVCEX²X¹³ (SEQ ID NO: 414) or a fragment thereof, where X, X², X³, X⁴, X⁵, X⁶, X⁷, X⁸, X⁹, X¹, X¹¹, X¹², and X¹³ are each individually any amino acid or amino acid analogue.

In some instances, a peptide of the disclosure can comprise the sequence GSX¹X²CEDCPX³HCX⁴X⁵X⁶X⁷X⁸X⁹AKCX¹NDX¹¹CVCEX¹²X¹³ (SEQ ID NO: 206) or a fragment thereof, where: X¹ is selected from V or A; X² is selected from S, T, or G; X³ is selected from D or E; X⁴ is selected from S or A; X⁵ is selected from T or Q; X⁶ is selected from Q or K; X⁷ is selected from K, N, or D; X⁸ is selected from A or Q; X⁹ is selected from R or Q; X¹⁰ is selected from D or E; X¹¹ is selected from K or R; X¹² is selected from P, S, or A; and X¹³ is selected from K, V, or I.

In some instances, a peptide of the disclosure can comprise the sequence X¹X²CEDCPX³HCX⁴X⁵X⁶X⁷X⁸X⁹AKCX¹NDX¹¹CVCEX¹²X (SEQ ID NO: 415) or a fragment thereof, where: X¹ is selected from V or A; X² is selected from S, T, or G; X³ is selected from D or E; X⁴ is selected from S or A; X⁵ is selected from T or Q; X⁶ is selected from Q or K; X⁷ is selected from K, N, or D; X⁸ is selected from A or Q; X⁹ is selected from R or Q; X¹⁰ is selected from D or E; X¹¹ is selected from K or R; X¹² is selected from P, S, or A; and X¹³ is selected from K, V, or I.

In some instances, a peptide of the disclosure can comprise the sequence GSX¹CX²PCFTTDHQX²ARRCDDCCGGRGRGX³CYGPQCX²CX⁴ (SEQ ID NO: 207) or a fragment thereof, where: X¹ is any amino acid or amino acid analogue except P or C; X² is independently selected from A, L, V, I, or M; X³ is selected from K or R; and X⁴ is any amino acid or amino acid analogue except C.

In some instances, a peptide of the disclosure can comprise the sequence X¹CX²PCFTTDHQX²ARRCDDCCGGRGRGX³CYGPQCX²CX⁴ (SEQ ID NO: 416) or a fragment thereof, where: X¹ is any amino acid or amino acid analogue except P or C; X² is independently selected from A, L, V, I, or M; X³ is selected from K or R; and X⁴ is any amino acid or amino acid analogue except C.

In some instances, a peptide of the disclosure can comprise the sequence GSMCMPCFTTDHRMAENCDICCGGDGRGXCYGPQCLCR (SEQ ID NO: 208) or a fragment thereof, where X is R or K.

In some instances, a peptide of the disclosure can comprise the sequence MCMPCFTTDHRMAENCDICCGGDGRGXCYGPQCLCR (SEQ ID NO: 417) or a fragment thereof, where X is R or K.

In some instances, a peptide of the disclosure can comprise the sequence GSXCMPCFTTXXXMXXXCDXCCGXXXXGXCXGPXCLCX (SEQ ID NO: 209) or a fragment thereof, where X can independently be any amino acid or amino acid analogue.

In some instances, a peptide of the disclosure can comprise the sequence XCMPCFTTXXXMXXXCDXCCGXXXXGXCXGPXCLCX (SEQ ID NO: 418) or a fragment thereof, where X can independently be any amino acid or amino acid analogue.

In some embodiments, a peptide of the present disclosure comprise a sequence having cysteine residues at one or more of positions 4, 5, 7, 8, 12, 18, 21, 22, 26, 28, 30, 35, or 37. For example, in certain embodiments, a peptide comprises a sequence having a cysteine residue at position 4. In certain embodiments, a peptide comprises a sequence having a cysteine residue at position 5. In certain embodiments, a peptide comprises a sequence having a cysteine residue at position 7. In certain embodiments, a peptide comprises a sequence having a cysteine residue at position 8. In certain embodiments, a peptide comprises a sequence having a cysteine residue at position 12. In certain embodiments, a peptide comprises a sequence having a cysteine residue at position 18. In certain embodiments, a peptide comprises a sequence having a cysteine residue at position 21. In certain embodiments, a peptide comprises a sequence having a cysteine residue at position 22. In certain embodiments, a peptide comprises a sequence having a cysteine residue at position 26. In certain embodiments, a peptide comprises a sequence having a cysteine residue at position 28. In certain embodiments, a peptide comprises a sequence having a cysteine residue at position 30. In certain embodiments, a peptide comprises a sequence having a cysteine residue at position 35. In certain embodiments, a peptide comprises a sequence having a cysteine residue at position 37. In some embodiments, the first cysteine residue in the sequence is disulfide bonded with the 4^{th} cysteine residue in the sequence, the 2^{nd} cysteine residue in the sequence is disulfide bonded to the 5^{th} cysteine residue in the sequence, and the 3^{rd} cysteine residue in the sequence is disulfide bonded to the 6^{th} cysteine residue in the sequence. In some embodiments, the 1^{st} cysteine residue in the sequence is disulfide bonded to the 4^{th} cysteine residue in the sequence, the second cysteine residue in the sequence is disulfide bonded to the 6^{th} cysteine residue in the sequence, the 3^{rd} cysteine residue in the sequence is disulfide bonded to the 7^{th} cysteine residue in the sequence, and the 5 cysteine residue in the sequence is disulfide bonded to the 8th cysteine residue in the sequence. Optionally, a peptide can comprise one disulfide bridge that passes through a ring formed by two other disulfide bridges, also known as a “two-and-through” structure system.

In some embodiments, a peptide of the present disclosure can comprise the sequence GSCXXCXXXXXXXXXXCXXCCXXXXXXXCXXXXCXC (SEQ ID NO: 200), where at least some or all of the cysteine residues form intramolecular disulfide bridges and X is any amino acid or amino acid analogue.

In some embodiments, a peptide of the present disclosure can comprise the sequence CXXCXXXXXXXXXXCXXCCXXXXXXXCXXXXCXC (SEQ ID NO: 409), where at least some or all of the cysteine residues form intramolecular disulfide bridges and X is any amino acid or amino acid analogue.

In some instances, the peptide can contain only one lysine residue, or no lysine residues. In some instances, some or all of the lysine residues in the peptide are replaced with arginine residues. In some instances, some or all of the methionine residues in the peptide are replaced by leucine or isoleucine. In some instances, some or all of the tryptophan residues in the peptide are replaced by phenylalanine or tyrosine. In some instances, some or all of the asparagine residues in the peptide are replaced by glutamine. In some cases, the N-terminus of the peptide is blocked, such as by an acetyl group. Alternatively or in combination, in some instances, the C-terminus of the peptide is blocked, such as by an amide group. In some embodiments, the peptide is modified by methylation on free amines. For example, full methylation may be accomplished through the use of reductive methylation with formaldehyde and sodium cyanoborohydride.

In some cases, the first two N-terminal amino acids shown (GS) in SEQ ID NO: 1-SEQ ID NO: 209, or such N-terminal amino acids (GS) can be absent, or substituted by any other one or two amino acids, as shown in SEQ ID NO: 210-SEQ ID NO: 418.

In some cases, the C-terminal Arg residues of a peptide is modified to another residue such as Ala, Asn, Asp, Gln, Glu, Gly, His, Leu, Lys, Met, Phe, Pro, Ser, Thr, Trp, Tyr, or Val. For example, the C-terminal Arg residue of a peptide can be modified to Ile. Alternatively, the C-terminal Arg residue of a peptide can be modified to any non-natural amino acid. This modification can prevent clipping of the C-terminal residue during expression, synthesis, processing, storage, in vitro, or in vivo including during treatment, while still allowing maintenance of a key hydrogen bond. A key hydrogen bond can be the hydrogen bond formed during the initial folding nucleation and is critical for forming the initial hairpin.

Generally, the NMR solution structures of related structural homologs can be used to inform mutational strategies that may improve the folding, stability, manufacturability, while maintaining a particular biological function. They can be used to predict the 3D pharmacophore of a group of structurally homologous scaffolds, as wells as to predict possible graft regions of related proteins to create chimeras with improved properties. For example, we have used this strategy to identify critical amino acid positions and loops that may be used to design drugs with improved properties or to correct deleterious mutations that complicate folding and manufacturability for the peptides of SEQ ID NO: 5, SEQ ID NO: 1, SEQ ID NO: 2, and SEQ ID NO: 3. TABLE 2 summarizes key amino acid positions and loops that have been used with some success as learned from SEQ ID NO: 5. In some aspects, the amino acids listed in the table below may be retained while other residues in the peptide sequences may be mutated to improve, change, remove, or otherwise modify function, homing, and activity of the peptide.

TABLE 2
Exemplary key amino acid positions and
loops according to the present disclosure.

| Amino Acid Position | Interacting Residues |
|---|---|
| T10 | H11, H12 |
| D19 | C22, G23, G24, G26, R27 |
| R38 | R27 |

With respect to the above residues in TABLE 2, it is understood that the positions and interacting residues above describe different but corresponding positions within any peptide sequence described herein. For example, the first two N-terminal amino acids shown (GS) in SEQ ID NO: 1-SEQ ID NO: 209 can be absent, or substituted by any other one or two amino acids, as shown in SEQ ID NO: 210-SEQ ID NO: 418, and in such peptides where the N-terminal amino acids (GS) are absent, amino acid position T10 would correspond to T8 with the interacting residues H11, H12 corresponding to H9, H10; amino acid position D19 would correspond to D17 with interacting residues C22, G23, G24, G26, and R27 corresponding to C20, G21, G22, G24, and R25, and amino acid position R38 would correspond to R36 with interacting residue R27 corresponding to R25. Additionally, the interacting residue at position 11 can be substituted with aspartic acid. Similarly, any variants of the peptides described herein for SEQ ID NO: 1-SEQ ID NO: 196 would have similarly corresponding residues.

Additionally, the comparison of the primary sequences and the tertiary sequences of two or more peptides can be used to reveal sequence and 3D folding patterns that can be leveraged to improve the peptides and parse out biological activity of these peptides. For example, comparing two different peptide scaffolds that cross the BBB or enter the CSF can lead to the identification of conserved pharmacophores that can guide engineering strategies, such as designing variants with improved folding properties. Important pharmacores, for example, can comprise aromatic residues, which can be important for protein-protein binding interactions.

In some instances, the peptide is any one of SEQ ID NO: 1-SEQ ID NO: 192 or a functional fragment thereof. In other embodiments, the peptide of the disclosure further comprises a peptide with 99%, 95%, 90%, 85%, or 80% sequence identity or homology to any one of SEQ ID NO: 1-SEQ ID NO: 192, or fragment thereof.

In other instances, the peptide can be a peptide that is homologous to any one of SEQ ID NO: 1-SEQ ID NO: 192, or a functional fragment thereof. The term “homologous” is used herein to denote peptides having at least 70%, at least 80%, at least 90%, at least 95%, or greater than 95% sequence identity or homology to a sequence of any one of SEQ ID NO: 1-SEQ ID NO: 192 or SEQ ID NO: 210-SEQ ID NO: 410, or a functional fragment thereof.

In still other instances, the variant nucleic acid molecules of a peptide of any one of SEQ ID NO: 1-SEQ ID NO: 192 or SEQ ID NO: 210-SEQ ID NO: 410 can be identified by either a determination of the sequence identity or homology of the encoded peptide amino acid sequence with the amino acid sequence of any one of SEQ ID NO: 1-SEQ ID NO: 192 or SEQ ID NO: 210-SEQ ID NO: 410, or by a nucleic acid hybridization assay. Such peptide variants can include nucleic acid molecules (1) that remain hybridized with a nucleic acid molecule having the nucleotide sequence of any one of SEQ ID NO: 1-SEQ ID NO: 192 or SEQ ID NO: 210-SEQ ID NO: 410 (or any complement of the previous sequences) under stringent washing conditions, in which the wash stringency is equivalent to 0.5×-2×SSC with 0.1% SDS at 55-65° C., and (2) that encode a peptide having at least 70%, at least 80%, at least 90%, at least 95% or greater than 95% sequence identity or homology to the amino acid sequence of any one of SEQ ID NO: 1-SEQ ID NO: 192 or SEQ ID NO: 210-SEQ ID NO: 410. Alternatively, peptide variants of any one of SEQ ID NO: 1-SEQ ID NO: 192 or SEQ ID NO: 210-SEQ ID NO: 410 can be characterized as nucleic acid molecules (1) that remain hybridized with a nucleic acid molecule having the nucleotide sequence of any one of SEQ ID NO: 1-SEQ ID NO: 192 or SEQ ID NO: 210-SEQ ID NO: 410 (or any complement of the previous sequences) under highly stringent washing conditions, in which the wash stringency is equivalent to 0.1×-0.2×SSC with 0.1% SDS at 50-65° C., and (2) that encode a peptide having at least 70%, at least 80%, at least 90%, at least 95% or greater than 95% sequence identity or homology to the amino acid sequence of any one of SEQ ID NO: 1-SEQ ID NO: 192 or SEQ ID NO: 210-SEQ ID NO: 410.

Percent sequence identity or homology is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (Id.). The sequence identity or homology is then calculated as: ([Total number of identical matches]/[length of the longer sequence plus the number of gaps introduced into the longer sequence in order to align the two sequences])(100).

Additionally, there are many established algorithms available to align two amino acid sequences. For example, the “FASTA” similarity search algorithm of Pearson and Lipman is a suitable protein alignment method for examining the level of sequence identity or homology shared by an amino acid sequence of a peptide disclosed herein and the amino acid sequence of a peptide variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 1) and a test sequence that has either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity or homology of nucleic acid molecules using a ratio as disclosed above. For nucleotide sequence comparisons, the ktup value can range between one to six, preferably from three to six, most preferably three, with other parameters set as described above.

Some examples of common amino acids that are a “conservative amino acid substitution” are illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Determination of amino acid residues that are within regions or domains that are critical to maintaining structural integrity can be determined. Within these regions one can determine specific residues that can be more or less tolerant of change and maintain the overall tertiary structure of the molecule. Methods for analyzing sequence structure include, but are not limited to, alignment of multiple sequences with high amino acid or nucleotide identity or homology and computer analysis using available software (e.g., the Insight II® viewer and homology modeling tools; MSI, San Diego, Calif.), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments determination of structure can typically be accompanied by evaluating activity of modified molecules.

In further embodiments, the peptide fragment comprises a contiguous fragment of any one of SEQ ID NO: 1-SEQ ID NO: 196 that is at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46 residues long, wherein the peptide fragment is selected from any portion of the peptide.

The peptides of the present disclosure can further comprise positively charged amino acid residues. In some cases, the peptide has at least 1 positively charged residue. In some cases, the peptide has at least 2 positively charged residues. In some cases, the peptide has at least 3 positively charged residues. In other cases, the peptide has at least 4 positively charged residues, at least 5 positively charged residues, at least 6 positively charged residues, at least 7 positively charged residues, at least 8 positively charged residues or at least 9 positively charged residues.

While the positively charged residues can be selected from any positively charged amino acid residues, in some embodiments, the positively charged residues are either K, or R, or a combination of K and R.

The peptides of the present disclosure can further comprise neutral amino acid residues. In some cases, the peptide has 35 or fewer neutral amino acid residues. In other cases, the peptide has 81 or fewer neutral amino acid residues, 70 or fewer neutral amino acid residues, 60 or fewer neutral amino acid residues, 50 or fewer neutral amino acid residues, 40 or fewer neutral amino acid residues, 36 or fewer neutral amino acid residues, 33 or fewer neutral amino acid residues, 30 or fewer neutral amino acid residues, 25 or fewer neutral amino acid residues, or 10 or fewer neutral amino acid residues.

The peptides of the present disclosure can further comprise negative amino acid residues. In some cases the peptide has 6 or fewer negative amino acid residues, 5 or fewer negative amino acid residues, 4 or fewer negative amino acid residues, 3 or fewer negative amino acid residues, 2 or fewer negative amino acid residues, or 1 or fewer negative amino acid residues. While negative amino acid residues can be selected from any neutral charged amino acid residues, in some embodiments, the negative amino acid residues are either E, or D, or a combination of both E and D.

At physiological pH, peptides can have a net charge, for example, of −5, −4, −3, −2, −1, 0, +1, +2, +3, +4, or +5. When the net charge is zero, the peptide can be uncharged or zwitterionic. In some embodiments, the peptide contains one or more disulfide bonds and has a positive net charge at physiological pH where the net charge can be +0.5 or less than +0.5, +1 or less than +1, +1.5 or less than +1.5, +2 or less than +2, +2.5 or less than +2.5, +3 or less than +3, +3.5 or less than +3.5, +4 or less than +4, +4.5 or less than +4.5, +5 or less than +5, +5.5 or less than +5.5, +6 or less than +6, +6.5 or less than +6.5, +7 or less than +7, +7.5 or less than +7.5, +8 or less than +8, +8.5 or less than +8.5, +9 or less than +9.5, +10 or less than +10. In some embodiments, the peptide has a negative net charge at physiological pH where the net charge can be −0.5 or less than −0.5, −1 or less than −1, −1.5 or less than −1.5, −2 or less than −2, −2.5 or less than −2.5, −3 or less than −3, −3.5 or less than −3.5, −4 or less than −4, −4.5 or less than −4.5, −5 or less than −5, −5.5 or less than −5.5, −6 or less than −6, −6.5 or less than −6.5, −7 or less than −7, −7.5 or less than −7.5, −8 or less than −8, −8.5 or less than −8.5, −9 or less than −9.5, −10 or less than −10. In some cases, the engineering of one or more mutations within a peptide yields a peptide with an altered isoelectric point, charge, surface charge, or rheology at physiological pH. Such engineering of a mutation to a peptide derived from a scorpion or spider can change the net charge of the complex, for example, by decreasing the net charge by 1, 2, 3, 4, or 5, or by increasing the net charge by 1, 2, 3, 4, or 5. In such cases, the engineered mutation may facilitate the ability of the peptide to cross the blood brain barrier. Suitable amino acid modifications for improving the rheology and potency of a peptide can include conservative or non-conservative mutations. A peptide can comprises at most 1 amino acid mutation, at most 2 amino acid mutations, at most 3 amino acid mutations, at most 4 amino acid mutations, at most 5 amino acid mutations, at most 6 amino acid mutations, at most 7 amino acid mutations, at most 8 amino acid mutations, at most 9 amino acid mutations, at most 10 amino acid mutations, or another suitable number as compared to the sequence of the venom or toxin component that the peptide is derived from. In other cases, a peptide, or a functional fragment thereof, comprises at least 1 amino acid mutation, at least 2 amino acid mutations, at least 3 amino acid mutations, at least 4 amino acid mutations, at least 5 amino acid mutations, at least 6 amino acid mutations, at least 7 amino acid mutations, at least 8 amino acid mutations, at least 9 amino acid mutations, at least 10 amino acid mutations, or another suitable number as compared to the sequence of the venom or toxin component that the peptide is derived from. In some embodiments, mutations can be engineered within a peptide to provide a peptide that has a desired charge or stability at physiological pH.

The present disclosure also encompasses multimers of the various peptides described herein. Examples of multimers include dimers, trimers, tetramers, pentamers, hexamers, heptamers, and so on. A multimer may be a homomer formed from a plurality of identical subunits or a heteromer formed from a plurality of different subunits. In some embodiments, a peptide of the present disclosure is arranged in a multimeric structure with at least one other peptide, or two, three, four, five, six, seven, eight, nine, ten, or more other peptides. In certain embodiments, the peptides of a multimeric structure each have the same sequence. In alternative embodiments, some or all of the peptides of a multimeric structure have different sequences.

The present disclosure further includes peptide scaffolds that, e.g., can be used as a starting point for generating additional peptides. In some embodiments, these scaffolds can be derived from a variety of knotted peptides or knottins. Some suitable peptides for scaffolds can include, but are not limited to, chlorotoxin, brazzein, circulin, stecrisp, hanatoxin, midkine, hefutoxin, potato carboxypeptidase inhibitor, bubble protein, attractin, α-GI, α-GID, μ-PIIIA, ω-MVIIA, w-CVID, X-MrIA, p-TIA, conantokin G, contulakin G, GsMTx4, margatoxin, shK, toxin K, chymotrypsin inhibitor (CTI), and EGF epiregulin core.

In some cases the peptide comprises the sequence of any one of SEQ ID NO: 1-SEQ ID NO: 192 or SEQ ID NO: 210-SEQ ID NO: 410. In some embodiments, the peptide sequence is flanked by additional amino acids. One or more additional amino acids can, for example, confer a desired in vivo charge, isoelectric point, chemical conjugation site, stability, or physiologic property to a peptide.

Two or more peptides can share a degree of sequence identity or homology and share similar properties in vivo. For instance, a peptide can share a degree of sequence identity or homology with any one of the peptides of SEQ ID NO: 1-SEQ ID NO: 192. In some cases, one or more peptides of the disclosure can have up to about 20% pairwise sequence identity or homology, up to about 25% pairwise sequence identity or homology, up to about 30% pairwise sequence identity or homology, up to about 35% pairwise sequence identity or homology, up to about 40% pairwise sequence identity or homology, up to about 45% pairwise sequence identity or homology, up to about 50% pairwise sequence identity or homology, up to about 55% pairwise sequence identity or homology, up to about 60% pairwise sequence identity or homology, up to about 65% pairwise sequence identity or homology, up to about 70% pairwise sequence identity or homology, up to about 75% pairwise sequence identity or homology, up to about 80% pairwise sequence identity or homology, up to about 85% pairwise sequence identity or homology, up to about 90% pairwise sequence identity or homology, up to about 95% pairwise sequence identity or homology, up to about 96% pairwise sequence identity or homology, up to about 97% pairwise sequence identity or homology, up to about 98% pairwise sequence identity or homology, up to about 99% pairwise sequence identity or homology, up to about 99.5% pairwise sequence identity or homology, or up to about 99.9% pairwise sequence identity or homology. In some cases, one or more peptides of the disclosure can have at least about 20% pairwise sequence identity or homology, at least about 25% pairwise sequence identity or homology, at least about 30% pairwise sequence identity or homology, at least about 35% pairwise sequence identity or homology, at least about 40% pairwise sequence identity or homology, at least about 45% pairwise sequence identity or homology, at least about 50% pairwise sequence identity or homology, at least about 55% pairwise sequence identity or homology, at least about 60% pairwise sequence identity or homology, at least about 65% pairwise sequence identity or homology, at least about 70% pairwise sequence identity or homology, at least about 75% pairwise sequence identity or homology, at least about 80% pairwise sequence identity or homology, at least about 85% pairwise sequence identity or homology, at least about 90% pairwise sequence identity or homology, at least about 95% pairwise sequence identity or homology, at least about 96% pairwise sequence identity or homology, at least about 97% pairwise sequence identity or homology, at least about 98% pairwise sequence identity or homology, at least about 99% pairwise sequence identity or homology, at least about 99.5% pairwise sequence identity or homology, at least about 99.9% pairwise sequence identity or homology with a second peptide. Various methods and software programs can be used to determine the homology between two or more peptides, such as NCBI BLAST, Clustal W, MAFFT, Clustal Omega, AlignMe, Praline, or another suitable method or algorithm.

Pairwise sequence alignment is used to identify regions of similarity that may indicate functional, structural and/or evolutionary relationships between two biological sequences (protein or nucleic acid). By contrast, multiple sequence alignment (MSA) is the alignment of three or more biological sequences. From the output of MSA applications, homology can be inferred and the evolutionary relationship between the sequences assessed. One of skill in the art would recognize as used herein, “sequence homology” and “sequence identity” and “percent (%) sequence identity” and “percent (%) sequence homology” have been used interchangeably to mean the sequence relatedness or variation, as appropriate, to a reference polynucleotide or amino acid sequence.

### Chemical Modifications

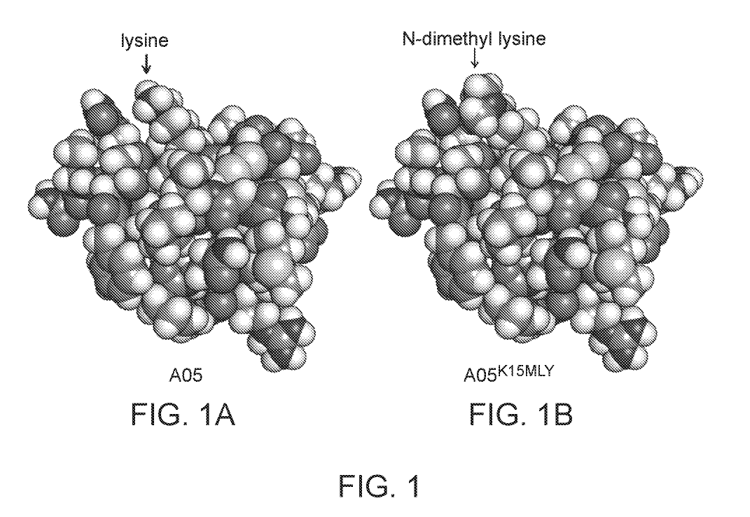

A peptide can be chemically modified one or more of a variety of ways. In some embodiments, the peptide can be mutated to add function, delete function, or modify the in vivo behavior. One or more loops between the disulfide linkages can be modified or replaced to include active elements from other peptides (such as described in Moore and Cochran, Methods in Enzymology, 503, p. 223-251, 2012). Amino acids can also be mutated, such as to increase half-life, modify, add or delete binding behavior in vivo, add new targeting function, modify surface charge and hydrophobicity, or allow conjugation sites. N-methylation is one example of methylation that can occur in a peptide of the disclosure. In some embodiments, the peptide is modified by methylation on free amines. For example, full methylation may be accomplished through the use of reductive methylation with formaldehyde and sodium cyanoborohydride. FIG. 1 illustrates a model of a peptide of SEQ ID NO: 1 with and without methylation.

A chemical modification can, for instance, extend the half-life of a peptide or change the biodistribution or pharmacokinetic profile. A chemical modification can comprise a polymer, a polyether, polyethylene glycol, a biopolymer, a polyamino acid, a fatty acid, a dendrimer, an Fc region, a simple saturated carbon chain such as palmitate or myristolate, or albumin. A polyamino acid can include, for example, a poly amino acid sequence with repeated single amino acids (e.g., poly glycine), and a poly amino acid sequence with mixed poly amino acid sequences (e.g, gly-ala-gly-ala (SEQ ID NO: 424)) that may or may not follow a pattern, or any combination of the foregoing.

In some embodiments, the peptides of the present disclosure may be modified such that the modification increases the stability and/or the half-life of the peptides. In some embodiments, the attachment of a hydrophobic moiety, such as to the N-terminus, the C-terminus, or an internal amino acid, can be used to extend half-life of a peptide of the present disclosure. In other embodiments, the peptide of the present disclosure can include post-translational modifications (e.g., methylation and/or amidation), which can affect, e.g., serum half-life. In some embodiments, simple carbon chains (e.g., by myristoylation and/or palmitylation) can be conjugated to the fusion proteins or peptides. In some embodiments, the simple carbon chains may render the fusion proteins or peptides easily separable from the unconjugated material. For example, methods that may be used to separate the fusion proteins or peptides from the unconjugated material include, but are not limited to, solvent extraction and reverse phase chromatography. The lipophilic moieties can extend half-life through reversible binding to serum albumin. The conjugated moieties can, e.g., be lipophilic moieties that extend half-life of the peptides through reversible binding to serum albumin. In some embodiments, the lipophilic moiety can be cholesterol or a cholesterol derivative including cholestenes, cholestanes, cholestadienes and oxysterols. In some embodiments, the peptides can be conjugated to myristic acid (tetradecanoic acid) or a derivative thereof. In other embodiments, the peptides of the present disclosure are coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin.

In some embodiments, the first two N-terminal amino acids (GS) of SEQ ID NO: 1-SEQ ID NO: 196 serve as a spacer or linker in order to facilitate conjugation or fusion to another molecule, as well as to facilitate cleavage of the peptide from such conjugated or fused molecules. In some embodiments, the fusion proteins or peptides of the present disclosure can be conjugated to other moieties that, e.g., can modify or effect changes to the properties of the peptides.

### Active Agent Conjugates

Peptides according to the present disclosure can be conjugated or fused to an agent for use in the treatment of tumors, cancers, and brain diseases and disorders. For example, in certain embodiments, the peptides described herein are fused to another molecule, such as an active agent that provides a functional capability. A peptide can be fused with an active agent through expression of a vector containing the sequence of the peptide with the sequence of the active agent. In various embodiments, the sequence of the peptide and the sequence of the active agent are expressed from the same Open Reading Frame (ORF). In various embodiments, the sequence of the peptide and the sequence of the active agent can comprise a contiguous sequence. The peptide and the active agent can each retain similar functional capabilities in the fusion peptide compared with their functional capabilities when expressed separately. In certain embodiments, examples of active agents include other peptides such as neurotensin peptide. Neurotensin is a 13 amino acid neuropeptide that can be involved in the regulation of luteinizing hormone and prolactin release, and can interact with the dopaminergic system, but does not cross the blood brain barrier. Therefore, the fusion of neurotensin peptide and one of the peptides described herein that can cross the blood brain barrier can produce a fusion peptide capable of crossing the blood barrier which can retain the functional capabilities of neurotensin peptide. For example, the DNA sequence of a peptide of the present disclosure is inserted into the gene of neurotensin to manufacture peptide-neurotensin fusions.

Furthermore, for example, in certain embodiments, the peptides described herein are attached to another molecule, such as an active agent that provides a functional capability. In some embodiments, 1, 2, 3, 4, 5, 6, 7, 8, 9, or 10 active agents can be linked to a peptide. Multiple active agents can be attached by methods such as conjugating to multiple lysine residues and/or the N-terminus, or by linking the multiple active agents to a scaffold, such as a polymer or dendrimer and then attaching that agent-scaffold to the peptide (such as described in Yurkovetskiy, A. V., Cancer Res 75(16): 3365-72 (2015). Examples of active agents include but are not limited to: a peptide, an oligopeptide, a polypeptide, a peptidomimetic, a polynucleotide, a polyribonucleotide, a DNA, a cDNA, a ssDNA, a RNA, a dsRNA, a micro RNA, an oligonucleotide, an antibody, a single chain variable fragment (scFv), an antibody fragment, an aptamer, a cytokine, an interferon, a hormone, an enzyme, a growth factor, a checkpoint inhibitor, a PD-1 inhibitor, a PD-L1 inhibitor, a CTLA4 inhibitor, a CD antigen, a chemokine, a neurotransmitter, an ion channel inhibitor, an ion channel activator, a G-protein coupled receptor inhibitor, a G-protein coupled receptor activator, a chemical agent, a radiosensitizer, a radioprotectant, a radionuclide, a therapeutic small molecule, a steroid, a corticosteroid, an anti-inflammatory agent, an immune modulator, a complement fixing peptide or protein, a tumor necrosis factor inhibitor, a tumor necrosis factor activator, a tumor necrosis factor receptor family agonist, a tumor necrosis receptor antagonist, a Tim-3 inhibitor, a protease inhibitor, an amino sugar, a chemotherapeutic, a cytotoxic chemical, a toxin, a tyrosine kinase inhibitor, an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, polyethylene glycol, a lipid, a dendrimer, a fatty acid, or an Fc region, or an active fragment or a modification thereof. In some embodiments, the peptide is covalently or non-covalently linked to an active agent, e.g., directly or via a linker. For example, cytotoxic molecules that can be usedinclude auristatins, MMAE, MMAF, dolostatin, auristatin F, monomethylaurstatin D, DM1, DM4, maytansinoids, maytansine, calicheamicins, N-acetyl-γ-calicheamicin, pyrrolobenzodiazepines, PBD dimers, doxorubicin, vinca alkaloids (4-deacetylvinblastine), duocarmycins, cyclic octapeptide analogs of mushroom amatoxins, epothilones, and anthracylines, CC-1065, taxanes, paclitaxel, cabazitaxel, docetaxel, SN-38, irinotecan, vincristine, vinblastine, platinum compounds, cisplatin, methotrexate, and BACE inhibitors. Additional examples of active agents are described in McCombs, J. R., AAPS J, 17(2): 339-51 (2015), Ducry, L., Antibody Drug Conjugates (2013), and Singh, S. K., Pharm Res. 32(11): 3541-3571 (2015). The peptide disclosed herein can be used to home, distribute to, target, directed to, accumulate in, migrate to, and/or bind to cancerous cells, and thus also be used for localizing the attached or fused active agent. Furthermore, knotted chlorotoxin peptide can be internalized in cells (Wiranowska, M., Cancer Cell Int., 11: 27 (2011)). Therefore, cellular internalization, subcellular localization, and intracellular trafficking after internalization of the active agent peptide conjugate or fusion peptide can be important factors in the efficacy of an active agent conjugate or fusion. (Ducry, L., Antibody Drug Conjugates (2013); and Singh, S. K., Pharm Res. 32(11): 3541-3571 (2015)). Exemplary linkers suitable for use with the embodiments herein are discussed in further detail below.

As compared to antibody-drug conjugates (e.g., Adcetris, Kadcyla, Mylotarg), in some aspects the peptide conjugated to an active agent as described herein may exhibit better penetration of solid tumors due to its smaller size. In other aspects, the peptide conjugated to an active agent as described herein may also be able better reach to brain tumors due to its ability to penetrate the BBB as compared to antibody-drug conjugates. In certain aspects, the peptide conjugated to an active agent as described herein may be able to carry different or higher doses of active agents as compared to antibody-drug conjugates. In still other aspects, the peptide conjugated to an active agent as described herein may have better site specific delivery of defined drug ratio as compared to antibody-drug conjugates. In other aspects, the peptide may be amenable to solvation in organic solvents (in addition to water), which may allow more synthetic routes for solvation and conjugation of a drug (which often has low aqueous solubility) and higher conjugation yields, higher ratios of drug conjugated to peptide (versus an antibody), and/or reduce aggregate/high molecular weight species formation during conjugation. Additionally, a unique amino acid residue(s) may be introduced into the peptide via a residue that is not otherwise present in the short sequence or via inclusion of a non-natural amino acid, allowing site specific conjugation to the peptide.

The peptides or fusion peptides of the present disclosure can also be conjugated to other moieties that can serve other roles, such as providing an affinity handle (e.g., biotin) for retrieval of the peptides from tissues or fluids. For example, peptides or fusion peptides of the present disclosure can also be conjugated to biotin. In addition to extension of half-life, biotin could also act as an affinity handle for retrieval of peptides or fusion peptides from tissues or other locations. In some embodiments, fluorescent biotin conjugates that can act both as a detectable label and an affinity handle can be used. Non limiting examples of commercially available fluorescent biotin conjugates include Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, Alexa fluor 488 biocytin, Alexa flour 546, Alexa Fluor 549, lucifer yellow cadaverine biotin-X, Lucifer yellow biocytin, Oregon green 488 biocytin, biotin-rhodamine and tetramethylrhodamine biocytin. In some other examples, the conjugates could include chemiluminescent compounds, colloidal metals, luminescent compounds, enzymes, radioisotopes, and paramagnetic labels. In some embodiments, the peptide described herein can also be attached to another molecule. For example, the peptide sequence also can be attached to another active agent (e.g., small molecule, peptide, polypeptide, polynucleotide, antibody, aptamer, cytokine, growth factor, neurotransmitter, an active fragment or modification of any of the preceding, fluorophore, radioisotope, radionuclide chelator, acyl adduct, chemical linker, or sugar, etc.). In some embodiments, the peptide can be fused with, or covalently or non-covalently linked to an active agent.

Additionally, more than one peptide sequence derived from a toxin or venom can be present on or fused with a particular peptide. A peptide can be incorporated into a biomolecule by various techniques. A peptide can be incorporated by a chemical transformation, such as the formation of a covalent bond, such as an amide bond. A peptide can be incorporated, for example, by solid phase or solution phase peptide synthesis. A peptide can be incorporated by preparing a nucleic acid sequence encoding the biomolecule, wherein the nucleic acid sequence includes a subsequence that encodes the peptide. The subsequence can be in addition to the sequence that encodes the biomolecule, or can substitute for a subsequence of the sequence that encodes the biomolecule.

### Detectable Agent Conjugates

A peptide can be conjugated to an agent used in imaging, research, therapeutics, theranostics, pharmaceuticals, chemotherapy, chelation therapy, targeted drug delivery, and radiotherapy. In some embodiments, a peptide is conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, or 10 detectable agents can be linked to a peptide. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that could be used as a conjugating molecule in the present disclosure include DyLight-680, DyLight-750, VivoTag-750, DyLight-800, IRDye-800, VivoTag-680, Cy5.5, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acradine orange or yellow, Alexa Fluors (e.g., Alexa Fluor 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other Suitable fluorescent dyes include, but are not limited to, fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US14/56177. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

Other embodiments of the present disclosure provide peptides conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of diseased cells (e.g., cancer cells) using both a therapeutic agent (e.g., drug) and electromagnetic energy (e.g., radiation or light) concurrently. In some embodiments, the peptide is fused with, or covalently or non-covalently linked to the agent, e.g., directly or via a linker. Exemplary linkers suitable for use with the embodiments herein are discussed in further detail below Linkers

Peptides according to the present disclosure that home, distribute to, target, migrate to, accumulate in, or are directed to cancerous or diseased cells or a specific brain region (e.g., the hippocampus, ventricular system, CSF) can be attached to another moiety (e.g., an active agent or an detectable agent), such as a small molecule, a second peptide, a protein, an antibody, an antibody fragment, an aptamer, polypeptide, polynucleotide, a fluorophore, a radioisotope, a radionuclide chelator, a polymer, a biopolymer, a fatty acid, an acyl adduct, a chemical linker, or sugar or other active agent or detectable agent described herein through a linker, or directly in the absence of a linker. A peptide that crosses the blood-brain barrier or blood CSF can be attached to another molecule, such as a small molecule, a second peptide, a protein, an antibody, an antibody fragment, an aptamer, a polypeptide, a fluorophore, a radioisotope, a radionuclide chelator, a polymer, a biopolymer, a fatty acid, an acyl adduct, a chemical linker, or sugar or other active agent or detectable agent described herein through a linker or directly, in the absence of a linker. In the absence of a linker, for example, an active agent or an detectable agent can be fused to the N-terminus or the C-terminus of a peptide to create an active agent or detectable agent fusion peptide. In other embodiments, the link can be made by a peptidic fusion via reductive alkylation.

Direct attachment is possible by covalent attachment of a peptide to a region of the other molecule. For example, an active agent or an detectable agent can be fused to the N-terminus or the C-terminus of a peptide to create an active agent or detectable agent fusion peptide. As another example, the peptide can be attached at the N-terminus, an internal lysine residue, or the C-terminus to a terminus of the amino acid sequence of the other molecule by a linker. If the attachment is at an internal lysine residue, the other molecule can be linked to the peptide at the epsilon amine of the internal lysine residue. For example, the internal lysine residues can be located at a position corresponding to amino acid residue 17 of SEQ ID NO: 37, amino acid residue 25 of SEQ ID NO: 37, or amino acid residue 29 of SEQ ID NO: 37 or similar residues of the disclosed peptide(s), such as any of the corresponding lysine residues in any one of SEQ ID NO: 1-SEQ ID NO: 196. As another example, the internal lysine residues can be located at a position corresponding to amino acid residue 15 of SEQ ID NO: 246, amino acid residue 23 of SEQ ID NO: 246, or amino acid residue 27 of SEQ ID NO: 246 or similar residues of the disclosed peptide(s), such as any of the corresponding lysine residues in any one of SEQ ID NO: 210-SEQ ID NO: 405. In some further examples, the peptide can be attached to the other molecule by a side chain, such as the side chain of a lysine, serine, threonine, cysteine, tyrosine, aspartic acid, a non-natural amino acid residue, or glutamic acid residue. A linker can be an amide bond, an ester bond, an ether bond, a carbamate bond, a carbonate bond, a carbon-nitrogen bond, a triazole, a macrocycle, an oxime bond, a hydrazone bond, a carbon-carbon single, double, or triple bond, a disulfide bond, a two carbon bridge between two cysteines, a three carbon bridge between two cysteines, or a thioether bond. In still other embodiments, the peptide comprises a non-natural amino acid, wherein the non-natural amino acid is an insertion, appendage, or substitution for another amino acid, and the peptide is linked to the active agent at the non-natural amino acid by a linker. In some embodiments, similar regions of the disclosed peptide(s) itself (such as a terminus of the amino acid sequence, an amino acid side chain, such as the side chain of a lysine, serine, threonine, cysteine, tyrosine, aspartic acid, a non-natural amino acid residue, or glutamic acid residue, via an amide bond, an ester bond, an ether bond, a carbamate bond, a carbon-nitrogen bond, a triazole, a macrocycle, an oxime bond, a hydrazone bond, a carbon-carbon single, double, or triple bond, a disulfide bond, a thioether bond, or other linker as described herein) may be used to link other molecules.

Attachment via a linker involves incorporation of a linker moiety between the other molecule and the peptide. The peptide and the other molecule can both be covalently attached to the linker. The linker can be cleavable, non-cleavable, self-immolating, hydrophilic, or hydrophobic. The linker has at least two functional groups, one bonded to the other molecule, and one bonded to the peptide, and a linking portion between the two functional groups. Some example linkers are described in Jain, N., Pharm Res. 32(11): 3526-40 (2015), Doronina, S. O., Bioconj Chem. 19(10): 1960-3 (2008), Pillow, T. H., J Med Chem. 57(19): 7890-9 (2014), Dorywalksa, M., Bioconj Chem. 26(4): 650-9 (2015), Kellogg, B. A., Bioconj Chem. 22(4): 717-27 (2011), and Zhao, R. Y., J Med Chem. 54(10): 3606-23 (2011).

Non-limiting examples of the functional groups for attachment include functional groups capable of forming, for example, an amide bond, an ester bond, an ether bond, a carbonate bond, a carbamate bond, a carbon-nitrogen bond, a triazole, a macrocycle, an oxime bond, a hydrazone bond, a carbon-carbon single, double, or triple bond, a disulfide bond or a thioether bond. Non-limiting examples of functional groups capable of forming such bonds include amino groups; carboxyl groups; aldehyde groups; azide groups; alkyne and alkene groups; ketones; hydrazides; hydrazines; acid halides such as acid fluorides, chlorides, bromides, and iodides; acid anhydrides, including symmetrical, mixed, and cyclic anhydrides; carbonates; carbonyl functionalities bonded to leaving groups such as cyano, succinimidyl, and N-hydroxysuccinimidyl; maleimides; linkers containing maleimide groups that are designed to hydrolyze; maleimidocaproyl; MCC ([N-maleimidomethyl]cyclohexane-1-carboxylate); N-ethylmaleimide; maleimide alkane; mc-vc-PABC; DUBA (DuocarmycinhydroxyBenzamide-Azaindole linker); SMCC Succinimidyl-4-(N-maleimidomethyl) cyclohexane-1-carboxylate; SPDP (N-succinimidyl-3-(2-pyridyldithio) propionate); SPDB N-succinimidyl-4-(2-pyridyldithio) butanoate; sulfo-SPDB N-succinimidyl-4-(2-pyridyldithio)-2-sulfo butanoate; SPP N-succinimidyl 4-(2-pyridyldithio)pentanoate; a dithiopyridylmaleimide (DTM); a hydroxylamine, a vinyl-halo group; haloacetamido groups; bromoacetamido; hydroxyl groups; sulfhydryl groups; and molecules possessing, for example, alkyl, alkenyl, alkynyl, allylic, or benzylic leaving groups, such as halides, mesylates, tosylates, triflates, epoxides, phosphate esters, sulfate esters, and besylates.

Non-limiting examples of the linking portion include alkylene, alkenylene, alkynylene, polyether, such as polyethylene glycol (PEG), polyester, polyamide, polyamino acids, polypeptides, cleavable peptides, Val-Cit, Phe-Lys, Val-Lys, Val-Ala, other peptide linkers as given in Doronina et al., 2008, linkers cleavable by beta glucuronidase, linkers cleavable by a cathepsin or by cathepsin B, D, E, H, L, S, C, K, O, F, V, X, or W, Val-Cit-p-aminobenzyloxycarbonyl, glucuronide-MABC, aminobenzylcarbamates, D-amino acids, and polyamine, any of which being unsubstituted or substituted with any number of substituents, such as halogens, hydroxyl groups, sulfhydryl groups, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, epoxides, charged groups, zwitterionic groups, and ester groups. Other non-limiting examples of reactions to link molecules together include click chemistry, copper-free click chemistry, HIPS ligation, Staudinger ligation, and hydrazine-iso-Pictet-Spengler.

Non-limiting examples of linkers include:

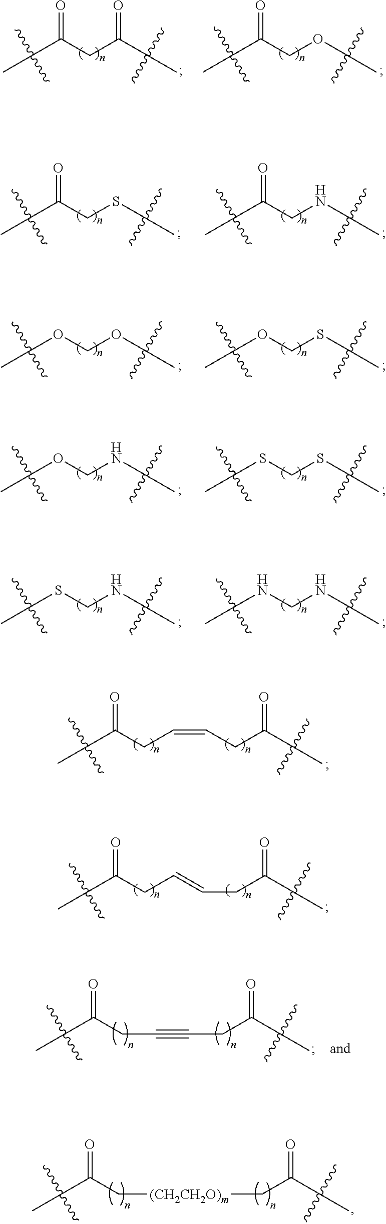

wherein each n is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50, or any linker as disclosed in Jain, N., Pharm Res. 32(11): 3526-40 (2015) or Ducry, L., Antibody Drug Conjugates (2013).

In some cases a linker can be a succinic linker, and a drug can be attached to a peptide via an ester bond or an amide bond with two methylene carbons in between. In other cases, a linker can be any linker with both a hydroxyl group and a carboxylic acid, such as hydroxy hexanoic acid or lactic acid.

In some embodiments, the linker can release the active agent in an unmodified form. In other embodiments, the active agent can be released with chemical modification. In still other embodiments, catabolism can release the active agent still linked to parts of the linker and/or peptide.

The linker may be a noncleavable linker or a cleavable linker. In some embodiments, the noncleavable linker can slowly release the conjugated moiety by an exchange of the conjugated moiety onto the free thiols on serum albumin. In some embodiments, the use of a cleavable linker can permit release of the conjugated moiety (e.g., a therapeutic agent) from the peptide, e.g., after targeting to the tumor or cancerous cell. In other embodiments, the use of a cleavable linker can permit the release of the conjugated therapeutic from the peptide after crossing the BBB and optionally after targeting to the specific brain region. In some cases the linker is enzyme cleavable, e.g., a valine-citrulline linker. In some embodiments, the linker contains a self-immolating portion. In other embodiments, the linker includes one or more cleavage sites for a specific protease, such as a cleavage site for matrix metalloproteases (MMPs), thrombin, cathepsins, or beta-glucuronidase. Alternatively or in combination, the linker is cleavable by other mechanisms, such as via pH, reduction, or hydrolysis.

The rate of hydrolysis or reduction of the linker can be fine-tuned or modified depending on an application. For example, the rate of hydrolysis of linkers with unhindered esters is faster compared to the hydrolysis of linkers with bulky groups next an ester carbonyl. A bulky group can be a methyl group, an ethyl group, a phenyl group, a ring, or an isopropyl group, or any group that provides steric bulk. In some cases, the steric bulk can be provided by the drug itself, such as by ketorolac when conjugated via its carboxylic acid. The rate of hydrolysis of the linker can be tuned according to the residency time of the conjugate in the target location. For example, when a peptide is cleared from a tumor, or the brain, relatively quickly, the linker can be tuned to rapidly hydrolyze. When a peptide has a longer residence time in the target location, a slower hydrolysis rate would allow for extended delivery of an active agent. “Programmed hydrolysis in designing paclitaxel prodrug for nanocarrier assembly” Sci Rep 2015, 5, 12023 Fu et al., provides an example of modified hydrolysis rates.

### Methods of Manufacture

Various expression vector/host systems can be utilized for the recombinant expression of peptides described herein. Non-limiting examples of such systems include microorganisms such as bacteria transformed with recombinant bacteriophage DNA, plasmid DNA or cosmid DNA expression vectors containing a nucleic acid sequence encoding peptides or peptide fusion proteins/chimeric proteins described herein, yeast transformed with recombinant yeast expression vectors containing the aforementioned nucleic acid sequence, insect cell systems infected with recombinant virus expression vectors (e.g., baculovirus) containing the aforementioned nucleic acid sequence, plant cell systems infected with recombinant virus expression vectors (e.g., cauliflower mosaic virus (CaMV), tobacco mosaic virus (TMV) or transformed with recombinant plasmid expression vectors (e.g., Ti plasmid) containing the aforementioned nucleic acid sequence, or animal cell systems infected with recombinant virus expression vectors (e.g., adenovirus, vaccinia virus) including cell lines engineered to contain multiple copies of the aforementioned nucleic acid sequence, either stably amplified (e.g., CHO/dhfr, CHO/glutamine synthetase) or unstably amplified in double-minute chromosomes (e.g., murine cell lines). Disulfide bond formation and folding of the peptide could occur during expression or after expression or both.

A host cell can be adapted to express one or more peptides described herein. The host cells can be prokaryotic, eukaryotic, or insect cells. In some cases, host cells are capable of modulating the expression of the inserted sequences, or modifying and processing the gene or protein product in the specific fashion desired. For example, expression from certain promoters can be elevated in the presence of certain inducers (e.g., zinc and cadmium ions for metallothionine promoters). In some cases, modifications (e.g., phosphorylation) and processing (e.g., cleavage) of peptide products can be important for the function of the peptide. Host cells can have characteristic and specific mechanisms for the post-translational processing and modification of a peptide. In some cases, the host cells used to express the peptides secrete minimal amounts of proteolytic enzymes.

In the case of cell- or viral-based samples, organisms can be treated prior to purification to preserve and/or release a target polypeptide. In some embodiments, the cells are fixed using a fixing agent. In some embodiments, the cells are lysed. The cellular material can be treated in a manner that does not disrupt a significant proportion of cells, but which removes proteins from the surface of the cellular material, and/or from the interstices between cells. For example, cellular material can be soaked in a liquid buffer, or, in the case of plant material, can be subjected to a vacuum, in order to remove proteins located in the intercellular spaces and/or in the plant cell wall. If the cellular material is a microorganism, proteins can be extracted from the microorganism culture medium. Alternatively, the peptides can be packed in inclusion bodies. The inclusion bodies can further be separated from the cellular components in the medium. In some embodiments, the cells are not disrupted. A cellular or viral peptide that is presented by a cell or virus can be used for the attachment and/or purification of intact cells or viral particles. In addition to recombinant systems, peptides can also be synthesized in a cell-free system prior to extraction using a variety of known techniques employed in protein and peptide synthesis.

In some cases, a host cell produces a peptide that has an attachment point for a drug. An attachment point could comprise a lysine residue, an N-terminus, a cysteine residue, a cysteine disulfide bond, or a non-natural amino acid. The peptide could also be produced synthetically, such as by solid-phase peptide synthesis, or solution-phase peptide synthesis. Peptide synthesis can be performed by fluorenylmethyloxycarbonyl (Fmoc) chemistry or by butyloxycarbonyl (Boc) chemistry. The peptide could be folded (formation of disulfide bonds) during synthesis or after synthesis or both. Peptide fragments could be produced synthetically or recombinantly. Peptide fragments can be then be joined together enzymatically or synthetically.

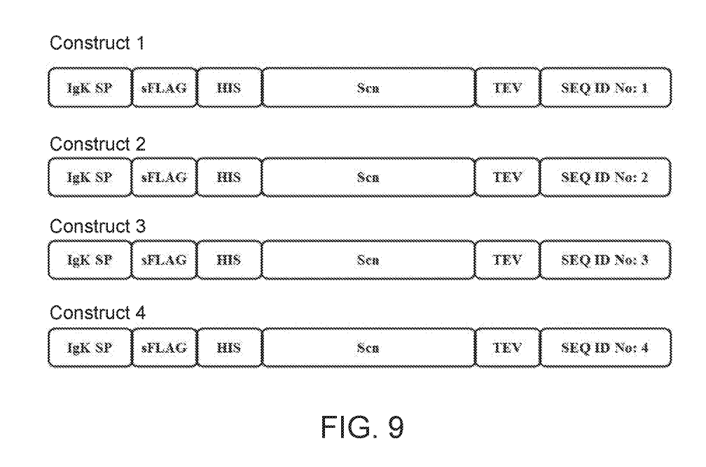

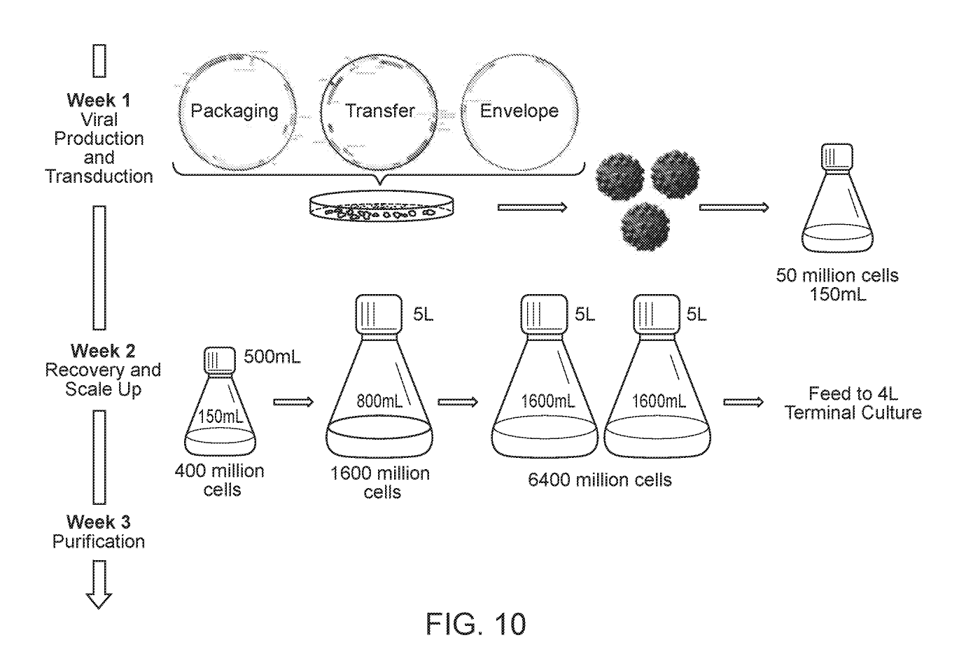

FIG. 10 illustrates a schematic of a method of manufacturing a construct that expresses a peptide of the disclosure, such as the constructs illustrated in FIG. 9 and as described throughout the disclosure and in SEQ ID NO: 1-SEQ ID NO: 196 provided herein.

In other aspects, the peptides of the present disclosure can be prepared by conventional solid phase chemical synthesis techniques, for example according to the Fmoc solid phase peptide synthesis method (“Fmoc solid phase peptide synthesis, a practical approach,” edited by W. C. Chan and P. D. White, Oxford University Press, 2000).

### Peptide Pharmaceutical Compositions

A pharmaceutical composition of the disclosure can be a combination of any peptide described herein with other chemical components, such as carriers, stabilizers, diluents, dispersing agents, suspending agents, thickening agents, antioxidants, solubilizers, buffers, osmolytes, salts, surfactants, amino acids, encapsulating agents, bulking agents, cryoprotectants, and/or excipients. The pharmaceutical composition facilitates administration of a peptide described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, and topical administration. A pharmaceutical composition can be administered in a local or systemic manner, for example, via injection of the peptide described herein directly into an organ, optionally in a depot.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide described herein in water-soluble form. Suspensions of peptides described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduces the aggregation of such peptides described herein to allow for the preparation of highly concentrated solutions. Alternatively, the peptides described herein can be lyophilized or in powder form for re-constitution with a suitable vehicle, e.g., sterile pyrogen-free water, before use. In some embodiments, a purified peptide is administered intravenously. A peptide described herein can be administered to a subject, home, target, migrate to, or be directed to an organ, e.g., the hippocampus and cross the blood brain barrier of a subject.

A peptide of the disclosure can be applied directly to an organ, or an organ tissue or cells, such as brain or brain tissue or cells, during a surgical procedure. The recombinant peptides described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of the peptide described herein described herein can be administered in pharmaceutical compositions to a subject suffering from a condition that affects the immune system. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a peptide described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of peptides described herein comprising the compounds described herein include formulating the peptide described herein with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

### Use of Peptides in Imaging and Surgical Methods

The present disclosure relates to peptides that home, distribute to, target, migrate to, accumulate in, or are directed to cancerous or diseased cells. The present disclosure relates to peptides that home, target, migrate to, accumulate in, or are directed to specific regions, tissues, structures, or cells within the body and methods of using such peptides. These peptides have the ability to bind to cross the blood brain barrier or blood CSF barrier, which makes them useful for a variety of applications. These abilities make them useful for a variety of applications. In particular, the peptides have applications in site-specific modulation of biomolecules to which the peptides are directed. End uses of such peptides include, for example, imaging, research, therapeutics, theranostics, pharmaceuticals, chemotherapy, chelation therapy, targeted drug delivery, and radiotherapy. Some uses can include targeted drug delivery and imaging.

In some embodiments, a peptide of the disclosure delivers a metal, a radioisotope, a dye, fluorophore, or another suitable material that can be used in imaging. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

In some embodiments, the fluorophore is a fluorescent agent emitting electromagnetic radiation at a wavelength between 650 nm and 4000 nm, such emissions being used to detect such agent. Non-limiting examples of fluorescent dyes that could be used as a conjugating molecule in the present disclosure include DyLight-680, DyLight-750, VivoTag-750, DyLight-800, IRDye-800, VivoTag-680, Cy5.5, ZW800, ZQ800, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acradine orange or yellow, Alexa Fluors (e.g., Alexa Fluor 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US14/56177 or another suitable material that can be used in imaging.

The present invention provides methods for intraoperative imaging and resection of a cancer, cancerous tissue, tumor tissue, cancerous cells, or diseased tissue using a peptide of the present disclosure conjugated with a detectable agent. In some aspects, the cancer, cancerous tissue, tumor tissue, or diseased tissue or cells of the foregoing is detectable by fluorescence imaging that allows for intraoperative visualization of the cancer, cancerous tissue, tumor tissue, cancerous cells, or diseased tissue using a peptide of the present disclosure. In some aspects, the peptide of the present disclosure is conjugated to one or more detectable agents. In a further embodiment, the detectable agent comprises a fluorescent moiety coupled to the peptide. In another embodiment, the detectable agent comprises a radionuclide. In some aspects, imaging is pre-operative imaging. In other aspects, imaging is achieved during open surgery. In further aspects, imaging is accomplished while using endoscopy or other non-invasive surgical techniques. In yet further aspects, imaging is performed after surgical removal of the cancer, cancerous tissue, tumor tissue, or diseased tissue or cells of the foregoing.

In some aspects, the present disclosure provides a method for detecting a cancer, cancerous tissue, tumor tissue or diseased tissue or cells of the foregoing, the method comprising the steps of contacting a tissue of interest with a peptide of the present disclosure, wherein the peptide is conjugated to a detectable agent and measuring the level of binding of the peptide, wherein an elevated level of binding is indicated by an increased detection of the detectable agent relative to normal tissue, which is indicative that the tissue is a cancer, cancerous tissue, tumor tissue or diseased tissue or cells of the foregoing.

### Treatment of Cancer

In one embodiment, the method includes administering an effective amount of a peptide of the present disclosure to a subject in need thereof.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. Compositions containing such agents or compounds can be administered for prophylactic, enhancing, and/or therapeutic treatments. An appropriate “effective” amount in any individual case may be determined using techniques, such as a dose escalation study.

The methods, compositions, and kits of this disclosure may comprise a method to prevent, treat, arrest, reverse, or ameliorate the symptoms of a condition. The treatment may comprise treating a subject (e.g., an individual, a domestic animal, a wild animal, or a lab animal afflicted with a disease or condition) with a peptide of the disclosure. The disease may be a cancer or tumor. In treating the disease, the peptide may contact the tumor or cancerous cells. The subject may be a human. Subjects can be humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants, and fetuses in utero.

Treatment may be provided to the subject before clinical onset of disease. Treatment may be provided to the subject after clinical onset of disease. Treatment may be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can comprise administering to a subject a pharmaceutical composition, such as one or more of the pharmaceutical compositions described throughout the disclosure. A treatment can comprise delivering a peptide of the disclosure to a subject, either intravenously, subcutaneously, intramuscularly, by inhalation, dermally, topically, orally, sublingually, intrathecally, transdermally, intranasally, via a peritoneal route, or directly into the brain, e.g., via and intracerebral ventrical route. A treatment can comprise administering a peptide-active agent complex to a subject, either intravenously, subcutaneously, intramuscularly, by inhalation, dermally, topically, orally, intrathecally, transdermally, intransally, parenterally, orally, via a peritoneal route, nasally, sublingually, or directly into the brain.

In some embodiments, the present disclosure provides a method for treating a cancer or tumor, the method comprising administering to a subject in need thereof an effective amount of a peptide of the present disclosure. One example of cancers or conditions that can be treated with a peptide of the disclosure is solid tumors. Further examples of cancers or conditions that can be treated with a peptide of the disclosure include triple negative breast cancer, breast cancer, breast cancer metastases, metastases of any cancers described herein, colon cancer, colon cancer metastases, sarcomas, acute lymphoblastic leukemia, acute myeloid leukemia, adrenocortical carcinoma, AIDS-related cancers such as Kaposi sarcoma, AIDS-related lymphoma, primary CNS lymphoma, anal cancer, appendix cancer, childhood astrocytomas, astrocytomas, childhood atypical teratoid/rhabdiod tumor, CNS atypical teratoid/rhabdiod tumor, atypical teratoid/rhabdiod tumor, basal cell carcinoma, skin cancer, bile duct cancer, bladder cancer, bone cancer, Ewing sarcoma family of tumors, osteosarcoma, chondroma, chondrosarcoma, primary and metastatic bone cancer, malignant fibrous histiocytoma, childhood brain stem glioma, brain stem glioma, brain tumor, brain and spinal cord tumors, central nervous system embryonal tumors, childhood central nervous system embryonal tumors, central nervous system germ cell tumors, childhood central nervous system germ cell tumors, craniopharyngioma, childhood craniopharyngioma, ependymoma, childhood ependymoma, breast cancer, bronchial tumors, childhood bronchial tumors, burkitt lymphoma, carcinoid tumor, gastrointestinal cancer, carcinoma of unknown primary, cardiac tumors, childhood cardiac tumors, primary lymphoma, cervical cancer, cholangiocarcinoma, chordoma, childhood chordoma, chronic lymphocytic leukemia, chronic myelogenous leukemia, chronic myeloproliferative neoplasms, colon cancer, colorectal cancer, cutaneous T cell lymphoma, ductal carcinoma in situ, endometrial cancer, esophageal cancer, esthesioneuroblastoma, childhood esthesioneuroblastoma, ewing sarcoma, extracranial germ cell tumor, childhood extracranial germ cell tumor, extragonadal germ cell tumor, eye cancer, intraocular melanoma, retinoblastoma, fallopian tube cancer, fibrous histiocytoma of bone, gallbladder cancer, gastric cancer, gastrointestinal carcinoid tumor, gastrointestinal stromal tumors, ovarian cancer, testicular cancer, gestational trophoblastic disease, glioma, hairy cell leukemia, head and neck cancer, hepatocellular cancer, histiocytosis, Langerhans cell histiocytosis, hodgkin lymphoma, hypopharyngeal cancer, intraocular melanoma, melanoma, melanoma metastases, islet cell tumors, pancreatic neuroendocrine tumors, kidney cancer, renal cell tumors, Wilms tumor, childhood kidney tumors, lip and oral cavity cancer, liver cancer, lung cancer, nonhodgkin lymphoma, macroglodulinemia, Waldenstrom macroglodulinemia, male breast cancer, merkel cell carcinoma, metastatic squamous neck cancer with occult primary, midline tract carcinoma involving NUT gene, mouth cancer, multiple endocrine neoplasia syndromes, childhood multiple endocrine neoplasia syndromes, multiple myeloma/plasma cell neoplasm, mycosis fungoides, myelodysplastic syndromes, myelodysplastic/myeloproliferative neoplasms, multiple myeloma, myloproliferative neoplasms, chronic myeloproliferative neoplasms, nasal cavity and paranasal sinus cancer, nasopharyngeal cancer, neuorblastoma, non-small cell lung cancer, oropharyngeal cancer, low malignant potential tumor, pancreatic cancer, pancreatic neuroendocrine tumors, papillomatosis, childhood papillomatosis, paraganglioma, paranasal sinus and nasal cavity cancer, parathyroid cancer, penile cancer, pheochromocytoma, pharyngeal cancer, pituitary tumor, pleuropulmonary blastoma, childhood pleuropulmonary blastoma, primary peritoneal cancer, prostate cancer, rectal cancer, pregnancy-related cancer, rhabdomyosarcoma, childhood rhabdomyosarcoma, salivary gland cancer, Sezary syndrome, small cell lung cancer, small intestine caner, soft tissue sarcoma, squamous cell carcinoma, testicular cancer, throat cancer, thymoma, thymic carcinoma, thyroid cancer, transitional cell cancer of the renal, pelvis, and ureter, uterine cancer, urethral cancer, endometrial cancer, uterine sarcoma, vaginal cancer, vascular tumors, and vulvar cancers.

In some embodiments, the peptide binds to potassium channels. In some embodiments the peptide binds to sodium channels. In some embodiments, the peptide blocks potassium channels and/or sodium channels, in some embodiments the peptide activates of potassium channels and/or sodium channels. In some embodiments, the peptide interacts with ion channels or chloride channels or calcium channels. In some embodiments the peptide interacts with nicotinic acetyl choline receptors, transient receptor potential channels, NMDA receptors, serotonin receptors, KIR channels, GABA channels, glycine receptors, glutamate receptors, acid sensing ion channels, K2P channels, Nav1.7, or purinergic receptors. In some embodiments, the peptide interacts with matrix metalloproteinase, inhibits cancer cell migration or metastases, or has antitumor activity. In some embodiments, the peptide interacts with calcium activated potassium channels. In some embodiments, the peptide has antibacterial, antifungal, or antiviral activity. In some embodiments, the peptide inhibits proteases. In some embodiments, the peptide interacts with channels that influence pain. In some embodiments, the peptide has other therapeutic effects on the tissue of an effected organ or structures thereof.

In some embodiments, the peptides of the present disclosure exhibit protease inhibitor activity. In certain embodiments, peptides are used to inhibit proteases of interest, such as coagulation-associated proteases (e.g., thrombin, factor 10a), metabolism-associated proteases (e.g., DPP-IV), cancer-associated proteases (e.g., matrix metalloproteinases, cathepsins), viral infection-associated proteases (e.g., HIV protease), and inflammation-associated proteases (e.g., tryptase, kallikrein).

In some embodiments, the peptides of the present disclosure can be modified to be anti-inflammatory, such as by incorporating properties of Immune Selective Anti-Inflammatory Derivatives (ImSAIDs). In certain embodiments, ImSAIDs are incorporated into or added onto peptides capable of targeting cancerous cells as described herein. FEG is an example of a key sequence that confers anti-inflammatory properties. Alternatively or in combination, peptides of the present disclosure can be conjugated to immune regulatory molecules to reverse, reduce, or limit inflammation.

In some embodiments, the peptides of the present disclosure are used to treat cancers. For example, in certain embodiments, the peptides provided herein are used to directly inhibit critical cancer-associated pathways such as RAS, MYC, PHF5A, BubR1, PKMYT1, or BuGZ.

In some aspects, the peptides of the present disclosure are conjugated to one or more therapeutic agents. In certain aspects, the therapeutic agent is a chemotherapeutic, anti-cancer drug, or anti-cancer agent selected from, but are not limited to: radioisotopes, toxins, enzymes, sensitizing drugs, nucleic acids, including interfering RNAs, antibodies, anti-angiogenic agents, cisplatin, platinum compounds, anti-metabolites, mitotic inhibitors, growth factor inhibitors, taxanes, paclitaxel, cabazitaxel, temozolomide, topotecan, fluorouracil, vincristine, vinblastine, 4-deacetylvinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane and amifostine, vinca alkaloids, cyclic octapeptide analogs of mushroom amatoxins, epothilones, and anthracylines, CC-1065, SN-38, and BACE inhibitors, and their equivalents, as well as photo-ablation agents. For example, in certain embodiments, a peptide of the present disclosure is conjugated to palbociclib, a CDK 4/6 inhibitor with limited ability to cross the blood brain barrier. As another example, in certain embodiments, a peptide of the present disclosure is conjugated to monomethyl auristatine E (MMAE), MMAF, auristatin, dolostatin, auristatin F, monomethylauristatin D, maytansinoid (e.g., DM-1, DM4, maytansine), pyrrolobenzodiazapine dimer, calicheamicin, N-acetyl-γ-calicheamicin, duocarmycin, anthracycline, a microtubule inhibitor, or a DNA damaging agent.

Optionally, certain embodiments of the present disclosure provide peptides conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cancer cells using both a therapeutic agent (e.g., drug) and electromagnetic energy (e.g., radiation or light) concurrently.

In certain embodiments, the peptide of the disclosure is mutated to home, distribute to, target, migrate to, accumulate in, or is directed to certain tissues but not to others, to change the strength or specificity of its function, or to gain or lose function, such as agonizing an ion channel or inhibiting a protease.

The present disclosure also encompasses the use of “tandem” peptides in which two or more peptides are conjugated or fused together. In certain embodiments, a tandem peptide comprises two or more knotted peptides conjugated or fused together, where at least one knotted peptide is capable of targeting to a specific region, while at least one other knotted peptide provides a specific therapeutic activity, such as a BIM analogue, as discussed above and herein.

In some embodiments, the present disclosure provides a method for treating a cancer, the method comprising administering to a subject in need thereof an effective amount of a peptide of the present disclosure.

In some embodiments, the present disclosure provides a method for treating a cancer, the method comprising administering to a patient in need thereof an effective amount of a pharmaceutical composition comprising a peptide of the present disclosure and a pharmaceutically acceptable carrier.

In some embodiments, the present disclosure provides a method for inhibiting invasive activity of cells, the method comprising administering an effective amount of a peptide of the present disclosure to a subject.

A peptide comprising the sequence of any of SEQ ID NO: 1-SEQ ID NO: 192 or SEQ ID NO: 210-SEQ ID NO: 401, and any peptide derivative or peptide-active agent as described herein, can be used to target upper GI disease and cancers (e.g., throat, oral, esophageal cancer, salivary glands, tonsils, pharynx, adenosarcomas, oral malignant melanoma head and neck cancer). A peptide comprising the sequence of any of SEQ ID NO: 1-SEQ ID NO: 192 or SEQ ID NO: 210-SEQ ID NO: 401, and any peptide derivative or peptide-active agent as described herein, can be used to additionally target gall bladder disease and cancers.

Venom or toxin derived peptide(s), peptides, modified peptides, labeled peptides, peptide-active agent conjugates and pharmaceutical compositions described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Such peptides described herein can also be administered to prevent (either in whole or in part), lessen a likelihood of developing, contracting, or worsening a condition. Amounts effective for this use can vary based on the severity and course of the disease or condition, previous therapy, the subject's health status, weight, and response to the drugs, and the calculations of the treating physician.

In some embodiments, the present disclosure provides a method of treating a tumor or cancerous cells of a subject, the method comprising administering to the subject a peptide comprising the sequence of any of SEQ ID NO: 1-SEQ ID NO: 196 or SEQ ID NO: 210-SEQ ID NO: 405, or a functional fragment thereof.

Multiple peptides described herein can be administered in any order or simultaneously. In some cases, multiple functional fragments of peptides derived from toxins or venom can be administered in any order or simultaneously. If simultaneously, the multiple peptides described herein can be provided in a single, unified form, such as an intravenous injection, or in multiple forms, such as subsequent intravenous dosages.

Peptides can be packaged as a kit. In some embodiments, a kit includes written instructions on the use or administration of the peptides.

### Treatment of Brain Tumors, and Other Brain Diseases and Disorders

In one embodiment, the method includes administering an effective amount of a peptide of the present disclosure to a subject in need thereof.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system.

Compositions containing such agents or compounds can be administered for prophylactic, enhancing, and/or therapeutic treatments. An appropriate “effective” amount in any individual case may be determined using techniques, such as a dose escalation study.

The methods, compositions, and kits of this disclosure may comprise a method to prevent, treat, arrest, reverse, or ameliorate the symptoms of a condition. The treatment may comprise treating a subject (e.g., an individual, a domestic animal, a wild animal, or a lab animal afflicted with a disease or condition) with a peptide of the disclosure. The disease may be a brain or spinal cord disease. In treating the disease, the peptide may cross the blood brain barrier or blood cerebrospinal fluid barrier of a subject. The subject may be a human. Subjects can be humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants, and fetuses in utero.

Treatment may be provided to the subject before clinical onset of disease. Treatment may be provided to the subject after clinical onset of disease. Treatment may be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment can comprise administering to a subject a pharmaceutical composition, such as one or more of the pharmaceutical compositions described throughout the disclosure. A treatment can comprise delivering a peptide of the disclosure to a subject, either intravenously, subcutaneously, intramuscularly, by inhalation, dermally, topically, orally, sublingually, intrathecally, transdermally, intranasally, via a peritoneal route, or directly into the brain, e.g., via and intracerebral ventrical route. A treatment can comprise administering a peptide-active agent complex to a subject, either intravenously, subcutaneously, intramuscularly, by inhalation, dermally, topically, orally, intrathecally, transdermally, intransally, parenterally, orally, via a peritoneal route, nasally, sublingually, or directly into the brain.

The activity of a plurality of brain regions, tissues, structures or cells can be modulated by a peptide of the disclosure. Some of the brain regions, tissues, structures include: a) the cerebrum, including cerebral cortex, basal ganglia (striatum), and olfactory bulb; b) the cerebellum, including dentate nucleus, interposed nucleus, fastigial nucleus, and vestibular nuclei; c) diencephalon, including thalamus, hypothalamus, and the posterior portion of the pituitary grand; and d) the brain-stem, including pons, substantia nigra, medulla oblongata; e) the temporal lobe, including the hippocampus and the dentate gyrus (including the subgranular zone); f) the ventricular system, including the lateral ventricles (right and left ventricles), third ventricle, fourth ventricle, intraventricular foramina, cerebral aqueduct, median aperture, right and left lateral apertures, choroid plexus, and the subventricular zone; g) the CSF and associated tissues, including the subarachnoid space, cisterns, sulci; h) the meninges, including the dura mater, arachnoid mater, and pia mater; i) the rostral migratory stream; j) neural stem cells, neural progenitor cells, and new neural cells; and k) any cells or cell types in (a)-(j) above. In some embodiments, the peptides of the present disclosure are capable of crossing the BBB or blood CSF barrier and accumulating in one or more specific brain regions, tissue, structures, or cells. For example, in certain embodiments, the peptides described herein home, target, are directed to, migrate to, or accumulate in the hippocampus, the CSF, the ventricular system, the meninges, or the rostral migratory stream, or combinations thereof.

In some embodiments, the present disclosure provides a method for treating a brain disease or condition, the method comprising administering to a subject in need thereof an effective amount of a peptide of the present disclosure. A brain disease or condition can be any neurodegenerative disease or lysosomal storage disease. A neurodegenerative disease can be any disease, state, or condition relating to the loss of structure or function of the central nervous system, including any disease, state or condition relating to the loss of structure or function of the central nervous system, including without limitation Alzheimer's disease, Parkinson's disease, Huntington's disease, Amyotrophic Lateral Sclerosis, Frontotemporal Dementia, Progressive Supranuclear Palsy and Corticobasal Degeneration. A lysosomal storage disease can be any disease, state, or condition relating to defects in lysosomal function, including, without limitation, Krabbe disease, Gaucher disease, Tay-Sachs disease, Niemann-Pick disease, Pompe disease, Hurler syndrome, and Hunter syndrome. Further examples of brain diseases or conditions that can be treated with a peptide of the disclosure include Acoustic Neuroma (Vestibular Schwannoma), Acute Subdural Hematomas, Addictions (e.g., alcoholism, drug addiction, nicotine or tobacco, etc.), Alzheimer's disease, Amyotrophic Lateral Sclerosis (ALS, or Lou Gehrig's Disease), Anaplastic Astrocytoma (AA), Anxiety and related disorders, Anorexia, Antisocial Personality Disorder, Aqueductal Stenosis, Arachnoid Cysts, Arnold Chiari Malformation, Arteriovenous Malformation (AVM), Astrocytoma, Autism, Ballism, bipolar disorders, Brain Aneurysm, Brain Attack, Brain Metastases, Brainstem Glioma, Bulimia, Carotid Stenosis, Catastrophic Epilepsy in Children, Cavernous Angioma, Cerebral Aneurysms, Cerebral Contusion and Intracerebral Hematoma, Cerebral Hemorrhage, Chiari Malformation, Chordomas, Chorea, Choroid Plexus Cyst, Chronic Subdural Hematomas, Colloid Cyst, Coma, Concussion, Cranial Gun Shot Wounds, Corticobasal Degeneration, Craniopharyngioma, Craniosynostosis, Cushing's Disease, Cyst (Epidermoid Tumor), Dementia, Depression and related disorders, eating disorders, weight loss and satiety, Diabetes, Dravet Syndrome, Ependymoma, Epilepsy, Epidural Hematomas Epilepsy, Essential Tremor, Extratemporal Lobe Epilepsies, Facet Joint Syndrome, Frontotemporal Dementia, Ganglioglioma, Gaucher disease, Germinoma, Glioblastoma Multiforme (GBM), Glioma, Glomus Jugulare Tumor, Glossopharyngeal Neuralgia, Hemangioblastomas, Hemi-Facial Spasm, Hydrocephalus, Huntington's disease, immune system disorders, Intracerebral Hemorrhage, Hurler syndrome, Hunter syndrome, Intracranial Hypotension, JPA (Juvenile Pilocytic Astrocytoma), Krabbe disease, Lennox-Gestaut Syndrome, Lipomyelomeningocele, Low-Grade Astocytoma (LGA), Lymphocytic Hypophysitis, Lymphoma, Medulloblastoma, Meningioma, Meningitis, Mesial Temporal Lobe Epilepsy, Metastatic Brain Tumors, Migraine, Mitochondrial Disease, Moyamoya Disease, multiple sclerosis, Multiple system atrophy (MSA), Niemann-Pick disease, Nelson's Syndrome, Neurocysticercosis, Neurodegenerative Disorders, Neurofibroma, neuropathic pain, Nonfunctional Pituitary Adenoma, Normal Pressure Hydrocephalus, obsessive-compulsive disorders, Oligodendroglioma, Optic Nerve Glioma, Osteomyelitis, Parkinson's disease, Paranoia and related disorders, Pediatric Hydrocephalus, Phantom Limb Pain, Pilocytic Astrocytoma, Pineal Tumor, Pineoblastoma, Pineocytoma, Pituitary Adenoma (Tumor), Pituitary Apoplexy, Pituitary Failure, Pompe disease, Postherpetic Neuralgia, Post-Traumatic Seizures, Post-Traumatic Stress Disorder, Primary CNS Lymphoma, Prolactinoma, Pseudotumor Cerebri, Progressive Supranuclear Palsy, Rathke's Cleft Cyst, Recurrent Adenomas, Rheumatoid Arthritis, Schizophrenia, Schwannomas, Scoliosis, Skull Fracture, Slit Ventricle Syndrome, Spasticity, Spontaneous Intracranial Hypotension, Stroke (Brain Attack, TIA), Subarachnoid Hemorrhage, Syrinx, Tay-Sachs disease, Thyrotroph (TSH) Secreting Adenomas, Torticollis, Transient Ischemic Attacks (TIA), Traumatic Brain Injury, Traumatic Hematomas, Trigeminal Neuralgia, Ventriculitis, Vestibular Schwannoma, depression, mood disorders, lysosomal storage diseases, memory disorders, learning disorders, disorders of spatial memory or navigation, stress-related disorders, post-traumatic stress disorder, pain, aging, hippocampal atrophy, brain infections including fungal infections and progressive Multifocal Leukoencepalopathy, or another brain disease or condition. In other cases, a peptide of the disclosure can be used to treat alcoholism, cigarette addiction, drug addiction, or anxiety.

In some embodiments, the peptide binds to potassium channels in the brain. In some embodiments the peptide binds to sodium channels in the brain. In some embodiments, the peptide blocks potassium channels and/or sodium channels, in some embodiments the peptide activates of potassium channels and/or sodium channels. In some embodiments, the peptide interacts with ion channels or chloride channels or calcium channels. In some embodiments the peptide interacts with nicotinic acetyl choline receptors, transient receptor potential channels, NMDA receptors, serotonin receptors, KIR channels, GABA channels, glycine receptors, glutamate receptors, acid sensing ion channels, K2P channels, Nav1.7, or purinergic receptors. In some embodiments, the peptide interacts with matrix metalloproteinase, inhibits cancer cell migration or metastases, or has antitumor activity. In some embodiments, the peptide interacts with calcium activated potassium channels. In some embodiments, the peptide has antibacterial, antifungal, or antiviral activity. In some embodiments, the peptide inhibits proteases. In some embodiments, the peptide interacts with channels that influence pain. In some embodiments, the peptide has other therapeutic effects on the brain or structures thereof.

In some embodiments, the peptides of the present disclosure are used to diagnose or treat a disease or condition associated with the hippocampus. The hippocampus is a critical brain structure involved in learning, memory, mood, and cognition. Changes in the hippocampus, including reduced volume and cellularity, reduced neuronal density, and defects in neurotransmitter function, are associated with initiation, persistence, and/or progression of disorders including late-life depression (Taylor); major depression and bipolar disorder (Drevets); post-traumatic stress disorder (PTSD) (Schmidt); Alzheimer disease (Nava-Mesa); and schizophrenia (Perez). Peptides of the current invention that target the hippocampus can be used to treat these diseases or to target therapeutically-active substances to treat these diseases amongst others. In some embodiments, the peptides are used to treat these diseases by acting on receptors such as GABA, NMDA, AMPA, dopamine, or serotonin receptors. The dentate gyrus in the hippocampus can also be a site of neurogenesis.

In some embodiments, the peptides of the present disclosure are used to diagnose or treat a disease or condition associated with the CSF or ventricular system. The CSF is a fluid that surrounds and circulates in the brain and spine that provides mechanical protection for the brain and plays a role in the homeostasis and metabolism of the central nervous system. CSF is produced by and circulated within the ventricular system. Diseases and conditions that are associated with the CSF or ventricular system include but are not limited to: antisocial personality disorder, cerebral hemorrhage, choroid plexus cyst, dementia, ependymoma, hydrocephalus, meningitis, multiple system atrophy (MSA), neurodegenerative disease (such as amyotrophic lateral sclerosis, Parkinson's disease, Alzheimer's disease, Huntington's disease) post-traumatic stress disorder, schizophrenia, subarachnoid hemorrhage, traumatic brain injury, and ventriculitis.

Peptides of the current disclosure that target the CSF or ventricular system can be used to treat these diseases or to target therapeutically-active substances to treat these diseases, amongst others. For example, in certain embodiments, the peptides of the present disclosure are used to modulate targets associated with a disease, such as mitochondrial deubiquitinase USP30 (e.g., for the treatment of Parkinsons' disease) or dual leucine zipper kinase (e.g., for the treatment of neurodegeneration). As another example, in certain embodiments, the peptides are conjugated to a therapeutic agent used to treat a neurodegenerative disease, such as Alzheimer's disease. Such drugs could also include galantamine, donzepil, tacrine, or even neurotoxins generally thought to be too toxic, such as sarin. Examples of therapeutic agents useful for treating neurodegenerative disease include but are not limited to: acetylcholinesterase inhibitors (e.g., rivastigimine), galantamine, donzepil, tacrine, and neurotoxins (e.g., sarin). This approach allows for treatment with lower dosages and reduced side effects in the periphery, compared to prior methods which utilize untargeted systemic delivery. In yet another example, in certain embodiments, peptides that home, distribute to, target, migrate to, accumulate in, or are directed to the ventricular space are used as radioprotectant (e.g., alone or as a conjugate to a radioprotective compound such as amifostine) during treatment of brain metastases with radiation.

In some embodiments, the peptides of the present disclosure are used to inhibit small-conductance, calcium-activated potassium channels (SK channels). Peptides that inhibit SK channels include members of the Toxin_6 class, for example. Optionally, such peptides may exhibit homing to specific brain regions, such as the ventricles. In certain embodiments, the peptides of the present disclosure have specificity for one or more SK channel subtypes, such as one or more of the SK1, SK2, SK3, or SK4 channel subtypes. In certain embodiments, inhibition of the SK3 subtype increases the frequency of firing in dopaminergic neurons, thus increasing levels of dopamine, which may ameliorate the physical symptoms of Parkinson's disease.

In some embodiments, the peptides of the present disclosure are used to affect (e.g., reduce, slow, or inhibit) the aggregation of proteins associated with neurodegenerative disease, such as tau, prion protein, amyloid beta, alpha synuclein, parkinin, or huntingtin.

In some embodiments, the peptides of the present disclosure are used to inhibit or activate one or more specific ion channels, and the inhibition or activation of the ion channels alleviates the symptoms of a range of diseases. TABLE 3 illustrates exemplary ion channels and associated diseases that may be treated in accordance with the compositions and methods presented herein.

TABLE 3
Exemplary ion channels and associated
diseases according to the present disclosure.

| Ion | | Gain (G) or | |
|---|---|---|---|
| Channel | | Loss (L) of | |
| Family | Channel | Function | Disease |
| Kᵢᵣ | Kᵢᵣ1.1 | L | Bartter's syndrome |
| | Kᵢᵣ2.1 | L | Andersen's syndrome |
| | Kᵢᵣ6.2 | L | congenital hyperinsulinism |
| | | G | neonatal diabetes |
| | SUR2 | L | dilated cardiomyopathy |
| Kᵥ | Kᵥ1.1 | L | episodic ataxia type 1 |
| | KCNQ1 | L | long QT syndrome |
| | | G | short QT syndrom |
| | KCNQ2 | L | benign neonatal febrile convulsions |
| | KCNQ4 | L | nonsyndromic deafness |
| | hERG | L | long QT syndrome |
| | | G | short QT syndrome |
| TRP | TRPP2 | | polycystic kidney disease |
| | TRPA1 | G | familial episodic pain syndrome |
| | TRPC6 | G | focal segmental glomerulosclerosis |
| CNG | CNGA1 | L | retinitis pigmentosa |
| K_{Ca} | BK | G | epilepsy |
| NAᵥ | NAᵥ1.1 | G | epilepsy |
| | | L | severe myoclonic epilepsy |
| | NAᵥ1.5 | G | long QT syndrome |
| | NAᵥ1.6 | L | cerebellar ataxia |
| | NAᵥ1.7 | G | erythromelalgia, paroxysmal |
| | | | extreme pain disorder |
| | | L | congenital indifference to pain |
| | NAᵥ2.1 | G | benign familial neonatal seizures |
| Caᵥ | Caᵥ1.2 | G | timothy syndrome |
| | Caᵥ2.1 | L | episodic ataxia type 2 |
| glycine | GLRA1 | L | stiff baby syndrome |
| receptors | | | |
| GABA | GABA_{A} | L | juvenile myoclonic epilepsy |
| AChR | CHRNA4 | L | autosomal dominant nocturnal |
| | | | frontal lobe epilepsy |

In some embodiments, the peptides of the present disclosure exhibit protease inhibitor activity. In certain embodiments, peptides capable of crossing the BBB are used to inhibit Alzheimer's associated proteases such as beta and gamma secretase. In alternative embodiments, peptides that may or may not be capable of crossing the BBB are used to inhibit other proteases of interest, such as coagulation-associated proteases (e.g., thrombin, factor 10a), metabolism-associated proteases (e.g., DPP-IV), cancer-associated proteases (e.g., matrix metalloproteinases, cathepsins), viral infection-associated proteases (e.g., HIV protease), and inflammation-associated proteases (e.g., tryptase, kallikrein).

In some embodiments, the peptides of the present disclosure can be modified to be anti-inflammatory, such as by incorporating properties of Immune Selective Anti-Inflammatory Derivatives (ImSAIDs). In certain embodiments, ImSAIDs are incorporated into or added onto peptides capable of targeting specific brain regions as described herein. FEG is an example of a key sequence that confers anti-inflammatory properties. Alternatively or in combination, peptides of the present disclosure can be conjugated to immune regulatory molecules to reverse, reduce, or limit inflammation.

In some aspects, the peptides of the present disclosure are conjugated to one or more therapeutic agents. In certain embodiments, the peptides described herein are used as conjugates to deliver therapeutic agents across the BBB or blood CSF barrier and optionally into specific regions, tissues, structures, or cells in the brain. Examples of such therapeutic agents include anti-inflammatory molecules (e.g., dexamethasone, prednisone, prednisolone, methyl prednisolone, or traimcinolone), antifungal agents (e.g., fluconazole, amphotericin B, ketoconazole, or abafungin), antiviral agents (e.g., acyclovir, cidofovir), growth factors (e.g., NGF or EGF), or anti-infective agents (e.g., ciprofloxacin, tetracycline, erythromycin, or streptomycin). For instance, in certain embodiments, a peptide of the present disclosure is conjugated to an antifungal agent in order to treat a fungal infection of the brain, which is otherwise highly difficult to treat using prior methods and compositions. As another example, in certain embodiments, a BBB-penetrating peptide of the present disclosure is conjugated to cidofovir in order to treat progressive multifocal leucoencephalopathy (PML) caused by the JC virus, which otherwise has no reliable treatment.

In some embodiments, the peptides of the present disclosure are used to treat brain cancer. For example, in certain embodiments, the peptides provided herein are used to directly inhibit critical cancer-associated pathways such as RAS, MYC, PHF5A, BubR1, PKMYT1, or BuGZ. Alternatively or in combination, the peptides of the present disclosure are used to carry a conjugated therapeutic agent across the BBB in order to treat brain cancer.

In further aspects, the therapeutic agent is a chemotherapeutic agent, anti-cancer drug, or anti-cancer agent selected from, but are not limited to: radioisotopes, toxins, enzymes, sensitizing drugs, nucleic acids, including interfering RNAs, antibodies, anti-angiogenic agents, cisplatin, platinum compounds, anti-metabolites, mitotic inhibitors, growth factor inhibitors, taxanes, paclitaxel, cabazitaxel, temozolomide, topotecan, fluorouracil, vincristine, vinblastine, 4-deacetylvinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane and amifostine, vinca alkaloids, cyclic octapeptide analogs of mushroom amatoxins, epothilones, and anthracylines, CC-1065, SN-38, and BACE inhibitors, and their equivalents, as well as photo-ablation agents. For example, in certain embodiments, a peptide of the present disclosure is conjugated to palbociclib, a CDK 4/6 inhibitor with limited ability to cross the BBB. As another example, in certain embodiments, a peptide of the present disclosure is conjugated to monomethyl auristatine E (MMAE), MMAF, an auristatin, dolostatin, auristatin F, monomethylauristatin D, a maytansinoid (e.g., DM-1, DM4, maytansine), a pyrrolobenzodiazapine dimer, N-acetyl-γ-calicheamicin, a calicheamicin, a duocarmycin, an anthracycline, a microtubule inhibitor, or a DNA damaging agent.

Optionally, certain embodiments of the present disclosure provide peptides conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cancer cells using both a therapeutic agent (e.g., drug) and electromagnetic energy (e.g., radiation or light) concurrently.

In certain embodiments, the peptide of the disclosure is mutated to retain ability to cross the BBB or the blood CSF barrier and home, distribute to, target, migrate to, accumulate in, or are directed to certain tissues, but to gain or lose function, such as agonizing an ion channel or inhibiting a protease. In other embodiments, the peptide of the disclosure is mutated to home, distribute to, target, migrate to, accumulate in, or is directed to certain tissues but not others, to change the strength or specificity of its function, or to gain or lose function.

The present disclosure also encompasses the use of “tandem” peptides in which two or more peptides are conjugated or fused together. In certain embodiments, a tandem peptide comprises two or more knotted peptides conjugated or fused together, where at least one knotted peptide is capable of crossing the BBB and optionally targeting to a specific brain region, while at least one other knotted peptide provides a specific therapeutic activity, such as a BIM analogue, as discussed above and herein.

In some embodiments, the present disclosure provides a method for treating a cancer, the method comprising administering to a subject in need thereof an effective amount of a peptide of the present disclosure.

In some embodiments, the present disclosure provides a method for treating a cancer, the method comprising administering to a patient in need thereof an effective amount of a pharmaceutical composition comprising a peptide of the present disclosure and a pharmaceutically acceptable carrier.

In some embodiments, the present disclosure provides a method for inhibiting invasive activity of cells, the method comprising administering an effective amount of a peptide of the present disclosure to a subject.

In some aspects, the present disclosure provides a method for detecting a cancer, cancerous tissue, or tumor tissue, the method comprising the steps of contacting a tissue of interest with a peptide of the present disclosure, wherein the peptide is conjugated to a detectable agent and measuring the level of binding of the peptide, wherein an elevated level of binding, relative to normal tissue, is indicative that the tissue is a cancer, cancerous tissue or tumor tissue.

The present invention provides methods for intraoperative imaging and resection of a cancer, cancerous tissue, or tumor tissue using a peptide of the present disclosure conjugated with a detectable agent. In some aspects, the cancer, cancerous tissue, or tumor tissue is detectable by fluorescence imaging that allows for intraoperative visualization of the cancer, cancerous tissue, or tumor tissue using a peptide of the present disclosure. In some aspects, the peptide of the present disclosure is conjugated to one or more detectable agents. In a further embodiment, the detectable agent comprises a fluorescent moiety coupled to the peptide. In another embodiment, the detectable agent comprises a radionuclide. In some aspects, imaging is achieved using open surgery. In further aspects, imaging is accomplished using endoscopy or other non-invasive surgical techniques.

In some cases, the peptide or peptide-active agent can be used to target cancer in the brain by crossing the BBB or blood CSF barrier and then having antitumor function, targeted toxicity, inhibiting metastases, etc. In other cases, the peptide or peptide-active agent can be used to label, detect, or image such brain lesions, including tumors and metastases amongst other lesions, which may be removed through various surgical techniques.

In addition, certain peptides of the disclosure can have additional applicability in diseases and conditions outside the brain. A peptide comprising the sequence of any of SEQ ID NO: 1-SEQ ID NO: 192 or SEQ ID NO: 210-SEQ ID NO: 401, and any peptide derivative or peptide-active agent as described herein, can be used to additionally target upper GI disease and cancers (e.g., throat, oral, esophageal cancer, salivary glands, tonsils, pharynx, adenosarconmas, oral malignant melanoma, head and neck cancer). A peptide comprising the sequence of any of SEQ ID NO: 1-SEQ ID NO: 192 or SEQ ID NO: 210-SEQ ID NO: 401, and any peptide derivative or peptide-active agent as described herein, can be used to additionally target gall bladder disease and cancers.

Venom or toxin derived peptide(s), peptides, modified peptides, labeled peptides, peptide-active agent conjugates and pharmaceutical compositions described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Such peptides described herein can also be administered to prevent (either in whole or in part), lessen a likelihood of developing, contracting, or worsening a condition. Amounts effective for this use can vary based on the severity and course of the disease or condition, previous therapy, the subject's health status, weight, and response to the drugs, and the calculations of the treating physician.

In some embodiments, the present disclosure provides a method of treating a brain condition of a subject, the method comprising administering to the subject a peptide comprising the sequence of any of SEQ ID NO: 1-SEQ ID NO: 196, or a functional fragment thereof.

Multiple peptides described herein can be administered in any order or simultaneously. In some cases, multiple functional fragments of peptides derived from toxins or venom can be administered in any order or simultaneously. If simultaneously, the multiple peptides described herein can be provided in a single, unified form, such as an intravenous injection, or in multiple forms, such as subsequent intravenous dosages.

Peptides can be packaged as a kit. In some embodiments, a kit includes written instructions on the use or administration of the peptides.

### EXAMPLES

The following examples are included to further describe some aspects of the present disclosure, and should not be used to limit the scope of the invention.

### Example 1

### Manufacture of Peptides

This example describes the manufacture of the peptides described herein. Peptides derived from knottin proteins of scorpions and spiders were generated in mammalian cell culture using a published methodology. (A. D. Bandaranayke, C. Correnti, B. Y. Ryu, M. Brault, R. K. Strong, D. Rawlings. 2011. Daedalus: a robust, turnkey platform for rapid production of decigram quantities of active recombinant proteins in human cell lines using novel lentiviral vectors. Nucleic Acids Research. (39)21, e143).

The peptide sequence was reverse-translated into DNA, synthesized, and cloned in-frame with siderocalin using standard molecular biology techniques. (M. R. Green, Joseph Sambrook. Molecular Cloning. 2012 Cold Spring Harbor Press.). The resulting construct was packaged into a lentivirus, transfected into HEK293 cells, expanded, isolated by immobilized metal affinity chromatography (IMAC), cleaved with tobacco etch virus protease, and purified to homogeneity by reverse-phase chromatography. Following purification, each peptide was lyophilized and stored frozen.

### Example 2

### Peptide Radiolabeling

This example describes the radiolabeling of peptides. Several knottins were radiolabeled by reductive methylation with ¹⁴C formaldehyde and sodium cyanoborohydride with standard techniques. The sequences were engineered to have the amino acids, “G” and “S” at the N terminus. See Methods in Enzymology V91:1983 p. 570 and JBC 254(11):1979 p. 4359. An excess of formaldehyde was used to ensure complete methylation (dimethylation of every free amine). The labeled peptides were isolated via solid-phase extraction on Strata-X columns (Phenomenex 8B-S 100-AAK), rinsed with water with 5% methanol, and recovered in methanol with 2% formic acid. Solvent was subsequently removed in a blowdown evaporator with gentle heat and a stream of nitrogen gas.

### Example 3

### Peptide Dosing

This example illustrates the dosing of peptide. Different dosages of the peptides were administered to Female Harlan athymic nude mice, weighing 20 g-25 g, via tail vein injection (n=2 mice per knottin). The experiment was done in duplicates. The kidneys were ligated to prevent renal filtration of the peptides. The peptides of SEQ ID NO: 1-SEQ ID NO: 4, SEQ ID NO: 34, SEQ ID NO: 35, SEQ ID NO: 55, SEQ ID NO: 5, SEQ ID NO: 36-SEQ ID NO: 37, and SEQ ID NO: 39 were radiolabeled by methylating lysines and the N-terminus so the actual binding agent may contain methyl or dimethyl lysine(s) and a methylated or dimethylated amino terminus (FIG. 1).

A target dosage of 20 nmol of each peptide was administered to separate flank sarcoma A204 tumor bearing Female Harlan athymic nude mice while anesthetized. The dosage was adjusted for whole body autoradiography. Each peptide (target dose of 20 nmol) was allowed to freely circulate within the animal for a target of three hours before the animals were euthanized and sectioned. Some animals died after receiving the peptides, possibly due to complications of the anesthesia and surgical procedure used to ligate the kidneys of each mice, or due to toxic effects of some peptides. The circulation time of each peptide varied from animal-to-animal. Fluoxetine (Prozac) was used as a positive control that crosses the BBB, in an animal that did not undergo kidney ligation. Inulin was used as a negative control, as this polysaccharide is known to not cross the BBB.

### Example 4

### Peptide Crossing the Blood Brain Barrier and Homing to the Brain

This example shows the peptide crossing the blood brain barrier (BBB) and/or the blood cerebral spinal fluid (CSF) barrier, and in some cases homing to specific locations within the brain. At the end of the dosing period, mice were frozen in a hexane/dry ice bath and then frozen in a block of carboxymethylcellulose. Thin, frozen sections of whole animal sagittal slices that include the brain, tumor, liver, kidney, lung, heart, spleen, pancreas, muscle, adipose, gall bladder, upper gastrointestinal track, lower gastrointestinal track, bone, bone marrow, reproductive track, eye, cartilage, stomach, skin, spinal cord, bladder, salivary gland, and other types of tissues were obtained with a microtome, allowed to dessicate in a freezer, and exposed to phosphoimager plates for about ten days.

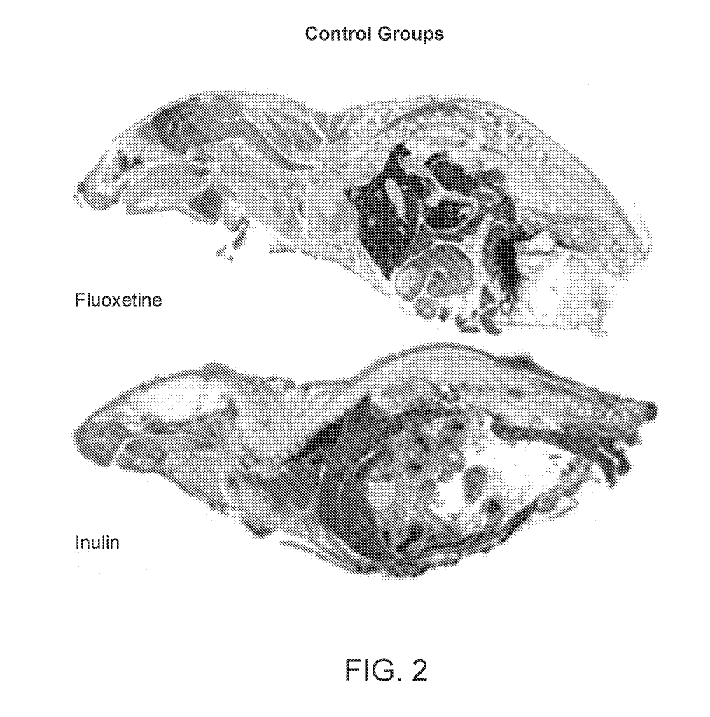

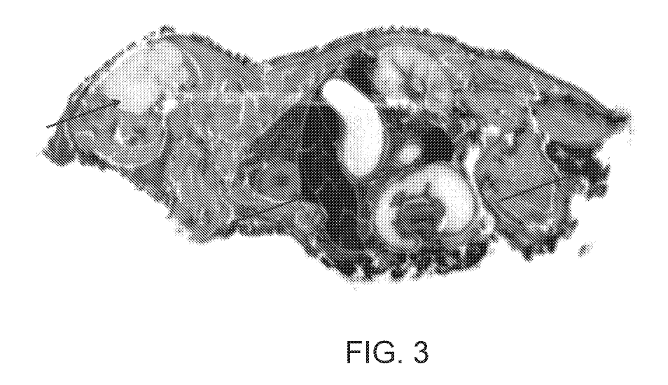

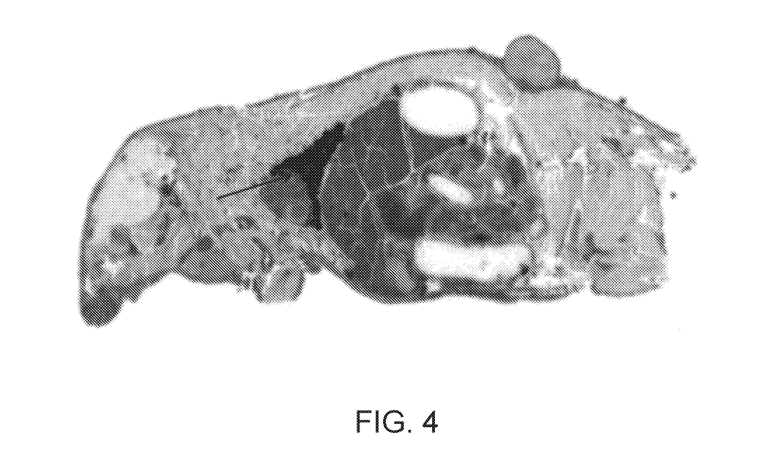

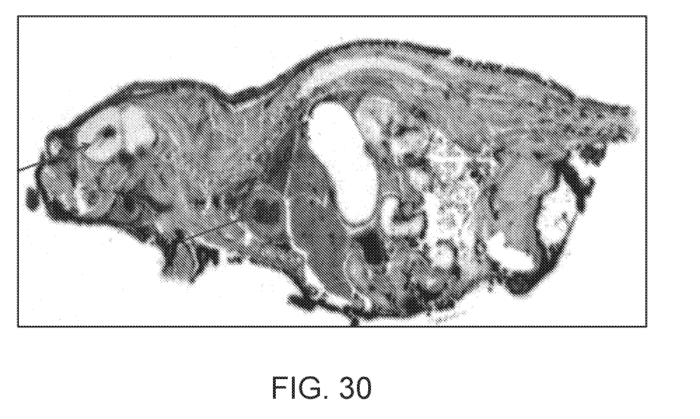

These plates were developed, and the signal (densitometry) from each organ was normalized to the signal found in the heart blood of each animal. A signal in tissue darker than the signal expected from blood in that tissue indicates accumulation in a region, tissue, structure or cell. Fluoxetine can cross the blood brain barrier, and is a positive control. Inulin cannot cross the blood brain barrier, and is a negative control. FIG. 2 illustrates ¹⁴C signal in the brain and other tissues for the fluoxetine (top) and inulin (bottom) control groups. FIG. 3 illustrates ¹⁴C signal in the brain and other tissues for radiolabeled peptides of SEQ ID NO: 1. FIG. 4 illustrates ¹⁴C signal in the brain and other tissues for radiolabeled peptides of SEQ ID NO: 3. FIG. 30 illustrates an autoradiographic image showing the ¹⁴C signal in the brain of a mouse treated with a peptide of SEQ ID NO: 55.

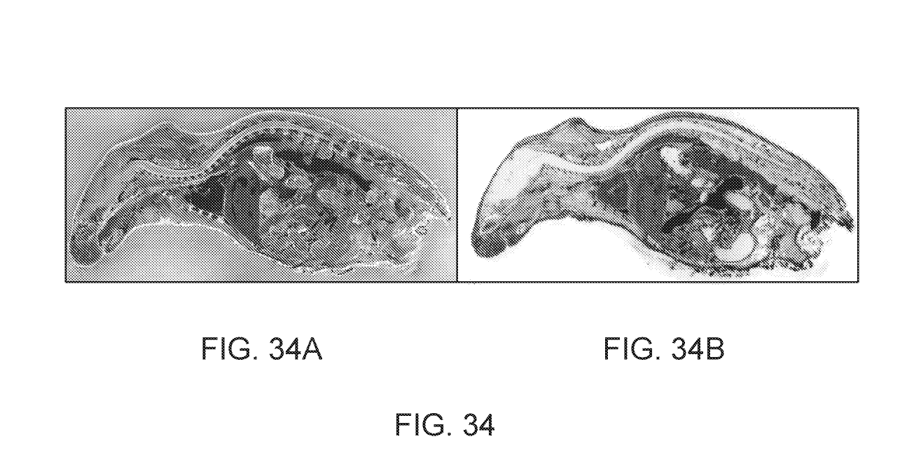

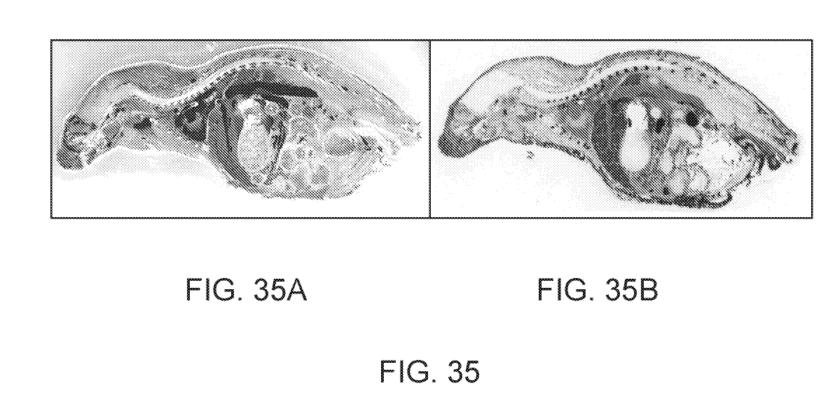

Furthermore, the brain contains approximately 3% blood. Thus when comparing the radioactivity signal per area in blood to that in the brain, a signal in the brain that is much higher than 3% of the signal in blood can be attributed to accumulation of the material in the brain through the BBB. A ratio of at least 10% diffuse signal in brain versus blood was chosen as a reference level for high penetration. The densitometric signal can also indicate high concentration within specification locations in the brain, which can indicate crossing the BBB and/or the blood CSF barrier. FIG. 34A shows a white light image of a frozen section of a mouse with ligated kidneys 3 hours after administration of 100 nmol of the radiolabeled SEQ ID NO: 39 peptide. FIG. 34B shows an autoradiographic image corresponding to FIG. 34A in which the ¹⁴C signal identifies the peptide distribution in the tissues of the mouse with ligated kidneys 3 hours after administration of 100 nmol of the radiolabeled SEQ ID NO: 39 peptide and the average brain/blood ratio of the radiolabeled SEQ ID NO: 39 peptide was determined to be 6.01%. FIG. 35A shows a white light image of a frozen section of a mouse with ligated kidneys 3 hours after administration of 100 nmol of the radiolabeled SEQ ID NO: 36 peptide. FIG. 35B shows an autoradiographic image corresponding to FIG. 35A in which the ¹⁴C signal identifies the peptide distribution in the tissues of the mouse with ligated kidneys 3 hours after administration of 100 nmol of the radiolabeled SEQ ID NO: 36 peptide and the average brain/blood ratio of the radiolabeled SEQ ID NO: 36 was determined to be 9.64%.

TABLE 4 lists a summary of the migration of each peptide to the brain. Numbers for blood indicate the densitometric signal in the heart blood. Numbers for the brain indicate the percentage of signal in that tissue compared to the signal detected in the heart blood.

TABLE 4
Summary of peptide migration in the blood or to the brain.

| Peptide | Blood | Brain |
|---|---|---|
| SEQ ID NO: 1 | 2203247460 | 14.53 |
| SEQ ID NO: 2 | 1327759044 | 11.27 |
| SEQ ID NO: 3 | 2858983560 | 10.76 |
| SEQ ID NO: 4 | 964585318.5 | 10.83 |
| SEQ ID NO: 34 | 6552245640 | 9.30 |
| SEQ ID NO: 5 | | 7.1 |
| SEQ ID NO: 35 | 4523239.203 | 6.95 |
| SEQ ID NO: 37 | 9232585.298 | 10.36 |
| SEQ ID NO: 55 | 1838144857.46255 | 6.86 |
| SEQ ID NO: 36 | 13350663.35 | 9.64 |
| SEQ ID NO: 39 | 46425721.51 | 6.01 |
| Inulin | 900918901.9 | 3.00 |
| GS-Hainantoxin | 1768833565 | 3.65 |
| Potassium Channel | 364827742 | 3.74 |
| Peptide | | |

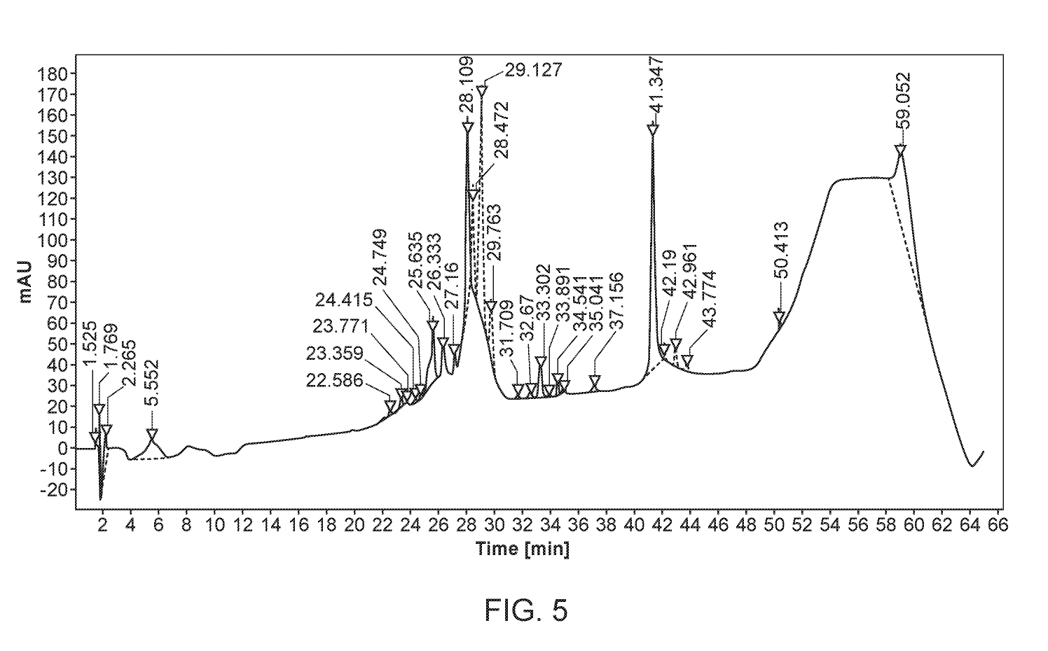

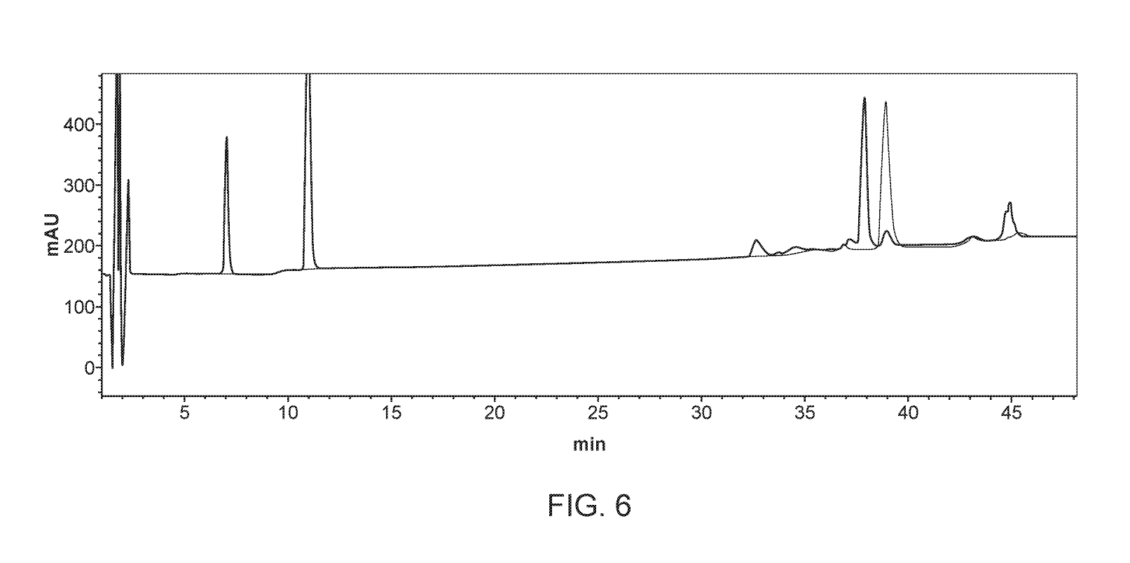

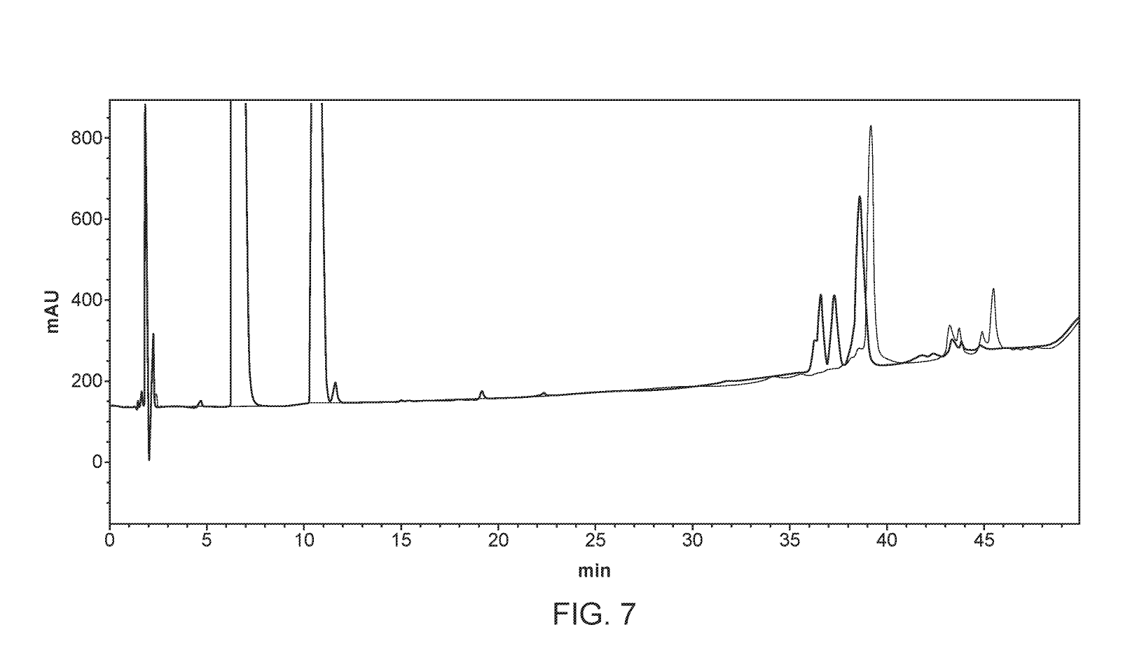

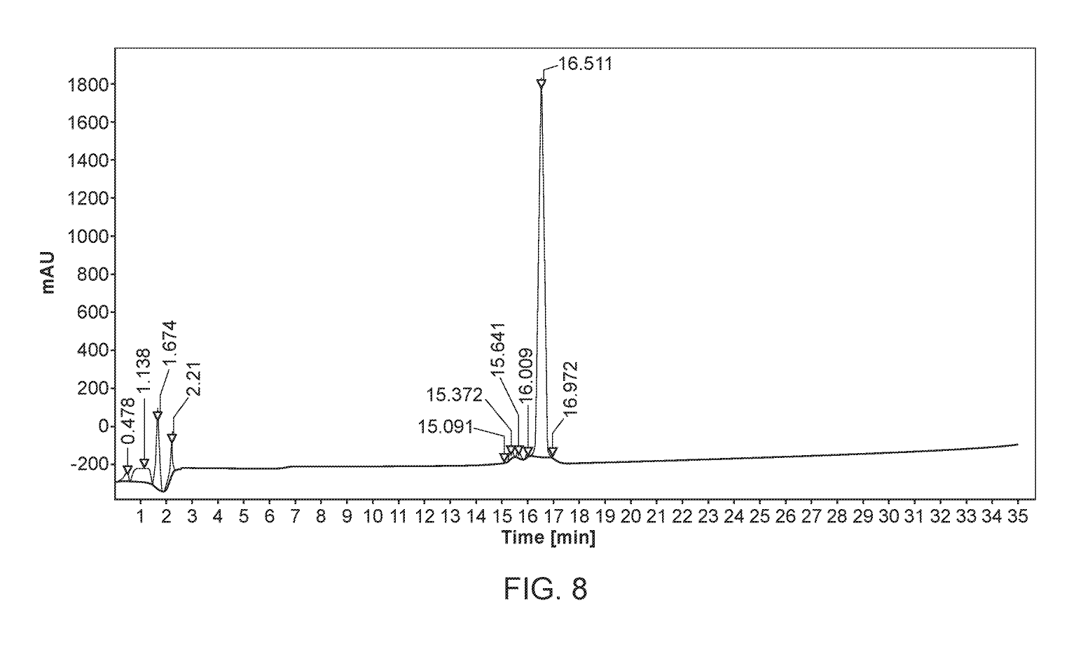

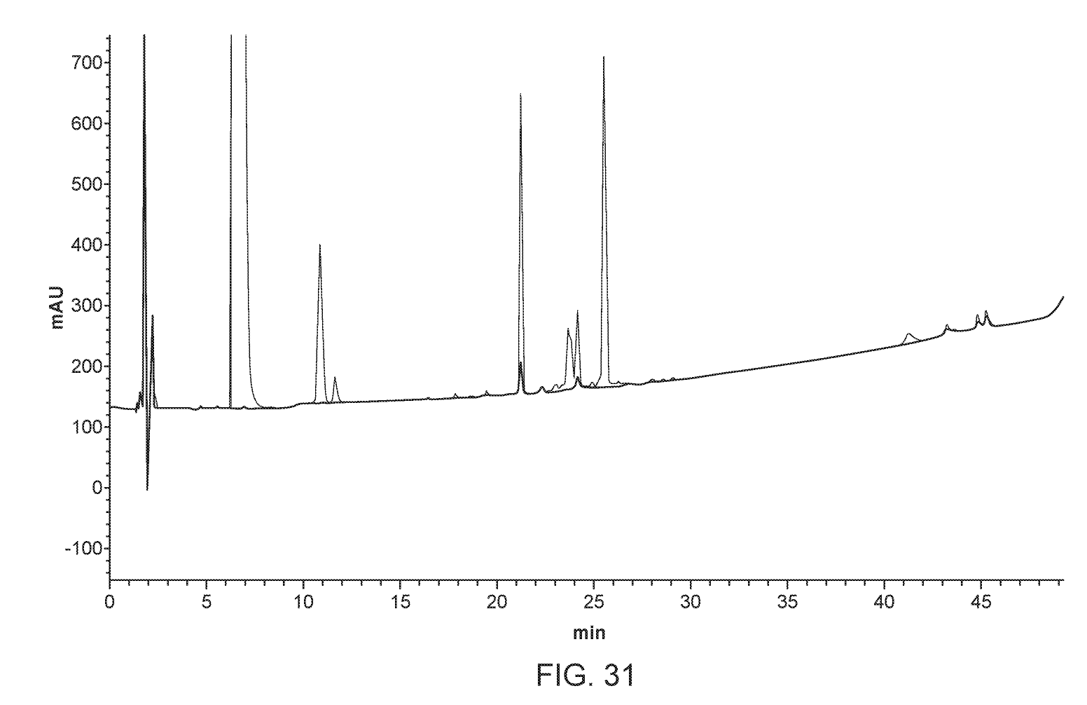

The peptides of SEQ ID NO: 1, SEQ ID NO: 2, SEQ ID NO: 3, SEQ ID NO: 4, SEQ ID NO: 34, SEQ ID NO: 36, and SEQ ID NO: 37 highly penetrate the blood brain barrier in comparison with the negative control peptides Inulin, GS-Hainantoxin GSKCLPPGKPCYGATQKIPCCGVCSHNNCT (SEQ ID NO: 419), and a potassium channel peptide. The peptides of SEQ ID NO: 5, SEQ ID NO: 35, SEQ ID NO: 39, and SEQ ID NO: 55 moderately penetrate the blood brain barrier in comparison with the negative control peptides Inulin, GS-Hainantoxin, and a potassium channel peptide. Peptides of SEQ ID NO: 35-SEQ ID NO: 39 were designed variants of the peptide of SEQ ID NO: 5, thus additionally illustrating that variants can be designed to be either highly or not highly penetrating of the blood brain barrier. Furthermore, the peptide of SEQ ID NO: 55 was found to migrate specifically to a region, tissue, structure or cells in the brain, potentially the hippocampus, the CSF, the ventricles, the meninges, and/or the rostral migratory stream (e.g., see Example 11). FIG. 5 through FIG. 8 and FIG. 31 illustrate the HPLC profiles of a peptide of SEQ ID NO: 1; SEQ ID NO: 2; SEQ ID NO: 3; SEQ ID NO: 4; and SEQ ID NO: 55 respectively.

### Example 5

### Peptide Administration and Homing with Therapeutic Agents

This example describes peptide administration and homing with therapeutic agents. A peptide of the disclosure is expressed recombinantly or chemically synthesized and then is conjugated to a drug. Alternatively, a peptide of the disclosure is fused during recombinant expression to a drug. A drug, such as cytotoxic chemotherapeutics (e.g., taxane, alkylating agents, or microtubule inhibitors), anti-sense (siRNA, dsRNA), anti-depressants, anti-psychotics, ion channel blockers, protease inhibitors, neurotransmitters, antivirals, antibiotics, antifungals, nerve growth factors, monoclonal antibodies, cytokines, or other drugs that may affect the brain such as tianeptine, phenytoin, fluoxetine, lithium, tricyclic antidepressants, antipsychotics, sodium valproate, mifepristone, antiseizure, vitamin A, antioxidant, neurogenesis promoters, selective serotonin reuptake inhibitors, serotonin/noradrenaline reuptake inhibitors, paroxetine, phenytoin, neurotrophic factors, neurturin, hormones, or testosterone is used in the drug-peptide conjugate or fusion. The drug-peptide conjugate is made using the technique described in Bioconjugate Techniques by Greg Hermanson. One or more drug-peptide conjugates or fusions are administered to a human or animal.

### Example 6

### Treatment of a Brain Infection with a Peptide-Conjugate of the Disclosure

This example describes the treatment of a brain infection with a peptide-conjugate. A peptide of the disclosure is expressed recombinantly or chemically synthesized and then is conjugated to an antifungal or antibacterial compound, such as Fluconazole, Rifampicin, Ciprofloxacin, or Azithromycin. Coupling of the drugs to any one of the peptides of SEQ ID NO: 1-SEQ ID NO: 196 or SEQ ID NO: 210-SEQ ID NO: 405 targets the antifungal or antibacterial compound into the brain. One or more antifungal or antibacterial peptide conjugates are administered to a human or animal.

### Example 7

### Targeting of a Peptide-Drug Conjugate to a Region of the Brain

A peptide of the disclosure is expressed is expressed recombinantly or chemically synthesized and then is conjugated to a drug, such as: memantine, tacrine, ravastigmine, or donzepil using the technique described in Bioconjugate Techniques by Greg Hermanson. The drugs mentioned above typically cross the blood brain barrier, nevertheless coupling of the drugs to a peptide of any one of SEQ ID NO: 55-SEQ ID NO: 79, SEQ ID NO: 127, SEQ ID NO: 130, SEQ ID NO: 152, SEQ ID NO: 158, SEQ ID NO: 160, or SEQ ID NO: 190 targets the drug to a hippocampus CSF, ventricular system, meninges, rostral migratory stream, or dentate gyrus of the subject. One or more drug-peptide conjugates are administered to a human or animal.

### Example 8

### Treatment of a Virus with a Peptide-Conjugate of the Disclosure

This example describes the treatment of a virus with a peptide-conjugate. Progressive multifocal leucoencepalopathy (PML) is a viral disease of the brain that is very hard to treat because the antivirals typically do not cross the blood brain barrier. A peptide of the disclosure is expressed recombinantly or chemically synthesized and then is conjugated to an anti-viral compound, such as Cidofovir or Cytarabine. Coupling of the drugs to any one of the peptides of SEQ ID NO: 1-SEQ ID NO: 196 or SEQ ID NO: 210-SEQ ID NO: 405 targets the antiviral compound into the brain. One or more antiviral peptide conjugates are administered to a human or animal.

### Example 9

### Treatment of a Brain Tumor with a Peptide-Conjugate of the Disclosure

This example describes the treatment of a brain tumor with a peptide-conjugate. Many chemotherapeutics do not cross the blood-brain barrier. A peptide of the disclosure is expressed recombinantly or chemically synthesized and then is conjugated to a chemotherapeutic compound, such as cyclophosphamide, doxorubicin, an auristatin (e.g., monomethyl auristatin E (MMAE), monomethyl auristatin F (MMAF), dolostatin, auristatin F, MMAD), a maytansinoid (e.g., DM1, DM4, maytansine), a pyrrolobenzodiazapine dimer, a calicheamicin (e.g., N-acetyl-γ-calicheamicin), a vinca alkyloid (e.g., 4-deacetylvinblastine), duocarmycin, cyclic peptide analogs of the mushroom amatoxins, epothilones, anthracyclines, CC-1065, taxanes (e.g., paclitaxel, docetaxel, cabazitaxel), SN-38, irinotecan, vincristine, a microtubule inhibitor, a DNA damaging agent, or teniposide, directly or via a linker. Coupling of the drugs to any one of the peptides of SEQ ID NO: 1-SEQ ID NO: 196 or SEQ ID NO: 210-SEQ ID NO: 405 targets the chemotherapeutic compound into the brain and optionally to the tumor. One or more chemotherapeutic-peptide conjugates are administered to a human or animal.

### Example 10

### Peptide Expression Using a Mammalian Expression System

This example describes the expression of peptides using a mammalian expression system. Peptides were expressed according to the methods described in Bandaranayake et al., Nucleic Acids Res. 2011 November; 39(21): e143. Peptides were cleaved from siderocalin using tobacco etch virus protease and purified by FPLC on a hydrophobic columns using a gradient of acetonitrile and 0.1% TFA. Peptides were then lyophilized and stored frozen.

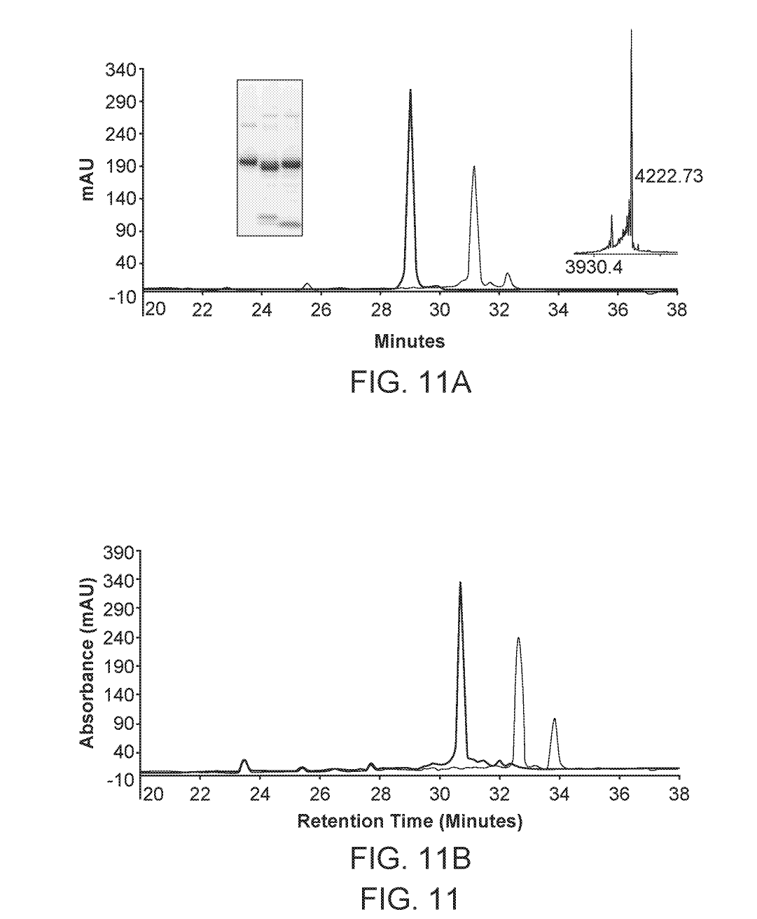

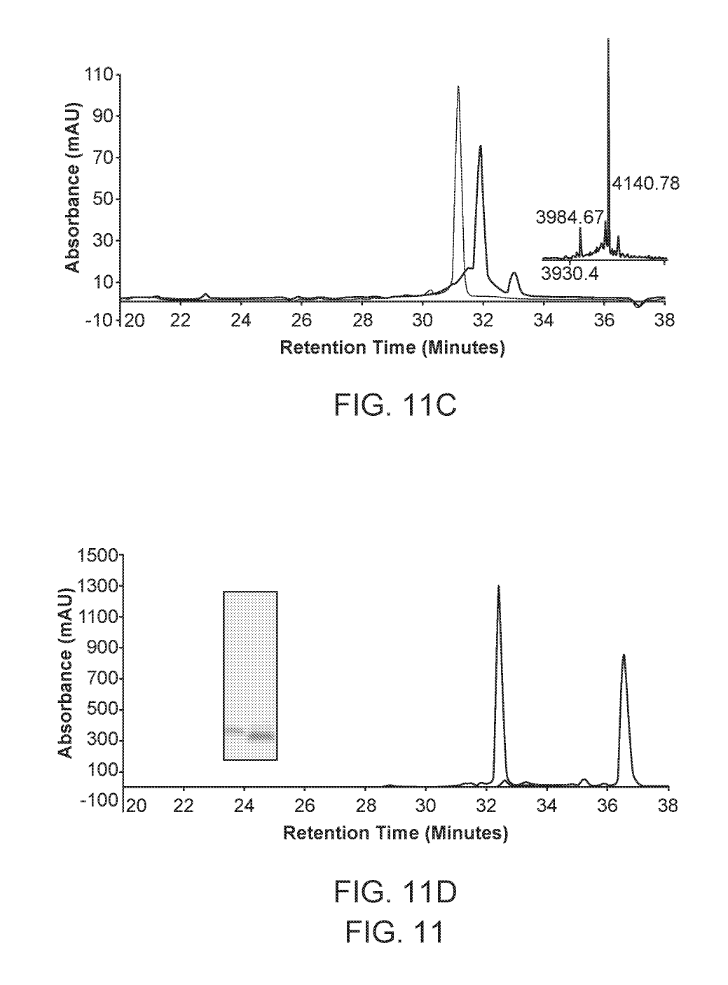

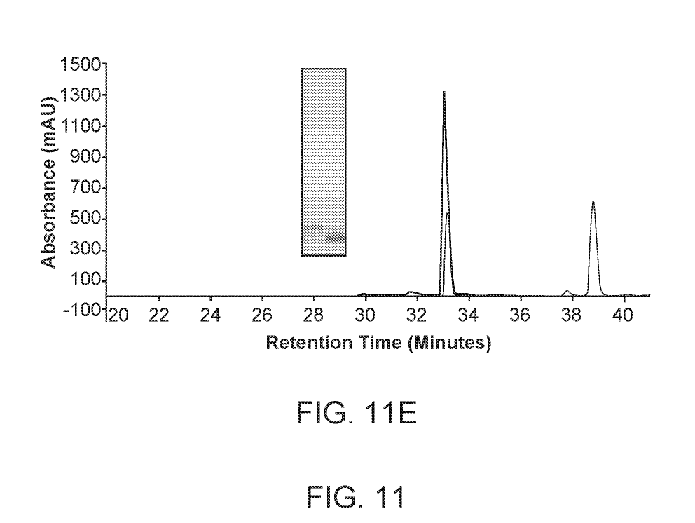

FIG. 11A through FIG. 11E illustrate quality control data from small scale (30 mL) mammalian expression studies of the peptides of SEQ ID NO: 4 (FIG. 11A), SEQ ID NO: 6 (FIG. 11B), SEQ ID NO: 17 (FIG. 11C), SEQ ID NO: 25 (FIG. 11D), and SEQ ID NO: 32 (FIG. 11E). The graphs illustrate HPLC traces on a hydrophobic column using a gradient of acetonitrile and 0.1% TFA. The darker trace is the native peptide, and the lighter trace is the peptide following reduction with 100 mM dithiothreitol. FIG. 11A, FIG. 11D, and FIG. 11E also include inset images showing nonreduced and reduced bands on SDS-PAGE gels. FIG. 11A and FIG. 11C also include MALDI mass spectrometry graphs providing the mass of the molecule and indicating that all the disulfides have been formed. Additionally, FIG. 11D illustrates that SEQ ID NO: 25 peptide has a greater purity in the reduced HPLC trace as compared to other peptides, such as SEQ ID NO: 4, SEQ ID NO: 6, and SEQ ID NO: 17 (FIG. 11A through FIG. 11C). This higher purity may be due to the presence of a C-terminal residue that is not Arg (i.e., the C-terminal residue here is Ile), which may prevent clipping.

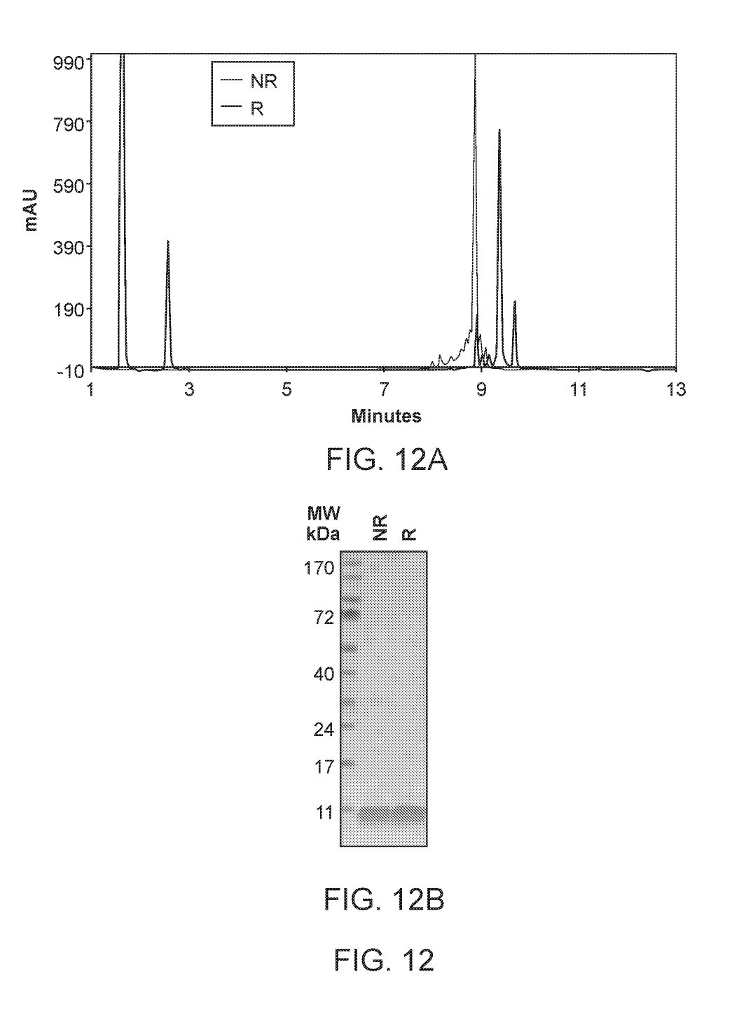

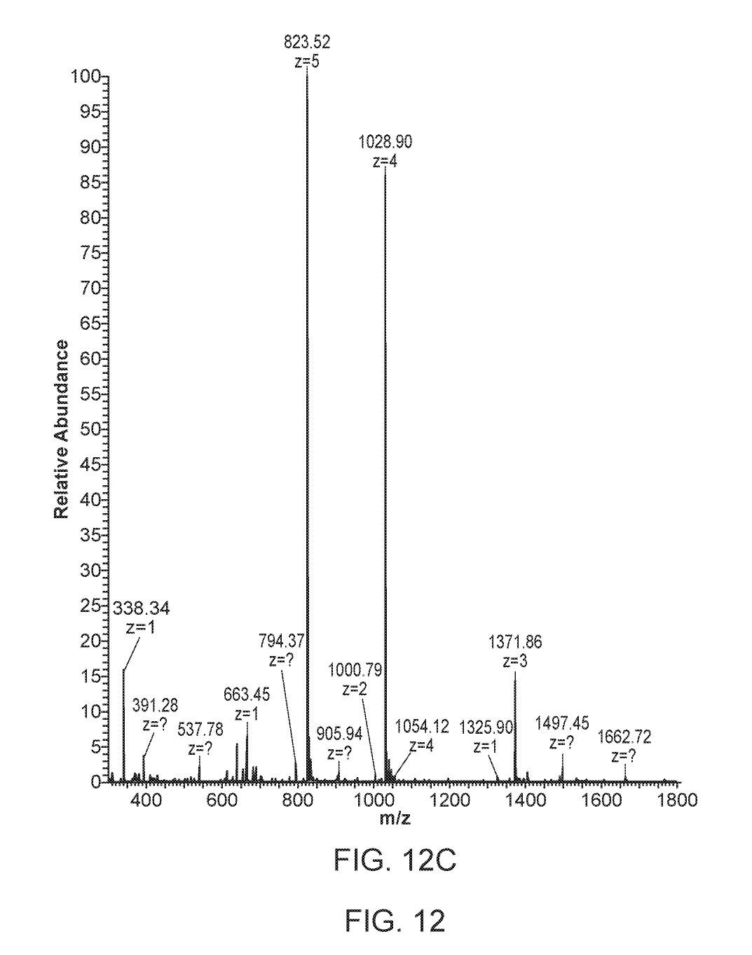

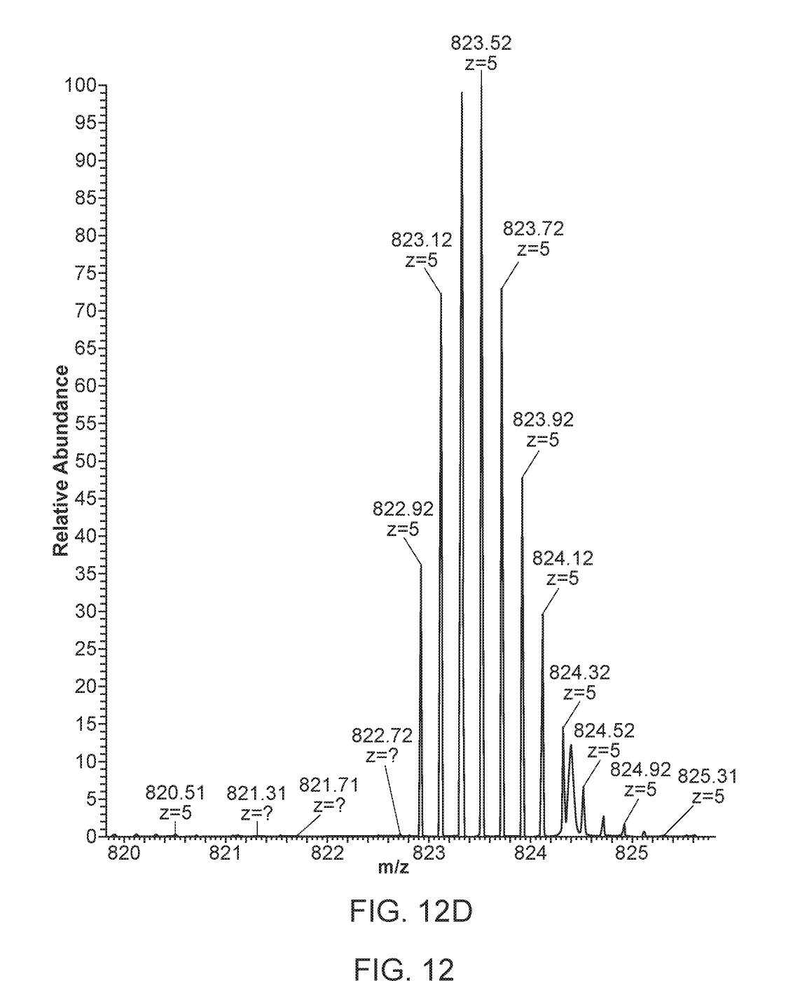

FIG. 12A through FIG. 12D illustrate quality control data from small-scale (30 mL) mammalian expression studies of the peptides of SEQ ID NO: 39 (FIG. 12A & FIG. 12B), and SEQ ID NO: 25 (FIG. 12C & FIG. 12D). The graph in FIG. 12A illustrates HPLC traces of SEQ ID NO: 39 on a hydrophobic column using a gradient of acetonitrile and 0.1% TFA. The darker trace is the native peptide, and the lighter trace is the peptide following reduction with 100 mM dithiothreitol. FIG. 12B is an image showing oxidized and reduced bands of peptides of SEQ ID NO: 41 on an SDS-PAGE gel. FIG. 12C shows the full spectra of a MALDI mass spectrometry graph of SEQ ID NO: 25 providing the mass of the molecule and indicating that all the disulfides have been formed. FIG. 12D shows a zoomed-in portion of FIG. 12C.

### Example 11

### Peptide Homing to Ventricles and Cerebral Spinal Fluid

This example illustrates homing of the peptide of SEQ ID NO: 55 to the ventricles and cerebral spinal fluid (CSF).

Different dosages of the peptides were administered to Female Harlan athymic nude mice, weighing 20 g-25 g, via tail vein injection (n=2 mice per knottin). The experiment was done in duplicates. The kidneys were ligated to prevent renal filtration of the peptides. Each peptide was radiolabeled by methylating lysines and the N-terminus, so the actual binding agent may contain methyl or dimethyl lysine(s) and a methylated or dimethylated amino terminus. A target dosage of 50-100 nmol of each peptide carrying 10-50 uCi of ¹⁴C was administered to Female Harlan athymic nude mice while anesthetized. Each peptide was allowed to freely circulate within the animal before the animals were euthanized and sectioned. Fluoxetine does cross the blood brain barrier (BBB), and was therefore used as a positive control in an animal that did not undergo kidney ligation. Inulin does not cross the BBB, and was therefore used as a negative control.

### Whole Body Autoradiography Images

For whole body autoradiography images, mice were frozen in a hexane/dry ice bath and then frozen in a block of carboxymethylcellulose at the end of the dosing period. Whole animal sagittal slices were prepared and frozen for imaging. These thin, frozen sections of tissues such as brain, tumor, liver, kidney, lung, heart, spleen, pancreas, muscle, adipose, gall bladder, upper gastrointestinal track, lower gastrointestinal track, bone, bone marrow, reproductive track, eye, cartilage, stomach, skin, spinal cord, bladder, salivary gland, and other types of tissues were obtained with a microtome, allowed to desiccate in a freezer, and exposed to phosphoimager plates for about 7 days. These plates were developed, and the signal (densitometry) from each organ was normalized to the signal found in the heart blood of each animal. A signal in tissue darker than the signal expected from blood in that tissue indicates accumulation in a region, tissue, structure or cell.

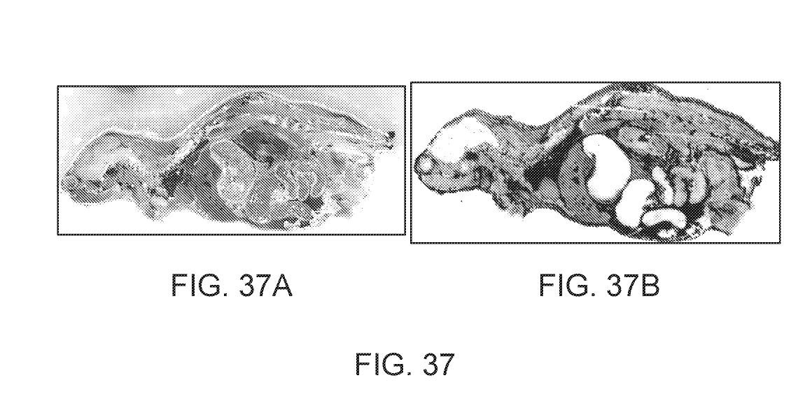

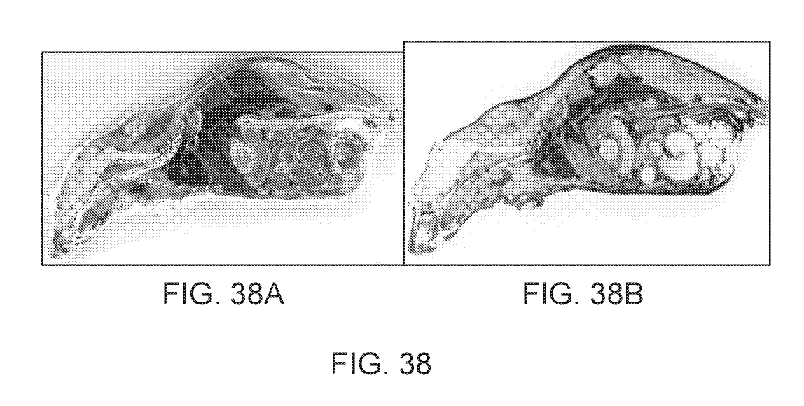

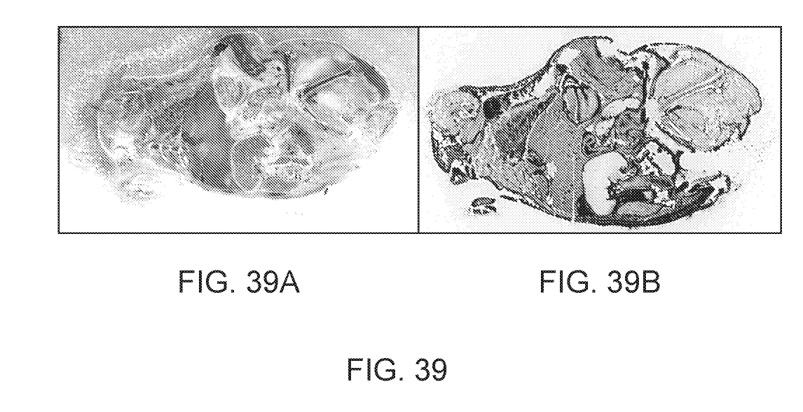

FIG. 37A illustrates a white light image of a frozen section of a mouse with ligated kidneys 3 hours after administration of 100 nmol of the radiolabeled first purified fraction (first HPLC peptide peak) of a peptide of SEQ ID NO: 55. FIG. 37B illustrates an autoradiographic image corresponding to FIG. 37A in which the ¹⁴C signal identifies the peptide distribution in the tissues of a mouse with ligated kidneys 3 hours after administration of 100 nmol of the radiolabeled first fraction (first HPLC peptide peak) of a peptide of SEQ ID NO: 55. FIG. 38A illustrates a white light image of a frozen section of a mouse with ligated kidneys 3 hours after administration of 100 nmol of the radiolabeled second purified fraction (second HPLC peptide peak of the HPLC from FIG. 37A) of a peptide of SEQ ID NO: 55. FIG. 38B illustrates an autoradiographic image corresponding to FIG. 38A in which the ¹⁴C signal identifies the peptide distribution in the tissues of the mouse with ligated kidneys 3 hours after administration of 100 nmol of the radiolabeled second purified fraction (second HPLC peptide peak of the HPLC from FIG. 37A) of a peptide of SEQ ID NO: 55. FIG. 39A shows a white light image of a frozen section of a mouse with ligated kidneys 3 hours after administration of 100 nmol of the radiolabeled SEQ ID NO: 83 peptide. FIG. 39B shows an autoradiographic image corresponding to FIG. 39A in which the ¹⁴C signal identifies the peptide distribution in the tissues of the mouse with ligated kidneys 3 hours after administration of 100 nmol of the radiolabeled SEQ ID NO: 83 peptide.

### Brain Section Autoradiography Images

For autoradiography images of sagittal and coronal brain sections, anesthetized mice were decapitated, brains were isolated and frozen in a hexane/dry ice bath and then frozen in a block of carboxymethylcellulose at the end of the dosing period. 40 um sections of the brain tissue were prepared every 0.5 mm that resulted in thin frozen sections being available for imaging. Thin, frozen sections were obtained with a cryotome, allowed to desiccate in a freezer, and exposed to phosphoimager plates for about 7 days before developing on a Raytest CR-35 scanner.

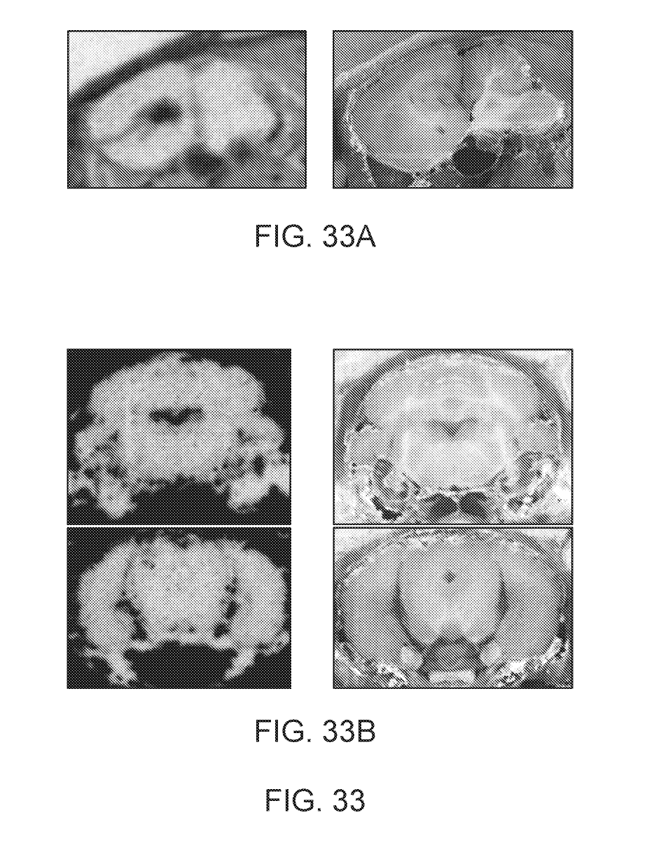

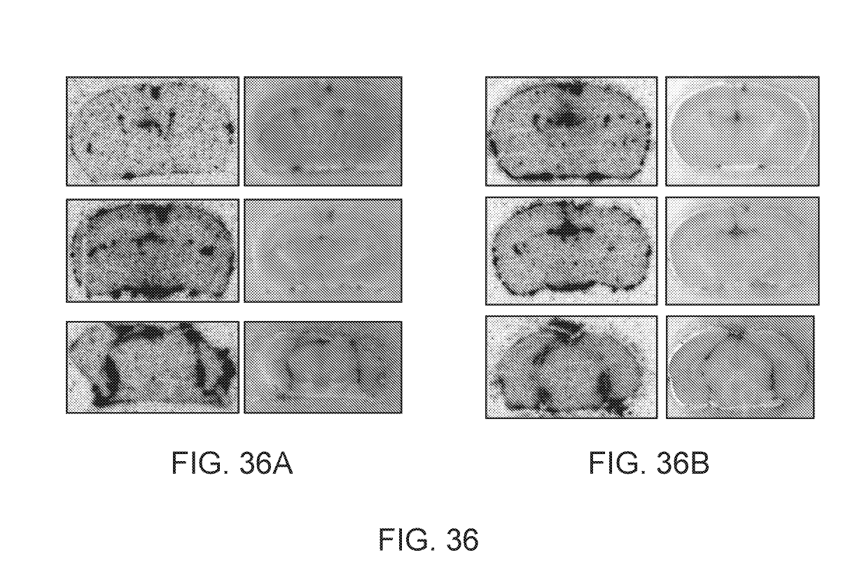

FIG. 33A and FIG. 33B show sagittal (FIG. 33A) and coronal (FIG. 33B) brain sections indicating localization of a peptide of SEQ ID NO: 55 to specific structures in the brain, such as ventricles and CSF. In each figure, the radioactivity scan is shown on the left, with dark areas having higher activity. Images of the tissue in normal light are shown on the right. FIG. 36A illustrates white light images of coronal brain sections of a mouse on the right and autoradiographic images that correspond to the white light images on the left. The ¹⁴C signal in the autographic images identifies the peptide distribution, indicating localization of the radiolabeled first purified fraction (first HPLC peptide peak) of a peptide of SEQ ID NO: 55, to specific structures in the brain, such as ventricles and CSF after administration of the peptide.

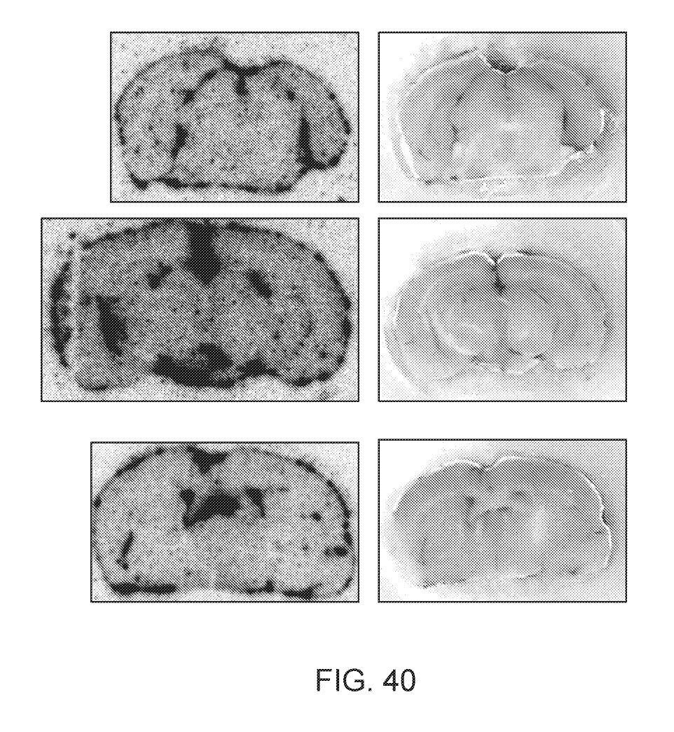

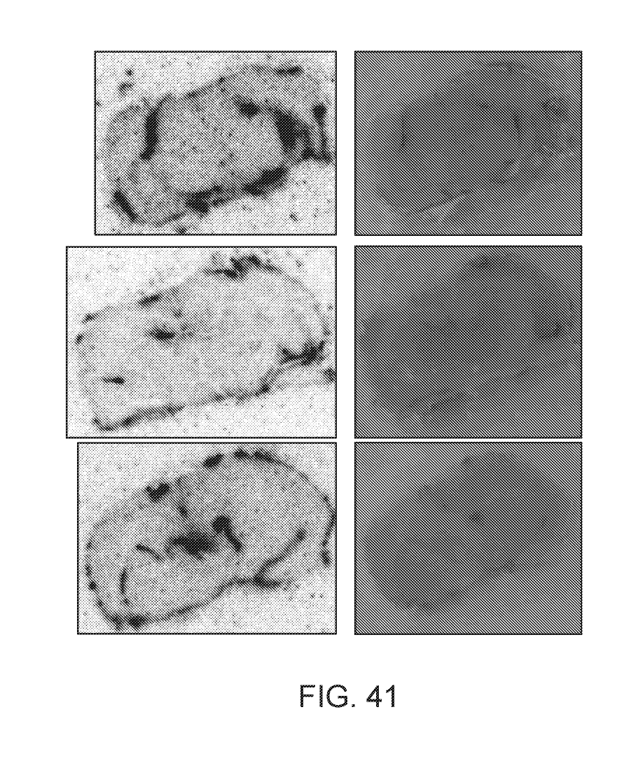

FIG. 36B illustrates white light images of coronal brain sections of a mouse on the right and autoradiographic images corresponding to the white light images on the left. The ¹⁴C signal in the autographic images identifies the peptide distribution, indicating localization of the second purified fraction (second HPLC peptide peak from the HPLC from FIG. 36A) of a peptide of SEQ ID NO: 55, to specific structures in the brain, such as ventricles and CSF after administration of the peptide. The ¹⁴C signal in the autographic images identifies the peptide distribution, indicating localization of the second purified peak of a peptide of SEQ ID NO: 55 to specific structures in the brain, such as ventricles and CSF. FIG. 40 shows white light images of coronal brain sections on the right and autoradiographic images that correspond to the white light images on the left. The ¹⁴C signal in the autographic images identifies the peptide distribution 3 hours after administration of the radiolabeled SEQ ID NO: 34 and indicates the localization of the peptide to specific structures in the brain, such as ventricles and CSF. FIG. 41 shows white light images of coronal brain sections on the right and autoradiographic images that correspond to the white light images on the left. The ¹⁴C signal in the autographic images identifies the peptide distribution 3 hours after administration of the radiolabeled SEQ ID NO: 83 and indicates the localization of the peptide to specific structures in the brain, such as ventricles and CSF.

### HPLC of Brain Tissues

For HPLC of brain tissue, harvested, frozen brains were homogenized to isolate protein in a buffer consisting of 1 mM Tris pH8, 150 mM NaCl, 1 mM EDTA, 25 mM Sucrose, and protease inhibitor cocktail in PBS. Each brain sample was added to 5 volumes (w:v) of buffer and homogenized in a locking, round bottom, 2 ml tube with a steel bead on a Qialyzer for 2 minutes at a frequency of 30/sec. Homogenized samples were centrifuged at 4 degrees Celsius for 30 minutes at 16,000 rpm on a TOMY TX-160 centrifuge or for 30 minutes at top speed on a table top centrifuge, and the soluble supernatant collected. The soluble supernatant was prepared for HPLC analysis by filtering through at 0.2 um syringe filter, using methanol to rinse the filter after passage of the sample. The filtered sample and methanol were collected and dried down on a blow-down evaporator using gaseous nitrogen. The dried sample was re-suspended in 125 ul.

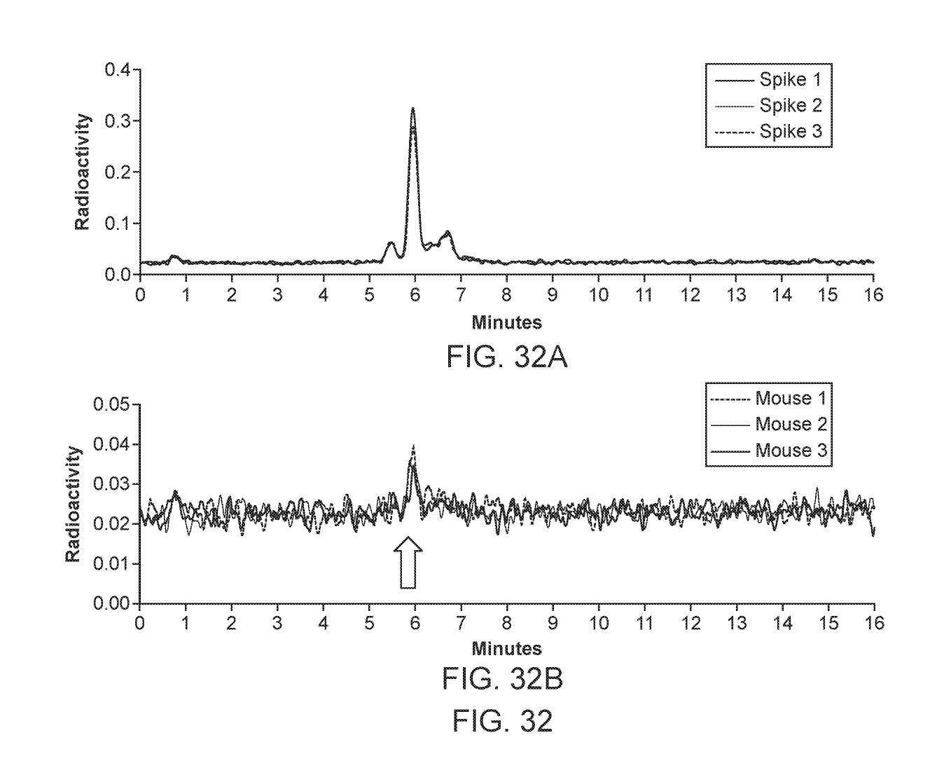

FIG. 32A and FIG. 32B illustrate HPLC radiograms of a ¹⁴C-labeled peptide of SEQ ID NO: 55 in whole brain homogenates. FIG. 32A shows the peptides spiked into a crude brain homogenate and run on a scintillation detector-equipped HPLC on a hydrophobic column using an acetonitrile gradient and 0.1% TFA. FIG. 32B shows a scintillation HPLC trace of three mouse brains following systemic administration of the radiolabeled peptide. Therefore, intact SEQ ID NO: 55 peptide was present in the brain of mice that were dosed intravenously with the peptide.

### Example 12

### Treatment of Dementia with a Peptide of the Disclosure

This example describes the treatment of dementia with a peptide of the present disclosure. One or more of the peptides of SEQ ID NO: 1-SEQ ID NO: 196 or SEQ ID NO: 210-SEQ ID NO: 405 are expressed recombinantly or chemically synthesized. Optionally, the peptides of SEQ ID NO: 1-SEQ ID NO: 196 or SEQ ID NO: 210-SEQ ID NO: 405 are mutated to bind to Tau before administration The peptides are administered to a human or animal. The peptides cross the blood brain barrier and specifically bind to Tau to prevent buildup of toxic Tau aggregates associated with various forms of dementia.

Alternatively, one or more of the peptides of SEQ ID NO: 1-SEQ ID NO: 196 or SEQ ID NO: 210-SEQ ID NO: 405 are expressed recombinantly or chemically synthesized, then conjugated to tacrine. One or more tacrine-peptide conjugates are administered to a human or animal. Coupling of tacrine to any one of the peptides of SEQ ID NO: 1-SEQ ID NO: 196 or SEQ ID NO: 210-SEQ ID NO: 405 targets tacrine into the brain, allowing for more delivery into the central nervous system (CNS), and less delivery in the periphery where it causes side effects.

### Example 13

### Treatment of a Neurodegenerative Disease with a Peptide-Conjugate of the Disclosure

This example describes the treatment of a neurodegenerative disease with a peptide-conjugate. A peptide of the disclosure is expressed recombinantly or chemically synthesized and then is conjugated (or expressed recombinantly and fused) to a growth factor, such as epidermal growth factor (EGF) that can regulate proliferation or recruitment of precursor cells to multiple sclerosis lesions and promote remyelination. Coupling of the drugs to the peptide of any one of SEQ ID NO: 1-SEQ ID NO: 196 or SEQ ID NO: 210-SEQ ID NO: 405 targets the growth factor into the brain. One or more growth factor-peptide conjugates are administered to a human or animal.

Additionally, a peptide of the disclosure is expressed recombinantly or chemically synthesized and then is conjugated to a therapeutic compound for treating a neurogenerative disease (e.g., Alzheimer's disease), such as an acetylcholinesterase inhibitor (e.g., rivastigimine), galantamine, donzepil, tacrin, or a neurotoxin (e.g., sarin). Coupling of the drugs to the peptide of any one of SEQ ID NO: 1-SEQ ID NO: 196 or SEQ ID NO: 210-SEQ ID NO: 405 targets the therapeutic compound into the brain. One or more therapeutic conjugates are administered to a human or animal.

### Example 14

### Modifying a Function of a Peptide of the Disclosure

This example illustrates the modification of a function of a peptide. A peptide of the disclosure is mutated at one or more residues in order to modify its activity. Such modification could include gaining or losing binding to certain ion channels, inhibition of certain proteases, or antimicrobial activity. The modified peptide is expressed recombinantly or chemically synthesized. One or more modified peptides are administered to a human or an animal. The modified peptide may be used directly as a therapeutic agent. Alternatively, the modified peptide is conjugated to a therapeutic agent to target the therapeutic agent into the brain.

### Example 15

### Treatment of a Neurodegenerative Disease with a Peptide of the Disclosure

This example describes the treatment of a neurodegenerative disease with a peptide. A peptide of the disclosure is expressed recombinantly or chemically synthesized and administered to a human or animal. The peptide crosses the BBB and agonizes or antagonizes an ion channel, such as potassium sodium channels, chloride channels, calcium channels, nicotinic acetyl choline receptors, transient receptor potential channels, NMDA receptors, serotonin receptors, KIR channels, GABA channels, glycine receptors, glutamate receptors, acid sensing ion channels, K2P channels, Nav1.7, or purinergic receptors, thereby providing a therapeutic effect.

### Example 16

### Whole-Body Autoradiography after Peptide Administration

This example shows whole-body autoradiographs of female Harlan athymic nude mice given different dosages of the radiolabeled peptides or radiolabeled peptide conjugates. The kidneys of each mouse were either left intact or ligated to prevent renal filtration of the peptides. The peptides or peptide conjugates were radiolabeled by methylating lysines and the N-terminus so the actual binding agent may contain methyl or dimethyl lysine(s) and a methylated or dimethylated amino terminus. The radiolabeled peptides were radiolabeled peptide with SEQ ID NO: 5, radiolabeled peptide with SEQ ID NO: 35, or radiolabeled peptide with SEQ ID NO: 37. The radiolabeled peptide conjugates were radiolabeled peptide with SEQ ID NO: 5 conjugated to Alexa 647 fluorescent dye (SEQ ID NO: 5-RA peptide conjugate), radiolabeled peptide with SEQ ID NO: 5 conjugated to MMAE (SEQ ID NO: 5-RZ peptide conjugate), or radiolabeled peptide with SEQ ID NO: 5 conjugated to DM-1 (SEQ ID NO: 5-RY peptide conjugate). SEQ ID NO: 5-RA peptide conjugate was made by conjugating Alexa 647 to a free amine on either the N-terminus or lysine with an NHS ester. SEQ ID NO: 5-RZ peptide conjugate was made by conjugating MMAE with a valine-citrulline-PABC linker to the K amino acid residue of the SEQ ID NO: 5 peptide with an NHS ester, such as shown in the compound of Structure (I) below:

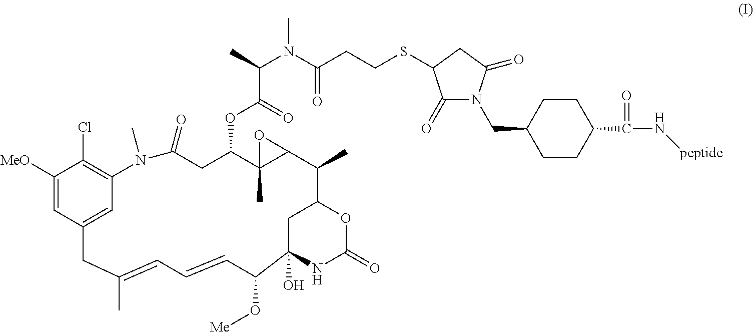

The SEQ ID NO: 5-RY peptide conjugate was made by conjugating DM-1 with a non-cleavable linker to the K amino acid residue of the SEQ ID NO: 5 peptide with an NHS ester, such as shown in the compound of Structure (II) below:

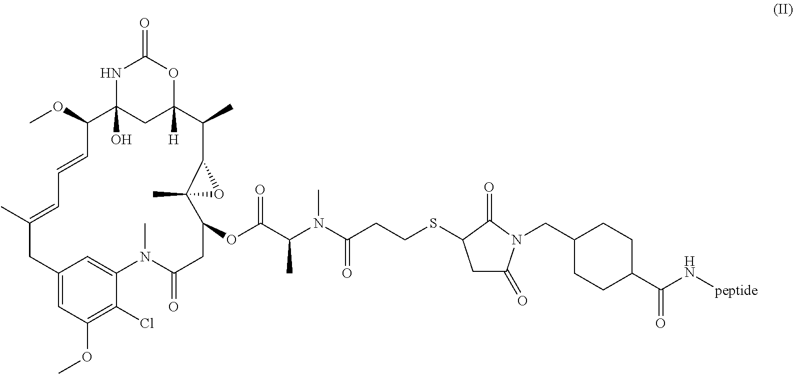

A target dosage of 9-14 nmol of each peptide carrying 2 uCi of ¹⁴C was administered to Female Harlan athymic nude mice while anesthetized.

More specifically, a dosage of 9 nmol of SEQ ID NO: 5-RA peptide conjugate was administered to four mice with intact kidneys. A dosage of 11 nmol of SEQ ID NO: 5-RZ peptide was administered to four mice with intact kidneys. A dosage of 12.8 nmol of radiolabeled SEQ ID NO: 5 peptide was administered to two mice with intact kidneys. A dosage of 14 nmol of SEQ ID NO: 5-RY peptide conjugate was administered to the four mice with intact kidneys. A dosage of 100 nmol of the radiolabeled SEQ ID NO: 35 peptide was administered to the two mice with ligated kidneys and two mice with intact kidneys. A dosage of 100 nmol of the radiolabeled SEQ ID NO: 37 peptide was administered to the two mice with ligated kidneys and two mice with intact kidneys. The target dosage of each peptide was administered to Female Harlan athymic nude mice while anesthetized. Mice that received the radiolabeled SEQ ID NO: 5 peptide, the SEQ ID NO: 5-RA peptide conjugate, the SEQ ID NO: 5-RZ peptide conjugate, or the SEQ ID NO: 5-RY peptide conjugate were RH-28 tumor bearing Female Harlan athymic mice. Each peptide was allowed to freely circulate within the animal before the animals were euthanized and sectioned. Two mice that received a 9 nmol dose of the SEQ ID NO: 5-RA peptide conjugate were euthanized three hours after administration of the peptide. Two mice that received a 9 nmol dose of the SEQ ID NO: 5-RA peptide conjugate were euthanized twenty-four hours after administration of the peptide. Two mice that received an 11 nmol dose of the SEQ ID NO: 5-RZ peptide conjugate were euthanized three hours after administration of the peptide. Two mice that received an 11 nmol dose of the SEQ ID NO: 5-RZ peptide conjugate were euthanized twenty-four hours after administration of the peptide. Two mice that received a 12.8 nmol dose of the radiolabeled SEQ ID NO: 5 peptide were euthanized three hours after administration of the peptide. Two mice that received a 14 nmol dose of the SEQ ID NO: 5-RY peptide conjugate were euthanized three hours after administration of the peptide. Two mice that received a 14 nmol dose of the SEQ ID NO: 5-RY peptide conjugate were euthanized twenty-four hours after administration of the peptide. Four mice that received a 100 nmol dose of the radiolabeled SEQ ID NO: 35 peptide were euthanized three hours after administration of the peptide. Four mice that received a 100 nmol dose of the SEQ ID NO: 37 peptide were euthanized three hours after administration of the peptide.

At the end of the dosing period, mice were frozen in a hexane/dry ice bath and then frozen in a block of carboxymethylcellulose. Whole animal sagittal slices were prepared that resulted in thin frozen sections being available for imaging. Thin, frozen sections of animal were obtained with a microtome, allowed to desiccate in a freezer, and exposed to phosphoimager plates for about ten days.

These plates were developed, and the signal (densitometry) from each organ was normalized to the signal found in the heart blood of each animal. A signal in tissue darker than the signal expected from blood in that tissue indicates peptide accumulation in a region, tissue, structure or cell.

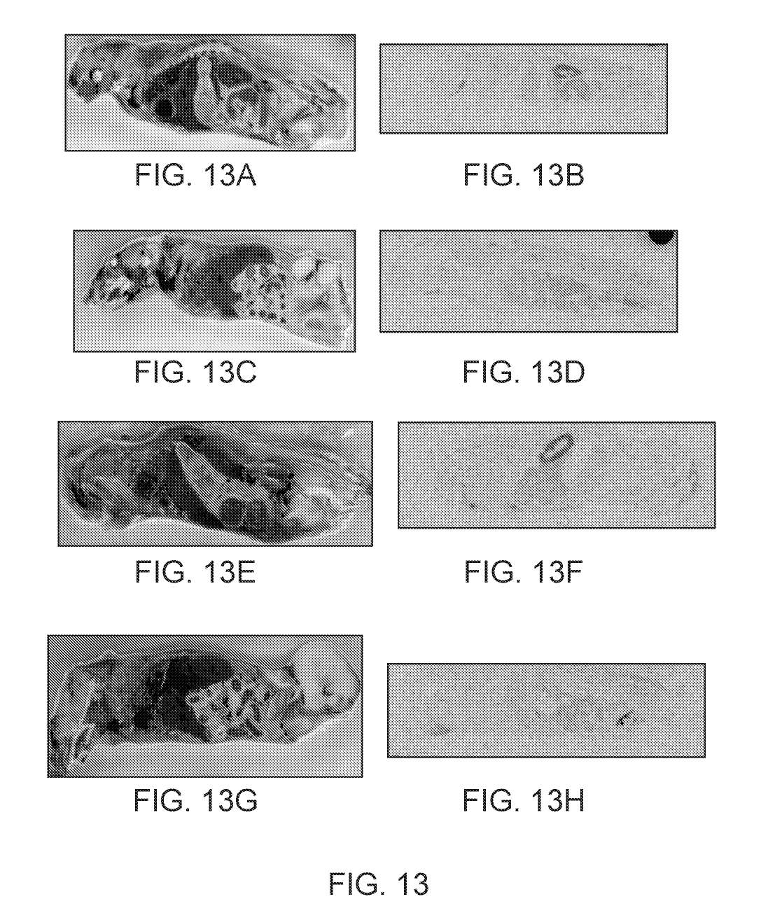

FIG. 13A illustrates a white light image of a frozen section of a mouse three hours after administration of 9 nmol of the SEQ ID NO: 5-RA peptide conjugate. FIG. 13B illustrates an autoradiographic image corresponding to FIG. 13A in which the ¹⁴C signal identifies the peptide distribution in the tissues, including the RH-28 tumor, of a mouse three hours after administration of 9 nmol of the SEQ ID NO: 5-RA peptide conjugate. FIG. 13C illustrates a white light image of a different frozen section of the same mouse as in FIG. 13A and FIG. 13B three hours after administration of 9 nmol of the SEQ ID NO: 5-RA peptide conjugate. FIG. 13D illustrates an autoradiographic image corresponding to FIG. 13C in which the ¹⁴C signal identifies the peptide distribution in the tissues, including the RH-28 tumor, three hours after administration of 9 nmol of the SEQ ID NO: 5-RA peptide conjugate. FIG. 13E illustrates a white light image of a frozen section of a different mouse than shown in FIG. 13A through FIG. 13D three hours after administration of 9 nmol of the SEQ ID NO: 5-RA peptide conjugate. FIG. 13F illustrates an autoradiographic image corresponding to FIG. 13E in which the ¹⁴C signal identifies the peptide distribution in the tissues, including in the RH-28 tumor, of a mouse three hours after administration of 9 nmol of the SEQ ID NO: 5-RA peptide conjugate. FIG. 13G illustrates a white light image of a different frozen section of the same mouse as in FIG. 13E and FIG. 13F three hours after administration of 9 nmol of the SEQ ID NO: 5-RA peptide conjugate. FIG. 13H illustrates an autoradiographic image corresponding to FIG. 13G in which the ¹⁴C signal identifies the peptide distribution in the tissues, including in the RH-28 tumor, three hours after administration of 9 nmol of the SEQ ID NO: 5-RA peptide conjugate.

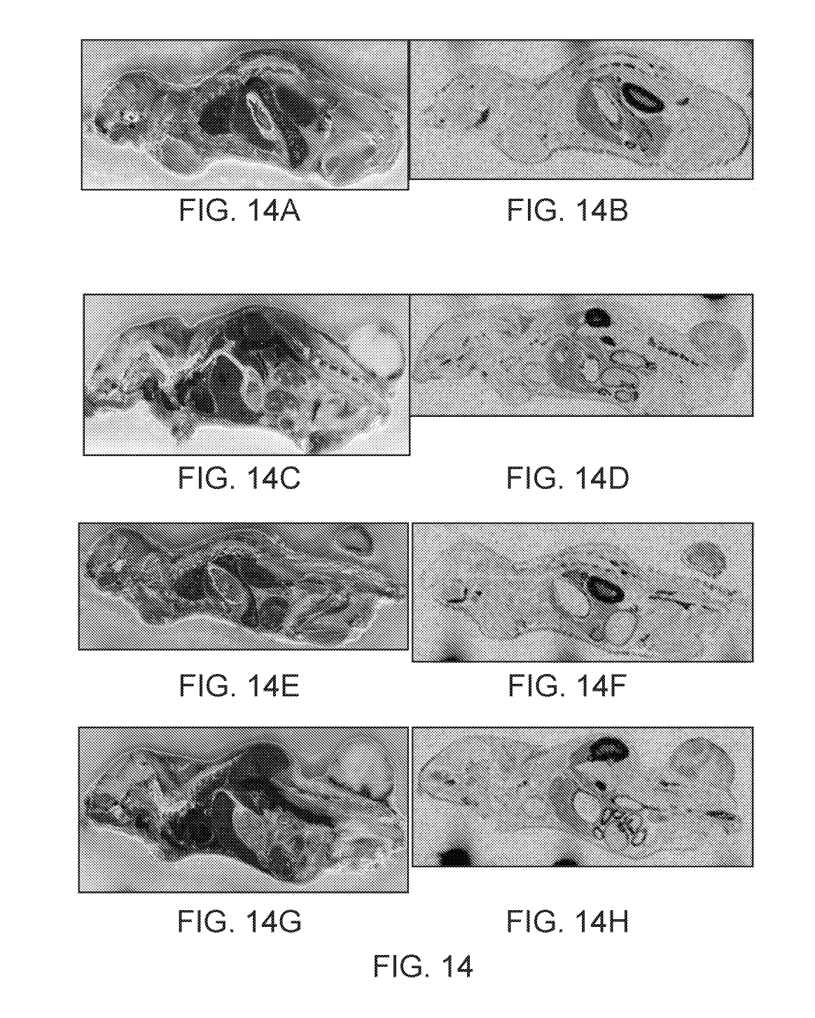

FIG. 14A illustrates a white light image of a frozen section of a mouse twenty-four hours after administration of 9 nmol of the SEQ ID NO: 5-RA peptide conjugate. FIG. 14B illustrates an autoradiographic image corresponding to FIG. 14A in which the ¹⁴C signal identifies the peptide distribution in the tissues, including in the RH-28 tumor, of a mouse twenty-four hours after administration of 9 nmol of the SEQ ID NO: 5-RA peptide conjugate. FIG. 14C illustrates a white light image of a different frozen section of the same mouse as in FIG. 14A and FIG. 14B twenty-four hours after administration of 9 nmol of the SEQ ID NO: 5-RA peptide conjugate. FIG. 14D illustrates an autoradiographic image corresponding to FIG. 14C in which the ¹⁴C signal identifies the peptide distribution in the tissues, including in the RH-28 tumor, twenty-four hours after administration of 9 nmol of the SEQ ID NO: 5-RA peptide conjugate. FIG. 14E illustrates a white light image of a frozen section of a different mouse than shown in FIG. 14A through FIG. 14D twenty-four hours after administration of 9 nmol of the SEQ ID NO: 5-RA peptide conjugate. FIG. 14F illustrates an autoradiographic image corresponding to FIG. 14E in which the ¹⁴C signal identifies the peptide distribution in the tissues, including the RH-28 tumor, of a mouse twenty-four hours after administration of 9 nmol of the SEQ ID NO: 5-RA peptide conjugate. FIG. 14G illustrates a white light image of a different frozen section of the same mouse as in FIG. 14E and FIG. 14F twenty-four hours after administration of 9 nmol of the SEQ ID NO: 5-RA peptide conjugate. FIG. 14H illustrates an autoradiographic image corresponding to FIG. 14G in which the ¹⁴C signal identifies the peptide distribution in the tissues, including in the RH-28 tumor, twenty-four hours after administration of 9 nmol of the SEQ ID NO: 5-RA peptide conjugate.

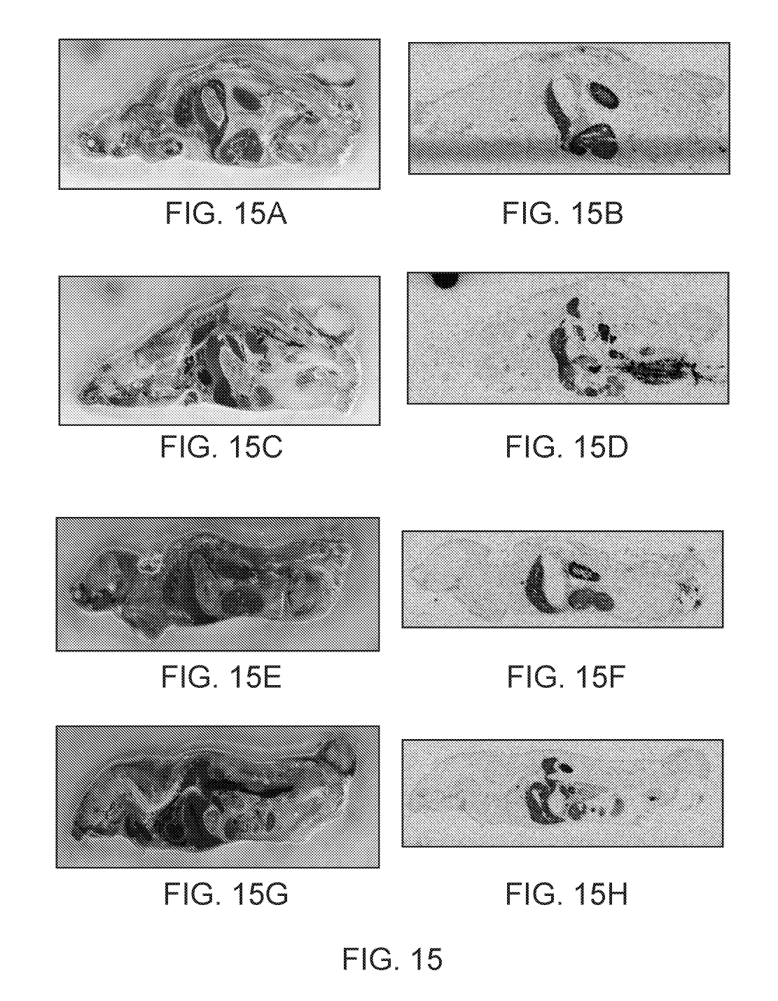

FIG. 15A illustrates a white light image of a frozen section of a mouse three hours after administration of 11 nmol of the SEQ ID NO: 5-RZ peptide conjugate. FIG. 15B illustrates an autoradiographic image corresponding to FIG. 15A in which the ¹⁴C signal identifies the peptide distribution in the tissues, including in the RH-28 tumor, of a mouse three hours after administration of 11 nmol of the SEQ ID NO: 5-RZ peptide conjugate. FIG. 15C illustrates a white light image of a different frozen section of the same mouse as in FIG. 15A and FIG. 15B three hours after administration of 11 nmol of the SEQ ID NO: 5-RZ peptide conjugate. FIG. 15D illustrates an autoradiographic image corresponding to FIG. 15C in which the ¹⁴C signal identifies the peptide distribution in the tissues, including in the RH-28 tumor, three hours after administration of 11 nmol of the SEQ ID NO: 5-RZ peptide conjugate. FIG. 15E illustrates a white light image of a frozen section of a different mouse than shown in FIG. 15A through FIG. 15D three hours after administration of 11 nmol of the SEQ ID NO: 5-RZ peptide conjugate.

FIG. 15F illustrates an autoradiographic image corresponding to FIG. 15E in which the ¹⁴C signal identifies the peptide distribution in the tissues, including in the RH-28 tumor, of a mouse three hours after administration of 11 nmol of the SEQ ID NO: 5-RZ peptide conjugate. FIG. 15G illustrates a white light image of a different frozen section of the same mouse as in FIG. 15E and FIG. 15F three hours after administration of 11 nmol of the SEQ ID NO: 5-RZ peptide conjugate. FIG. 15H illustrates an autoradiographic image corresponding to FIG. 15G in which the ¹⁴C signal identifies the peptide distribution in the tissues, including in the RH-28 tumor, three hours after administration of 11 nmol of the SEQ ID NO: 5-RZ peptide conjugate.

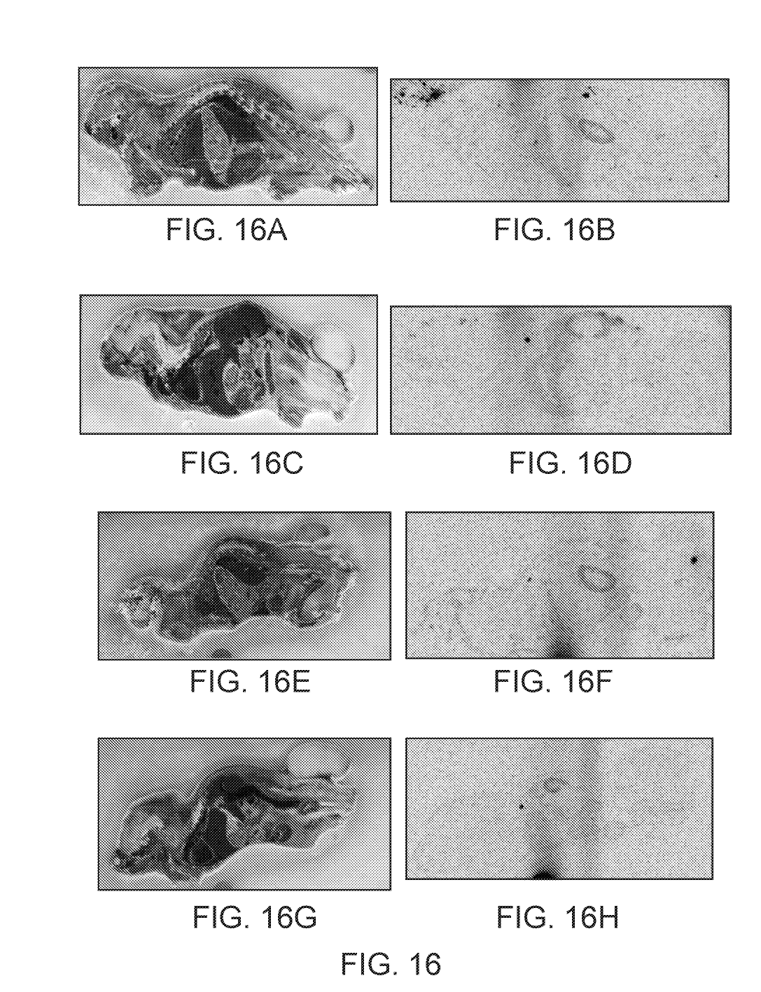

FIG. 16A illustrates a white light image of a frozen section of a mouse twenty-four hours after administration of 11 nmol of the SEQ ID NO: 5-RZ peptide conjugate. FIG. 16B illustrates an autoradiographic image corresponding to FIG. 16A in which the ¹⁴C signal identifies the peptide distribution in the tissues, including in the RH-28 tumor, of a mouse twenty-four hours after administration of 11 nmol of the SEQ ID NO: 5-RZ peptide conjugate. FIG. 16C illustrates a white light image of a different frozen section of the same mouse as in FIG. 16A and FIG. 16B twenty-four hours after administration of 11 nmol of the SEQ ID NO: 5-RZ peptide conjugate. FIG. 16D illustrates an autoradiographic image corresponding to FIG. 16C in which the ¹⁴C signal identifies the peptide distribution in the tissues, including in the RH-28 tumor, twenty-four hours after administration of 11 nmol of the SEQ ID NO: 5-RZ peptide conjugate. FIG. 16E illustrates a white light image of a frozen section of a different mouse than shown in FIG. 16A through FIG. 16D twenty-four hours after administration of 11 nmol of the SEQ ID NO: 5-RZ peptide conjugate. FIG. 16F illustrates an autoradiographic image corresponding to FIG. 16E in which the ¹⁴C signal identifies the peptide distribution in the tissues, including in the RH-28 tumor, of a mouse twenty-four hours after administration of 11 nmol of the SEQ ID NO: 5-RZ peptide conjugate peptide. FIG. 16G illustrates a white light image of a different frozen section of the same mouse as in FIG. 16E and FIG. 16F twenty-four hours after administration of 11 nmol of the SEQ ID NO: 5-RZ peptide conjugate. FIG. 16H illustrates an autoradiographic image corresponding to FIG. 16G in which the ¹⁴C signal identifies the peptide distribution in the tissues, including in the RH-28 tumor, twenty-four hours after administration of 11 nmol of the SEQ ID NO: 5-RZ peptide conjugate.

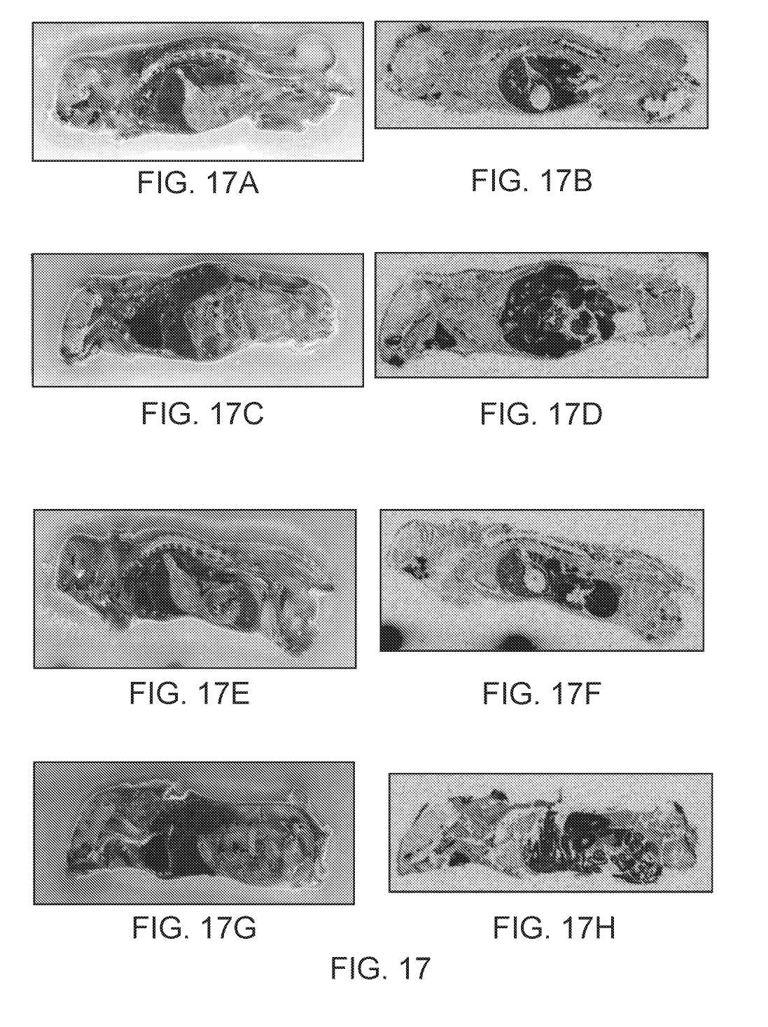

FIG. 17A illustrates a white light image of a frozen section of a mouse three hours after administration of 12.8 nmol of the radiolabeled SEQ ID NO: 5 peptide. FIG. 17B illustrates an autoradiographic image corresponding to FIG. 17A in which the ¹⁴C signal identifies the peptide distribution the tissues, including in the RH-28 tumor, of a mouse three hours after administration of 12.8 nmol of the radiolabeled SEQ ID NO: 5 peptide. FIG. 17C illustrates a white light image of a different frozen section of the same mouse as in FIG. 17A and FIG. 17B three hours after administration of 12.8 nmol of the radiolabeled SEQ ID NO: 5 peptide. FIG. 17D illustrates an autoradiographic image corresponding to FIG. 17C in which the ¹⁴C signal identifies the peptide distribution in the tissues, including in the RH-28 tumor, three hours after administration of 12.8 nmol of the radiolabeled SEQ ID NO: 5 peptide. FIG. 17E illustrates a white light image of a frozen section of a different mouse than shown in FIG. 17A through FIG. 17D three hours after administration of 12.8 nmol of the radiolabeled SEQ ID NO: 5 peptide. FIG. 17F illustrates an autoradiographic image corresponding to FIG. 17E in which the ¹⁴C signal identifies the peptide distribution in the tissues, including in the RH-28 tumor, of a mouse three hours after administration of 12.8 nmol of the radiolabeled SEQ ID NO: 5 peptide. FIG. 17G illustrates a white light image of a different frozen section of the same mouse as in FIG. 17E and FIG. 17F three hours after administration of 12.8 nmol of the radiolabeled SEQ ID NO: 5 peptide. FIG. 17H illustrates an autoradiographic image corresponding to FIG. 17G in which the ¹⁴C signal identifies the peptide distribution in the tissues, including in the RH-28, tumor three hours after administration of 12.8 nmol of the radiolabeled SEQ ID NO: 5 peptide.

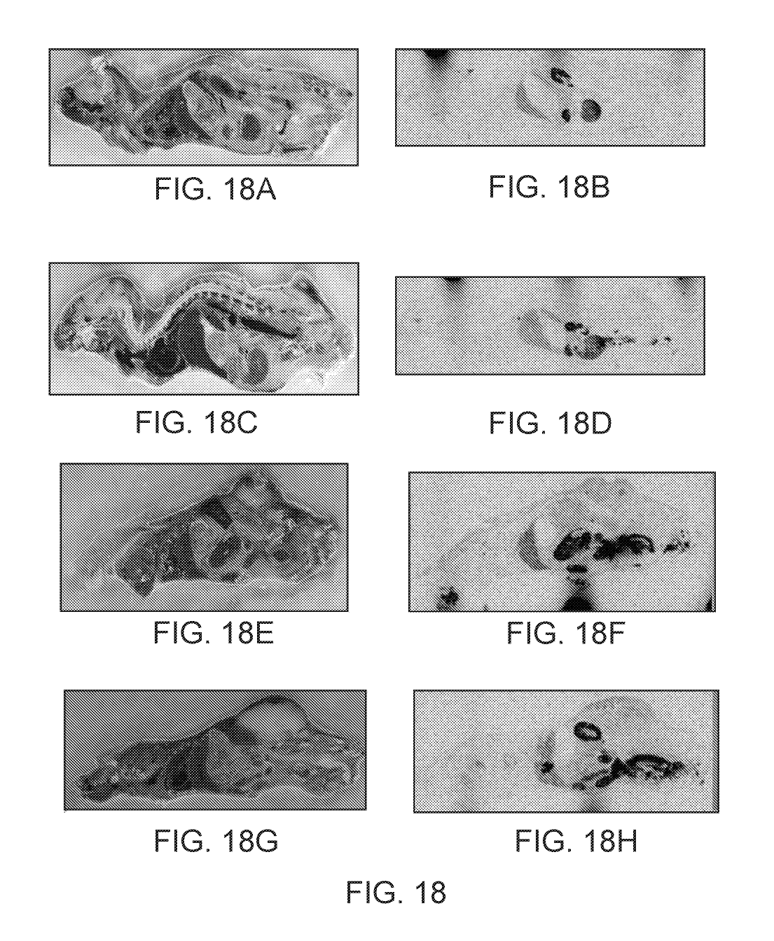

FIG. 18A illustrates a white light image of a frozen section of a mouse three hours after administration of 14 nmol of the SEQ ID NO: 5-RY peptide conjugate. FIG. 18B illustrates an autoradiographic image corresponding to FIG. 18A in which the ¹⁴C signal identifies the peptide distribution in the tissues, including in the RH-28 tumor, of a mouse three hours after administration of 14 nmol of the SEQ ID NO: 5-RY peptide conjugate. FIG. 18C illustrates a white light image of a different frozen section of the same mouse as in FIGS. 18A and 18B three hours after administration of 14 nmol of the SEQ ID NO: 5-RY peptide conjugate. FIG. 18D illustrates an autoradiographic image corresponding to FIG. 18C in which the ¹⁴C signal identifies the peptide distribution in the tissues, including in the RH-28 tumor, three hours after administration of 14 nmol of the SEQ ID NO: 5-RY peptide conjugate. FIG. 18E illustrates a white light image of a frozen section of a different mouse than shown in FIG. 18A through FIG. 18D three hours after administration of 14 nmol of the SEQ ID NO: 5-RY peptide conjugate. FIG. 18F illustrates an autoradiographic image corresponding to FIG. 18E in which the ¹⁴C signal identifies the peptide distribution in the tissues, including in the RH-28 tumor of a mouse three hours after administration of 14 nmol of the SEQ ID NO: 5-RY peptide conjugate. FIG. 18G illustrates a white light image of a different frozen section of the same mouse as in FIG. 18E and FIG. 18F three hours after administration of 14 nmol of the SEQ ID NO: 5-RY peptide conjugate. FIG. 18H illustrates an autoradiographic image corresponding to FIG. 18G in which the ¹⁴C signal identifies the peptide distribution in the tissues, including in the RH-28 tumor, three hours after administration of 14 nmol of the SEQ ID NO: 5-RY peptide conjugate.

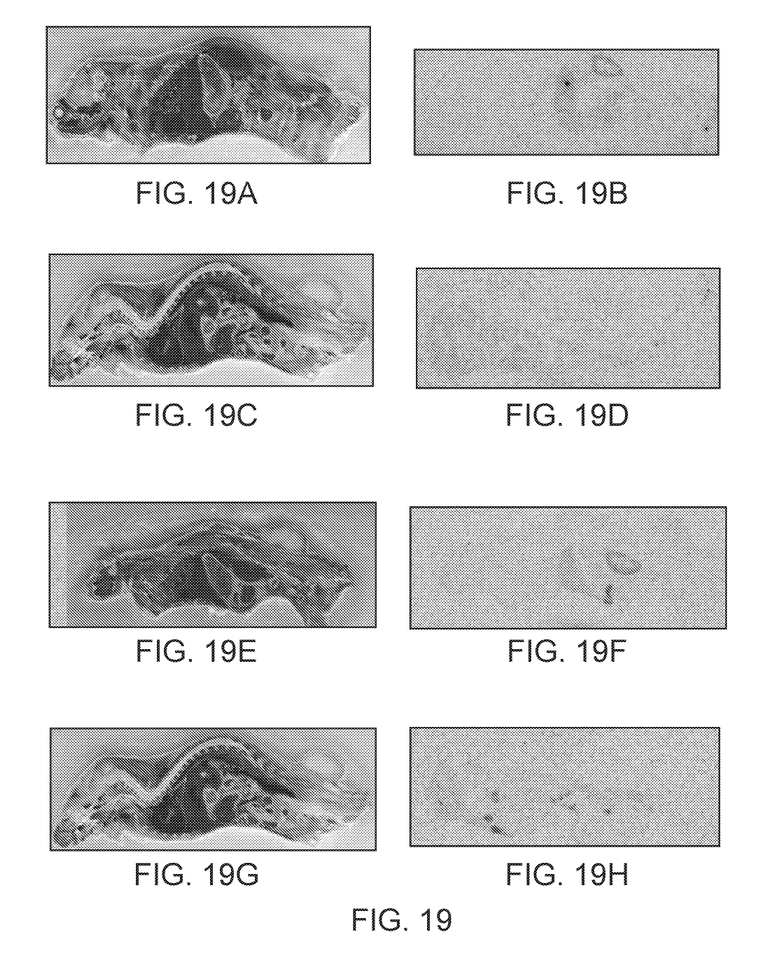

FIG. 19A illustrates a white light image of a frozen section of a mouse twenty-four hours after administration of 14 nmol of the SEQ ID NO: 5-RY peptide conjugate. FIG. 19B illustrates an autoradiographic image corresponding to FIG. 19A in which the ¹⁴C signal identifies the peptide distribution in the tissues, including in the RH-28 tumor, of a mouse twenty-four hours after administration of 14 nmol of the SEQ ID NO: 5-RY peptide conjugate. FIG. 19C illustrates a white light image of a different frozen section of the same mouse as in FIG. 19A and FIG. 19B twenty-four hours after administration of 14 nmol of the SEQ ID NO: 5-RY peptide conjugate. FIG. 19D illustrates an autoradiographic image corresponding to FIG. 19C in which the ¹⁴C signal identifies the peptide distribution in the tissues, including in the RH-28 tumor, twenty-four hours after administration of 14 nmol of the SEQ ID NO: 5-RY peptide conjugate. FIG. 19E illustrates a white light image of a frozen section of a different mouse than shown in FIG. 19A through FIG. 19D twenty-four hours after administration of 14 nmol of the SEQ ID NO: 5-RY peptide conjugate. FIG. 19F illustrates an autoradiographic image corresponding to FIG. 19E in which the ¹⁴C signal identifies the peptide distribution in the tissues, including in the RH-28 tumor, of a mouse twenty-four hours after administration of 14 nmol of the SEQ ID NO: 5-RY peptide conjugate. FIG. 19G illustrates a white light image of a different frozen section of the same mouse as in FIG. 19E and FIG. 19F twenty-four hours after administration of 14 nmol of the SEQ ID NO: 5-RY peptide conjugate. FIG. 19H illustrates an autoradiographic image corresponding to FIG. 19G in which the ¹⁴C signal identifies the peptide distribution in the tissues, including in the RH-28 tumor, twenty-four hours after administration of 14 nmol of the SEQ ID NO: 5-RY peptide conjugate.

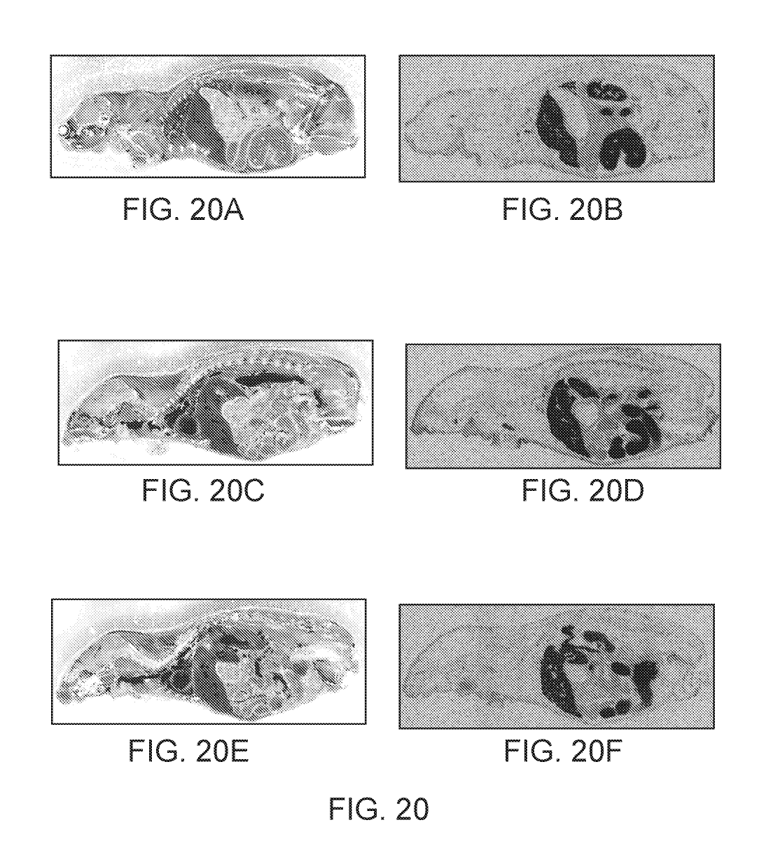

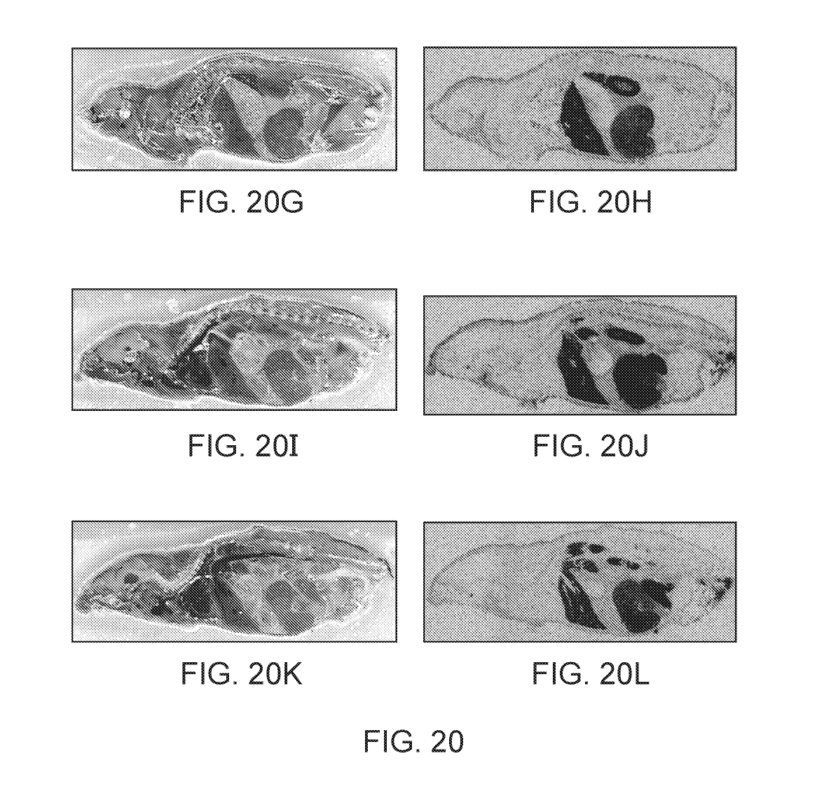

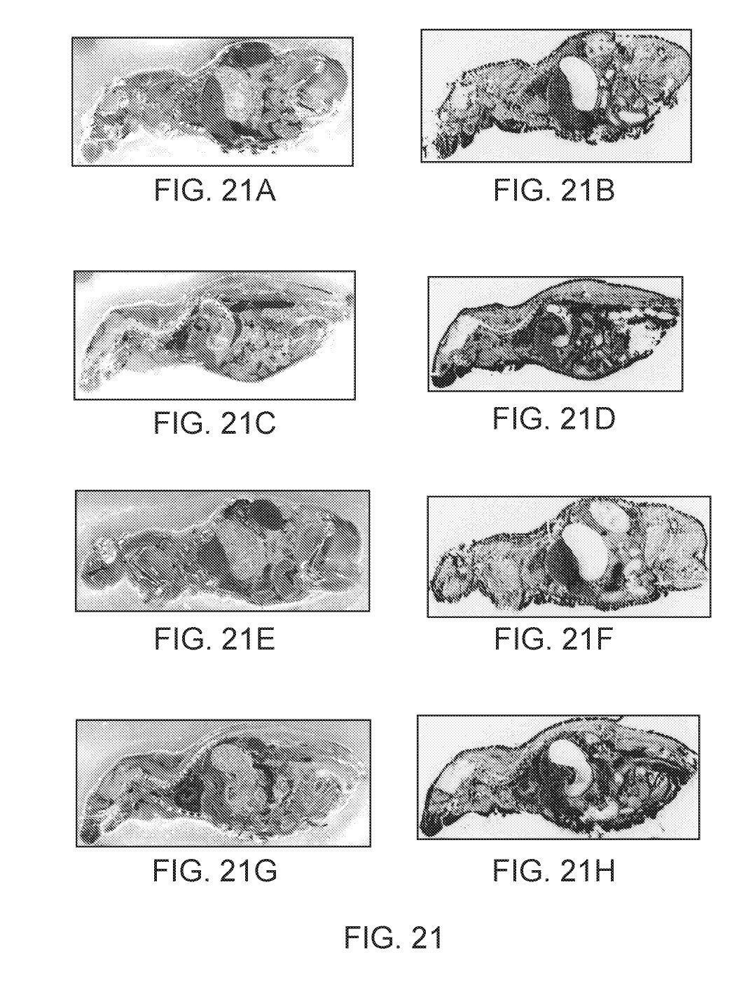

FIG. 20A illustrates a white light image of a frozen section of a mouse with intact kidneys three hours after administration of 100 nmol of the radiolabeled SEQ ID NO: 37 peptide. FIG. 20B illustrates an autoradiographic image corresponding to FIG. 20A in which the ¹⁴C signal identifies the peptide distribution in the tissues of a mouse with intact kidneys three hours after administration of 100 nmol of the radiolabeled SEQ ID NO: 37 peptide. FIG. 20C illustrates a white light image of a different frozen section of the same mouse as in FIG. 20A and FIG. 20B three hours after administration of 100 nmol of the radiolabeled SEQ ID NO: 37 peptide. FIG. 20D illustrates an autoradiographic image corresponding to FIG. 20C in which the ¹⁴C signal identifies the peptide distribution in the tissues of the mouse three hours after administration of 100 nmol of the radiolabeled SEQ ID NO: 37 peptide. FIG. 20E illustrates a white light image of a different frozen section of the same mouse as in FIG. 20A through FIG. 20D three hours after administration of 100 nmol of the radiolabeled SEQ ID NO: 37 peptide. FIG. 20F illustrates an autoradiographic image corresponding to FIG. 20E in which the ¹⁴C signal identifies the peptide distribution in the tissues of the mouse three hours after administration of 100 nmol of the radiolabeled SEQ ID NO: 37 peptide. FIG. 20G illustrates a white light image of a frozen section of a different mouse with intact kidneys than shown in FIG. 20A through FIG. 20F three hours after administration of 100 nmol of the radiolabeled SEQ ID NO: 37 peptide. FIG. 20H illustrates an autoradiographic image corresponding to FIG. 20G in which the ¹⁴C signal identifies the peptide distribution tissues of a mouse three hours after administration of 100 nmol of the radiolabeled SEQ ID NO: 37 peptide. FIG. 20I illustrates a white light image of a different frozen section of the same mouse as in FIG. 20G and FIG. 20H three hours after administration of 100 nmol of the radiolabeled SEQ ID NO: 37 peptide. FIG. 20J illustrates an autoradiographic image corresponding to FIG. 20I in which the ¹⁴C signal identifies the peptide distribution in the tissues three hours after administration of 100 nmol of the radiolabeled SEQ ID NO: 37 peptide. FIG. 20K illustrates a white light image of a different frozen section of the same mouse as in FIG. 20G through FIG. 20J three hours after administration of 100 nmol of the radiolabeled SEQ ID NO: 37 peptide. FIG. 20L illustrates an autoradiographic image corresponding to FIG. 20K in which the ¹⁴C signal identifies the peptide distribution in the tissues three hours after administration of 100 nmol of the radiolabeled SEQ ID NO: 37 peptide.

FIG. 21A illustrates a white light image of a frozen section of a mouse with ligated kidneys three hours after administration of 100 nmol of the radiolabeled SEQ ID NO: 37 peptide. FIG. 21B illustrates an autoradiographic image corresponding to FIG. 21A in which the ¹⁴C signal identifies the peptide distribution in the tissues of a mouse with intact kidneys three hours after administration of 100 nmol of the radiolabeled SEQ ID NO: 37 peptide. FIG. 21C illustrates a white light image of a different frozen section of the same mouse as in FIG. 21A and FIG. 21B three hours after administration of 100 nmol of the radiolabeled SEQ ID NO: 37 peptide. FIG. 21D illustrates an autoradiographic image corresponding to FIG. 21C in which the ¹⁴C signal identifies the peptide distribution in the tissues of the mouse three hours after administration of 100 nmol of the radiolabeled SEQ ID NO: 37 peptide. FIG. 21E illustrates a white light image of a frozen section of a different mouse with ligated kidneys than shown in FIG. 21A through FIG. 21D three hours after administration of 100 nmol of the radiolabeled SEQ ID NO: 37 peptide. FIG. 21F illustrates an autoradiographic image corresponding to FIG. 21E in which the ¹⁴C signal identifies the peptide distribution in the tissues of a mouse three hours after administration of 100 nmol of the radiolabeled SEQ ID NO: 37 peptide. FIG. 21G illustrates a white light image of a different frozen section of the same mouse as in FIG. 21E and FIG. 21F three hours after administration of 100 nmol of the radiolabeled SEQ ID NO: 37 peptide. FIG. 21H illustrates an autoradiographic image corresponding to FIG. 21G in which the ¹⁴C signal identifies the peptide distribution in the tissues three hours after administration of 100 nmol of the radiolabeled SEQ ID NO: 37 peptide.

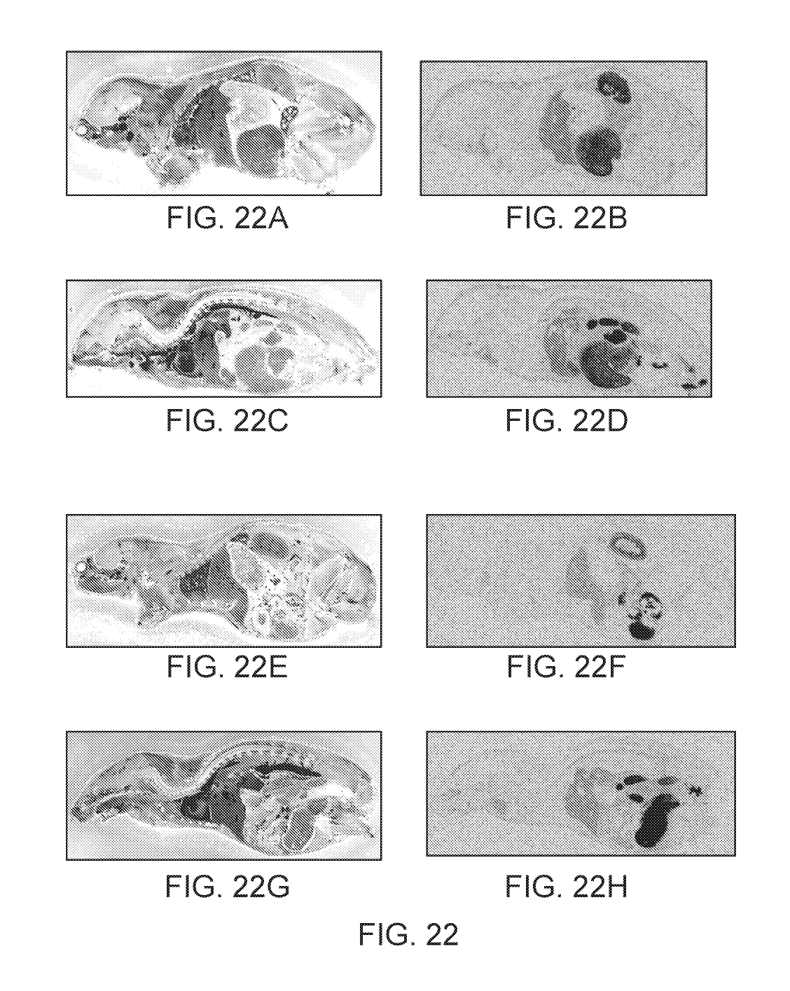

FIG. 22A illustrates a white light image of a frozen section of a mouse with intact kidneys three hours after administration of 100 nmol of the radiolabeled SEQ ID NO: 35 peptide. FIG. 22B illustrates an autoradiographic image corresponding to FIG. 22A in which the ¹⁴C signal identifies the peptide distribution in the tissues of a mouse with intact kidneys three hours after administration of 100 nmol of the radiolabeled SEQ ID NO: 35 peptide. FIG. 22C illustrates a white light image of a different frozen section of the same mouse as in FIG. 22A and FIG. 22B three hours after administration of 100 nmol of the radiolabeled SEQ ID NO: 35 peptide. FIG. 22D illustrates an autoradiographic image corresponding to FIG. 22C in which the ¹⁴C signal identifies the peptide distribution in the tissues of the mouse three hours after administration of 100 nmol of the radiolabeled SEQ ID NO: 35 peptide. FIG. 22E illustrates a white light image of a frozen section of a different mouse with intact kidneys than shown in FIG. 22A through FIG. 22D three hours after administration of 100 nmol of the radiolabeled SEQ ID NO: 35 peptide. FIG. 22F illustrates an autoradiographic image corresponding to FIG. 22E in which the ¹⁴C signal identifies the peptide distribution in the tissues of a mouse three hours after administration of 100 nmol of the radiolabeled SEQ ID NO: 35 peptide. FIG. 22G illustrates a white light image of a different frozen section of the same mouse as in FIG. 22E and FIG. 22F three hours after administration of 100 nmol of the radiolabeled SEQ ID NO: 35 peptide. FIG. 22H illustrates an autoradiographic image corresponding to FIG. 22G in which the ¹⁴C signal identifies the peptide distribution in the tissues three hours after administration of 100 nmol of the radiolabeled SEQ ID NO: 35 peptide.

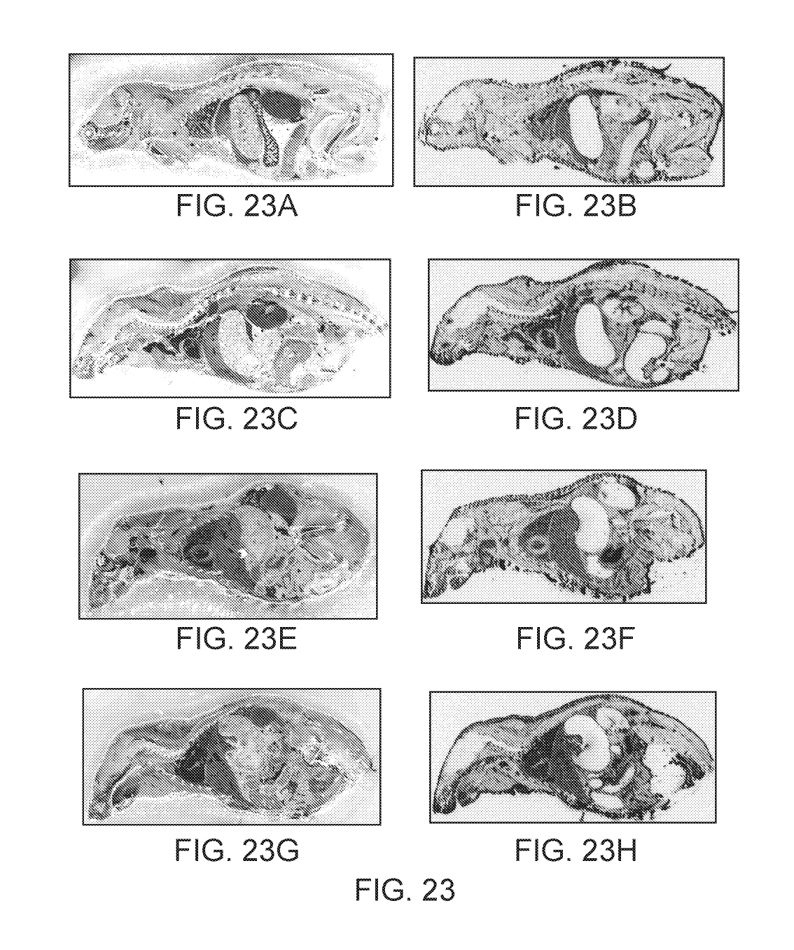

FIG. 23A illustrates a white light image of a frozen section of a mouse with ligated kidneys three hours after administration of 100 nmol of the radiolabeled SEQ ID NO: 35 peptide. FIG. 23B illustrates an autoradiographic image corresponding to FIG. 23A in which the ¹⁴C signal identifies the peptide distribution in the tissues of a mouse with ligated kidneys three hours after administration of 100 nmol of the radiolabeled SEQ ID NO: 35 peptide. FIG. 23C illustrates a white light image of a different frozen section of the same mouse as in FIG. 23A and FIG. 23B three hours after administration of 100 nmol of the radiolabeled SEQ ID NO: 35 peptide. FIG. 23D illustrates an autoradiographic image corresponding to FIG. 23C in which the ¹⁴C signal identifies the peptide distribution in the tissues of the mouse three hours after administration of 100 nmol of the radiolabeled SEQ ID NO: 35 peptide. FIG. 23E illustrates a white light image of a frozen section of a different mouse with ligated kidneys than shown in FIG. 23A through FIG. 23D three hours after administration of 100 nmol of the radiolabeled SEQ ID NO: 35 peptide. FIG. 23F illustrates an autoradiographic image corresponding to FIG. 23E in which the ¹⁴C signal identifies the peptide distribution in the tissues of a mouse three hours after administration of 100 nmol of the radiolabeled SEQ ID NO: 35 peptide. FIG. 23G illustrates a white light image of a different frozen section of the same mouse as in FIG. 23E and FIG. 23F three hours after administration of 100 nmol of the radiolabeled SEQ ID NO: 35 peptide. FIG. 23H illustrates an autoradiographic image corresponding to FIG. 23G in which the ¹⁴C signal identifies the peptide distribution in the tissues three hours after administration of 100 nmol of the radiolabeled SEQ ID NO: 35 peptide. These figures illustrate that the peptide conjugated to a drug can target the drug to tumor tissue.

### Example 17

### Peptide Half-life after Administration

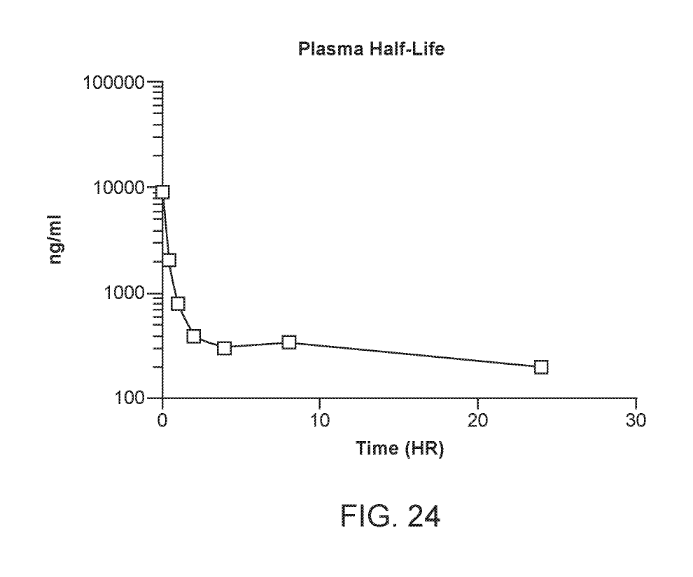

This example describes an analysis of the half-life of the SEQ ID NO: 5 peptide after administration. A target dosage of 12.8 nmol of the radiolabeled SEQ ID NO: 5 peptide carrying 2 uCi of ¹⁴C was intravenously administered per mouse. A cardiac puncture to obtain blood for half-life analysis was taken at 5 minutes, 30 minutes, 1 hour, 2 hours, 4 hours, 8 hours, and 24 hours after administration of the peptide. Blood from each time point was processed to plasma and then analyzed for ¹⁴C by liquid scintillation counting, which was used to determine the half-life of radiolabeled SEQ ID NO: 5 peptide. FIG. 24 shows a graph of the half-life of the radiolabeled SEQ ID NO: 5 peptide.

### Example 18

### Distribution of Peptide to Ewing's Sarcoma

This example shows the distribution of peptides after administration to animals with A763 Ewing's Sarcoma. SEQ ID NO: 4, Imperatoxin GSDCLPHLRRCRADNDCCGRRCRRRGTNAERRCR (SEQ ID NO: 421), Conotoxin CVIC GSCRGRGQSCSRLMYDCCTGSCSRRGRC (SEQ ID NO: 422), or SEQ ID NO: 54 are expressed recombinantly or chemically synthesized and then the N-terminus of the peptide is conjugated to Alexflour647(AF647) to create the SEQ ID NO: 4-A conjugated peptide (SEQ ID NO: 4-A peptide conjugate), Imperatoxin-A conjugated peptide (Imperatoxin-A conjugate), Conotoxin CVIC-A conjugated peptide (Conotoxin-A conjugate), or SEQ ID NO: 54-A conjugated peptide (SEQ ID NO: 54-A peptide conjugate), respectively.

A target dosage of 10 nmol of each peptide-conjugate was administered to separate flank A673 Ewing's Sarcoma tumor bearing Female Harlan athymic nude mice while anesthetized. Each peptide-conjugate was allowed to freely circulate within the animal for 4 hours before the animals were euthanized. The tumors, kidneys, liver, heart, lymph nodes, brain, spleen, skeletal muscle, lymph nodes, and lung were excised from each animal, and imaged using IVIS Spectrum.

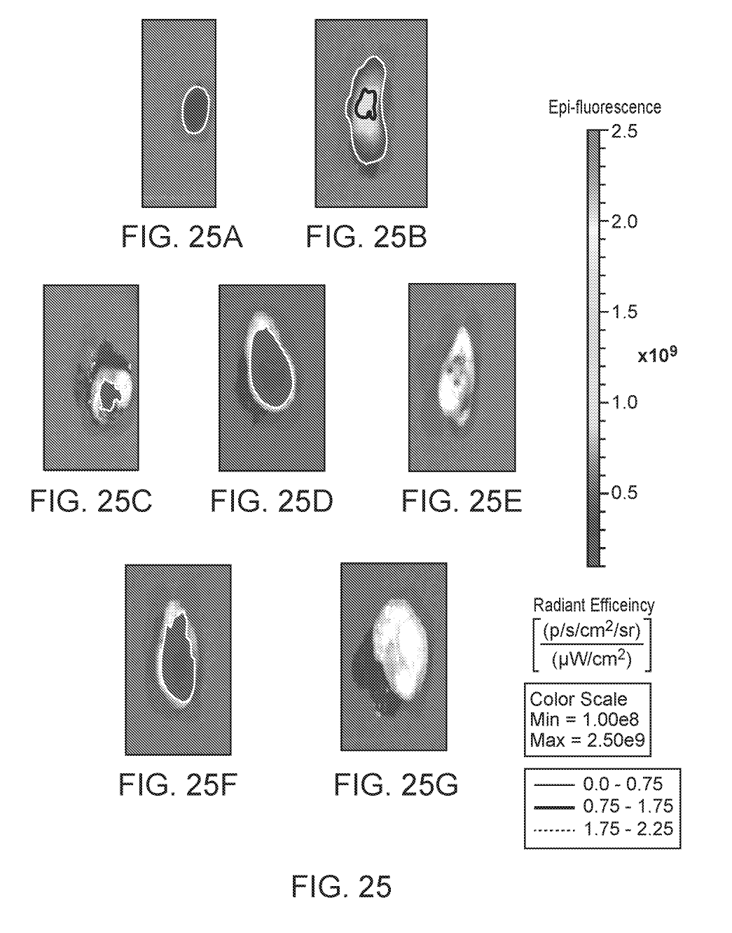

FIG. 25A shows a near-infrared fluorescence image of Ewing's Sarcoma tumor excised from a mouse after administration of 10 nmol of SEQ ID NO: 4-A peptide conjugate. FIG. 25B shows a near-infrared fluorescence image of Ewing's Sarcoma tumor excised from a different mouse than in FIG. 25A after administration of 10 nmol of SEQ ID NO: 4-A peptide conjugate. FIG. 25C shows a near-infrared fluorescence image of Ewing's Sarcoma tumor excised from a mouse after administration of 10 nmol of Imperatoxin-A conjugate. FIG. 25D shows a near-infrared fluorescence image of Ewing's Sarcoma tumor excised from a different mouse than in FIG. 25C after administration 10 nmol of Imperatoxin-A conjugate. FIG. 25E shows a near-infrared fluorescence image of Ewing's Sarcoma tumor excised from a mouse after administration of 10 nmol of Imperatoxin-A conjugate. FIG. 25F shows a near-infrared fluorescence image of Ewing's Sarcoma tumor excised from a mouse after administration of 10 nmol of SEQ ID NO: 54-A peptide conjugate. FIG. 25G shows a near-infrared fluorescence image of Ewing's Sarcoma tumor excised from a mouse that did not receive any peptide as a negative control. Tissue fluorescence indicates the presence of the peptide-conjugate.

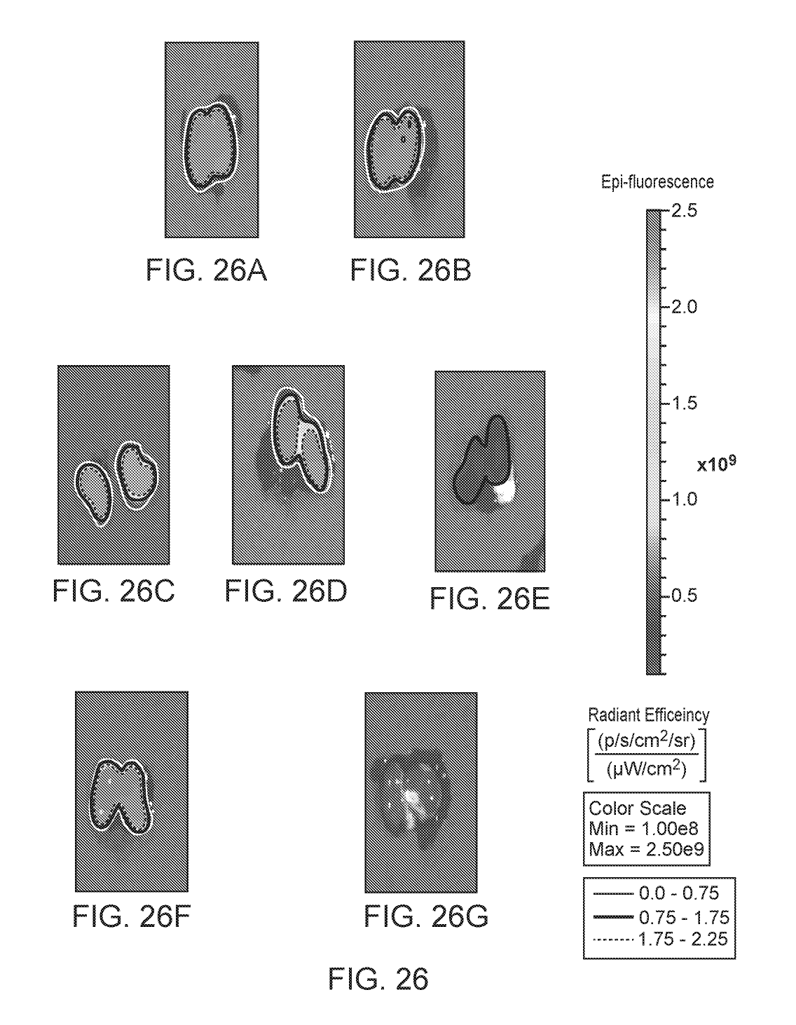

FIG. 26A shows a near-infrared fluorescence image of the kidneys excised from a mouse after administration of 10 nmol of SEQ ID NO: 4-A peptide conjugate. FIG. 26B shows a near-infrared fluorescence image of the kidneys excised from a different mouse than in FIG. 26A after administration of 10 nmol of SEQ ID NO: 4-A peptide conjugate. FIG. 26C shows a near-infrared fluorescence image of the kidneys excised from a mouse after administration of 10 nmol of Imperatoxin-A conjugate. FIG. 26D shows a near-infrared fluorescence image of the kidneys excised from a different mouse than in FIG. 26C after administration of 10 nmol of Imperatoxin-A conjugate. FIG. 26E shows a near-infrared fluorescence image of the kidneys excised from a mouse after administration 10 nmol of Conotoxin-A conjugate. FIG. 26F shows a near-infrared fluorescence image of the kidneys excised from a mouse after administration of 10 nmol of SEQ ID NO: 54-A peptide conjugate. FIG. 26G shows a near-infrared fluorescence image of the kidneys excised from a mouse that did not receive any peptide as a negative control. Tissue fluorescence indicates the presence of the peptide-conjugate.

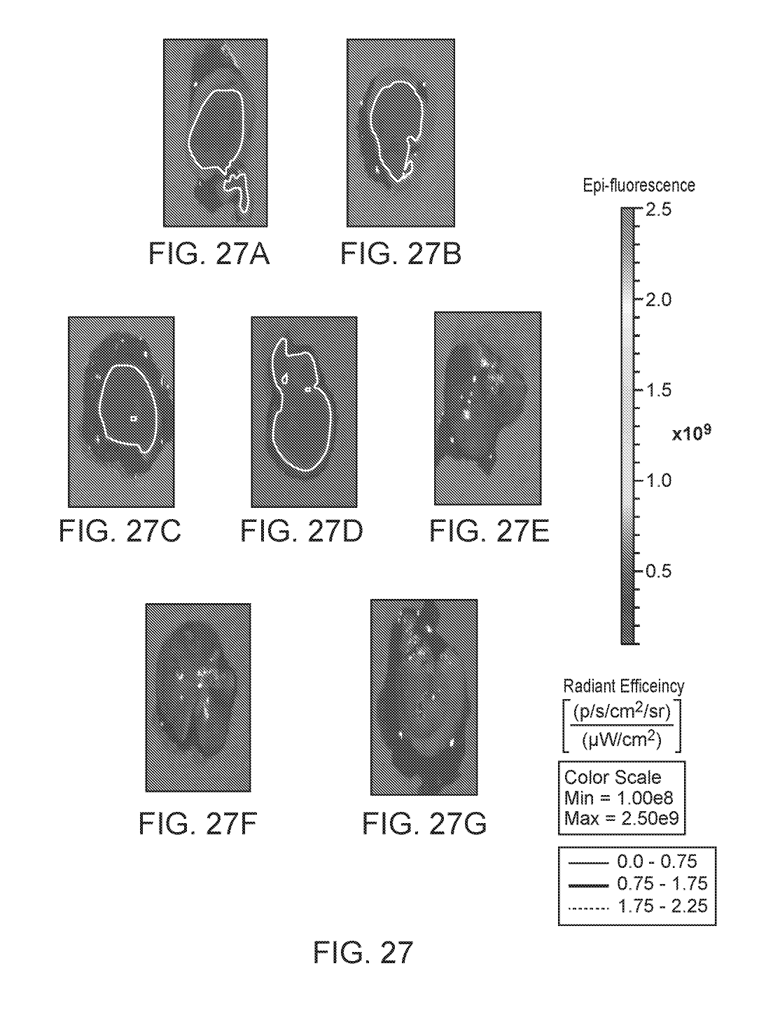

FIG. 27A shows a near-infrared fluorescence image of the liver excised from a mouse after administration of 10 nmol of SEQ ID NO: 4-A peptide conjugate. FIG. 27B shows a near-infrared fluorescence image of the liver excised from a different mouse than in FIG. 27A after administration of 10 nmol of SEQ ID NO: 4-A peptide conjugate. FIG. 27C shows a near-infrared fluorescence image of the liver excised from a mouse after administration of 10 nmol of Imperatoxin-A conjugate. FIG. 27D shows a near-infrared fluorescence image of the liver excised from a different mouse than in FIG. 27C after administration of 10 nmol of Imperatoxin-A conjugate. FIG. 27E shows a near-infrared fluorescence image of the liver excised from a mouse after administration of 10 nmol of Conotoxin-A conjugate. FIG. 27F shows a near-infrared fluorescence image of the liver excised from a mouse after administration of 10 nmol of SEQ ID NO: 54-A peptide conjugate. FIG. 27G shows a near-infrared fluorescence image of the liver excised from a mouse that did not receive any peptide as a negative control. Tissue fluorescence indicates the presence of the peptide-conjugate.

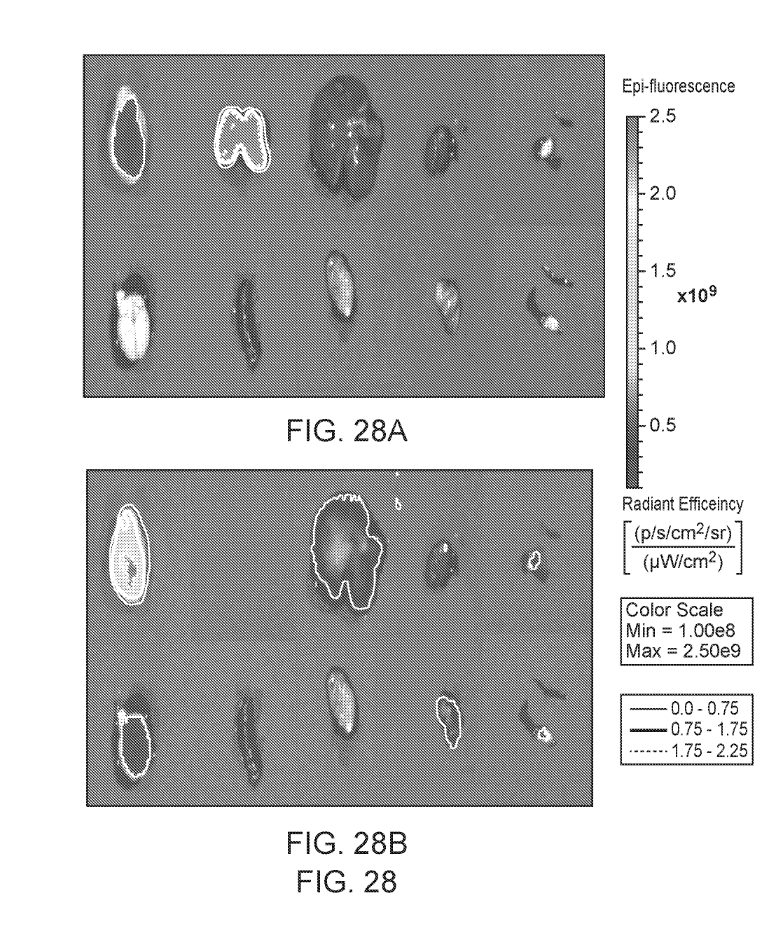

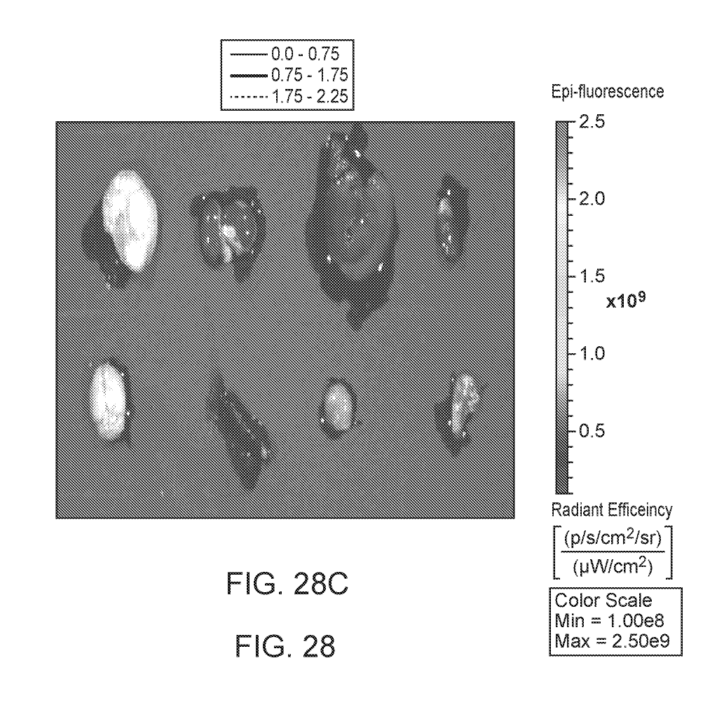

FIG. 28A shows a near-infrared fluorescence image of different tissues that were excised after the administration of 10 nmol of SEQ ID NO: 54-A peptide conjugate. The tissues on the top row from left to right are tumor, kidneys, liver, heart, and the draining lymph node. The tissues on the bottom row from left to right are brain, spleen, skeletal muscle, lung, and the lateral lymph node. Tissue fluorescence indicates the presence of the peptide-conjugate. FIG. 28B shows the near-infrared fluorescence image of FIG. 28A of different tissues that were excised after the administration of 10 nmol of SEQ ID NO: 54-A peptide conjugate, but the image was taken without the kidneys. The tissues on the top row from left to right are tumor, liver, heart, and the draining lymph node. The tissues on the bottom row from left to right are brain, spleen, skeletal muscle, lung, and the lateral lymph node. Tissue fluorescence indicates the presence of the peptide-conjugate. FIG. 28C shows a near-infrared fluorescence image of different tissues that were excised from a mouse that did not receive any peptide as a negative control. The tissues on the top row from left to right are tumor, kidneys, liver, and heart. The tissues on the bottom row from left to right are brain, spleen, skeletal muscle, and lung. Tissue fluorescence indicates the presence of the peptide-conjugate.

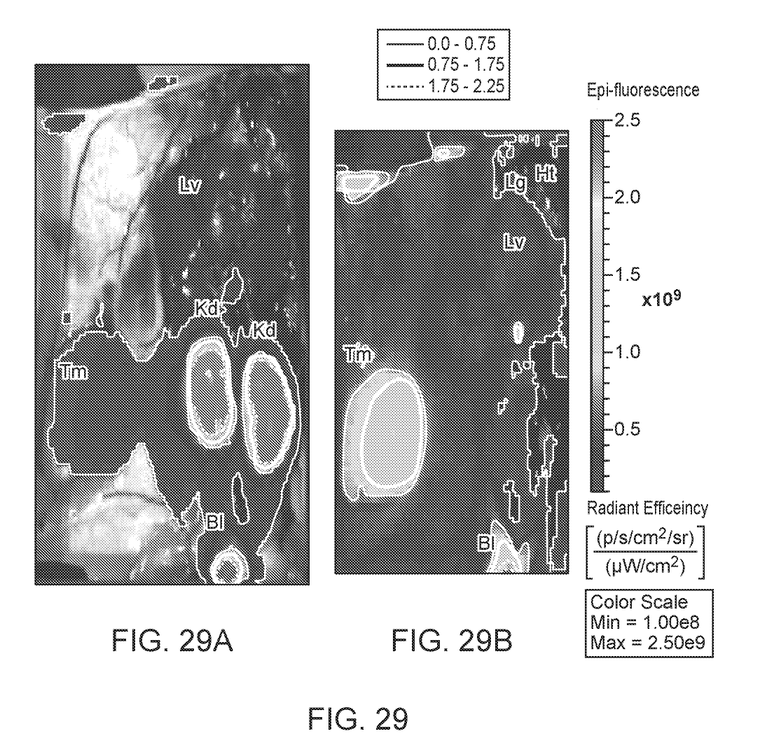

FIG. 29A shows an ex vivo near infrared image of the internal body cavity of a mouse that was euthanized 4 hours after administration of 10 nmol of SEQ ID NO: 54-A peptide conjugate. Lv indicates the location of the liver. Tm indicates the location of the tumor. Kd indicates the location of the kidneys. B1 indicates the location of the bladder. Tissue fluorescence indicates the presence of SEQ ID NO: 54-A peptide conjugate. FIG. 29B shows an ex vivo near infrared image of the internal body cavity of a mouse that was euthanized 4 hours after administration of 10 nmol of SEQ ID NO: 54-A peptide conjugate as shown in FIG. 29A, but with the kidneys removed. Lv indicates the location of the liver. Tm indicates the location of the tumor. B1 indicates the location of the bladder. Ht indicates the location of the heart. Lg indicates the location of the lung. Tissue fluorescence indicates the presence of SEQ ID NO: 54-A peptide conjugate.

### Example 19

### Treatment of Ewing's Sarcoma with a Peptide of the Disclosure

This example describes the use of the peptides described herein to treat Ewing's Sarcoma.

A peptide of the disclosure is expressed recombinantly or chemically synthesized and then is conjugated to a chemotherapeutic drug, such as cyclophosphamide, doxorubicin, an auristatin (e.g., monomethyl auristatin E (MMAE), monomethyl auristatin F (MMAF), dolostatin, auristatin F, MMAD), a maytansinoid (e.g., DM1, DM4, maytansine), a pyrrolobenzodiazapine dimer, a calicheamicin (e.g., N-acetyl-γ-calicheamicin), a vinca alkyloid (e.g., 4-deacetylvinblastine), duocarmycin, cyclic peptide analogs of the mushroom amatoxins, epothilones, anthracyclines, CC-1065, taxanes (e.g., paclitaxel, docetaxel, cabazitaxel), SN-38, irinotecan, vincristine, vinblastine, platinum compounds (e.g., cisplatin), methotrexate, a microtubule inhibitor, ifosfamide, etoposide, fenretinide, a DNA damaging agent, or teniposide, directly or via a cleavable or noncleavable linker. Coupling of the chemotherapeutic drug to the peptide of any one of SEQ ID NO: 1-SEQ ID NO: 192 or SEQ ID NO: 210-SEQ ID NO: 401 targets the drug to Ewing's Sarcoma. One or more chemotherapeutic drug-peptide conjugates are administered to a human or animal.

### Example 20

### Treatment of Glioblastoma with a Peptide-Conjugate of the Disclosure

This example describes the use of the peptides described herein to treat glioblastoma. A peptide of the disclosure is expressed recombinantly or chemically synthesized and then is conjugated to a chemotherapeutic drug, such as cyclophosphamide, doxorubicin, an auristatin (e.g., monomethyl auristatin E (MMAE), monomethyl auristatin F (MMAF), dolostatin, auristatin F, MMAD), a maytansinoid (e.g., DM1, DM4, maytansine), a pyrrolobenzodiazapine dimer, a calicheamicin (e.g., N-acetyl-γ-calicheamicin), a vinca alkyloid (e.g., 4-deacetylvinblastine), duocarmycin, cyclic peptide analogs of the mushroom amatoxins, epothilones, anthracyclines, CC-1065, taxanes (e.g., paclitaxel, docetaxel, cabazitaxel), SN-38, irinotecan, vincristine, vinblastine, platinum compounds (e.g., cisplatin), methotrexate, a microtubule inhibitor, temozolomide, carmustine, topotecan, radioisotopes, palbociclib, a DNA damaging agent, or teniposide, directly or via a cleavable or noncleavable linker. Coupling of the chemotherapeutic drug to the peptide of any one of SEQ ID NO: 1-SEQ ID NO: 196 or SEQ ID NO: 210-SEQ ID NO: 405 targets the drug to the glioblastoma. One or more chemotherapeutic drug-peptide conjugates are administered to a human or animal.

### Example 21

### Treatment of Triple-Negative Breast Cancer with a Peptide-Conjugate of the Disclosure

This example describes the use of the peptides described herein to treat triple-negative breast cancer. A peptide of the disclosure is expressed recombinantly or chemically synthesized and then is conjugated to a chemotherapeutic drug, such as cyclophosphamide, doxorubicin, an auristatin (e.g., monomethyl auristatin E (MMAE), monomethyl auristatin F (MMAF), dolostatin, auristatin F, MMAD), a maytansinoid (e.g., DM1, DM4, maytansine), a pyrrolobenzodiazapine dimer, a calicheamicin (e.g., N-acetyl-γ-calicheamicin), a vinca alkyloid (e.g., 4-deacetylvinblastine), duocarmycin, cyclic peptide analogs of the mushroom amatoxins, epothilones, anthracyclines, CC-1065, taxanes (e.g., paclitaxel, docetaxel, cabazitaxel), SN-38, irinotecan, vincristine, vinblastine, platinum compounds (e.g., cisplatin), methotrexate, a microtubule inhibitor, iniparib, carboplatin, a DNA damaging agent, or teniposide, directly or via a cleavable or noncleavable linker. Coupling of the chemotherapeutic drug to the peptide of any one of SEQ ID NO: 1-SEQ ID NO: 192 or SEQ ID NO: 210-SEQ ID NO: 401 targets the drug to the triple-negative breast cancer. One or more chemotherapeutic drug-peptide conjugates are administered to a human or animal.

### Example 22

### Peptide Administration with Detectable Agents

This example describes peptide administration with detectable agents. A peptide of the disclosure is expressed recombinantly or chemically synthesized and then is conjugated to a detectable agent, such as a fluorophore, a near-infrared fluorescence dye, a contrast agent, a nanoparticle, a metal-containing nanoparticle, a metal chelate, an X-ray contrast agent, a PET agent, a radioisotope, or a radionuclide chelator. One or more detectable agent-peptide conjugates are administered to a human or animal.

### Example 23

### Treatment of Metastatic Colon Cancer with a Peptide-Conjugate of the Disclosure

This example describes the use of the peptides described herein to treat metastatic colon cancer. A peptide of the disclosure is expressed recombinantly or chemically synthesized and then is conjugated to a chemotherapeutic drug, such as cyclophosphamide, doxorubicin, an auristatin (e.g., monomethyl auristatin E (MMAE), monomethyl auristatin F (MMAF), dolostatin, auristatin F, MMAD), a maytansinoid (e.g., DM1, DM4, maytansine), a pyrrolobenzodiazapine dimer, a calicheamicin (e.g., N-acetyl-γ-calicheamicin), a vinca alkyloid (e.g., 4-deacetylvinblastine), duocarmycin, cyclic peptide analogs of the mushroom amatoxins, epothilones, anthracyclines, CC-1065, taxanes (e.g., paclitaxel, docetaxel, cabazitaxel), SN-38, irinotecan, vincristine, vinblastine, platinum compounds (e.g., cisplatin), methotrexate, a microtubule inhibitor, capecitabine, fluorouracil, irinotecan, oxaliplatin, a DNA damaging agent, or teniposide, directly or via a cleavable or noncleavable linker. Coupling of the chemotherapeutic drug to the peptide of any one of SEQ ID NO: 1-SEQ ID NO: 192 or SEQ ID NO: 210-SEQ ID NO: 401 targets the drug to the metastatic colon cancer. One or more chemotherapeutic drug-peptide conjugates are administered to a human or animal.

### Example 24

### Treatment of Non-Brain Cancer with a Mutated Peptide

This example describes a mutated peptide that is used for a non-brain cancer. A peptide of the disclosure is mutated. This mutation prevents the mutated peptide from crossing the blood brain barrier. The mutated peptide expressed recombinantly or chemically synthesized and then is conjugated to a chemotherapeutic drug, such as cyclophosphamide, doxorubicin, an auristatin (e.g., monomethyl auristatin E (MMAE), monomethyl auristatin F (MMAF), dolostatin, auristatin F, MMAD), a maytansinoid (e.g., DM1, DM4, maytansine), a pyrrolobenzodiazapine dimer, a calicheamicin (e.g., N-acetyl-γ-calicheamicin), a vinca alkyloid (e.g., 4-deacetylvinblastine), duocarmycin, cyclic peptide analogs of the mushroom amatoxins, epothilones, anthracyclines, CC-1065, taxanes (e.g., paclitaxel, docetaxel, cabazitaxel), SN-38, irinotecan, vincristine, vinblastine, platinum compounds (e.g., cisplatin), methotrexate, a microtubule inhibitor, capecitabine, fluorouracil, irinotecan, oxaliplatin, a DNA damaging agent, or teniposide, directly or via a cleavable or noncleavable linker. Coupling of the chemotherapeutic drug to the mutated peptide of any one of SEQ ID NO: 1-SEQ ID NO: 192 or SEQ ID NO: 210-SEQ ID NO: 401 targets the drug to a non-brain cancer. For example, the mutated peptide drug conjugate is targeted to triple negative breast cancer. One or more chemotherapeutic drug-peptide conjugates are administered to a human or animal.

### Example 25

### Methods of Altering the Half-Life of a Peptide Drug Conjugate

This example describes four methods for increasing the half-life of a peptide drug conjugate to improve the delivery of the peptide drug conjugate to a tumor and to increase peptide drug conjugate exposure time at the tumor.

In the first method, a peptide of the disclosure is expressed recombinantly or chemically synthesized and then is conjugated to a chemotherapeutic drug, such as cyclophosphamide, doxorubicin, MMAE, DM1, calicheamicin, taxol, or teniposide, via a cleavable or noncleavable linker. Additionally, the peptide drug conjugate includes a moiety such as a fatty acid (e.g., a palmitic acid), a hydrocarbon chain or a polymer (e.g., polyethylene glycol), which is conjugated at the linker region. Coupling of the chemotherapeutic drug to the peptide of any one of SEQ ID NO: 1-SEQ ID NO: 196 or SEQ ID NO: 210-SEQ ID NO: 405 targets the drug to the tumor. One or more chemotherapeutic drug-peptide conjugates are administered to a human or animal. The half-life of the peptide drug conjugate is increased by the addition of the moiety, improves the delivery of the peptide drug conjugate to the tumor and increases the peptide drug exposure time at the tumor after administration to a patient to treat the tumor. For example, the half-life of peptides is increased from minutes to hours when conjugated to Alexa MMAE or taxol.

In the second method, the half-life of a peptide drug conjugate is altered by mutating the peptide. The peptide of any one of SEQ ID NO: 1-SEQ ID NO: 196 or SEQ ID NO: 210-SEQ ID NO: 405 is mutated to increase albumin binding. The mutated peptide is expressed recombinantly or chemically synthesized and then is conjugated to a chemotherapeutic drug, such as cyclophosphamide, doxorubicin, MMAE, DM1, calicheamicin, taxol, or teniposide, directly or via a cleavable or noncleavable linker. Coupling of the chemotherapeutic drug to the peptide of any one of SEQ ID NO: 1-SEQ ID NO: 196 or SEQ ID NO: 210-SEQ ID NO: 405 targets the drug to the cancer. One or more chemotherapeutic drug-peptide conjugates are administered to a human or animal. The half-life of the peptide drug conjugate is increased by the mutation in the peptide, which improves the delivery of the peptide drug conjugate to the tumor and increases the peptide drug exposure time after administration to a patient to treat the tumor.

In the third method, the half-life of a peptide drug conjugate is altered by additionally conjugating a lipophilic moiety to the peptide drug conjugate. The peptide is expressed recombinantly or chemically synthesized and then is conjugated to a chemotherapeutic drug, such as cyclophosphamide, doxorubicin, MMAE, DM1, calicheamicin, taxol, or teniposide, directly or via a cleavable or noncleavable linker. The peptide drug conjugate is then conjugated to a lipophilic moiety, such as Alexa 679 or Cy5.5, directly or via a cleavable or noncleavable linker. Coupling of the chemotherapeutic drug to the peptide of any one of SEQ ID NO: 1-SEQ ID NO: 196 or SEQ ID NO: 210-SEQ ID NO: 405 targets the drug to the cancer. One or more chemotherapeutic drug-peptide conjugates are administered to a human or animal. The half-life of the peptide drug conjugate is increased by the conjugation to a lipophilic moiety, which improves the delivery of the peptide drug conjugate to the tumor and increases the peptide drug exposure time after administration to a patient to treat the tumor. For example, the half-life of the peptide drug conjugate is increased when conjugated to Alexa 679 or Cy5.5.

In the fourth method, the half-life of a peptide drug conjugate is altered by additionally conjugating an Fc portion of an antibody to the peptide drug conjugate. The peptide is expressed recombinantly or chemically synthesized and then is conjugated to a chemotherapeutic drug, such as cyclophosphamide, doxorubicin, or teniposide, directly or via a cleavable or noncleavable linker. The peptide drug conjugate is then conjugated to an Fc portion of an antibody directly or via a cleavable or noncleavable linker. Coupling of the chemotherapeutic drug to the peptide of any one of SEQ ID NO: 1-SEQ ID NO: 196 or SEQ ID NO: 210-SEQ ID NO: 405 targets the drug to the cancer. One or more chemotherapeutic drug-peptide conjugates are administered to a human or animal. The half-life of the peptide drug conjugate is increased by the conjugation to an Fc portion of an antibody, which improves the delivery of the peptide drug conjugate to the tumor and increases the peptide drug exposure time after administration to a patient to treat the tumor. For example, the half-life of the peptide drug conjugate is increased to days when conjugated to an Fc portion of an antibody.

### Example 26

### Conjugation of Drugs to Multiple Sites on a Peptide

This example describes the conjugation of a drug at multiple sites on a peptide containing at least three lysine residues. A peptide of the disclosure with at least three lysine residues is expressed recombinantly or chemically synthesized and then 4 chemotherapeutic drug molecules are separately conjugated to the peptide at 4 distinct sites. These sites are distinct lysines and the N-terminus of peptide.

### Example 27

### Conjugation of Multiple Drug Molecules to a Peptide

This example describes the conjugation of multiple drug molecules to a peptide. Four chemotherapeutic drug molecules are attached to a branched or polymeric backbone. A peptide of the disclosure is expressed recombinantly or chemically synthesized, and then the branched or polymeric backbone containing the four molecules of chemotherapeutic drug is conjugated to the peptide. The peptide is any one of SEQ ID NO: 1-SEQ ID NO: 196 or SEQ ID NO: 210-SEQ ID NO: 405.

### Example 28

### Linkers in Peptide Conjugate of the Disclosure

This example describes the use of a cleavable linker or a non-cleavable linker to attach a chemotherapeutic drug to a peptide. A peptide of the disclosure is expressed recombinantly or chemically synthesized and then is conjugated to a chemotherapeutic drug via an ester. This linker is a cleavable linker, and upon ester hydrolysis over time or cleavage by serum and cellular esterases, the chemotherapeutic drug is released from the peptide conjugate. Additionally, a peptide of the disclosure is expressed recombinantly or chemically synthesized and then is conjugated to MMAE with the valine-citrulline Cathepsin B cleavable linker. Alternatively, a peptide of the disclosure is expressed recombinantly or chemically synthesized and then is conjugated to a chemotherapeutic drug via a maleimide linker. This linker is a noncleavable linker. The chemotherapeutic drug is slowly released by the linker through an exchange onto free thiols on serum albumin. Additionally, a peptide of the disclosure is expressed recombinantly or chemically synthesized and then is conjugated to a chemotherapeutic drug via an NHS ester to produce an amide, which creates a non-cleavable linker.

### Example 29

### Co-Administration of a Peptide Conjugate and Radiotherapy

This example describes the coadministration of the peptides described herein and radiotherapy to tumors. A peptide of the disclosure is expressed recombinantly or chemically synthesized and then is conjugated to a chemotherapeutic drug, directly or via a cleavable or noncleavable linker. Coupling of the chemotherapeutic drug to the peptide of any one of SEQ ID NO: 1-SEQ ID NO: 196 or SEQ ID NO: 210-SEQ ID NO: 405 targets the drug to the tumor. One or more chemotherapeutic drug-peptide conjugates are administered to a human or animal while the human or animal is also being treated with radiotherapy targeting the tumor.

### Example 30

### Peptide Homing to Tumors

This example describes peptide homing to tumors. A peptide of the disclosure was labeled with a Cyanine 5.5 (Cy5.5) NHS ester near infrared fluorophore under aqueous conditions. Peptides of the disclosure were dissolved to 2 mg/mL in 50 mM sodium bicarbonate, pH 8.3. Cy5.5 NHS ester was dissolved at 10 mg/mL in anhydrous dimethylsulfoxide.

Approximately 0.1 molar equivalents of dye were added to the aqueous peptide solution at 10 minute intervals with thorough mixing. Analytical HPLC monitored at 214 and 678 nm was used to assess the completion of the reaction. Once complete, the mono-labeled product was purified using semi-preparative HPLC. The presence of a single Cy5.5-conjugated product was confirmed by mass spectrometry.

Dosages of the peptides were administered to Female Harlan athymic nude mice, weighing 20 g-25 g, via tail vein injection (n=3 mice per peptide).

Each peptide was chemically conjugated to Cy5.5 at the N-terminus or to a reactive lysine via an active NHS ester on the dye. A dosage of 10 nmol of each peptide carrying one fluorophore molecule was administered to Female Harlan athymic nude mice bearing tumors. The resulting fluorescence was normalized based on the fluorescence of the solution. The tumors were subcutaneous xenografts of either Colo205 (a human colon cancer cell line), MDA-MB231 (a triple-negative human breast cancer cell line), or U87 (a human glioma cell line), which were implanted in the flank of the mouse. Each peptide and each control was allowed to freely circulate within the animal before the animals were euthanized.

At the end of the 24-hour dosing period, mice were euthanized by CO₂ asphyxiation. Blood, tumor, muscle, kidney, liver, spleen, and colon tissue were harvested and placed in PBS on ice. The tissue was then scanned on the Odyssey CLx scanner (LI-COR) using the 700 nm channel, 21 micron resolution, and the Auto intensity settings. The signal intensity within a region of interest (ROI) was measured using the Image Studio software version 5.2 (LI-COR). All ROI's were the same size.

TABLE 5 lists whole organ near-infrared fluorescence quantification data in triplicate of Cy5.5 labeled peptides of SEQ ID NO: 25, SEQ ID NO: 32, SEQ ID NO: 17, SEQ ID NO: 6, SEQ ID NO: 37, SEQ ID NO: 35, or SEQ ID NO: 36 from Colo205 tumor-bearing Female Harlan athymic mice. Each row in TABLE 5 represents data from a single mouse (signal for each tissue is averaged over 3 ROIs per tissue).

TABLE 5
Whole organ near-infrared fluorescence quantification data for peptides of the
present disclosure conjugated to Cy5.5 in Colo205 tumor-bearing mice. Tumor/muscle
is the ratio of the signal in the tumor to the signal in the muscle.

| Peptide Conjugated | | | | | | |
|---|---|---|---|---|---|---|
| to Cy5.5 | Tumor | Colon | Liver | Spleen | Muscle | Tumor/Muscle |
| SEQ ID NO: 25 | 3380000 | 3023333.3 | 6426666.7 | 1913333.3 | 592000 | 5.7094595 |
| SEQ ID NO: 25 | 2233333.3 | 3210000 | 9326666.7 | 1870000 | 762333.33 | 2.9296021 |
| SEQ ID NO: 25 | 2093333.3 | 2603333.3 | 7376666.7 | 1523333.3 | 660333.33 | 3.1701161 |
| SEQ ID NO: 32 | 5210000 | 5373333.3 | 13933333 | 2346666.7 | 1529666.7 | 3.4059708 |
| SEQ ID NO: 32 | 3393333.3 | 5346666.7 | 15466667 | 2640000 | 1293333.3 | 2.6237113 |
| SEQ ID NO: 32 | 3543333.3 | 4546666.7 | 14433333 | 2796666.7 | 1073333.3 | 3.3012422 |
| SEQ ID NO: 17 | 3746666.7 | 4606666.7 | 18766667 | 4140000 | 1340000 | 2.7960199 |
| SEQ ID NO: 17 | 4943333.3 | 5340000 | 18466667 | 4153333.3 | 1636666.7 | 3.0203666 |
| SEQ ID NO: 17 | 6860000 | 4263333.3 | 16800000 | 3923333.3 | 1360000 | 5.0441176 |
| SEQ ID NO: 6 | 3846666.7 | 4996666.7 | 14166667 | 1800000 | 1098666.7 | 3.5012136 |
| SEQ ID NO: 6 | 6143333.3 | 6287500 | 16900000 | 2206666.7 | 780333.33 | 7.872704 |
| SEQ ID NO: 6 | 6333333.3 | 4790000 | 17100000 | 2013333.3 | 904000 | 7.0058997 |
| SEQ ID NO: 37 | 6646666.7 | 3703333.3 | 8753333.3 | 2213333.3 | 1083666.7 | 6.1334974 |
| SEQ ID NO: 37 | 9890000 | 4010000 | 9173333.3 | 2343333.3 | 807666.67 | 12.245151 |
| SEQ ID NO: 37 | 7570000 | 4040000 | 15100000 | 5746666.7 | 1526666.7 | 4.9585153 |
| SEQ ID NO: 35 | 6526666.7 | 6460000 | 12733333 | 2843333.3 | 1710000 | 3.8167641 |
| SEQ ID NO: 35 | 6780000 | 8023333.3 | 13966667 | 2470000 | 1076666.7 | 6.2972136 |
| SEQ ID NO: 35 | 16566667 | 9543333.3 | 18433333 | 3926666.7 | 1593333.3 | 10.39749 |
| SEQ ID NO: 36 | 19133333 | 11933333 | 34466667 | 11300000 | 2506666.7 | 7.6329787 |
| SEQ ID NO: 36 | 15466667 | 10913333 | 24366667 | 10500000 | 2233333.3 | 6.9253731 |
| SEQ ID NO: 36 | 19100000 | 12666667 | 35333333 | 14733333 | 2600000 | 7.3461538 |

TABLE 6 lists whole organ fluorescence quantification data of Cy5.5 labeled peptides of SEQ ID NO: 25, SEQ ID NO: 32, SEQ ID NO: 17, SEQ ID NO: 6, SEQ ID NO: 37, SEQ ID NO: 35, or SEQ ID NO: 36 from MDA-MB231 tumor-bearing Female Harlan athymic mice. Each row in TABLE 6 represents data from a single mouse (signal for each tissue is averaged over 3 ROIs per tissue).

TABLE 6
Whole organ fluorescence quantification data for peptides of the present
disclosure conjugated to Cy5.5 in MDA-MB-231 tumor-bearing mice.
Tumor/muscle is the ratio of the signal in the tumor to the signal in the muscle.

| Peptide Conjugated | | | | | | |
|---|---|---|---|---|---|---|
| to Cy5.5 | Tumor | Colon | Liver | Spleen | Muscle | Tumor/Muscle |
| SEQ ID NO: 25 | 3210000 | 2316666.67 | 7876666.67 | 1593333.33 | 824666.67 | 3.892482 |
| SEQ ID NO: 25 | 4830000 | 2856666.67 | 8083333.33 | 1460000 | 647000 | 7.465224 |
| SEQ ID NO: 25 | 4363333.33 | 2116666.67 | 7273333.33 | 1440000 | 490666.67 | 8.892663 |
| SEQ ID NO: 32 | 9813333.33 | 5303333.33 | 18166666.7 | 3046666.67 | 1716666.7 | 5.716505 |
| SEQ ID NO: 32 | 8773333.33 | 5390000 | 16700000 | 3713333.33 | 1450000 | 6.050575 |
| SEQ ID NO: 32 | 9796666.67 | 6963333.33 | 18300000 | 3540000 | 1866666.7 | 5.248214 |
| SEQ ID NO: 17 | 3313333.33 | 2346666.67 | 13400000 | 2283333.33 | 923666.67 | 3.587153 |
| SEQ ID NO: 17 | 5500000 | 3130000 | 12766666.7 | 2500000 | 706333.33 | 7.786692 |
| SEQ ID NO: 17 | 6210000 | 3636666.67 | 15833333.3 | 3006666.67 | 688000 | 9.026163 |
| SEQ ID NO: 6 | 5940000 | 5080000 | 13400000 | 1650000 | 1316000 | 4.513678 |
| SEQ ID NO: 6 | 5806666.67 | 4650000 | 13700000 | 1840000 | 1130000 | 5.138643 |
| SEQ ID NO: 6 | 3406666.67 | 3430000 | 12233333.3 | 1576666.67 | 839333.33 | 4.058777 |
| SEQ ID NO: 37 | 6740000 | 2946666.67 | 7910000 | 1686666.67 | 594000 | 11.3468 |
| SEQ ID NO: 37 | 5370000 | 2836666.67 | 9440000 | 1850000 | 903666.67 | 5.942457 |
| SEQ ID NO: 37 | 7966666.67 | 4123333.33 | 8163333.33 | 1373333.33 | 969000 | 8.221534 |
| SEQ ID NO: 35 | 9056666.67 | 6296666.67 | 17233333.3 | 2756666.67 | 1410000 | 6.423168 |
| SEQ ID NO: 35 | 14666666.7 | 8340000 | 21300000 | 3473333.33 | 1740000 | 8.429119 |
| SEQ ID NO: 36 | 2103333.33 | 1560000 | 5186666.67 | 1683333.33 | 517000 | 4.068343 |
| SEQ ID NO: 36 | 1032500 | 688333.333 | 1933333.33 | 643333.333 | 125666.67 | 8.21618 |

TABLE 7 lists whole organ fluorescence quantification data of peptides of SEQ ID NO: 25, SEQ ID NO: 32, SEQ ID NO: 17, SEQ ID NO: 6, SEQ ID NO: 37, SEQ ID NO: 35, or SEQ ID NO: 36 conjugated to Cy5.5, from U87 tumor-bearing Female Harlan athymic mice. Each row in TABLE 7 represents data from a single mouse (signal for each tissue is averaged over 3 ROIs per tissue).

TABLE 7
Whole organ fluorescence quantification data of peptides according to the
present disclosure conjugated to Cy5.5 in U87 tumor-bearing mice.
Tumor/muscle is the ratio of the signal in the tumor to the signal in the muscle.

| Peptide Conjugated | | | | | | |
|---|---|---|---|---|---|---|
| to Cy5.5 | Tumor | Colon | Liver | Spleen | Muscle | Tumor/Muscle |
| SEQ ID NO: 25 | 4490000 | 3380000 | 10766667 | 2126667 | NA | NA |
| SEQ ID NO: 25 | 6756667 | 2943333 | 8483333 | 1500000 | 748000 | 9.032976827 |
| SEQ ID NO: 25 | 8073333 | 3500000 | 12366667 | 2483333 | 830666.7 | 9.719101124 |
| SEQ ID NO: 32 | 10173333 | 7316667 | 24200000 | 4513333 | NA | NA |
| SEQ ID NO: 32 | 17166667 | 8343333 | 32466667 | 5870000 | 2756667 | 6.22732769 |
| SEQ ID NO: 32 | 5973333 | 6500000 | 18166667 | 3220000 | 1286667 | 4.642487047 |
| SEQ ID NO: 17 | 12166667 | 5626667 | 24233333 | 4786667 | 1140000 | 10.67251462 |
| SEQ ID NO: 17 | 8390000 | 3913333 | 16266667 | 2913333 | 1099333 | 7.63189812 |
| SEQ ID NO: 17 | 7723333 | 4803333 | 16233333 | 3563333 | 1010667 | 7.64182058 |
| SEQ ID NO: 6 | 4823333 | 4110000 | 17366667 | 2020000 | 1090000 | 4.425076453 |
| SEQ ID NO: 6 | 6983333 | 5956667 | 18666667 | 2423333 | 1856667 | 3.761220826 |
| SEQ ID NO: 6 | 7510000 | 5373333 | 20666667 | 2830000 | 1566667 | 4.793617021 |
| SEQ ID NO: 37 | 8920000 | 3316667 | 1.10E+07 | 2923333 | 1270333 | 7.021779061 |
| SEQ ID NO: 37 | 8640000 | 2456667 | 9176667 | 1873333 | 1065333 | 8.110137672 |
| SEQ ID NO: 37 | 10286667 | 2553333 | 9016667 | 1896667 | 1014667 | 10.13797635 |
| SEQ ID NO: 35 | 12066667 | 4880000 | 13733333 | 3423333 | 1686667 | 7.154150198 |
| SEQ ID NO: 35 | 20900000 | 4980000 | 13900000 | 4030000 | 1780000 | 11.74157303 |
| SEQ ID NO: 35 | 2.30E+07 | 5970000 | 18066667 | 4230000 | 1903333 | 12.08406305 |
| SEQ ID NO: 36 | 26500000 | 16033333 | 45500000 | 11800000 | 3166667 | 8.368421053 |
| SEQ ID NO: 36 | 28933333 | 28433333 | 32866667 | 6786667 | 3470000 | 8.338136407 |
| SEQ ID NO: 36 | 15700000 | 12400000 | 39300000 | 7663333 | 2180000 | 7.201834862 |

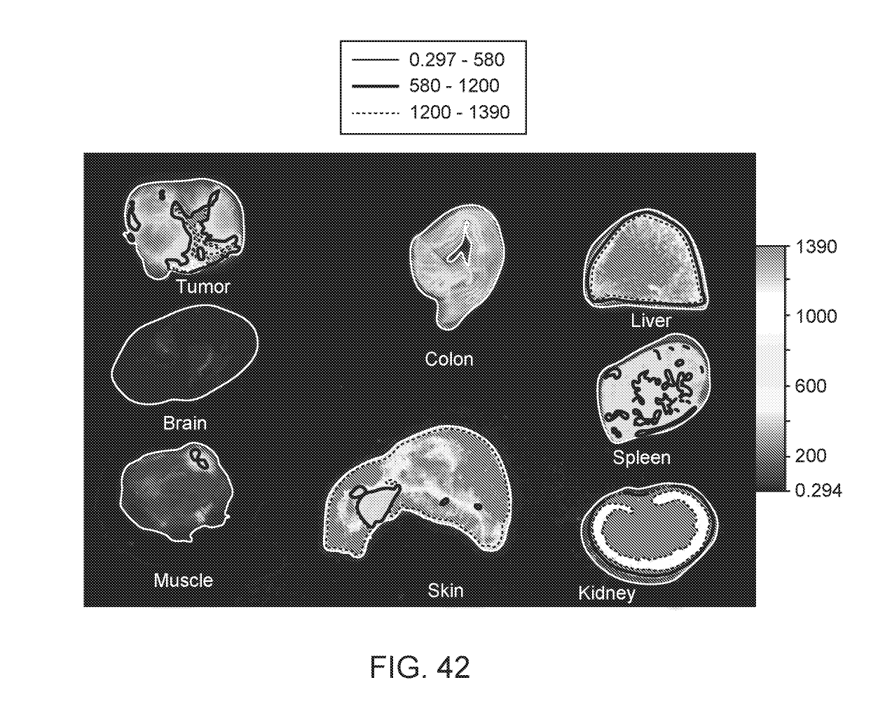

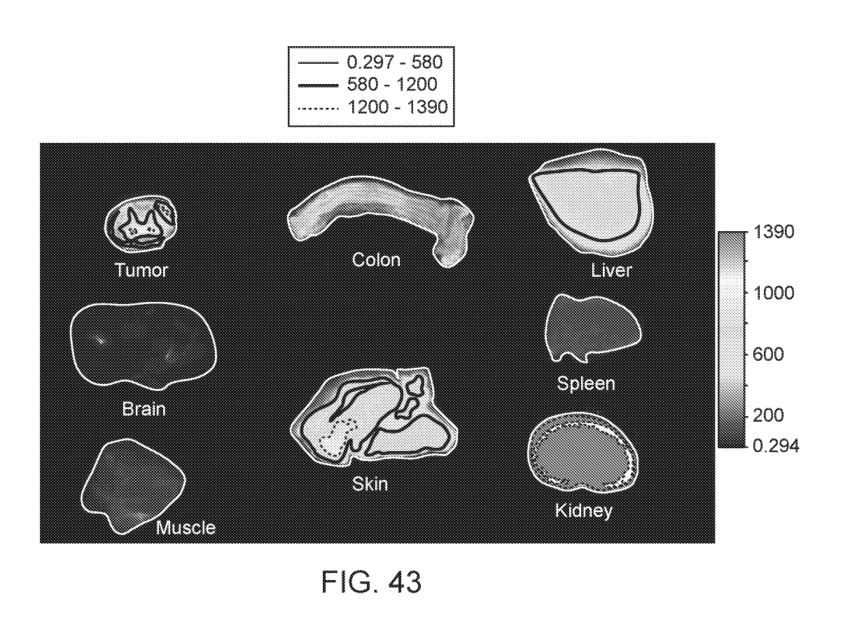

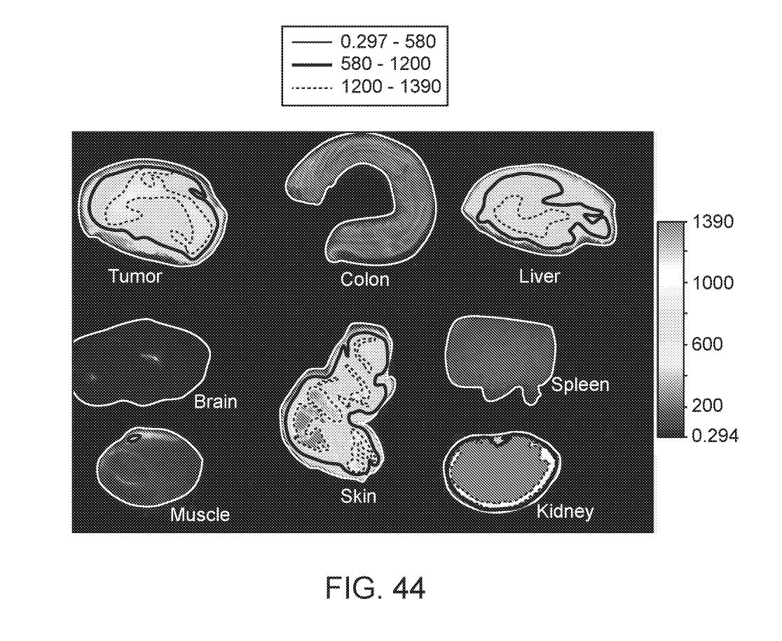

FIG. 42 shows a near-infrared fluorescence image of Colo205 tumor (top left), colon (top middle), liver (top right), brain (middle left), spleen (middle right), muscle (bottom left), skin (bottom middle), and kidney (bottom right), illustrating distribution of a peptide of SEQ ID NO: 37 conjugated to Cy5.5 after administration in Colo205 tumor-bearing Female Harlan athymic mice. FIG. 43 shows a near-infrared fluorescence image of MDA-MB-231 tumor (top left), colon (top middle), liver (top right), brain (middle left), spleen (middle right), muscle (bottom left), skin (bottom middle), and kidney (bottom right), illustrating distribution of a peptide of SEQ ID NO: 37 conjugated to Cy5.5 after administration in MDA-MB-231 tumor-bearing Female Harlan athymic mice. FIG. 44 shows a near-infrared fluorescence image of U87 tumor (top left), colon (top middle), liver (top right), brain (middle left), spleen (middle right), muscle (bottom left), skin (bottom middle), and kidney (bottom right), illustrating distribution of a peptide of SEQ ID NO: 37 conjugated to Cy5.5 after administration in U87 tumor-bearing Female Harlan athymic mice.

The peptides of SEQ ID NO: 25, SEQ ID NO: 32, SEQ ID NO: 17, SEQ ID NO: 6, SEQ ID NO: 37, SEQ ID NO: 35, and SEQ ID NO: 36 were detected in each tumor and had a tumor/muscle ratio greater than or equal to 2, which indicates that each of the peptides can home to multiple tumor types. Tumor/muscle is ratio that can be used to evaluate homing to tumor because other organs like liver, spleen, and colon are potential routes of elimination of the peptides.

The peptides of SEQ ID NO: 25, SEQ ID NO: 32, SEQ ID NO: 17, and SEQ ID NO: 6 each have a single lysine residue, which indicates the conjugation with the Cy5.5 dye may have occurred on the lysine residue and/or on the N-terminus. The peptides of SEQ ID NO: 37, SEQ ID NO: 35, and SEQ ID NO: 36 have no lysine residues and are identical to peptides of SEQ ID NO: 25, SEQ ID NO: 32, and SEQ ID NO: 17 with the exception of the mutated lysine residue, which indicates that the conjugation with the Cy5.5 dye may have occurred on the N-terminus for SEQ ID NO: 37, SEQ ID NO: 35, and SEQ ID NO: 36. Because all of peptides of SEQ ID NO: 25, SEQ ID NO: 32, SEQ ID NO: 17, SEQ ID NO: 6, SEQ ID NO: 37, SEQ ID NO: 35, and SEQ ID NO: 36 were detected in the tumors, this indicates that both the internal lysine residues and the N-terminal position are permissible locations for modification and conjugation, such as by attached a cytotoxic molecule to the peptide for treatment of cancer. Moreover, SEQ ID NO: 35-SEQ ID NO: 37 generally exhibited higher homing to tumor than SEQ ID NO: 17, SEQ ID NO: 25, and SEQ ID NO: 32, indicating that conjugation to the N-terminus may be advantageous for tumor homing. Consequently, mutating the lysine's in SEQ ID NO: 17, SEQ ID NO: 25, and SEQ ID NO: 32 was shown enhance their tumor-homing capability, which suggests that the N-terminus may be a preferred position for conjugation of active agents generally.

### Example 31

### Method to Determine Improved Peptide Variants

This example shows a method for determining ways to improve peptide variants by comparing and analyzing the primary sequences and tertiary structures of scaffold peptides. The primary sequences and the tertiary structures of the SEQ ID NO: 211 peptide and SEQ ID NO: 212 were compared. Both of these peptides are from the Theraphotoxin family and are thought to be BBB penetrators. This method was used to identify portions of the sequence of SEQ ID NO: 211 or SEQ ID NO: 212 that potentially could be grafted onto other members of the Theraphotoxin family and turn non-BBB penetrating members of that family into BBB penetrators.

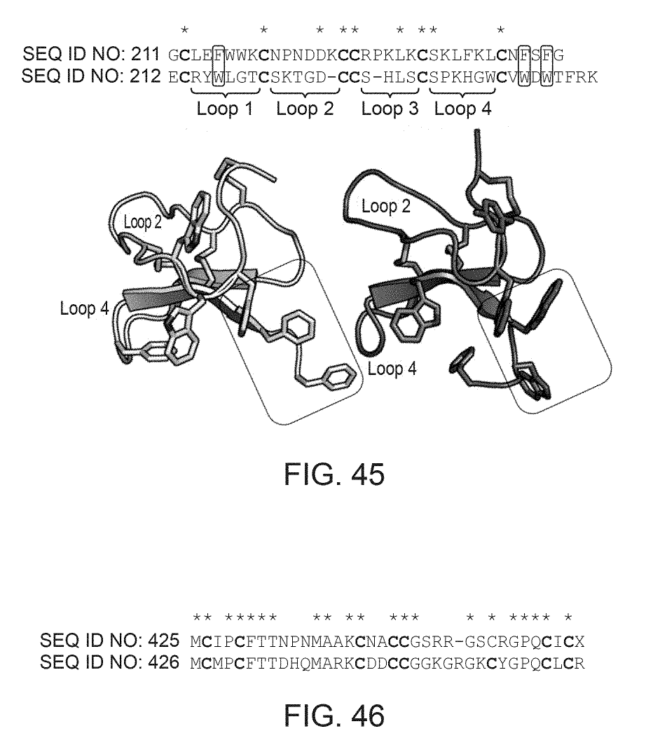

FIG. 45 shows sequences of SEQ ID NO: 211 aligned with SEQ ID NO: 212 with annotation of the location of loops, and their corresponding 3D structures, with the SEQ ID NO: 211 structure on the left and the SEQ ID NO: 212 structure on the right. The sequence alignment of the two scaffolds was used to identify an aromatic pharmacophore, which may be important for protein-protein binding interactions and for BBB penetration. Based on examination of the sequences and 3D conformation of SEQ ID NO: 211 and SEQ ID NO: 212, residues F5, F32, and F34 in SEQ ID NO: 211 and residues W5, W30, and W32 may be important residues for the BBB penetration properties of these peptides. In addition, W6 in SEQ ID NO: 211 and W27 in SEQ ID NO: 212 may also be important residues for BBB penetration. This comparison also showed both SEQ ID NO: 211 and SEQ ID NO: 212 have a much higher percentage of aromatic residues in their sequence than typical knottins, and therefore could potentially be important for BBB penetrating properties of these peptides. Additionally, comparison of structural homology led to the design of variants with improved folding properties. For example, mutations in loop (Loop 4), which forms an essential β-hairpin, were made in the variants to improve folding, and specific mutations in any of the loops (Loops 1-4) were shown to have a dramatic effect on the behavior and activity of the variants. The comparison also showed that the longer length of SEQ ID NO: 211 Loop 2 could be important for folding since SEQ ID NO: 212 has a shorter loop and folds less well in expression.

Additionally, the primary sequences and structure guided homology was used to compare SEQ ID NO: 425 peptide and SEQ ID NO: 426 peptide. These peptides are from the chlorotoxin family, and members or analogs of the chlorotoxin family may also be BBB penetrators.

FIG. 46 shows the sequence alignment of SEQ ID NO: 425 and SEQ ID NO: 426 with the location of the loops annotated, which was used to identify positions important to the folding and stability of the SEQ ID NO: 425. For example, the adding of an additional residue at the C-terminus can dramatically change the stability of the scaffold. As a result, variants made with additional residue appended after the C-terminal Cys residue of SEQ ID NO: 425 folded better than SEQ ID NO: 425. However, the identity of the appended residue was important. Variants with an appended Arg were less favorable, which may be due to enzymatic clipping, as compared with Asn, which folded well. Additionally, the length of loop 2 was shown to be permissive of folding at different lengths, indicating loop 2 can be altered to improve manufacturability and stability or to introduce new biological activities.

### Example 32

### Analysis of Homologs and Design of Variants

This example describes the analysis of homologs and design of variants. Homologs of peptides of SEQ ID NO: 1, SEQ ID NO: 2, SEQ ID NO: 3, and SEQ ID NO: 55 were identified from the Uniprot database. Some of these peptides were then expressed using the methods of EXAMPLE 1 and were expressed with GS amino acids appended to the N-terminus. Using information gathered from the analysis of structure, sequence alignments, and/or test expression of the homologs or the literature, variants of peptides of SEQ ID NO: 1, SEQ ID NO: 2, SEQ ID NO: 3, and SEQ ID NO: 55 were designed and expressed.

Exemplary sequences of homologs of a peptide of SEQ ID NO: 1 were peptides of SEQ ID NO: 85-SEQ ID NO: 110. Exemplary sequences of designed variants of a peptide of SEQ ID NO: 1 were peptides of SEQ ID NO: 111-SEQ ID NO: 133.

Exemplary sequences of homologs of a peptide of SEQ ID NO: 2 were peptides of SEQ ID NO: 134-SEQ ID NO: 138. Exemplary sequences of designed variants of a peptide of SEQ ID NO: 2 were peptides of SEQ ID NO: 139-SEQ ID NO: 162.

Exemplary sequences of homologs of a peptide of SEQ ID NO: 3 were peptides of SEQ ID NO: 163-SEQ ID NO: 168. Exemplary sequences of designed variants of a peptide of SEQ ID NO: 3 were peptides of SEQ ID NO: 169-SEQ ID NO: 192.

Exemplary sequences of homologs of a peptide of SEQ ID NO: 55 were peptides of SEQ ID NO: 56-SEQ ID NO: 63, SEQ ID NO: 65-SEQ ID NO: 70, SEQ ID NO: 71-SEQ ID NO: 72, SEQ ID NO: 74-SEQ ID NO: 79. Exemplary sequences of designed variants of a peptide of SEQ ID NO: 55 were peptides of SEQ ID NO: 83 and SEQ ID NO: 84.

### Example 33

### Peptide-Neurotensin Peptide Fusions

This example describes peptide-neurotensin peptide fusions. Neurotensin is a 13 amino acid neuropeptide ELYENKPRRPYIL (SEQ ID NO: 420) that is implicated in the regulation of luteinizing hormone and prolactin release and has significant interaction with the dopaminergic system.

Neurotensin peptide was fused with peptides of SEQ ID NO: 83, SEQ ID NO: 37, and SEQ ID NO: 98, and was expressed using the methods of EXAMPLE 1 to yield peptides of SEQ ID NO: 193-SEQ ID NO: 196. Neurotensin peptide was also fused with GSS-Hadrucalcin EKDCIKHLQRCRENKDCCSKKCSRRGTNPEKRCR (SEQ ID NO: 423) to yield a peptide of SEQ ID NO: 197.

### Example 34

### Peptide-Neurotensin Peptide Fusions

This example describes a method of use of peptide-neurotensin peptide fusions.

Neurotensin peptide does not cross the blood brain barrier, but is instead produced and signals within the central nervous system. Therefore, neurotensin peptide is fused to a peptide as described herein that can cross the blood brain barrier. The peptide-neurotensin peptide fusions of SEQ ID NO: 193-SEQ ID NO: 196 are expressed and are administered to a subject in need thereof. After administration, the blood brain barrier of the subject is crossed by peptide-neurotensin peptide fusions of SEQ ID NO: 193-SEQ ID NO: 196 and pain or other neurotensin deficient indications are treated. The subject can be a human.

### Example 35

### Biodistribution of Peptides

This example describes biodistribution of peptides. At the end of the dosing period, mice were frozen in a hexane/dry ice bath and then frozen in a block of carboxymethylcellulose. Thin, frozen sections of whole animal sagittal slices that include the brain, tumor, liver, kidney, lung, heart, spleen, pancreas, muscle, adipose, gall bladder, upper gastrointestinal track, lower gastrointestinal track, bone, bone marrow, reproductive tract, eye, stomach, spinal cord, bladder, salivary gland, and other types of tissues were obtained with a microtome, allowed to dessicate in a freezer, and exposed to phosphoimager plates for about ten days.

These plates were developed, and the signal (densitometry) from each organ was normalized to the signal found in the heart blood of each animal. A signal in tissue darker than the signal expected from blood in that tissue indicates accumulation in a region, tissue, structure or cell.

TABLE 8 lists a summary of the migration of each peptide to liver and lung. Numbers for the liver or lung indicate the percentage of signal in that tissue compared to the signal detected in the heart blood.

TABLE 8
Summary of peptide migration to the liver or lung.

| Peptide | Liver | Lung |
|---|---|---|
| SEQ ID NO: 1 | 129.99 | 89.98 |
| SEQ ID NO: 2 | 507.95 | 148.59 |
| SEQ ID NO: 3 | 109.00 | 181.48 |
| SEQ ID NO: 4 | 97.22 | 75.34 |
| SEQ ID NO: 34 | 82.37 | 118.19 |
| SEQ ID NO: 35 | 82.80 | 106.60 |
| SEQ ID NO: 37 | 95.22 | 110.28 |
| SEQ ID NO: 55 | 51.65 | 84.67 |
| Inulin | 126.76 | 242.98 |
| GS-Hainantoxin | 70.61 | 85.11 |
| Potassium Channel Peptide | 39.89 | 79.28 |
| SEQ ID NO: 36 | 128.72 | 104.62 |
| SEQ ID NO: 39 | 84.20 | 76.27 |

TABLE 9 lists a summary of the migration of each peptide to the spleen, pancreas, muscle, adipose tissue, gall bladder, upper gastrointestinal tract, and lower gastrointestinal tract.

TABLE 9
Summary of peptide migration to the spleen, pancreas, muscle, adipose
tissue, gall bladder, upper gastrointestinal tract, or lower gastrointestinal
tract. Numbers indicate the percentage of signal in the indicated tissue
compared to that of the signal from blood in the heart.

| | | | | | Gall | Upper | Lower |
|---|---|---|---|---|---|---|---|
| Peptide | Spleen | Pancreas | Muscle | Adipose | bladder | GI | GI |
| SEQ ID NO: 1 | 83.62 | 92.54 | 36.56 | 4.78 | | 793.25 | 9.87 |
| SEQ ID NO: 2 | 90.64 | 89.66 | 68.00 | 46.92 | 158.65 | 624.30 | 33.29 |
| SEQ ID NO: 3 | 43.16 | 37.76 | 25.47 | 6.20 | 86.20 | 69.16 | 17.04 |
| SEQ ID NO: 4 | 61.45 | 53.54 | 31.18 | 11.15 | | 184.09 | 16.04 |
| SEQ ID NO: 55 | Low | Low | 26.87 | 6.47 | ND | 128.14 | 9.42 |
| SEQ ID NO: 34 | 54.28 | 30.35 | 34.13 | 11.69 | 35.52 | 216.32 | 20.73 |
| SEQ ID NO: 5 | | | | | | | |
| SEQ ID NO: 35 | 51.29 | 31.85 | 27.73 | | | 136.03 | 5.50 |
| SEQ ID NO: 37 | 61.64 | | 32.79 | | | 214.82 | 25.27 |
| Inulin | | | 19.80 | 5.98 | 18.65 | 382.27 | 10.92 |
| Potassium Channel | 47.05 | 36.09 | 17.68 | 10.52 | 30.20 | 104.52 | 5.61 |
| Peptide | | | | | | | |
| SEQ ID NO: 36 | | | 27.65 | | | 353.31 | 19.48 |
| SEQ ID NO: 39 | | | 23.56 | | | 210.26 | 9.87 |
| GS-Hainantoxin | | | 20.63 | 5.05 | 315.85 | 387.02 | 10.65 |

TABLE 10 lists a summary of the migration of each peptide to the reproductive tract, skin, and salivary gland of each animal. Numbers for the reproductive tract, skin, or salivary gland indicate the percentage of signal in that tissue compared to the signal detected in the heart blood.

TABLE 10
Summary of peptide migration to the
reproductive tract, or salivary gland.

| Peptide | Reproductive | Salivary gland |
|---|---|---|
| SEQ ID NO: 1 | | 62.08 |
| SEQ ID NO: 2 | 165.31 | 150.77 |
| SEQ ID NO: 3 | 60.79 | 47.89 |
| SEQ ID NO: 4 | 139.65 | 50.84 |
| SEQ ID NO: 34 | 229.69 | 57.57 |
| SEQ ID NO: 55 | Low | |
| SEQ ID NO: 5 | | |
| SEQ ID NO: 35 | 136.01 | |
| SEQ ID NO: 37 | 44.50 | |
| Inulin | 129.37 | 27.58 |
| Potassium Channel Peptide | 123.96 | 28.46 |
| SEQ ID NO: 36 | 160.48 | |
| SEQ ID NO: 39 | 62.19 | |
| GS-Hainantoxin | 77.98 | 16.59 |

TABLE 11 lists a summary of the migration of each peptide to the spinal cord and bladder of each animal. High, moderate, and low indicate a comparison of radioactivity signal per area in blood to that in the tissue. ND indicates the peptide was not detected in that tissue.

TABLE 11
Summary of peptide migration to the spinal cord or bladder.

| Peptide | Spinal Cord | Bladder |
|---|---|---|
| SEQ ID NO: 1 | moderate | ND |
| SEQ ID NO: 2 | Low | Low |
| SEQ ID NO: 3 | Low | ND |
| SEQ ID NO: 4 | Low | ND |
| SEQ ID NO: 55 | Low | Low |

While preferred embodiments of the present invention have been shown and described herein, it will be apparent to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-113. (canceled)

114. A peptide conjugate comprising an active agent linked to a peptide, the peptide comprising an amino acid sequence having at least 80% sequence identity to SEQ ID NO: 37 or a functional fragment thereof, the peptide further comprising:
(a) at least 30 amino acid residues;
(b) at least 8 cysteine residues; and
(c) a plurality of disulfide bridges formed between the cysteine residues.

115. The peptide conjugate of claim 114, wherein the peptide comprises at least 36 amino acid residues.

116. The peptide conjugate of claim 114, wherein the peptide further comprises a disulfide through a disulfide knot.

117. The peptide conjugate of claim 114, wherein the active agent is selected from the group consisting of: a peptide, a polypeptide, a polynucleotide, an antibody, a single chain variable fragment, an antibody fragment, a cytokine, a hormone, a growth factor, a checkpoint inhibitor, an immune modulator, a neurotransmitter, a chemical agent, a cytotoxic molecule, a toxin, a radiosensitizer, a radioprotectant, a therapeutic small molecule, a nanoparticle, a liposome, a polymer, a dendrimer, a fatty acid, a peptidomimetic, a complement fixing peptide or protein, a polyethylene glycol, a lipid, an Fc region, an anti-inflammatory agent, an antifungal agent, an antiviral agent, an anti-infective agent, a chemotherapeutic agent, a detectable agent, a neurotensin peptide, or any combination thereof.

118. The peptide conjugate according to claim 114, wherein the active agent comprises a chemotherapeutic agent.

119. The peptide conjugate according to claim 114, wherein the active agent comprises an antibody or an antibody fragment.

120. The peptide conjugate according to claim 114, wherein the active agent comprises a neurotransmitter.

121. The peptide conjugate according to claim 114, wherein the active agent comprises a neurotensin peptide.

122. A pharmaceutical composition comprising the peptide conjugate of claim 114 and a pharmaceutically acceptable carrier.

123. The pharmaceutical composition of claim 122, wherein the pharmaceutical composition is formulated for an injection.

124. A method of treating a condition, the method comprising:
administering a peptide conjugate to a subject in need thereof, the peptide conjugate comprising an active agent linked to a peptide, the peptide comprising an amino acid sequence having at least 80% sequence identity to SEQ ID NO: 37 or a functional fragment thereof, the peptide further comprising:
(a) at least 30 amino acid residues;
(b) at least 8 cysteine residues; and
(c) a plurality of disulfide bridges formed between the cysteine residues, and
delivering the active agent to a region in a central nervous system using the peptide conjugate.

125. The method of claim 124, wherein the peptide further comprises a disulfide through a disulfide knot.

126. The method of claim 124, wherein the region in the central nervous system is a region of a brain or cerebrospinal fluid.

127. The method of claim 126, wherein the region of the brain is selected from the group consisting of: ventricles, cerebrospinal fluid, hippocampus, meninges, rostral migratory system, dentate gyrus, subventricular zone, or any combination thereof.

128. The method of claim 124, wherein the delivering comprises targeting a cancerous or diseased tissue, structure, or cell in the region of the central nervous system.

129. The method of claim 124, wherein the condition is a brain condition.

130. The method of claim 129, wherein the brain condition is memory loss or memory function, Alzheimer's disease, Parkinson's disease, multiple system atrophy, schizophrenia, epilepsy, progressive multifocal leukoencephalopathy, fungal infection, depression, bipolar disorder, post-traumatic stress disorder, stroke, traumatic brain injury, infection, multiple sclerosis, or brain cancer.

131. The method of claim 124, wherein the condition is epilepsy and the active agent comprises a neurotransmitter.

132. The method of claim 124, wherein the condition is pain and the active agent comprises a neurotensin peptide.

133. The method of claim 124, wherein the condition is a neurodegenerative disease and the peptide conjugate reduces aggregation of a protein associated with the neurodegenerative disease.

134. The method of claim 133, wherein the protein associated with the neurodegenerative disease is Tau or amyloid beta peptide.

135. The method of claim 129, wherein the active agent comprises an antibody or an antibody fragment.

136. The method of claim 129, wherein the delivering comprises the peptide conjugate binding to an ion channel.

137. The method of claim 124, further comprising imaging the region in the central nervous system.

138. A method of treating a tumor or a cancer, the method comprising:
administering a peptide conjugate to a subject in need thereof, the peptide conjugate comprising an active agent linked to a peptide, the peptide comprising an amino acid sequence having at least 80% sequence identity to SEQ ID NO: 37 or a functional fragment thereof, the peptide further comprising:
(a) at least 30 amino acid residues;
(b) at least 8 cysteine residues; and
(c) a plurality of disulfide bridges formed between the cysteine residues, and
delivering the active agent to the tumor or a cancerous cell in the subject in need thereof using the peptide conjugate.

139. The method of claim 138, wherein the peptide further comprises a disulfide through a disulfide knot.

140. The method of claim 138, wherein the tumor or the cancerous cell is from a brain cancer, a colon cancer, a triple-negative breast cancer, a metastatic cancer, or a sarcoma.

141. The method of claim 138, wherein the tumor or the cancer is glioblastoma.

142. The method of claim 138, wherein the cancer is a brain cancer and the peptide conjugate inhibits a pathway associated with the brain cancer.

143. The method of claim 138, wherein the cancer is a brain cancer and the active agent comprises a chemotherapeutic agent.

144. The method of claim 143, wherein the chemotherapeutic agent comprises an antibody or an antibody fragment.

145. The method of claim 143, wherein the chemotherapeutic agent is selected from the group consisting of: a checkpoint inhibitor, a chemical agent, a cytotoxic molecule, or a toxin.

146. The method of claim 138, further comprising imaging the tumor or the cancerous cell.

147. The method of claim 138, further comprising removing the tumor or the cancerous cell.
